(19) United States
(12) Patent Application Publication
(10) Pub. No.: US 2014/0298508 A1
(43) Pub. Date: Oct. 2, 2014

(54) ALPHA-MANNOSIDASES FROM PLANTS AND METHODS FOR USING THE SAME

(71) Applicants: Nikolai Valeryevitch Ivanov, Neuchatel (CH); Prisca Camponini, Villars-Burquin (CH); Dionisius Florack, Le Landeron (CH); Karen Oishi, Neuchatel (CH)

(72) Inventors: Nikolai Valeryevitch Ivanov, Neuchatel (CH); Prisca Camponini, Villars-Burquin (CH); Dionisius Florack, Le Landeron (CH); Karen Oishi, Neuchatel (CH)

(73) Assignee: PHILIP MORRIS PRODUCTS S.A., Neuchatel, ` (CH)

(21) Appl. No.: 13/996,735

(22) PCT Filed: Dec. 23, 2011
(86) PCT No.: PCT/EP11/73954

§ 371 Date: Aug. 13, 2013

(30) Foreign Application Priority Data
Dec. 23, 2010 (EP) 10196758.6

(51) Int. Cl. C12N 9/24

(52) U.S. Cl. CPC C12N 9/2491
USPC 800/260; 435/414; 800/317.3; 435/69.1; 435/183; 435/69.4; 435/69.6; 536/23.2; 435/209; 435/6.18; 530/395; 800/278; 435/468; 435/69.5

(57) ABSTRACT

The present invention is directed to alpha-mannosidase sequences from plants and the use thereof, especially genomic nucleotide sequences containing the regulatory elements controlling their expression, intron and exon sequences and polynucleotide sequences coding for alpha-mannosidase enzymes. Such plants with modified alpha-mannosidase activity can be used for the production of glycoproteins having an altered saccharide composition of great benefit. The present invention also relates to the use of these alpha-mannosidase enzymes for hydrolyzing mannoses.

The present invention is directed to alpha-mannosidase sequences from plants, especially genomic nucleotide sequences containing the regulatory elements controlling their expression, intron and exon sequences and polynucleotide sequences coding for alpha-mannosidase enzymes. The present invention is also directed to the use of these sequences for modifying the expression of one or more alpha-mannosidases in plants for the generation of plants having increased or reduced alpha-mannosidase activity. Such plants with modified alpha-mannosidase activity can be used for the production of glycoproteins having an altered saccharide composition of great benefit. The present invention also relates to the use of these alpha-mannosidase enzymes for hydrolyzing mannoses.

Recombinant expression of proteins that can be used therapeutically, for example, in humans constitutes an important application of transgenic plants. A major hurdle in the production of glycoproteins in plants however is the presence of plant specific beta-1,2-xylose and alpha-1,3-fucose saccharides on an N-glycan of a glycoprotein produced by a plant, as these plant-specific saccharides are known to be highly immunogenic. Asparagine-linked- or N-glycosylation involves the addition of a polysaccharide or N-glycan to a protein, which is referred to as a glycoprotein. The N-glycosylation process involves a number of sequential enzymatic steps and is highly similar in plants and mammals. N-glycosylation starts with the addition of a precursor Glc3-Man9-GlcNAc2 oligosaccharide onto an asparagine (Asn or N) residue resulting in a Glc3-Man9-GlcNAc2-Asn N-glycosylated protein, wherein Glc is a glucose, Man is a mannose and GlcNAc is an N-acetylglucosamine. This precursor is then sequentially processed, first in the endoplasmic reticulum by a number of enzymes starting with three glucosidases, glucosidase I, II and III resulting in a Man9-GlcNAc2-Asn N-glycosylated protein. Next, one or more alpha-mannosidase I enzymes further trim the high-mannose Man9-GlcNAc2-Asn N-glycan subsequently to a Man8-GlcNAc2-Asn, Man7-GlcNAc2-Asn, Man6-GlcNAc2-Asn and finally a Man5-GlcNAc2-Asn N-glycan. In the Golgi network the Man5-GlcNAc2-Asn undergoes further processing and maturation. The first step in maturation involves the conversion of the high mannose Man5-GlcNAc2-Asn N-glycan to a hybrid-type N-glycan by the addition of an N-acetylglucosamine to the reducing end resulting in a GlcNAc-Man5-GlcNAc2-Asn N-glycan through the activity of N-acetylglucosaminyltransferase I. The next step in maturation involves hydrolyzing the GlcNAc-Man5-GlcNAc2-Asn to a GlcNAc-Man4-GlcNAc2-Asn and ultimately to a GlcNAc-Man3-GlcNAc2-Asn N-glycan by one or more an alpha-mannosidase II enzymes. Next, an additional GlcNAc is added by the N-acetylglucosaminyltransferase II enzyme to result in a GlcNAc2-Man3-GlcNAc2-Asn N-glycan. Up to this point, the N-glycosylation pathway is similar in mammals and plants. In mammals, an alpha-1,6-fucose (Fuc) is then added to the first GlcNAc at the non-reducing end to result in GlcNAc2-Man3-Fuc(α1,6)-GlcNAc2-Asn, and one or more beta-1,4-galactoses (Gal) and alpha-2,3-sialic acid (NeuAc) residues through the action of a beta-1,4-galactosyltransferase and alpha-2,3-sialyltransferase, respectively, resulting in a NeuAc2-Gal2-GlcNAc2-Man3-Fuc(α1,6)-GlcNAc2-Asn N-glycan. In plants, a xylose (Xyl) is added to the core mannose in beta-1,2-linkage and an alpha-1,3-fucose to the first GlcNAc at the non-reducing end resulting in a GlcNAc2-Man3-Xyl-Fuc(α1,3)-GlcNAc2-Asn N-glycan.

Alpha-mannosidases hydrolyse oligomannosidic N-glycan structures and consist of endoplasmic reticulum-resident alpha-mannosidases and Golgi-resident alpha-mannosidases. Alpha-mannosidase I (EC 3.2.1.113) is an alpha-1,2-mannosidase (α(1,2)-mannosidase) that hydrolyses the oligomannosidic Man9 to Man5 N-glycans in the endoplasmatic reticulum and cis-Golgi. Alpha-mannosidase II (EC 3.2.1.114) is exclusively a Golgi-resident alpha-mannosidase and highly specific for alpha-1,3-mannose (α1,3-mannose) and alpha-1,6-mannose (α1,6-mannose) and hydrolyses the oligomannosidic Man5 and Man4 hybrid-type N-glycans to Man3 N-glycans. However, given the potential of producing recombinant proteins in plants, methods for preventing the addition of plant-specific saccharides onto a glycoprotein in a plant as described hereinabove are not presently available.

There is therefore an unmet need for methods to prevent the addition of such plant-specific saccharides onto a glycoprotein, particularly an N-glycan of a glycoprotein in a plant. Particularly, it is desirable to obtain plants and plant cells which are capable of producing glycoproteins which substantially lack alpha-1,3-linked fucose and beta-1,2-linked xylose residues on an N-glycan of a glycoprotein. This unmet need is addressed and solved by the present invention by providing polynucleotides, polypeptides and methods as defined by the features of independent claims. Preferred embodiments are subject of the dependent claims.

The polynucleotides, polypeptides and methods according to the invention now make it possible to manufacture heterologous glycoproteins containing variable amounts of mannoses on the N-glycan of the glycoprotein in plant cells, plants or parts thereof, that lack plant specific beta-1,2-xylose and alpha-1,3-fucose. Particularly, the transgenic plant cells, plants or parts thereof exhibit a modified amount of mannoses on the N-glycan of a glycoprotein, compared to control counterparts and may be used for the manufacture of heterologous glycoproteins for the purpose of making a pharmaceutical composition. Pharmaceutical composition comprising such plant-produced glycoproteins can thus have favourable immunogenic properties for use in human subjects and improved efficacy.

### DEFINITIONS

The technical terms and expressions used within the scope of this application are generally to be given the meaning commonly applied to them in the pertinent art of plant and molecular biology. All of the following term definitions apply to the complete content of this application. The word “comprising” does not exclude other elements or steps, and the indefinite article “a” or “an” does not exclude a plurality. A single step may fulfil the functions of several features recited in the claims. The terms “essentially”, “about”, “approximately” and the like in connection with an attribute or a value particularly also define exactly the attribute or exactly the value, respectively. The term “about” in the context of a given numerate value or range refers to a value or range that is within 20%, within 10%, or within 5% of the given value or range.

The term “polynucleotide” as used herein refers to a polymer of nucleotides, which may be unmodified or modified deoxyribonucleic acid (DNA) or ribonucleic acid (RNA). Accordingly, a polynucleotide can be, without limitation, a genomic DNA, complementary DNA (cDNA), mRNA, or antisense RNA. Moreover, a polynucleotide can be single-stranded or double-stranded DNA, DNA that is a mixture of single-stranded and double-stranded regions, a hybrid molecule comprising DNA and RNA, or a hybrid molecule with a mixture of single-stranded and double-stranded regions. In addition, the polynucleotide can be composed of triple-stranded regions comprising DNA, RNA, or both. A polynucleotide can contain one or more modified bases, such as phosphothioates, and can be a peptide nucleic acid (PNA). Generally, polynucleotides provided by this invention can be assembled from isolated, amplified, or cloned fragments of cDNA, genome DNA, exon sequences, intron sequences, oligonucleotides, or individual nucleotides, or a combination of the foregoing. Although the polynucleotide sequences described herein are shown as DNA sequences, the sequences include their corresponding RNA sequences, and their complementary DNA or RNA sequences, including the reverse complements thereof.

The term “NtMNS1a polynucleotide” as used herein refers to a polymer of nucleotides comprising, consisting or consisting essentially of the isolated NtMNS1a gene designated herein as SEQ ID NO:1, SEQ ID NO:2, SEQ ID NO:30, or SEQ ID NO:94, the NtMNS1a exon sequences designated herein as SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 or SEQ ID NO:29, and NtMNS1a intron sequences designated herein as SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26 or SEQ ID NO:28. This term also encompasses polynucleotides with substantial homology or sequence similarity or substantial identity to any of SEQ ID NO:1 to SEQ ID NO:30; fragments of SEQ ID NO:1, SEQ ID NO:2, SEQ ID NO:30, or SEQ ID NO:94, and fragments of SEQ ID NO:1, SEQ ID NO:2, SEQ ID NO:30 and SEQ ID NO:94, with substantial homology or sequence similarity or substantial identity thereto.

As described herein, the variant may have at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMNS1a gene. Although the NtMNS1a polynucleotide sequences described herein are shown as DNA sequences, the sequences include their corresponding RNA sequences, and their complementary DNA or RNA sequences, including the reverse complement or complements thereof.

The term “NtMNS1b polynucleotide” as used herein refers to a polymer of nucleotides comprising, consisting or consisting essentially of the isolated NtMNS1b gene designated herein as SEQ ID NO:32, SEQ ID NO:33, SEQ ID NO:61, or SEQ ID NO:96, the NtMNS1b exon sequences designated herein as SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58 or SEQ ID NO:60, and NtMNS1b intron sequences designated herein as SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57 or SEQ ID NO:59. This term also encompasses polynucleotides with substantial homology or sequence similarity or substantial identity to any of SEQ ID NO:32 to SEQ ID NO:61; fragments of SEQ ID NO:32, SEQ ID NO:33, SEQ ID NO:61, or SEQ ID NO:96, and fragments of SEQ ID NO:32, SEQ ID NO:33, SEQ ID NO:61, and SEQ ID NO:96, with substantial homology or sequence similarity or substantial identity thereto. As described herein, the variant may have at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMNS1b gene. Although the NtMNS1b polynucleotide sequences described herein are shown as DNA sequences, the sequences include their corresponding RNA sequences, and their complementary DNA or RNA sequences, including the reverse complement or complements thereof.

As used herein, the term “NtMNS2 polynucleotide” as used herein refers to a polymer of nucleotides comprising, consisting or consisting essentially of the isolated NtMNS2 gene designated herein as SEQ ID NO:63, SEQ ID NO:64 or SEQ ID NO:92, the NtMNS2 exon sequences designated herein as SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89 or SEQ ID NO:91, and NtMNS2 intron sequences designated herein as SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88 or SEQ ID NO:90. This term also encompasses polynucleotides with substantial homology or sequence similarity or substantial identity to any of SEQ ID NO:63 to SEQ ID NO:92; fragments of SEQ ID NO:63, SEQ ID NO:64 or SEQ ID NO:92, and fragments of SEQ ID NO:63, SEQ ID NO:64 and SEQ ID NO:92 with substantial homology or sequence similarity or substantial identity thereto.

As described herein, the variant may have at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 95%, 96%, 97%, 98%, or 99% sequence identity to the sequence of the isolated NtMNS2 gene. Although the NtMNS2 polynucleotide sequences described herein are shown as DNA sequences, the sequences include their corresponding RNA sequences, and their complementary DNA or RNA sequences, including the reverse complement or complements thereof.

As used herein, the term “nucleotide sequence” refers to the base sequence of a polymer of nucleotides, including but not limited to ribonucleotides and deoxyribonucleotides.

As used herein, the term “NtMan1.4 polynucleotide” as used herein refers to a polymer of nucleotides comprising, consisting or consisting essentially of the isolated NtMan1.4 gene designated herein as SEQ ID NO:98.

As described herein, the variant may have at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMan1.4 gene. Although the NtMan1.4 polynucleotide sequences described herein are shown as DNA sequences, the sequences include their corresponding RNA sequences, and their complementary DNA or RNA sequences, including the reverse complement or complements thereof.

The term “isolated” as used herein relates to an entity that is taken from its natural milieu, but does not connote any degree of purification.

As used herein, the term “gene sequence” as used herein refers to the nucleotide sequence of a nucleic acid molecule or polynucleotide that encodes a polypeptide or a biologically active RNA, and encompasses the nucleotide sequence of a partial coding sequence that only encodes a fragment of a protein. A gene sequence can also include sequences having a regulatory function on expression of a gene that are located upstream or downstream relative to the coding sequence such as but not limited to untranslated leader sequences and promoter and terminator sequences, as well as intron and exon sequences of a gene.

The term “NtMNS1a polypeptide” refers to a polypeptide comprising, consisting or consisting essentially of an amino acid sequence encoded by the isolated NtMNS1a gene or a polypeptide designated herein as SEQ ID NO:31 and SEQ ID NO:95, respectively. This term also encompasses polypeptides with substantial homology or sequence similarity or substantial identity to SEQ ID NO:31 and SEQ ID NO:95; fragments of SEQ ID NO:31 and SEQ ID NO:95; and fragments of SEQ ID NO:31 and SEQ ID NO:95 with substantial homology or sequence similarity or substantial identity thereto. The NtMNS1a polypeptide includes sequences comprising a sufficient or substantial degree of identity or similarity to SEQ ID NO:31 and SEQ ID NO:95, respectively, that can hydrolyze mannoses. NtMNS1a polypeptide also include variants or mutants produced by introducing any type of alterations such as but not limited to insertions, deletions, or substitutions of amino acids; changes in glycosylation states including N-glycosylation; changes that affect refolding or isomerizations, three-dimensional structures, or self-association states, which can be deliberately engineered or isolated naturally. NtMNS1a polypeptide may be in linear form or cyclized using known methods. As described herein, the variant may have at least 50%, 55%, 60%, 70%, 71%, 72%, 73%, 74%, 75%, but particularly at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the sequence of the NtMNS1a polypeptide or at least 84%, 85%, 86%, 87%, 88%, 89%, 90%, 91%, 92%, 93%, 94%, 95%, 96%, 97%, 98%, or 99% similarity to the sequence of the NtMNS1a polypeptide.

The term “NtMNS1b polypeptide” refers to a polypeptide comprising, consisting or consisting essentially of an amino acid sequence encoded by the isolated NtMNS1a gene or a polypeptide designated herein as SEQ ID NO:62 and SEQ ID NO:97, respectively. This term also encompasses polypeptides with substantial homology or sequence similarity or substantial identity to SEQ ID NO:62 and SEQ ID NO:97; fragments of SEQ ID NO: 62 and SEQ ID NO:97; and fragments of SEQ ID NO:62 and SEQ ID NO:97 with substantial homology or sequence similarity or substantial identity thereto. The NtMNS1b polypeptide includes sequences comprising a sufficient or substantial degree of identity or similarity to SEQ ID NO:62 and SEQ ID NO:97, respectively, that can hydrolyze mannoses. NtMNS1b polypeptide also include variants or mutants produced by introducing any type of alterations such as but not limited to insertions, deletions, or substitutions of amino acids; changes in glycosylation states including N-glycosylation; changes that affect refolding or isomerizations, three-dimensional structures, or self-association states, which can be deliberately engineered or isolated naturally. NtMNS1b polypeptide may be in linear form or cyclized using known methods. As described herein, the variant may have at least 50%, 55%, 60%, 70%, 71%, 72%, 73%, 74%, but particularly at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the sequence of the NtMNS1b polypeptide or at least 84%, 85%, 86%, 87%, 88%, 89%, 90%, 91%, 92%, 93%, 94%, 95%, 96%, 97%, 98%, or 99% similarity to the sequence of the NtMNS1b polypeptide.

The term “NtMNS2 polypeptide” refers to a polypeptide comprising, consisting or consisting essentially of an amino acid sequence encoded by the isolated NtMNS2 gene or a polypeptide designated herein as SEQ ID NO:93. This term also encompasses polypeptides with substantial homology or sequence similarity or substantial identity to SEQ ID NO:93; fragments of SEQ ID NO:93; and fragments of SEQ ID NO:93 with substantial homology or sequence similarity or substantial identity thereto. The NtMNS2 polypeptide includes sequences comprising a sufficient or substantial degree of identity or similarity to SEQ ID NO:93 that can hydrolyze mannoses. NtMNS2 polypeptide also include variants or mutants produced by introducing any type of alterations such as but not limited to insertions, deletions, or substitutions of amino acids; changes in glycosylation states including N-glycosylation; changes that affect refolding or isomerizations, three-dimensional structures, or self-association states, which can be deliberately engineered or isolated naturally. NtMNS2 polypeptide may be in linear form or cyclized using known methods. As described herein, the variant may have at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the sequence of the NtMNS2 polypeptide or at least 83%, 84%, 85%, 86%, 87%, 88%, 89%, 90%, 91%, 92%, 93%, 94%, 95%, 96%, 97%, 98%, or 99% similarity to the sequence of the NtMNS2 polypeptide.

The term “NtMan1.4 polypeptide” refers to a polypeptide comprising, consisting or consisting essentially of an amino acid sequence encoded by the isolated NtMan1.4 gene or a polypeptide designated herein as SEQ ID NO:99. This term also encompasses polypeptides with substantial homology or sequence similarity or substantial identity to SEQ ID NO:99; fragments of SEQ ID NO:99; and fragments of SEQ ID NO:99 with substantial homology or sequence similarity or substantial identity thereto. The NtMan1.4 polypeptide includes sequences comprising a sufficient or substantial degree of identity or similarity to SEQ ID NO:99 that can hydrolyze mannoses. NtMan1.4 polypeptide also include variants or mutants produced by introducing any type of alterations such as but not limited to insertions, deletions, or substitutions of amino acids; changes in glycosylation states including N-glycosylation; changes that affect refolding or isomerizations, three-dimensional structures, or self-association states, which can be deliberately engineered or isolated naturally. NtMan1.4 polypeptide may be in linear form or cyclized using known methods. As described herein, the variant may have at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the sequence of the NtMan1.4 polypeptide or at least 83%, 84%, 85%, 86%, 87%, 88%, 89%, 90%, 91%, 92%, 93%, 94%, 95%, 96%, 97%, 98%, or 99% similarity to the sequence of the NtMan1.4 polypeptide.

The term “NtMNS1a gene sequence” refers to the nucleotide sequence of a nucleic acid molecule or polynucleotide that encodes the NtMNS1a polypeptide of SEQ ID NO:31 and SEQ ID NO:95, respectively, or a biologically active RNA, and encompasses the nucleotide sequence of a partial coding sequence that only encodes a fragment of the NtMNS1a polypeptide. A gene sequence can also include sequences having a regulatory function on expression of a gene that are located upstream or downstream relative to the coding sequence such as but not limited to untranslated leader sequences and promoter and terminator sequences, as well as intron and exon sequences of a gene.

The term “NtMNS1b gene sequence” refers to the nucleotide sequence of a nucleic acid molecule or polynucleotide that encodes the NtMNS1b polypeptide of SEQ ID NO:62 and SEQ ID NO:97, respectively, or a biologically active RNA, and encompasses the nucleotide sequence of a partial coding sequence that only encodes a fragment of the NtMNS1b polypeptide. A gene sequence can also include sequences having a regulatory function on expression of a gene that are located upstream or downstream relative to the coding sequence such as but not limited to untranslated leader sequences and promoter and terminator sequences, as well as intron and exon sequences of a gene.

The term “NtMNS2 gene sequence” refers to the nucleotide sequence of a nucleic acid molecule or polynucleotide that encodes the NtMNS2 polypeptide of SEQ ID NO:93 or a biologically active RNA, and encompasses the nucleotide sequence of a partial coding sequence that only encodes a fragment of the NtMNS2 polypeptide. A gene sequence can also include sequences having a regulatory function on expression of a gene that are located upstream or downstream relative to the coding sequence such as but not limited to untranslated leader sequences and promoter and terminator sequences, as well as intron and exon sequences of a gene.

The term “NtMan1.4 gene sequence” refers to the nucleotide sequence of a nucleic acid molecule or polynucleotide that encodes the NtMan1.4 polypeptide of SEQ ID NO:99 or a biologically active RNA, and encompasses the nucleotide sequence of a partial coding sequence that only encodes a fragment of the NtMan1.4 polypeptide. A gene sequence can also include sequences having a regulatory function on expression of a gene that are located upstream or downstream relative to the coding sequence such as but not limited to untranslated leader sequences and promoter and terminator sequences, as well as intron and exon sequences of a gene.

The term “vector” as used herein refers to a nucleic acid vehicle that comprises a combination of DNA components for enabling the transport of nucleic acid, nucleic acid constructs and nucleic acid conjugates and the like. Suitable vectors include episomes capable of extra-chromosomal replication such as circular, double-stranded DNA plasmids; linearized double-stranded DNA plasmids; binary vectors capable of transferring T-DNA to a plant cell nucleus; and other vectors of any origin.

The term “expression vector” refers to a nucleic acid vehicle that comprises a combination of DNA components for enabling the expression of nucleic acid, nucleic acid constructs and nucleic acid conjugates and the like. Suitable expression vectors include episomes capable of extra-chromosomal replication such as circular, double-stranded DNA plasmids; linearized double-stranded DNA plasmids; binary vectors capable of transferring T-DNA to a plant cell nucleus; and other functionally equivalent expression vectors of any origin. An expression vector comprises at least a promoter positioned upstream and operably-linked to a nucleic acid, nucleic acid constructs or nucleic acid conjugate, as defined below.

The term “construct” refers to a double-stranded, recombinant DNA fragment comprising NtMNS1a, NtMNS1b,r NtMNS2, or NtMan1.4 polynucleotides. The construct comprises a “template strand” base-paired with a complementary “sense or coding strand.” A given construct can be inserted into a vector in two possible orientations, either in the same (or sense) orientation or in the reverse (or anti-sense) orientation with respect to the orientation of a promoter positioned within a vector, such as an expression vector and especially a binary expression vector.

The term “template strand” refers to the strand comprising a sequence that complements that of the “sense or coding strand” of a DNA duplex, such as a NtMNS1a, NtMNS1b,r NtMNS2, or NtMan1.4 genomic fragment, NtMNS1a, NtMNS1b, NtMNS2, or NtMan1.4 cDNA, or NtMNS1a, NtMNS1b, NtMNS2, or NtMan1.4 construct, or any DNA fragment comprising a nucleic acid sequence that can be transcribed by RNA polymerase. During transcription, RNA polymerase can translocate along the template strand in the 3′ to 5′ direction during nascent RNA synthesis.

The term “sense strand” used interchangeably herein with the term “coding strand” refers to the strand comprising a sequence that complements that of the template strand in a DNA duplex. For example, the sequence of the sense strand (“sense sequence”) for the identified NtMNS1a genomic clone is designated as SEQ ID NO:1 or SEQ ID NO:2. For example, if the sense strand comprises a hypothetical sequence 5′-TAATCCGGT-3′, then the substantially identical corresponding sequence within a hypothetical target mRNA is 5′-UAAUCCGGU-3′.

The term “reverse complementary sequence” refers to the sequence that complements the “sense sequence” of interest such as for example an exon sequence positioned within the same strand, in the same orientation with respect to the sense sequence. For example, if a strand comprises a hypothetical sequence 5′-TAATCCGGT-3′, then the reverse complementary sequence 5′-ACCGGATTA-3′ may be operably-linked to the sense sequence, separated by a spacer sequence.

The term “NtMNS1a RNA transcript” used interchangeably with “NtMNS1a RNA,” includes polyribonucleic acid molecules produced within a host plant cell of interest, resulting from the transcription of the endogenous NtMNS1a gene of for example SEQ ID NO:1, SEQ ID NO:2, SEQ ID NO:30, or SEQ ID NO:94. Thus, this term includes any RNA species or RNA variants produced as transcriptional products from NtMNS1a including those RNA species or RNA variants that have sufficient similarity at the structural or functional level. For example, NtMNS1a RNA transcripts include: (1) pre-mRNAs and mRNAs produced from the transcription of the isolated NtMNS1a gene of for example SEQ ID NO:1, SEQ ID NO:2, SEQ ID NO:30, or SEQ ID NO:94; (2) pre-mRNAs and mRNAs produced from the transcription of any genes having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMNS1a gene such as other distinct genes substantially identical to the identified NtMNS1a gene and encoding related isoforms of alpha-mannosidase I enzymes; and (3) pre-mRNAs and mRNAs produced from the transcription of alleles of the NtMNS1a gene. The NtMNS1a RNA transcripts include RNA variants produced as a result of alternative RNA splicing reactions of heteronuclear RNAs (“hnRNAs”) of a particular NtMNS1a gene, mRNA variants resulting from such alternative RNA splicing reactions, and any intermediate RNA variants.

The term “NtMNS1b RNA transcript” used interchangeably with “NtMNS1b RNA,” includes polyribonucleic acid molecules produced within a host plant cell of interest, resulting from the transcription of the endogenous NtMNS1a gene of for example SEQ ID NO:32, SEQ ID NO:33, SEQ ID NO:61, or SEQ ID NO:96. Thus, this term includes any RNA species or RNA variants produced as transcriptional products from NtMNS1b including those RNA species or RNA variants that have sufficient similarity at the structural or functional level. For example, NtMNS1b RNA transcripts include: (1) pre-mRNAs and mRNAs produced from the transcription of the isolated NtMNS1b gene of for example SEQ ID NO:32, SEQ ID NO:33, SEQ ID NO:61, or SEQ ID NO:96; (2) pre-mRNAs and mRNAs produced from the transcription of any genes having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMNS1b gene such as other distinct genes substantially identical to the identified NtMNS1b gene and encoding related isoforms of alpha-mannosidase I enzymes; and (3) pre-mRNAs and mRNAs produced from the transcription of alleles of the NtMNS1b gene. The NtMNS1b RNA transcripts include RNA variants produced as a result of alternative RNA splicing reactions of heteronuclear RNAs (“hnRNAs”) of a particular NtMNS1b gene, mRNA variants resulting from such alternative RNA splicing reactions, and any intermediate RNA variants.

The term “NtMNS2 RNA transcript” used interchangeably with “NtMNS2 RNA,” includes polyribonucleic acid molecules produced within a host plant cell of interest, resulting from the transcription of the endogenous NtMNS2 gene of for example SEQ ID NO:63, SEQ ID NO:64 or SEQ ID NO:92. Thus, this term includes any RNA species or RNA variants produced as transcriptional products from NtMNS2 including those RNA species or RNA variants that have sufficient similarity at the structural or functional level. For example, NtMNS2 RNA transcripts include: (1) pre-mRNAs and mRNAs produced from the transcription of the isolated NtMNS2 gene of for example SEQ ID NO:63, SEQ ID NO:64 or SEQ ID NO:92; (2) pre-mRNAs and mRNAs produced from the transcription of any genes having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMNS2 gene such as other distinct genes substantially identical to the identified NtMNS2 gene and encoding related isoforms of alpha-mannosidase I enzymes; and (3) pre-mRNAs and mRNAs produced from the transcription of alleles of the NtMNS2 gene. The NtMNS2 RNA transcripts include RNA variants produced as a result of alternative RNA splicing reactions of heteronuclear RNAs (“hnRNAs”) of a particular NtMNS2 gene, mRNA variants resulting from such alternative RNA splicing reactions, and any intermediate RNA variants.

The term “NtMan1.4 RNA transcript” used interchangeably with “NtMan1.4 RNA,” includes polyribonucleic acid molecules produced within a host plant cell of interest, resulting from the transcription of the endogenous NtMan1.4 gene of for example SEQ ID NO:98. Thus, this term includes any RNA species or RNA variants produced as transcriptional products from NtMan1.4 including those RNA species or RNA variants that have sufficient similarity at the structural or functional level. For example, NtMan1.4 RNA transcripts include: (1) pre-mRNAs and mRNAs produced from the transcription of the isolated NtMan1.4 gene of for example SEQ ID NO:98; (2) pre-mRNAs and mRNAs produced from the transcription of any genes having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence of the isolated NtMan1.4 gene such as other distinct genes substantially identical to the identified NtMan1.4 gene and encoding related isoforms of alpha-mannosidase I enzymes; and (3) pre-mRNAs and mRNAs produced from the transcription of alleles of the NtMan1.4 gene. The NtMan1.4 RNA transcripts include RNA variants produced as a result of alternative RNA splicing reactions of heteronuclear RNAs (“hnRNAs”) of a particular NtMan1.4 gene, mRNA variants resulting from such alternative RNA splicing reactions, and any intermediate RNA variants.

The term “upstream” refers to a relative direction or position with respect to a reference element along a linear polynucleotide sequence, which indicates a direction or position towards the 5′ end of the polynucleotide sequence. “Upstream” may be used interchangeably with the “5′ end of a reference element.”

The term “operably-linked” refers to the joining of distinct DNA elements, fragments, or sequences to produce a functional transcriptional unit or a functional expression vector. The term “promoter” refers to a nucleic acid element or sequence, typically positioned upstream and operably-linked to a double-stranded DNA fragment such as a NtMNS1a, NtMNS1b, NtMNS2, or NtMan1.4 cDNA of SEQ ID NO:30, SEQ ID NO: 94, SEQ ID NO:61, SEQ ID NO: 96, SEQ ID NO: 92, or SEQ ID NO: 98, respectively, or an RNAi construct. In case of the latter construct, a suitable promoter enables the transcriptional activation of a NtMNS1a, NtMNS1b, NtMNS2, or NtMan1.4 RNAi construct by recruiting the transcriptional complex, including the RNA polymerase and various factors, to initiate RNA synthesis. Promoters can be derived entirely from regions proximate to a native gene of interest, or can be composed of different elements derived from different native promoters or synthetic DNA segments.

The term “enhancer” refers to a nucleic acid molecule, or a nucleic acid sequence, that can recruit transcriptional regulatory proteins such as transcriptional activators, to enhance transcriptional activation by increasing promoter activity. Suitable enhancers can be derived from regions proximate to a native promoter of interest (homologous sources) or can be derived from non-native contexts (heterologous sources) and operably-linked to any promoter of interest within NtMNS1a, NtMNS1b, NtMNS2, or NtMan1.4 constructs, such as cDNA expression vectors or RNAi expression vectors, to enhance the activity or the tissue-specificity of a promoter. Some enhancers can operate in any orientation with respect to the orientation of a transcription unit. For example, enhancers may be positioned upstream or downstream of a transcriptional unit comprising a promoter and a NtMNS1a, NtMNS1b, NtMNS2, or NtMan1.4 construct.

The term “plant” as used herein, this term refers to any plant at any stage of its life cycle or development, and its progenies.

The term “plant cell” as used herein refers to a structural and physiological unit of a plant. The plant cell may be in form of a protoplast without a cell wall, an isolated single cell or a cultured cell, or as a part of higher organized unit such as but not limited to, plant tissue, a plant organ, or a whole plant.

The term “plant cell culture” refers to cultures of plant cells such as but not limited to, protoplasts, cell culture cells, cells in cultured plant tissues, cells in explants, and pollen cultures.

The term “plant material” refers to any solid, liquid or gaseous composition, or a combination thereof, obtainable from a plant, including leaves, stems, roots, flowers or flower parts, fruits, pollen, egg cells, zygotes, seeds, cuttings, secretions, extracts, cell or tissue cultures, or any other parts or products of a plant.

The term “plant tissue” relates to a group of plant cells organized into a structural or functional unit. Any tissue of a plant in planta or in culture is included. This term includes, but is not limited to, whole plants, plant organs, and seeds.

The term “plant organ” relates to a distinct or a differentiated part of a plant such as a root, stem, leaf, flower bud or embryo.

The term “heterologous sequence” refers to a biological sequence that does not occur naturally in the context of a given genome in a cell or an organism of interest, such as but not limited to the nuclear genome, a plastid genome or a mitochondrial genome.

The term “heterologous protein” refers to a protein that is produced by a cell but does not occur naturally in that cell. For example, the heterologous protein produced in a plant cell can be a mammalian or human protein. A heterologous protein may contain one or more oligosaccharide chains such as N-glycans covalently attached to the polypeptide backbone in a co-translational or post-translational modification.

The term “N-glycan” refers to a carbohydrate or oligosaccharide chain that is attached to an asparagine (Asn or N) residue that is part of a Asn-Xaa-Ser or Asn-Xaa-Thr sequence motif in the protein backbone, wherein Xaa can be any amino acid except for a proline, Ser is a serine and Thr a threonine amino acid and Asn is the asparagine on the protein backbone.

The term “N-glycosylation” refers to a process that starts with the transfer of a specific dolichol (Dol) lipid-linked precursor oligosaccharide, Dol-PP-GlcNAc2-Man9-Glc3, from the dolichol moiety in the endoplasmatic reticulum membrane onto the free amino group of an asparagine residue (Asn) being part of a Asn-Xaa-Ser or Asn-Xaa-Thr motif in the protein backbone, resulting in a Glc3-Man9-GlcNAc2-Asn glycosylated protein. The abbreviations “Man”, as used herein, refers to mannose; “GlcNAc” refers to N-acetylglucosamine; “Glc” refers to glucose; “Xyl” refers to xylose; “Fuc” refers to fucose; “Gal” refers to galactose and “NeuAc” to sialic acid. The suffix 2 in GlcNAc2 refers to the presence of 2 N-acetylglucosamine residues; the suffix 3 in Man3 refers to the presence of 3 mannoses and Man5 refers to five mannoses. The addition alpha-1,3 or α(1,3) refers to the linkage of the respective saccharide to the next in-line saccharide on the N-glycan.

The term “non-reducing end of an N-glycan” refers to the part of the N-glycan that is attached to the asparagine of the protein backbone.

The term “reducing end of an N-glycan” refers to the part of the N-glycan opposite of the non-reducing end and freely accessible to reduction by hydrolysis.

The term “alpha-mannosidase I” refers to class I alpha-mannosidases (EC 3.2.1.113) which are inverting glycosyl hydrolases that are highly specific for α(1,2)-mannose residues.

The term “alpha-mannosidase II” refers to class II alpha-mannosidases (EC 3.2.1.114) which are inverting glycosyl hydrolases that are highly specific for α(1,3)- and α(1,6)-mannose residues and typically reside in the Golgi apparatus.

The terms “beta-1,2-xylosyltransferase”, or “β(1,2)-xylosyltransferase” refers to a xylosyltransferase designated EC2.4.2.38 that adds a xylose in beta-1,2-linkage (β(1,2)-Xyl) onto the beta-1,4-linked mannose (β(1,4)-Man) of the trimannosyl (Man3) core structure of a N-glycan of a glycoprotein.

The term “alpha-1,3-fucosyltransferase” or“α(1,3)-fucosyltransferase” refers to a fucosyltransferase designated EC2.4.1.214 that adds a fucose in alpha-1,3-linkage (α(1,3)-fucose) onto the proximal N-acetylglucosamine residue at the non-reducing end of an N-glycan.

The term “N-acetylglucosaminyltransferase I” refers to an enzyme designated EC2.4.1.101 that adds an N-acetylglucosamine to a mannose on the 1-3 arm of a Man5-GlcNAc2-Asn oligomannosyl receptor.

The term “reduce”, or“reduced” refers to a reduction of from about 10% to about 99%, or a reduction of at least 10%, at least 20%, at least 25%, at least 30%, at least 40%, at least 50%, at least 60%, at least 70%, at least 75%, at least 80%, at least 90%, at least 95%, at least 98%, or up to 100%, of a quantity or an activity, such as but not limited to enzyme activity, transcriptional activity, ribonucleic acid and protein expression.

The term “increase” or “increased” refers to an increase of from about 10% to about 1000%, or an increase of at least 10%, at least 20%, at least 30%, at least 40%, at least 50%, at least 75%, at least 100%, at least 200%, at least 250%, at least 500%, at least 750%, or up to 1000%, of a quantity or an activity, such as but not limited to enzyme activity, transcriptional activity, ribonucleic acid and protein expression.

The term “inhibit” or “inhibited” refers to a reduction of from about 95%, to about 100%, or a reduction of at least 95%, at least 96%, at least 97%, at least 98%, at least 99%, but particularly of 100%, of a quantity or an activity, such as but not limited to enzyme activity, transcriptional activity, ribonucleic acid and protein expression.

As used herein, the term “substantially inhibit” or “substantially inhibited” refers to a reduction of from about 80% to about 100%, or a reduction of at least 80%, at least 90%, at least 95%, at least 98%, or up to 100%, of a quantity or an activity, such as but not limited to enzyme activity, transcriptional activity, ribonucleic acid and protein expression.

As used herein, the term “substantial increase” or “substantially increased” refers to an increase of from about 100% to about 1000%, or an increase of at least 100%, at least 200%, at least 250%, at least 300%, at least 400%, at least 500%, or up to 1000%, of a quantity or an activity, such as but not limited to enzyme activity, transcriptional activity, ribonucleic acid and protein expression.

The term “genome editing” or “genome editing technology” refers to any method for modifying a nucleotide sequence in the genome of an organism, such as but not limited to, zinc finger nuclease-mediated mutagenesis, chemical mutagenesis, radiation mutagenesis, or meganuclease-mediated mutagenesis.

The term “zinc finger nuclease” refers to a protein consisting of a zinc finger DNA-binding domain and a DNA-cleavage domain. The zinc finger DNA-binding domain can be natural or engineered to target a specific polynucleotide or gene sequence. Upon binding to the target polynucleotide or nucleic acid, a zinc finger nuclease makes a break that activates an endogenous DNA repair machinery resulting in a modified polynucleotide or nucleotide sequence.

The term “meganuclease” refers to a protein with endodeoxyribonuclease activity that recognizes a specific binding site of approximately 12 to 40 basepairs. Meganuclease can be genetically engineered to bind to a specific site. Upon binding, meganucleases make a DNA break which can activate DNA repair resulting in homologous recombination.

The term “exon” as used herein refers to a nucleotide sequence that is represented in the mature form of an RNA molecule after either portions of a precursor RNA (introns) have been removed by cis-splicing or when two or more precursor RNA molecules have been ligated by trans-splicing. The mature RNA molecule can be a messenger RNA or a functional form of a non-coding RNA such as rRNA or tRNA. Depending on the context, exon can refer to the sequence in the DNA or its RNA transcript.

The term “intron” as used herein refers to a nucleotide sequence within a gene that is not translated into protein. These non-coding sections are transcribed to precursor mRNA (pre-mRNA) and some other RNAs (such as long noncoding RNAs), and subsequently removed by a process called splicing during the processing to mature RNA. After intron splicing, the mRNA consists only of exon derived sequences, which are translated into a protein.

The term “percent identity” or “sequence identity” in the context of two or more nucleotide sequences or amino acid sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. The term “identity” is used herein in the context of a nucleotide sequence or amino acid sequence to describe two sequences that are at least 50%, at least 55%, at least 60%, particularly of at least 70%, particularly of at least 71%, particularly of at least 72%, particularly of at least 73%, particularly of at least 74%, particularly of at least 75% more particularly of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%, identical to one another.

If two sequences which are to be compared with each other differ in length, sequence identity preferably relates to the percentage of the nucleotide residues of the shorter sequence which are identical with the nucleotide residues of the longer sequence. As used herein, the percent identity between two sequences is a function of the number of identical positions shared by the sequences (that is % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W.R. Pearson (1990), Methods in Enzymology 183, 63-98). For this purpose, the “default” parameter settings may be used. Alternatively, the percentage identity of two sequences may be determined by comparing sequence information using the EMBOSS needle computer program (Rice et al. (2000) Trends in Genetics 16:276-277). EMBOSS needle reads two input sequences and writes their optimal global sequence alignment to file. It uses the Needleman-Wunsch alignment algorithm (Needleman and Wunsch (1970) J. Mol. Biol. 48: 443-453) to find the optimum alignment (including gaps) of two sequences along their entire length. The identity value is the percentage of identical matches between the two sequences over the reported aligned region (including any gaps in the length).

If the two nucleotide sequences to be compared by sequence comparison, differ in identity refers to the shorter sequence and that part of the longer sequence that matches the shorter sequence. In other words, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence.

For example, nucleotide or amino acid sequences which have at least 50%, at least 55%, at least 60%, particularly of at least 70%, particularly of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% A identity to the herein-described nucleotide or amino acid sequences, may represent alleles, derivatives or variants of these sequences which preferably have a similar biological function. They may be either naturally occurring variations, for instance allelic sequences, sequences from other ecotypes, varieties, species, etc., or mutations. The mutations may have formed naturally or may have been produced by deliberate mutagenesis methods, such as those disclosed in the present invention. Furthermore, the variations may be synthetically produced sequences. The allelic variants may be naturally occurring variants or synthetically produced variants or variants produced by recombinant DNA techniques. Deviations from the above-described polynucleotides may have been produced, for example, by deletion, substitution, addition, insertion or recombination or insertion and recombination. The term “addition” refers to adding at least one nucleic acid residue or amino acid to the end of the given sequence, whereas “insertion” refers to inserting at least one nucleic acid residue or amino acid within a given sequence.

Another indication that two nucleic acid sequences are substantially identical is that the two polynucleotides hybridize to each other under stringent conditions. The phrase: “hybridizing specifically to” refers to the binding, duplexing, or hybridizing of a molecule only to a particular nucleotide sequence under stringent conditions when that sequence is present in a complex mixture (for example total cellular) DNA or RNA. “Bind(s) substantially” refers to complementary hybridization between a nucleic acid probe and a target nucleic acid and embraces minor mismatches that can be accommodated by reducing the stringency of the hybridization media to achieve the desired detection of the target nucleic acid sequence.

Polynucleotide sequences which are capable of hybridizing with the polynucleotide sequences provided herein can, for instance, be isolated from genomic DNA libraries or cDNA libraries of plants. Particularly, such polynucleotides are from plant origin, particularly preferred from a plant belonging to the genus of Nicotiana. Alternatively, such nucleotide sequences can be prepared by genetic engineering or chemical synthesis.

Such polynucleotide sequences being capable of hybridizing may be identified and isolated by using the polynucleotide sequences described herein, or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Nucleotide sequences comprising the same or substantially the same nucleotide sequences as indicated in the listed SEQ ID NOs, or parts or fragments thereof, can, for instance, be used as hybridization probes. The fragments used as hybridization probes can also be synthetic fragments which are prepared by usual synthesis techniques, the sequence of which is substantially identical with that of a nucleotide sequence according to the invention.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point for the specific sequence at a defined ionic strength and pH. Typically, under “stringent conditions” a probe will hybridize to its target subsequence, but to no other sequences.

The thermal melting point is the temperature (under defined ionic strength and pH) at which 50% of the target sequence hybridizes to a perfectly matched probe. Very stringent conditions are selected to be equal to the melting temperature (Tₘ) for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2 times SSC wash at 65° C. for 15 minutes (see Sambrook, infra, for a description of SSC buffer). Often, a high stringency wash is preceded by a low stringency wash to remove background probe signal. An example of medium stringency wash for a duplex of, for example, more than 100 nucleotides, is 1 times SSC at 45° C. for 15 minutes. An example low stringency wash for a duplex of, for example, more than 100 nucleotides, is 4-6 times SSC at 40° C. for 15 minutes. For short probes (for example, about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, for example when a copy of a nucleic acid is created using the maximum codon degeneracy permitted by the genetic code.

As disclosed herein, the invention provides methods for modifying the nucleotide sequence in a plant or a plant cell, resulting in a plant or a plant cell that exhibits a reduction, an inhibition or a substantial inhibition of the enzyme activity of the alpha mannosidase, or a reduced level of expression of the alpha mannosidase. The reduction, an inhibition or a substantial inhibition in enzyme activity or the change in expression level is relative to that in a naturally occurring plant cell, an unmodified plant cell, or a plant cell not modified by a method of the invention, any one of which can be used as a control. A comparison of enzyme activities or expression levels against such a control can be carried out by any methods known in the art.

Many methods known in the art can be used to mutate the nucleotide sequence of a alpha mannosidase gene of the invention. Methods that introduce a mutation randomly in a gene sequence can be, without being limited to, chemical mutagenesis, such as but not limited to EMS mutatagenesis and radiation mutagenesis. Methods that introduce a targeted mutation into a gene sequence, such as the NtMNS1a, NtMNS1b, or NtMSN2 gene sequences, include but are not limited to various genome editing technologies, particularly zinc finger nuclease-mediated mutagenesis, tilling (targeting induced local lesions in genomes, as described in McCallum et al., Plant Physiol, June 2000, Vol. 123, pp. 439-442 and Henikoff et al., Plant Physiology 135:630-636 (2004)), homologous recombination, oligonucleotide-directed mutagenesis, and meganuclease-mediated mutagenesis. Many methods known in the art for screening mutated gene sequences can be used to identify or confirm a mutation.

A method of the invention thus comprises modifying a sequence that encodes alpha mannosidase of the invention in a plant cell by applying mutagenesis such as chemical mutagenesis or radiation mutagenesis. Another method of the invention comprises modifying a target site in a sequence that encodes an alpha mannosidases of the invention by applying genome editing technology, such as but not limited to zinc finger nuclease-mediated mutagenesis, “tilling” (targeting induced local lesions in genomes), homologous recombination, oligonucleotide-directed mutagenesis and meganuclease-mediated mutagenesis.

Given that multiple alpha mannosidases, variants and alleles, may be active in a plant cell, to achieve a reduction, substantial inhibition or complete inhibition of the enzyme activities, it is contemplated that more than one gene sequences encoding alpha mannosidases are to be modified in the plant cell. In preferred embodiments of the invention, the modifications are produced by applying one or more genome editing technologies that are known in the art. A modified plant cell of the invention can be produced by a number of strategies.

Modified plant cells or modified plants of the invention can be identified by the production of a mutant alpha mannosidase that has a molecular weight which is different from the alpha mannosidase produced in an unmodified plant or plant cell. The mutant alpha mannosidase can be a truncated form or an elongated form of the alpha mannosidase produced in an unmodified plant or plant cell, and can be used as a marker to aid identification of a modified plant or plant cell. The truncation or elongation of the polypeptide typically results from the introduction of a stop codon in the coding sequence or a shift in the reading frame resulting in the use of a stop codon in an alternative reading frame. Alternatively, such mutant alpha-mannosidases can result from mutations in the intron-exon boundary or boundaries of the alpha-mannosidase genome sequence resulting in an altered splicing of the respective intron-exon sequences. Alternative splicing of a pre-mRNA can result in an altered cDNA that can be truncated or elongated. The elongation can be an insertion in the polypeptide sequence.

Another strategy for producing a modified plant or plant cells comprising more than one modified alpha mannosidase gene sequences involves crossing two different plants, wherein each of the two plants comprises one or more different modified alpha mannosidase gene sequences. The modified plants used in a crossing can be produced by methods of the invention as described above.

The modified plants and plant cells that are used in crossings or genome modification as described above can be identified or selected by (i) a reduced or undetectable activity of one or more alpha mannosidases; (ii) a reduced or undetectable expression of one or more alpha mannosidases; (iii) a reduced or undetectable level of alpha-1,3-linked fucose, beta-1,2-linked xylose, or both or residues thereof, on the N-glycan of plant proteins or heterologous protein(s); or (iv) an increase or accumulation of high mannose-type N-glycan, in the modified plant or plant cells.

The present invention relates to aspects and embodiments as set forth in the accompanying claims.

In one aspect, there is provided a polynucleotide comprising, consisting or consisting essentially of a nucleotide sequence having the genomic sequences of NtNMS1a, NtMNS1b, or NtMNS2, or SEQ ID NO:1, SEQ ID NO: 2, SEQ ID NO: 32, SEQ ID NO: 33, SEQ ID NO: 63 or SEQ ID NO: 64; or a part thereof. In one embodiment, the invention relates to a polynucleotide comprising, consisting or consisting essentially of a nucleotide sequence having at least 76% sequence identity to the genomic sequences of NtNMS1a, NtMNS1b, or NtMNS2, or SEQ ID NO:1, SEQ ID NO: 2, SEQ ID NO: 32, SEQ ID NO: 33, SEQ ID NO: 63 or SEQ ID NO: 64; or a part thereof. The invention also provides a polynucleotide comprising, consisting or consisting essentially of a nucleotide sequence having the gene sequences of NtNMS1a, NtMNS1b, NtMNS2, or NtMan1.4, or any of SEQ ID NO:30, SEQ ID NO: 94, SEQ ID NO:61, SEQ ID NO: 96, SEQ ID NO: 92, or SEQ ID NO: 98,; or a part thereof. In one embodiment, the invention relates to a polynucleotide comprising, consisting or consisting essentially of a nucleotide sequence having at least 88% sequence identity to the gene sequences of NtNMS1a, NtMNS1b, NtMNS2, or NtMan1.4, or any of SEQ ID NO:30, SEQ ID NO: 94, SEQ ID NO:61, SEQ ID NO: 96, SEQ ID NO: 92, or SEQ ID NO: 98; or a part thereof. The invention also provides a polynucleotide comprising, consisting or consisting essentially of one or more coding sequence(s) of NtNMS1a, NtMNS1b, NtMNS2, or NtMan1.4, or a nucleotide sequence encoding a polypeptide comprising, consisting of or consisting essentially of an amino acid sequence having at least 76% sequence identity to SEQ ID NO: 31, SEQ ID NO: 95, SEQ ID NO: 62, SEQ ID NO: 97, SEQ ID NO: 93, or SEQ ID NO: 99, or a part thereof. The invention also provides a polynucleotide that deviates from the nucleotide sequence of the aforementioned coding sequence(s) by the degeneracy of the genetic code; or a part thereof. The invention also provides a polynucleotide the complementary strand of which hybridizes to a nucleic acid probe consisting of the nucleotide sequence of any of (i)-(iii), or any of SEQ ID NO's: 3 to 29, SEQ ID NO's: 34 to 60; or SEQ ID NO's: 65 to 91. Preferably, the aforementioned polynucleotide encodes a polypeptide which exhibits mannose hydrolyzing activity.

The invention also provides a polypeptide selected from the group consisting of (i) a polypeptide comprising, consisting or consisting essentially of an amino acid sequence having the sequences set forth in SEQ ID NO: 31, SEQ ID NO: 95, SEQ ID NO: 62, SEQ ID NO: 97, SEQ ID NO: 93, or SEQ ID NO: 99, or a part thereof; (ii) a polypeptide comprising, consisting or consisting essentially of an amino acid sequence having at least 76% sequence identity to any of the sequences set forth in SEQ ID NO: 31, SEQ ID NO: 95, SEQ ID NO: 62, SEQ ID NO: 97, SEQ ID NO: 93, or SEQ ID NO: 99, or a part thereof; (iii) a polypeptide expressed by a nucleotide sequence according to (i)-(v) of claim 1; (iv) a polypeptide expressed by a nucleotide sequence set forth in SEQ ID NO: 2, SEQ ID NO: 30, SEQ ID NO: 94, SEQ ID NO: 33, SEQ ID NO: 61, SEQ ID NO: 96, SEQ ID NO: 64, SEQ ID NO: 92, SEQ ID NO: 98, or a part thereof. Preferably, the aforementioned polypeptide, or part thereof, has mannose hydrolyzing activity

In a further aspect, there is provided a use of any of the polynucleotides or polypeptides comprising the foregoing sequences to identify a molecule that binds the nucleic acid molecule or polypeptide. There is also provided a deoxyribonucleic acid oligonucleotide, a ribonucleic acid oligonucleotide, a zinc finger nuclease or a meganuclease that specifically binds to any of SEQ ID Nos: 1 to 30, 32 to 61, or 63 to 92; or SEQ ID Nos: 94, 96 or 98. In a further aspect, there is provided a polypeptide, a protein, an antibody or an antibody fragment that binds to SEQ ID NO:31, SEQ ID NO:62 or SEQ ID NO:93, or to SEQ ID NO: 95, 97 or 99.

The general use of zinc finger nuclease-mediated mutagenesis is known in the art and described in patent publications, such as but not limited to, WO02057293, WO02057294, WO0041566, WO0042219, and WO2005084190, which are incorporated herein by reference in its entirety. The general use of meganuclease-mediated mutagenesis is known in the art and described in patent publications, such as but not limited to, WO96/14408, WO2003025183, WO2003078619, WO2004067736, WO2007047859, and WO2009059195, which are incorporated herein by reference in its entirety.

In a further aspect, there is provided a method for reducing alpha-mannosidase I levels in at least a part of a plant, comprising the step of reducing the expression of NtMNS1a, NtMNS1b, NtMNS2, or NtMan1.4, or a combination thereof, and the activity of the NtMNS1a, NtMNS1b, NtMNS2, or NtMan1.4 polypeptide, or a combination thereof, or the activity of the polypeptide encoded by the NtMNS1a, NtMNS1b, NtMNS2, or NtMan1.4 gene sequence or a combination thereof, as compared to a control plant in which the expression of NtMNS1a, NtMNS1b, NtMNS2, or NtMan1.4, or the activity of the NtMNS1a, NtMNS1b, NtMNS2, or NtMan1.4 protein or polypeptide, has not been decreased.

In one aspect, there is provided a method for reducing alpha-mannosidase I levels in at least a part of a plant, comprising the step of reducing
a) the expression of NtMNS1a and NtMNS1b and the activity of the NtMNS1a and the NtMNS1b polypeptide; or the activity of the polypeptide encoded by the NtMNS1a and the NtMNS1b gene sequence; or
b) the expression of NtMNS1a and NtMNS2 and the activity of the NtMNS1a and NtMNS2 polypeptide, or the activity of the polypeptide encoded by the NtMNS1a and NtMNS2 gene sequence; or
c) the expression of NtMNS1a and NtMan1.4 and the activity of the NtMNS1a and NtMan1.4 polypeptide, or the activity of the polypeptide encoded by the NtMNS1a and NtMan1.4 gene sequence; or
d) the expression of NtMNS1b and NtMNS2 and the activity of the NtMNS1b and NtMNS2 polypeptide, or the activity of the polypeptide encoded by the NtMNS1b and NtMNS2 gene sequence; or
e) the expression of NtMNS1b and NtMan1.4 and the activity of the NtMNS1b and NtMan1.4 polypeptide, or the activity of the polypeptide encoded by the NtMNS1b and NtMan1.4 gene sequence; or
f) the expression of NtMNS2 and NtMan1.4 and the activity of the NtMNS2 and NtMan1.4 polypeptide, or the activity of the polypeptide encoded by the NtMNS2 and NtMan1.4 gene sequence;
   as compared to a control plant in which the expression of NtMNS1a, NtMNS1b, NtMNS2, and NtMan1.4, or the activity of the NtMNS 1a, NtMNS 1b, NtMNS2 and NtMan1.4 protein or polypeptide, has not been decreased.

In one aspect, there is provided a method for reducing alpha-mannosidase I levels in at least a part of a plant, comprising the step of reducing
(a) the expression of NtMNS1a and NtMNS1b and NtMNS2, and the activity of the NtMNS1a and the NtMNS1b and the NtMNS2 polypeptide, or the activity of the polypeptide encoded by the NtMNS1a and the NtMNS1b and the NtMNS2 gene sequence, or
(b) the expression of NtMNS1a and NtMNS2 and NtMan1.4, and the activity of the NtMNS1a and NtMNS2 and NtMan1.4 polypeptide, or the activity of the polypeptide encoded by the NtMNS1a and NtMNS2 and NtMan1.4 gene sequence, or
(c) the expression of NtMNS1a and NtMNS1b and NtMan1.4, and the activity of the NtMNS1a and NtMNS1b and NtMan1.4 polypeptide, or the activity of the polypeptide encoded by the NtMNS1a and NtMNS1b and NtMan1.4 gene sequence, or
(d) the expression of NtMNS1b and NtMNS2 and NtMan1.4, and the activity of the NtMNS1b and NtMNS2 and NtMan1.4 polypeptide, or the activity of the polypeptide encoded by the NtMNS1b and NtMNS2 and NtMan1.4 gene sequence, or
   as compared to a control plant in which the expression of NtMNS1a, NtMNS1b, NtMNS2, and NtMan1.4, or the activity of the NtMNS1a, NtMNS1b, NtMNS2 and NtMan1.4 protein or polypeptide, has not been decreased.

In one aspect, there is provided a method for reducing alpha-mannosidase I levels in at least a part of a plant, comprising the step of reducing the expression of NtMNS1a and NtMNS1b and NtMNS2 and NtMan1.4, and the activity of the NtMNS1a and the NtMNS1b and the NtMNS2 and the NtMan1.4 polypeptide, or the activity of the polypeptide encoded by the NtMNS1a and the NtMNS1b and the NtMNS2 and the NtMan1.4 gene sequence, as compared to a control plant in which the expression of NtMNS1a, NtMNS1b, NtMNS2, and NtMan1.4, or the activity of the NtMNS1a, NtMNS1b, NtMNS2 and NtMan1.4 protein or polypeptide, has not been decreased.

In a specific aspect, there is provided a method for reducing alpha-mannosidase I activity of a plant cell according to the invention and as describe herein in the preceding embodiments, comprising the step of modifying the polynucleotide sequence in the genome of a plant cell, wherein the polynucleotide sequence comprises (i) a nucleotide sequence as shown in SEQ ID Nos: 1 to 30, 32 to 61 or 63 to 92, (ii) a nucleotide sequence that is at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence as shown in the SEQ ID Nos: 1 to 30, 32 to 61 or 63 to 92 (iii) a nucleotide sequence that allows a polynucleotide probe consisting of the nucleotide sequence of (i) or (ii), or a complement thereof, to hybridize, particularly under stringent conditions, and reducing the activity of the NtMNS1a, NtMNS1b, NtMNS2 or NtMan1.4 polypeptide, in the nuclear genome of a plant cell. In another aspect, there is provided a method for reducing alpha-mannosidase I activity of a plant cell, comprising the step of introducing into a plant cell, a polynucleotide sequence of any of SEQ ID Nos: 1 to 30, 32 to 61 or 63 to 92, or SEQ ID Nos: 94, 96 or 98, or a fragment thereof, in an expressable manner in sense or anti-sense orientation, and reducing the activity of the NtMNS1a, NtMNS1b, NtMNS2 or NtMan1.4 polypeptide.

In another aspect, there is provided a method for reducing alpha-mannosidase I activity of a plant cell, comprising the step of introducing into, or expressing in a plant cell, a ribonucleic acid complementary or partially complementary to any of SEQ ID Nos: 1 to 30, 32 to 61 or 63 to 92, or SEQ ID Nos: 94, 96 or 98 and reducing the activity of the NtMNS1a, NtMNS1b, NtMNS2 or NtMan1.4 polypeptide.

In another aspect, there is provided a method for reducing alpha-mannosidase I activity of a plant cell, comprising the step of introducing into, or expressing in a plant cell, a ribonucleic acid complementary or partially complementary to any of SEQ ID Nos: 1 to 30, 32 to 61 or 63 to 92, or SEQ ID Nos: 94, 96 or 98, and reducing the activity of the NtMNS1a and the NtMNS1b or of the NtMNS1a and the NtMNS2, or of the NtMNS1a and NtMan1.4, or of the NtMNS1b and the NtMNS2, or of the NtMNS1b and NtMan1.4, or of the NtMNS2 and NtMan1.4 polypeptide.

In another aspect, there is provided a method for reducing alpha-mannosidase I activity of a plant cell, comprising the step of introducing into, or expressing in a plant cell, a ribonucleic acid complementary or partially complementary to any of SEQ ID Nos: 1 to 30, 32 to 61 or 63 to 92, or SEQ ID Nos: 94, 96 or 98, and reducing the activity of the NtMNS1a and the NtMNS1b or of the NtMNS1a and the NtMNS2, or of the NtMNS1a and NtMan1.4, or of the NtMNS1b and the NtMNS2, or of the NtMNS1b and NtMan1.4, or of the NtMNS2 and NtMan1.4polypeptide.

In another aspect, there is provided a method for reducing alpha-mannosidase I activity of a plant cell, comprising the step of introducing into, or expressing in a plant cell, a ribonucleic acid complementary or partially complementary to any of SEQ ID Nos: 1 to 30, 32 to 61 or 63 to 92, or SEQ ID Nos: 94, 96 or 98, and reducing the activity of the NtMNS1a and the NtMNS1b and the NtMNS2 and the NtMan1.4 polypeptide.

In another aspect, there is provided a method for reducing alpha-mannosidase I activity of a plant cell, comprising the step of introducing into a plant cell, a molecule that specifically binds to any of SEQ ID Nos: 1 to 99.

In a further aspect, there is provided a method for reducing alpha-mannosidase I activity of a plant cell, comprising the step of introducing into a plant cell, a deoxyribonucleic acid oligonucleotide, a ribonucleic acid oligonucleotide, a polypeptide, a protein, an antibody or an antibody fragment, a zinc finger protein or a meganuclease that specifically binds to any of SEQ ID Nos: 1 to 30, 32 to 61 or 63 to 92; or SEQ ID Nos: 94, 96 or 98; or a polypeptide, a protein, an antibody or an antibody fragment that binds to SEQ ID NO:31, SEQ ID NO:62 or SEQ ID:93, or to SEQ ID NO:95, SEQ ID NO:97 or SEQ ID NO:99, and reducing the activity of NtMNS1a, NtMNS1b,NtMNS2 or NtMan1.4. In a further aspect, there is provided a method for reducing alpha-mannosidase I activity of a plant cell, comprising the step of introducing into a plant cell, a deoxyribonucleic acid oligonucleotide, a ribonucleic acid oligonucleotide, a polypeptide, a protein, an antibody or an antibody fragment, a zinc finger protein or a meganuclease that specifically binds to any of SEQ ID Nos: 1 to 30, 32 to 61 or 63 to 92; or to SEQ ID Nos: 94, 96 or 98; or a polypeptide, a protein, an antibody or an antibody fragment that binds to SEQ ID NO:31, SEQ ID NO:62 or SEQ ID:93, or to SEQ ID NO:95, SEQ ID NO:97 or SEQ ID NO:99, and reducing the activity of the NtMNS1a and the NtMNS1b or of the NtMNS1a and the NtMNS2, or of the NtMNS1a and NtMan1.4, or of the NtMNS1b and the NtMNS2, or of the NtMNS1b and NtMan1.4, or of the NtMNS2 and NtMan1.4 polypeptide.

In a further aspect, there is provided a method for reducing alpha-mannosidase I activity of a plant cell, comprising the step of introducing into a plant cell, a deoxyribonucleic acid oligonucleotide, a ribonucleic acid oligonucleotide, a polypeptide, a protein, an antibody or an antibody fragment, a zinc finger protein or a meganuclease that specifically binds to any of SEQ ID Nos: 1 to 30, 32 to 61 or 63 to 92; or to SEQ ID Nos: 94, 96 or 98; or a polypeptide, a protein, an antibody or an antibody fragment that binds to SEQ ID NO:31, SEQ ID NO:62 or SEQ ID:93, or to SEQ ID NO:95, SEQ ID NO:97 or SEQ ID NO:99, and reducing the activity of the NtMNS1a and the NtMNS1b and the NtMNS2 polypeptide, or of the NtMNS1a and NtMNS2 and NtMan1.4 polypeptide, or of the NtMNS1a and NtMNS1b and NtMan1.4 polypeptide, or of the NtMNS1b and NtMNS2 and NtMan1.4 polypeptide.

In a further aspect, there is provided a method for reducing alpha-mannosidase I activity of a plant cell, comprising the step of introducing into a plant cell, a deoxyribonucleic acid oligonucleotide, a ribonucleic acid oligonucleotide, a polypeptide, a protein, an antibody or an antibody fragment, a zinc finger protein or a meganuclease that specifically binds to any of SEQ ID Nos: 1 to 30, 32 to 61 or 63 to 92; or to SEQ ID Nos: 94, 96 or 98; or a polypeptide, a protein, an antibody or an antibody fragment that binds to SEQ ID NO:31, SEQ ID NO:62 or SEQ ID:93, or to SEQ ID NO:95, SEQ ID NO:97 or SEQ ID NO:99, and reducing the activity of the NtMNS1a and the NtMNS1b and the NtMNS2 and the NtMan1.4 polypeptide.

In a further aspect, there is provided a method for increasing alpha-mannosidase I levels in at least a part of a plant, comprising the step of increasing the expression of NtMNS1a, NtMNS1b, NtMNS2, or NtMan1.4, or a combination thereof, and the activity of the NtMNS1a, NtMNS1b, NtMNS2, or NtMan1.4 polypeptide, or a combination thereof, or the activity of the polypeptide encoded by the NtMNS1a, NtMNS1b, NtMNS2, or NtMan1.4 gene sequence or a combination thereof, as compared to a control plant in which the expression of NtMNS1a, NtMNS1b, NtMNS2, and NtMan1.4, and the activity of the NtMNS1a, NtMNS1b, NtMNS2, and NtMan1.4 protein or polypeptide, has not been altered.

In one aspect, there is provided a method for increasing alpha-mannosidase I levels in at least a part of a plant, comprising the step of increasing the
a) the expression of NtMNS1a and NtMNS1b and the activity of the NtMNS1a and the NtMNS1b polypeptide; or the activity of the polypeptide encoded by the NtMNS1a and the NtMNS1b gene sequence; or
b) the expression of NtMNS1a and NtMNS2 and the activity of the NtMNS1a and NtMNS2 polypeptide, or the activity of the polypeptide encoded by the NtMNS1a and NtMNS2 gene sequence; or
c) the expression of NtMNS1a and NtMan1.4 and the activity of the NtMNS1a and NtMan1.4 polypeptide, or the activity of the polypeptide encoded by the NtMNS1a and NtMan1.4 gene sequence; or
d) the expression of NtMNS1b and NtMNS2 and the activity of the NtMNS1b and NtMNS2 polypeptide, or the activity of the polypeptide encoded by the NtMNS1b and NtMNS2 gene sequence; or
e) the expression of NtMNS1b and NtMan1.4 and the activity of the NtMNS1b and NtMan1.4 polypeptide, or the activity of the polypeptide encoded by the NtMNS1b and NtMan1.4 gene sequence; or
(f) the expression of NtMNS2 and NtMan1.4 and the activity of the NtMNS2 and NtMan1.4 polypeptide, or the activity of the polypeptide encoded by the NtMNS2 and NtMan1.4 gene sequence;
   as compared to a control plant in which the expression of NtMNS1a, NtMNS1b, NtMNS2, and NtMan1.4, or the activity of the NtMNS1a, NtMNS1b, NtMNS2 and NtMan1.4 protein or polypeptide, has not been altered.

In one aspect, there is provided a method for increasing alpha-mannosidase I levels in at least a part of a plant, comprising the step of increasing the
(a) the expression of NtMNS1a and NtMNS1b and NtMNS2, and the activity of the NtMNS1a and the NtMNS1b and the NtMNS2 polypeptide, or the activity of the polypeptide encoded by the NtMNS1a and the NtMNS1b and the NtMNS2 gene sequence, or
(b) the expression of NtMNS1a and NtMNS2 and NtMan1.4, and the activity of the NtMNS1a and NtMNS2 and NtMan1.4 polypeptide, or the activity of the polypeptide encoded by the NtMNS1a and NtMNS2 and NtMan1.4 gene sequence, or
(c) the expression of NtMNS1a and NtMNS1b and NtMan1.4, and the activity of the NtMNS1a and NtMNS1b and NtMan1.4 polypeptide, or the activity of the polypeptide encoded by the NtMNS1a and NtMNS1b and NtMan1.4 gene sequence, or
(d) the expression of NtMNS1b and NtMNS2 and NtMan1.4, and the activity of the NtMNS1b and NtMNS2 and NtMan1.4 polypeptide, or the activity of the polypeptide encoded by the NtMNS1b and NtMNS2 and NtMan1.4 gene sequence, or
   as compared to a control plant in which the expression of NtMNS1a, NtMNS1b, NtMNS2, and NtMan1.4, or the activity of the NtMNS1a, NtMNS1b, NtMNS2 and NtMan1.4 protein or polypeptide, has not been altered.

In one aspect, there is provided a method for increasing alpha-mannosidase I levels in at least a part of a plant, comprising the step of increasing the expression of NtMNS1a and NtMNS1b and NtMNS2 and NtMan1.4, and the activity of the NtMNS1a and the NtMNS1b and the NtMNS2 and the NtMan1.4 polypeptide, or the activity of the polypeptide encoded by the NtMNS1a and the NtMNS1b and the NtMNS2 and the NtMan1.4 gene sequence, as compared to a control plant in which the expression of NtMNS1a, NtMNS1b, NtMNS2, and NtMan1.4, or the activity of the NtMNS1a, NtMNS1b, NtMNS2 and NtMan1.4 protein or polypeptide, has not been altered.

In a specific aspect, there is provided a method for reducing alpha-mannosidase I activity of a plant cell according to the invention and as describe herein in the preceding embodiments, comprising the step of modifying the polynucleotide in the genome of a plant cell by a genome editing or genome engineering technology, the genome editing or genome engineering technology selected from the list comprising zinc finger nuclease-mediated mutagenesis, chemical-induced mutagenesis, radiation mutagenesis, homologous recombination, oligonucleotide-mediated mutagenesis or meganuclease-mediated mutagenesis, wherein the polynucleotide sequence comprises (i) a nucleotide sequence as shown in SEQ ID Nos: 1, SEQ ID NO:32 or SEQ ID NO:63, (ii) a nucleotide sequence that is at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence as shown in the SEQ ID Nos: 1, SEQ ID NO:32 or SEQ ID NO:63 (iii) a nucleotide sequence that allows a polynucleotide probe consisting of the nucleotide sequence of (i) or (ii), or a complement thereof, to hybridize, particularly under stringent conditions.

In one aspect, the invention relates to the use of a nucleotide sequence according to the invention as defined herein in the various embodiments, or a part thereof, for identifying a target site in
a. a first target nucleotide sequence in a genomic region comprising a coding sequence for an alpha-mannosidase I; or
b. the first target nucleotide sequence of a) and a second target nucleotide sequence in a genomic region comprising a coding sequence for an alpha-mannosidase I; or
c. the first target nucleotide sequence of a), the second target nucleotide sequence of b) and a third target nucleotide sequence in a genomic region comprising a coding sequence for an alpha-mannosidase I;
d. the first target nucleotide sequence of a), the second target nucleotide sequence of b) the third target nucleotide sequence of c) and a fourth target nucleotide sequence in a genomic region comprising a coding sequence for an alpha-mannosidase I;
e. all target nucleotide sequences a), b), c) and d);
   for modification such that the activity or the expression of alpha-mannosidase I in the modified plant cell comprising the modification is altered relative to an unmodified plant cell, wherein the alpha-mannosidase I is selected from the group consisting of NtMNS1a, NtMNS1b, NtMNS2, and NtMan1.4, and wherein the first, second, third and fourth target alpha-mannosidases I are different from each other.

In a specific aspect of the invention, the first, second, third and/or fourth target nucleotide sequence of the modified Nicotiana tabacum plant cell or the Nicotiana tabacum plant according to the invention and as described herein in the various embodiments, has
(i) at least 76% sequence identity to SEQ ID Nos: 1 to 30, 32 to 61 or 63 to 92; or to SEQ ID Nos: 94, 96 or 98, but particularly to SEQ ID NO:1, SEQ ID NO: 2, SEQ ID NO: 32, SEQ ID NO: 33, SEQ ID NO: 63 or SEQ ID NO: 64; or a part thereof;
(ii) at least 88% sequence identity to any of SEQ ID Nos: 1 to 30, 32 to 61 or 63 to 92; or to SEQ ID Nos: 94, 96 or 98, but particularly SEQ ID NO:30, SEQ ID NO: 94, SEQ ID NO:61, SEQ ID NO: 96, SEQ ID NO: 92, or SEQ ID NO: 98; or a part thereof.

In another specific aspect of the invention, the first, second, third and/or fourth target nucleotide sequence of the modified Nicotiana tabacum plant cell or the Nicotiana tabacum plant according to the invention and as described herein in the various embodiments comprises, essentially comprises or consists of
(i) SEQ ID Nos: 1 to 30, 32 to 61 or 63 to 92; or to SEQ ID Nos: 94, 96 or 98, particularly SEQ ID NO:1, SEQ ID NO: 2, SEQ ID NO: 32, SEQ ID NO: 33, SEQ ID NO: 63 or SEQ ID NO: 64; or a part thereof;
(ii) SEQ ID NO:30, SEQ ID NO: 94, SEQ ID NO:61, SEQ ID NO: 96, SEQ ID NO: 92, or SEQ ID NO: 98; or a part thereof.

In a specific aspect, a nucleotide sequence as defined herein in the various embodiments may be used for making a non-natural meganuclease protein that selectively cleaves a genomic DNA molecule at a site within a nucleotide sequence as defined herein.

In another specific aspect, a nucleotide sequence as defined herein in the various embodiments may be used for making a zinc finger nuclease that introduces a double-stranded break in at least one of the target nucleotide sequences as defined herein. In a further aspect, there is provided a plant cell with altered alpha-mannosidase I activity, particularly with reduced or increased alpha-mannosidase I activity, particularly a plant cell resulting from the method according to the invention as described herein in the various embodiments.

In particular, the present invention relates to a genetically modified Nicotiana tabacum plant cell, or a Nicotiana tabacum plant comprising the modified plant cells, wherein the modified plant cell comprises at least a modification of a first target nucleotide sequence in a genomic region comprising a coding sequence for an alpha-mannosidase I selected from the group consisting of NtMNS1a, NtMNS1b, NtMNS2, and NtMan1.4, and/or an allelic variant thereof, such that (i) the activity or the expression of alpha-mannosidase I in the modified plant cell is altered relative to an unmodified plant cell.

In one aspect, said modified Nicotiana tabacum plant cell or Nicotiana tabacum plant comprises in addition to (a) the modification of a first target nucleotide sequence, (b) at least a modification of a second target nucleotide sequence in a genomic region comprising a coding sequence for an alpha-mannosidase I, or (c) at least a modification of a third target nucleotide sequence in a genomic region comprising a coding sequence for an alpha-mannosidase I, or (d) at least a modification of a fourth target nucleotide sequence in a genomic region comprising a coding sequence for an alpha-mannosidase I, or a combination of (a) and (b), (a) and (c), (a) and (d), (b) and (c), (b) and (d), or (c) and (d); or (a) and (b) and (c), (a) and (b) and (d), (a) and (c) and (d), or (b) and (c) and (d), or (a) and (b) and (c) and (d), wherein the alpha-mannosidase I is selected from the group consisting of NtMNS1a, NtMNS1b, NtMNS2, and NtMan1.4, and wherein the first, second, third and fourth alpha-mannosidases I are different from each other.

In a specific aspect of the invention, the first, second, third and/or fourth target nucleotide sequence of the modified Nicotiana tabacum plant cell or the Nicotiana tabacum plant according to the invention and as described herein in the various embodiments, has
(i) at least 76% sequence identity to SEQ ID Nos: 1 to 30, 32 to 61 or 63 to 92; or to SEQ ID Nos: 94, 96 or 98, but particularly to SEQ ID NO:1, SEQ ID NO: 2, SEQ ID NO: 32, SEQ ID NO: 33, SEQ ID NO: 63 or SEQ ID NO: 64; or a part thereof;
(ii) at least 88% sequence identity to any of SEQ ID Nos: 1 to 30, 32 to 61 or 63 to 92; or to SEQ ID Nos: 94, 96 or 98, but particularly SEQ ID NO:30, SEQ ID NO: 94, SEQ ID NO:61, SEQ ID NO: 96, SEQ ID NO: 92, or SEQ ID NO: 98; or a part thereof.

In another specific aspect of the invention, the first, second, third and/or fourth target nucleotide sequence of the modified Nicotiana tabacum plant cell or the Nicotiana tabacum plant according to the invention and as described herein in the various embodiments comprises, essentially comprises or consists of
(i) SEQ ID Nos: 1 to 30, 32 to 61 or 63 to 92; or to SEQ ID Nos: 94, 96 or 98, particularly SEQ ID NO:1, SEQ ID NO: 2, SEQ ID NO: 32, SEQ ID NO: 33, SEQ ID NO: 63 or SEQ ID NO: 64; or a part thereof;
(ii) SEQ ID NO:30, SEQ ID NO: 94, SEQ ID NO:61, SEQ ID NO: 96, SEQ ID NO: 92, or SEQ ID NO: 98; or a part thereof.

In various embodiments of the invention provides a modified Nicotiana tabacum plant cell or Nicotiana tabacum plant according to the invention and as described herein in the various embodiments, wherein the activity or the expression of alpha-mannosidase I in the modified plant cell is (a) reduced or (b) increased relative to an unmodified plant cell.

Also contemplated within the present invention are progeny plants that can be obtained from the modified Nicotiana tabacum plant according to the invention and as described herein in the various embodiments, wherein said progeny plant comprises a modification in at least one of the target sequences as defined herein in the various embodiments, wherein the activity or the expression of the alpha-mannosidase I is altered, particularly increased or reduced, relative to an unmodified plant cell.

The increase in activity as compared to the control plant may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200% or 300% or more, which includes an increase in transcriptional activity or protein expression or both. The reduction in activity as compared to the control plant may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or 100%, which includes a reduction in transcriptional activity or protein expression or both.

The increase in mannose content as compared to a control plant may be from about 5% to about 100%, or an increase of at least 10° A), at least 20%, at least 25%, at least 30%, at least 40%, at least 50%, at least 60%, at least 70%, at least 75%, at least 80%, at least 90%, at least 95%, at least 98%, or up to 100% or more—such as 200% or 300% or more.

The decrease in mannose content as compared to a control plant may be from about 5% to about 100%, or a decrease of at least 10%, at least 20%, at least 25%, at least 30%, at least 40%, at least 50%, at least 60%, at least 70%, at least 75%, at least 80%, at least 90%, at least 95%, at least 98%, or up to 100%.

In a further aspect, there is provided a non-natural or modified alfalfa, duckweed, rice, maize or carrot plant cell, or a plant cell of a plant belonging to the genus Nicotiana, particularly Nicotiana benthamiana, N. sylvestris, N. excelsior, N. exigua, N. tomentosiformis, N. rustica, N. otophora or N. tabacum, or a variety, line, selection or cultivar thereof, with modified alpha-mannosidase activity and reduced or increased alpha-mannosidase I activity compared to a control plant, particularly a plant cell resulting from the method according to the invention as described herein in the various embodiments.

In one embodiment, the modified, i.e., the genetically modified, Nicotiana tabacum plant cell, or a Nicotiana tabacum plant, including the progeny thereof, comprising the modified plant cells according to the invention and as described herein in the various embodiments is Nicotiana tabacum cultivar PM132, the seeds of which were deposited on 6 Jan. 2011 at NCIMB Ltd (an International Depositary Authority under the Budapest Treaty, located at Ferguson Building, Craibstone Estate, Bucksburn, Aberdeen, AB21 9YA, United Kingdom) under accession number NCIMB 41802. In another embodiment, the modified, i.e., the genetically modified, Nicotiana tabacum plant cell, or a Nicotiana tabacum plant, including the progeny thereof, comprising the modified plant cells according to the invention and as described herein is Nicotiana tabacum line PM016, the seeds of which were deposited under accession number NCIMB 41798; Nicotiana tabacum line PM021, the seeds of which were deposited under accession number NCIMB 41799; Nicotiana tabacum line PM092, the seeds of which were deposited under accession number NCIMB 41800; Nicotiana tabacum line PM102, the seeds of which were deposited under accession number NCIMB 41801; Nicotiana tabacum line PM204, the seeds of which were deposited on 6 Jan. 2011 at NCIMB Ltd. under accession number NCIMB 41803; Nicotiana tabacum line PM205, the seeds of which were deposited under accession number NCIMB 41804; Nicotiana tabacum line PM215, the seeds of which were deposited under accession number NCIMB 41805; Nicotiana tabacum line PM216, the seeds of which were deposited under accession number NCIMB 41806; and Nicotiana tabacum line PM217, the seeds of which were deposited under accession number NCIMB 41807.

Also provided herein is a method for producing a Nicotiana tabacum plant cell or of a Nicotiana tabacum plant comprising the modified plant cells capable of producing humanized glycoproteins, the method comprising:
(i) modifying in the genome of a tobacco plant cell
   a. a first target nucleotide sequence in a genomic region comprising a coding sequence for an alpha-mannosidase I; or
   b. the first target nucleotide sequence of a) and a second target nucleotide sequence in a genomic region comprising a coding sequence for an alpha-mannosidase I; or
   c. the first target nucleotide sequence of a), the second target nucleotide sequence of b) and a third target nucleotide sequence in a genomic region comprising a coding sequence for an alpha-mannosidase I;
   d. the first target nucleotide sequence of a), the second target nucleotide sequence of b) and the third target nucleotide sequence of c) and a fourth target nucleotide sequence in a genomic region comprising a coding sequence for an alpha-mannosidase I;
   e. all target nucleotide sequences a), b), c) and d);
(ii) identifying and, optionally, selecting a modified plant or plant cell comprising the modification in the target nucleotide sequence;
(iii) optionally breeding the modified plant with another Nicotiana plant,
   wherein the alpha-mannosidase I is selected from the group consisting of NtMNS1a, NtMNS1b, NtMNS2, and NtMan1.4 and wherein the first, second, third and fourth target alpha-mannosidases I are different from each other and wherein the activity or the expression of alpha-mannosidase I in the modified plant cell comprising the modification is altered relative to an unmodified plant cell such that the glycoproteins produced by said modified plant cell substantially lack alpha-1,3-linked fucose and beta-1,2-linked xylose on its N-glycan as compared to a glycoprotein obtained from an unmodified plant cell.

In a specific aspect, the first, second, third and/or fourth target nucleotide sequence has
(i) at least 76% sequence identity to SEQ ID Nos: 1 to 30, 32 to 61 or 63 to 92; or to SEQ ID Nos: 94, 96 or 98, but particularly to SEQ ID NO:1, SEQ ID NO: 2, SEQ ID NO: 32, SEQ ID NO: 33, SEQ ID NO: 63 or SEQ ID NO: 64; or a part thereof;
(ii) at least 88% sequence identity to any of SEQ ID Nos: 1 to 30, 32 to 61 or 63 to 92; or to SEQ ID Nos: 94, 96 or 98, but particularly SEQ ID NO:30, SEQ ID NO: 94, SEQ ID NO:61, SEQ ID NO: 96, SEQ ID NO: 92, or SEQ ID NO: 98; or a part thereof.

In another specific aspect, the first, second, third and/or fourth target nucleotide sequence comprises, essentially comprises or consists of
(i) SEQ ID Nos: 1 to 30, 32 to 61 or 63 to 92; or to SEQ ID Nos: 94, 96 or 98, particularly SEQ ID NO:1, SEQ ID NO: 2, SEQ ID NO: 32, SEQ ID NO: 33, SEQ ID NO: 63 or SEQ ID NO: 64; or a part thereof;
(ii) SEQ ID NO:30, SEQ ID NO: 94, SEQ ID NO:61, SEQ ID NO: 96, SEQ ID NO: 92, or SEQ ID NO: 98; or a part thereof.

It is further contemplated herein, that the modification of the genome of a tobacco plant or plant cell comprises the steps of
a. identifying in the target nucleotide sequence of a Nicotiana tabacum plant or plant cell and, optionally, in at least one allelic variant thereof, a target site,
b. designing, based on the nucleotide sequence as defined in claim 8 or 9, a mutagenic oligonucleotide capable of recognizing and binding at or adjacent to said target site, and
c. binding the mutagenic oligonucleotide to the target nucleotide sequence in the genome of a tobacco plant or plant cell under conditions such that the genome is modified.

In a further aspect, there is provided a method for producing a glycoprotein, comprising the steps of introducing into a non-natural or modified plant cell with increased or reduced alpha-mannosidase I activity compared to a control plant, particularly into a plant cell according to the invention as described herein in the various embodiments, an expression construct comprising a polynucleotide sequence encoding the target glycoprotein, culturing the plant cell for a time period sufficient to produce the target glycoprotein and optionally, regenerating a plant from said plant cell, or harvesting the glycoprotein from the modified plant cell or plant comprising the modified plant cells. In a specific aspect, the present invention relates to a method for producing a heterologous protein, said method comprising:

(a) introducing into a modified Nicotiana tabacum plant cell or plant as defined in any one of claims 1 to 6 an expression construct comprising a nucleotide sequence that encodes a heterologous glycoprotein, particularly an antigen for making a vaccine, a cytokine, a hormone, a coagulation protein, an apolipoprotein, an enzyme for replacement therapy in human, an immunoglobulin or a fragment thereof; and culturing the modified plant cell that comprises the expression construct such that the heterologous glycoprotein is produced, wherein said glycoprotein substantially lacks alpha-1,3-linked fucose and beta-1,2-linked xylose on its N-glycan as compared to a glycoprotein obtained from an unmodified plant cell. (b) optionally, regenerating a plant from the plant cell, and growing the plant and its progenies, and (c) optionally harvesting the glycoprotein.

In a further aspect, there is provided a plant composition comprising a glycoprotein obtained from modified plant cells or a plant comprising modified plant cells, particularly from modified plant cells or a plant comprising modified plant cells according to the invention and as described herein in the various embodiments, characterized in that the glycoprotein has an increase or a decrease in the amount of mannoses on the N-glycan of the glycoprotein as compared to the same glycoprotein obtained from a control plant. In a specific aspect, the invention provides a plant composition comprising a heterologous glycoprotein, obtainable from a plant comprising modified plant cells as defined herein in the various embodiments, wherein the glycoprotein substantially lacks alpha-1,3-linked fucose and beta-1,2-linked xylose on its N-glycan as compared to a glycoprotein obtained from an unmodified plant cell.

In a further aspect, there is provided a substantially pure glycoprotein obtained from a plant composition comprising said glycoprotein and obtained from modified plant cells or a plant comprising modified plant cells, particularly from modified plant cells or a plant comprising modified plant cells according to the invention and as described herein in the various embodiments, characterized in that the glycoprotein has an increase or a decrease in the amount of mannoses on the N-glycan of the glycoprotein as compared to the same glycoprotein obtained from a control plant with normal levels of alpha-mannosidase I activity.

In one embodiment of the invention, a first gene sequence encoding a first alpha mannosidase or a fragment thereof, in a plant cell is modified, followed by identification or isolation of modified plant cells that exhibit a reduced activity of the first alpha mannosidase. The modified plant cells comprising a modified first alpha mannosidase gene are then subject to mutagenesis, wherein a second gene sequence encoding a second alpha mannosidase or a fragment thereof is modified. This is followed by identification or isolation of modified plant cells that exhibit a reduced activity of the second alpha mannosidase, or a further reduction of the alpha mannosidase activity relative to that of cells that carry only the first modification. Modified plant cells can be isolated after identification. The modified plant cell obtained at this stage comprises two modifications in two gene sequences that encode two alpha mannosidases, or two variants or alleles of an alpha mannosidase.

In another embodiment of the invention, a first gene sequence encoding a first alpha-mannosidase I or a fragment thereof, in a plant cell is modified, and a second gene sequence encoding a second alpha-mannosidase I or a fragment thereof, in a different plant cell is modified, followed by identification or isolation of the first and second modified plant cell, that exhibit a reduced activity of the first and second alpha-mannosidase I. Plants comprising the modified plant cells comprising the modified first and second alpha-mannosidase I, can be crossed to obtain a progeny comprising two modifications in two alpha-mannosidase I gene sequences that encode two alpha-mannosidases I, or two variants or alleles of an alpha-mannosidase I.

In one aspect, the two gene sequences encoding a first alpha mannosidase and a second alpha mannosidase are selected from the group consisting of NtMNS1a, NtMNS1b, NtMNS2, and NtMan1.4, or are variants or alleles thereof as described herein in the various embodiments.

In a specific aspect, the two gene sequences encode the NtMNS1a and the NtMNS1b or of the NtMNS1a and the NtMNS2, or of the NtMNS1a and NtMan1.4, or of the NtMNS1b and the NtMNS2, or of the NtMNS1b and NtMan1.4, or of the NtMNS2 and NtMan1.4 polypeptide, or variants or alleles thereof as described herein in the various embodiments.

In one aspect, the invention relates to a modified plant cell comprising three modifications in three gene sequences that encode three alpha mannosidases, or three variants or alleles of an alpha mannosidase as described herein in the various embodiments.

In a specific aspect, the three gene sequences encode the NtMNS1a and the NtMNS1b and the NtMNS2 polypeptide, or of the NtMNS1a and NtMNS2 and NtMan1.4 polypeptide, or of the NtMNS1a and NtMNS1b and NtMan1.4 polypeptide, or of the NtMNS1b and NtMNS2 and NtMan1.4 polypeptide, or variants or alleles thereof as described herein in the various embodiments.

In one aspect, the invention relates to a modified plant cell comprising four modifications in four gene sequences that encode four alpha mannosidases, or four variants or alleles of an alpha mannosidase as described herein in the various embodiments.

In a specific aspect, the four gene sequences encode the NtMNS1a and the NtMNS1b and the NtMNS2 and the NtMan1.4 polypeptide.

In a further aspect, there is provided a pharmaceutical composition comprising a glycoprotein with an increase or a decrease in the amount of mannoses on the N-glycan of the glycoprotein, obtained from a plant with a modified alpha-mannosidase I activity, particularly a plant according to the invention and as described herein in the preceding embodiments, as compared to the same glycoprotein obtained from a normal plant with normal levels of alpha-mannosidase I activity.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. The carrier can be a parenteral carrier, more particularly a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, for example, polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; nonionic surfactants such as polysorbates, poloxamers, or PEG; or all

In a further aspect, there is provided an expression vector comprising a polynucleotide or a nucleic acid construct of any of SEQ ID Nos:1 to 30, 32 to 61 or 63 to 92, or SEQ ID Nos: 94, 96 or 98.

According to the invention, producing modified and non-naturally occurring plant cells and plants (including cells, biomass, seed and leaves obtained therefrom), in which the amount of alpha-mannosidase I is altered, provides a number of advantages.

By way of example, the plant cells or plants, including transgenic and non-naturally occurring tobacco plant cells or plants, can be cultivated or grown for the manufacture of heterologous glycoproteins containing variable amounts of mannoses on the N-glycan of the glycoprotein.

By way of further example, transgenic and non-naturally occurring plants (including cells, biomass, seed and leaves obtained therefrom) exhibit a modified amount of mannoses on the N-glycan of a glycoprotein, compared to control counterparts and may be used for the manufacture of heterologous glycoproteins for the purpose of making a pharmaceutical composition.

The pharmaceutical composition, as used herein, comprising a glycoprotein as mentioned herein above in the various embodiments with a modified amount of mannoses may be more efficacious, especially antigen that can be used in a vaccine, since antigen presenting cells can bind to high mannose potentially resulting in a heightened immune response. For certain antibodies that are produced in plants, the high mannose present can lead to an increased antibody-dependent cellular cytotoxicity. Suitable plants that can be manipulated according to the disclosed methods include plants cultivatable for the manufacture of recombinant proteins, including but not limited to tobacco, relatives of tobacco and belonging to the genus Nicotiana, corn, alfalfa, duckweed, carrots, and mosses.

The polynucleotide, polypeptide and the method according to the invention is described in more details herein above and below by way of exemplary embodiments and with reference to the SEQUENCE INFORMATION, in which:

SEQUENCE 1 (SEQ ID NO: 1) shows the NtMNS1a polynucleotide in which the 5′ and 3′ UTR regions are in lowercase letters and underlined; exons are shown in capital letters; introns are shown in lower-case letters; and start and stop codons are shown in capital bold letters and underlined.
SEQUENCE 30 (SEQ ID NO: 30) shows the NtMNS1a cDNA sequence.
SEQUENCE 31 (SEQ ID NO: 31) shows the NtMNS1a protein sequence
SEQUENCE 32 (SEQ ID NO: 32) shows the NtMNS1b polynucleotide in which the 5′ and 3′ UTR regions are in lowercase letters and underlined; exons are shown in capital letters; introns are shown in lower-case letters; and start and stop codons are shown in capital bold letters and underlined.
SEQUENCE 61 (SEQ ID NO: 61) shows the NtMNS1b cDNA sequence
SEQUENCE 62 (SEQ ID NO: 62) shows the NtMNS1b protein sequence
SEQUENCE 63 (SEQ ID NO: 63) shows the NtMNS2 polynucleotide in which the 5′ and 3′ UTR regions are in lowercase letters and underlined; exons are shown in capital letters; introns are shown in lower-case letters; and start and stop codons are shown in capital bold letters and underlined.
Table 1 shows the percentage identity and similarity of the NtMNS predicted protein sequences compared to the closest plant sequences AtMNS1 and AtMNS2 using EMBOSS needle. NtMNS1a is the predicted protein of SEQ ID NO:30; NtMNS1b is the predicted protein of SEQ ID NO:61 and NtMNS2 is the predicted protein of SEQ ID NO:92. AtMNS1 is the predicted protein of a putative Arabidopsis thaliana mannosyl-oligosaccharide 1,2-alpha-mannosidase (At1g51590) and NtMNS2 is the predicted protein of a putative Athaliana mannosidase (At3g21160) as reported (Kajiura et al. (2010) Glycobiology 20: 235-247).
Table 2 shows the identity (%) of SEQ (SEQ ID NO:) and database entries (best match) using local pairwise alignments using the program EMBOSS water, the sequence (SEQ) length in basepairs and the number of identical basepairs in the best local alignment.

Further aspects and embodiments relating to the present invention are detailed descripted in the following:

### Alpha-Mannosidases.

Class I alpha-mannosidases or alpha-mannosidase I enzymes (EC 3.2.1.113) were first described in microsomes from mung bean (Forsee (1985) Arch. Biochem. Biophys. 242: 48-57). The enzyme that was purified from mung bean had specific α(1,2)-mannosidase activity but no sequence information was provided. The first putative plant alpha-mannosidase I gene, named Gm-Man1, was cloned in 1999 from soybean (Glycine max) by Nebenführ (Nebenführ et al. (1999) Plant Physiol. 121: 1127-1142; GenBank accession no. AF126550). A fusion protein of this putative alpha-mannosidase I and green fluorescent protein revealed its presence in cis-Golgi stacks when overexpressed in tobacco (Nebenführ (1999), supra) but its enzymatic activity and role in N-glycan biosynthesis has not been reported. The Arabidopsis thaliana genome sequencing project revealed a number of putative alpha-mannosidase I sequences: MNS1 (At1g51590), MNS2 (At3g21160), MNS3 (At1g30000), MNS4 (At5g43710) and MNS5 (At1g27520). The predicted full-length cDNA sequences of these are known and this sequence information is present in GenBank.

MNS1 and MNS2 appeared to be Golgi-resident alpha-mannosidases whereas MNS3 was localized in the endoplasmatic reticulum (Liebminger et al. (2009) The Plant Cell 21: 3850-3867). Where MNS3 cleaved only one α(1,2)-mannose from a Man9-GlcNAc2 substrate, MNS1 and MNS2 cleaved three α(1,2)-mannoses from Man8-GlcNAc2 to Man5-GlcNAc. Mutations in MNS1, MNS2 and MNS3 and combinations thereof in Arabidopsis resulted in aberrant N-glycans and showed that these genes are essential for early N-glycan processing, root development and cell wall biosynthesis in Arabidopsis (Liebminger et al. (2009), supra).

NtMNS Tobacco Alpha-Mannosidase Polynucleotides.

As shown in the SEQUENCE INFORMATION, the NtMNS1a genomic clone of SEQ ID NO:1 with 5′ and 3′ untranslated regions, or SEQ ID NO:2 without 5′ and 3′ untranslated regions, comprises 14 exons and 13 introns: exon 1 (SEQ ID NO:3), exon 2 (SEQ ID NO:5), exon 3 (SEQ ID NO:7), exon 4 (SEQ ID NO:9), exon 5 (SEQ ID NO:11), exon 6 (SEQ ID NO:13), exon 7 (SEQ ID NO:15), exon 8 (SEQ ID NO:17), exon 9 (SEQ ID NO:19), exon 10 (SEQ ID NO:21), exon 11 (SEQ ID NO:23), exon 12 (SEQ ID NO:25), exon 13 (SEQ ID NO:27), exon 14 (SEQ ID NO:29), intron 1 (SEQ ID NO:4), intron 2 (SEQ ID NO:6), intron 3 (SEQ ID NO:8), intron 4 (SEQ ID NO:10), intron 5 (SEQ ID NO:12), intron 6 (SEQ ID NO:14), intron 7 (SEQ ID NO:16), intron 8 (SEQ ID NO:18), intron 9 (SEQ ID NO:20), intron 10 (SEQ ID NO:22), intron 11 (SEQ ID NO:24), intron 12 (SEQ ID NO:26) and intron 13 (SEQ ID NO:28). The NtMNS1b genomic clone of SEQ ID NO:32 with 5′ and 3′ untranslated regions, or SEQ ID NO:33 without 5′ and 3′ untranslated regions, comprises 14 exons and 13 introns: exon 1 (SEQ ID NO:34), exon 2 (SEQ ID NO:36), exon 3 (SEQ ID NO:38), exon 4 (SEQ ID NO:40), exon 5 (SEQ ID NO:42), exon 6 (SEQ ID NO:44), exon 7 (SEQ ID NO:46), exon 8 (SEQ ID NO:48), exon 9 (SEQ ID NO:50), exon 10 (SEQ ID NO:52), exon 11 (SEQ ID NO:54), exon 12 (SEQ ID NO:56), exon 13 (SEQ ID NO:58), exon 14 (SEQ ID NO:60), intron 1 (SEQ ID NO:35), intron 2 (SEQ ID NO:37), intron 3 (SEQ ID NO:39), intron 4 (SEQ ID NO:41), intron 5 (SEQ ID NO:43), intron 6 (SEQ ID NO:45), intron 7 (SEQ ID NO:47), intron 8 (SEQ ID NO:49), intron 9 (SEQ ID NO:51), intron 10 (SEQ ID NO:53), intron 11 (SEQ ID NO:55), intron 12 (SEQ ID NO:57) and intron 13 (SEQ ID NO:59). The NtMNS2 genomic clone of SEQ ID NO:63 with 5′ and 3′ untranslated regions, or SEQ ID NO:64 without 5′ and 3′ untranslated regions, comprises 14 exons and 13 introns: exon 1 (SEQ ID NO:65), exon 2 (SEQ ID NO:67), exon 3 (SEQ ID NO:69), exon 4 (SEQ ID NO:71), exon 5 (SEQ ID NO:73), exon 6 (SEQ ID NO:75), exon 7 (SEQ ID NO:77), exon 8 (SEQ ID NO:79), exon 9 (SEQ ID NO:81), exon 10 (SEQ ID NO:83), exon 11 (SEQ ID NO:85), exon 12 (SEQ ID NO:87), exon 13 (SEQ ID NO:89), exon 14 (SEQ ID NO:91), intron 1 (SEQ ID NO:66), intron 2 (SEQ ID NO:68), intron 3 (SEQ ID NO:70), intron 4 (SEQ ID NO:72), intron 5 (SEQ ID NO:74), intron 6 (SEQ ID NO:76), intron 7 (SEQ ID NO:78), intron 8 (SEQ ID NO:80), intron 9 (SEQ ID NO:82), intron 10 (SEQ ID NO:84), intron 11 (SEQ ID NO:86), intron 12 (SEQ ID NO:88) and intron 13 (SEQ ID NO:90).

Various embodiments are directed to polynucleotides comprising independently the sequences of the NtMNS1a, NtMNS1b and NtMNS2 locus, namely SEQ ID NO:1, SEQ ID NO:2, SEQ ID NO:32, SEQ ID NO:33, SEQ ID NO:63 and SEQ ID NO:64; the sequences of fragments of SEQ ID NO:1, SEQ ID NO:2, SEQ ID NO:32, SEQ ID NO:33, SEQ ID NO:63 or SEQ ID NO:64, or variants thereof, or the sequences of intron or exons of NtMNS1a, NtMNS1b and NtMNS2, including the sequences set forth in SEQ ID Nos:3 to 29, 34 to 60 and 65 to 91.

Various embodiments are directed to polynucleotides comprising the sequences of fragments of SEQ ID NO:1, SEQ ID NO:2, SEQ ID NO:32, SEQ ID NO:33, SEQ ID NO:63 and SEQ ID NO:64, which can each comprises, depending on the size of the individual exon or intron, less than about 5 kb, 4 kb, 3 kb, 2 kb, 1 kb, 0.9 kb, 0.8 kb, 0.7 kb, 0.6 kb, 0.5 kb, 0.4 kb, 0.3 kb, 0.2 kb, or 0.1 kb of nucleotide sequences. In other embodiments, the polynucleotide is about 10-20, 21-50, 51-100, 101-200, 201-400, 401-750, 751-1000; 1001-1250, or 1251-1500 bases in length.

Various embodiments are directed to NtMNS1a, NtMNS1b and NtMNS2 polynucleotide variants comprising at least least 50%, 55%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1, SEQ ID NO:2, SEQ ID NO:32, SEQ ID NO:33, SEQ ID NO:63 or SEQ ID NO:64, or fragments of SEQ ID NO:1, SEQ ID NO:2, SEQ ID NO:32, SEQ ID NO:33, SEQ ID NO:63 or SEQ ID NO:64.

Various embodiments are directed to variants of the exon(s) or intron(s) of NtMNS1a, NtMNS1b or NtMNS2 intron, comprising at least 50%, 55%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of SEQ ID Nos:3 to 29, 34 to 60 or 65 to 91, or fragments thereof. See Table 2 which shows the minimum percentage of sequence identity of the variants of each of SEQ ID NO: 1 to 32, 34 to 63 or 65 to 91.

Various embodiments are directed to polynucleotides having sequences that complement that of NtMNS1a, NtMNS1b or NtMNS2 polynucleotide variants comprising at least 50%, 55%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1, SEQ ID NO:2, SEQ ID NO:32, SEQ ID NO:33, SEQ ID NO:63 or SEQ ID NO:64, or fragments of SEQ ID NO:1, SEQ ID NO:2, SEQ ID NO:32, SEQ ID NO:33, SEQ ID NO:63 or SEQ ID NO:64. Various embodiments are directed to polynucleotides that can specifically hybridize, under moderate to highly stringent conditions, to polynucleotides comprising SEQ ID NO:1, SEQ ID NO:2, SEQ ID NO:32, SEQ ID NO:33, SEQ ID NO:63 and SEQ ID NO:64, or fragments of SEQ ID NO:1, SEQ ID NO:2, SEQ ID NO:32, SEQ ID NO:33, SEQ ID NO:63 and SEQ ID NO:64.

Various embodiments are directed to polynucleotides representing NtMNS1a, NtMNS1b NtMNS2, and NtMan1.4 cDNA sequences, comprising SEQ ID NO:30, SEQ ID NO: 94, SEQ ID NO:61, SEQ ID NO: 96, SEQ ID NO: 92, or SEQ ID NO: 98, fragments of SEQ ID NO:30, SEQ ID NO: 94, SEQ ID NO:61, SEQ ID NO: 96, SEQ ID NO: 92, or SEQ ID NO: 98, or variants thereof.

Various embodiments are directed to polynucleotides representing the NtMNS1a, NtMNS1b and NtMNS2 coding exon sequences, comprising NtMNS1a exon 1 (SEQ ID NO:3), exon 2 (SEQ ID NO:5), exon 3 (SEQ ID NO:7), exon 4 (SEQ ID NO:9), exon 5 (SEQ ID NO:11), exon 6 (SEQ ID NO:13), exon 7 (SEQ ID NO:15), exon 8 (SEQ ID NO:17), exon 9 (SEQ ID NO:19), exon 10 (SEQ ID NO:21), exon 11 (SEQ ID NO:23), exon 12 (SEQ ID NO:25), exon 13 (SEQ ID NO:27), exon 14 (SEQ ID NO:29); NtMNS1b exon 1 (SEQ ID NO:34), exon 2 (SEQ ID NO:36), exon 3 (SEQ ID NO:38), exon 4 (SEQ ID NO:40), exon 5 (SEQ ID NO:42), exon 6 (SEQ ID NO:44), exon 7 (SEQ ID NO:46), exon 8 (SEQ ID NO:48), exon 9 (SEQ ID NO:50), exon 10 (SEQ ID NO:52), exon 11 (SEQ ID NO:54), exon 12 (SEQ ID NO:56), exon 13 (SEQ ID NO:58), exon 14 (SEQ ID NO:60); and NtMNS2 exon 1 (SEQ ID NO:65), exon 2 (SEQ ID NO:67), exon 3 (SEQ ID NO:69), exon 4 (SEQ ID NO:71), exon 5 (SEQ ID NO:73), exon 6 (SEQ ID NO:75), exon 7 (SEQ ID NO:77), exon 8 (SEQ ID NO:79), exon 9 (SEQ ID NO:81), exon 10 (SEQ ID NO:83), exon 11 (SEQ ID NO:85), exon 12 (SEQ ID NO:87), exon 13 (SEQ ID NO:89) and exon 14 (SEQ ID NO:91).

As will be understood by the person skilled in the art, a linear DNA has two possible orientations: the 5′ to 3′ direction and the 3′ to 5′ direction. For example, if a reference sequence is positioned in the 5′ to 3′ direction, and if a second sequence is positioned in the 5′ to 3′ direction within the same polynucleotide, then the reference sequence and the second sequence are orientated in the same direction, or have the same orientation. Typically, a promoter sequence and a gene of interest under the regulation or regulatory control of the given promoter, are positioned in the same orientation. However, with respect to the reference sequence positioned in the 5′ to 3′ direction, if a second sequence is positioned in the 3′ to 5′ direction within the same polynucleotide, then the reference sequence and the second sequence are orientated in anti-sense direction, or have anti-sense orientation. Two sequences having anti-sense orientations with respect to each other can be alternatively described as having the same orientation, if the reference sequence (5′ to 3′ direction) and the reverse complementary sequence of the reference sequence (reference sequence positioned in the 5′ to 3′) are positioned within the same polynucleotide. The sequences set forth herein are shown in the 5′ to 3′ direction.

NtMNS Polypeptides.

NtMNS polypeptides include NtMNS1a, NtMNS1b,NtMNS2 and NtMan1.4 polypeptides and variants produced by introducing any type of alterations such as insertions, deletions, or substitutions of amino acids, changes in glycosylation states, changes that affect refolding or isomerizations, three-dimensional structures, or self-association states, which can be deliberately engineered or naturally. NtMNS1a, NtMNS1b,NtMNS2 and NtMan1.4 polypeptides comprise at least 10, at least 20, at least 30, or at least 40 contiguous amino acids.

Various embodiments are directed to NtMNS1a, NtMNS1b,NtMNS2 and NtMan1.4 polypeptides encoded by a polynucleotide sequence comprising, consisting of consisting essentially of SEQ ID NO:1, SEQ ID NO:2, SEQ ID NO:30, SEQ ID NO:32, SEQ ID NO:33, SEQ ID NO:61, SEQ ID NO:63, SEQ ID NO:64 or SEQ ID NO:92, or SEQ ID NO:94, SEQ ID NO:96 or SEQ ID NO:98, fragments of SEQ ID NO:1, SEQ ID NO:2, SEQ ID NO:30, SEQ ID NO:32, SEQ ID NO:33, SEQ ID NO:61, SEQ ID NO:63, SEQ ID NO:64 or SEQ ID NO:92, or SEQ ID NO:94, SEQ ID NO:96 or SEQ ID NO:98, or variants thereof.

Various embodiments are directed to NtMNS1a, NtMNS1b,NtMNS2 or NtMan1.4 polypeptide variants comprising at least 50%, 55%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:31, SEQ ID NO:62 or SEQ ID NO:93, or SEQ ID NO:95, SEQ ID NO:97 or SEQ ID NO:99, or fragments of SEQ ID NO:31, SEQ ID NO:62 or SEQ ID NO:93, or SEQ ID NO:95, SEQ ID NO:97 or SEQ ID NO:99.

Mutant polypeptide variants of NtMNS1a, NtMNS1b,NtMNS2 and NtMan1.4 are also encompassed by the claims and are disclosed herein.

Zinc Finger Proteins Binding to NtMNS Polynucleotides.

A zinc finger DNA-binding domain or motif consists of approximately 30 amino acids that fold into a beta-beta-alpha (ββα) structure of which the alpha-helix (α-helix) inserts into the DNA double helix. An “alpha-helix” (α-helix) refers to a motif in the secondary structure of a protein that is either right- or left-handed coiled in which the hydrogen of each N—H group of an amino acid is bound to the C═O group of an amino acid at position −4 relative to the first amino acid. A “beta-barrel” (β-barrel) as used herein refers to a motif in the secondary structure of a protein comprising two beta-strands (β-strands) in which the first strand is hydrogen bound to a second strand to form a closed structure. A “beta-beta-alpha” (ββα) structure” as used herein refers to a structure in a protein that consists of a β-barrel comprising two anti-parallel β-strands and one α-helix. The term “zinc finger DNA-binding domain” refers to a protein domain that comprises a zinc ion and is capable of binding to a specific three basepair DNA sequence. The term “non-natural zinc finger DNA-binding domain” refers to a zinc finger DNA-binding domain that does not occur in the cell or organism comprising the DNA which is to be modified.

The key amino acids within a zinc finger DNA-binding domain or motif that bind the three basepair sequence within the target DNA, are amino acids −1, +1, +2, +3, +4, +5 and +6 relative to the beginning of the alpha-helix (α-helix). The amino acids at position −1, +1, +2, +3, +4, +5 and +6 relative to the beginning of the α-helix of a zinc finger DNA-binding domain or motif can be modified while maintaining the beta-barrel (β-barrel) backbone to generate new DNA-binding domains or motifs that bind a different three basepair sequence. Such a new DNA-binding domain can be a non-natural zinc finger DNA-binding domain. In addition to the three basepair sequence recognition by the amino acids at position −1, +1, +2, +3, +4, +5 and +6 relative to the start of the α-helix, some of these amino acids can also interact with a basepair outside the three basepair sequence recognition site. By combining two, three, four, five, six or more zinc finger DNA-binding domains or motifs, a zinc finger protein can be generated that specifically binds to a longer DNA sequence. For example, a zinc finger protein comprising two zinc finger DNA-binding domains or motifs can recognize a specific six basepair sequence and a zinc finger protein comprising four zinc finger DNA-binding domains or motifs can recognize a specific twelve basepair sequence. A zinc finger protein can comprise two or more natural zinc finger DNA-binding domains or motifs or two or more non-natural zinc finger DNA-binding domains or motifs derived from a natural or wild-type zinc finger protein by truncation or expansion or a process of site-directed mutagenesis coupled to a selection method such as, but not limited to, phage display selection, bacterial two-hybrid selection or bacterial one-hybrid selection or any combination of natural and non-natural zinc finger DNA-binding domains. “Truncation” as used within this context refers to a zinc finger protein that contains less than the full number of zinc finger DNA-binding domains or motifs found in the natural zinc finger protein. “Expansion” as used within this context refers to a zinc finger protein that contains more than the full number of zinc finger DNA-binding domains or motifs found in the natural zinc finger protein. Techniques for selecting a polynucleotide sequence within a genomic sequence for zinc finger protein binding are known in the art and can be used in the present invention.

WO98/54311 discloses methods for the design of zinc finger protein domains which bind specific nucleotide sequences which are unique to a target gene. It has been calculated that a sequence comprising 18 nucleotides is sufficient to specify an unique location in the genome of higher organisms. Typically, therefore, zinc finger protein domains contain 6 zinc fingers, each with its specifically designed alpha helix for interaction with a particular triplet. However, in some instances, a shorter or longer nucleotide target sequence may be desirable. Thus, the zinc finger domains in the proteins may contain from 2 to 12 fingers—such as 3 to 8 fingers, 5 to 7 fingers, or 6 fingers.

Methods for designing and identifying a zinc finger protein with the desired nucleic acid binding characteristics also include those described in WO98/53060, which reports a method for preparing a nucleic acid binding protein of the Cys2-His2 zinc finger class capable of binding to a nucleic acid quadruplet in a target nucleic acid sequence.

Zinc finger proteins of use in the present invention may comprise at least one zinc finger polypeptide linked via a linker, preferably a flexible linker, to at least a second DNA binding domain, which optionally is a second zinc finger polypeptide. The zinc finger protein may contain more than two DNA-binding domains, as well as one or more regulator domains. The zinc finger polypeptides may be engineered to recognize a selected target site in the gene of choice.

In one embodiment, the zinc finger protein comprises a framework (or backbone) derived from a naturally occurring zinc finger protein. Framework (or backbone) derived from any naturally occurring zinc finger protein can be used. For example, the zinc finger protein comprising a framework (or backbone) derived from a zinc finger protein comprising a C2H2 motif can be used.

In another specific embodiment, the zinc finger protein comprises a framework (or backbone) derived from a zinc finger protein that is naturally functional in plant cells. For example, the zinc finger protein may comprise a C3H zinc finger, a QALGGH motif, a RING-H2 zinc finger motif, a 9 amino acid C2H2 motif, a zinc finger motif of Arabidopsis LSD1 and a zinc finger motif of BBF/D of domain proteins.

Various embodiments are directed to zinc finger proteins that specifically bind to NtMNS1a, NtMNS1b and NtMNS2 polynucleotides, comprising SEQ ID NO:1, SEQ ID NO:2, SEQ ID NO:32, SEQ ID NO:33, SEQ ID NO:63 and SEQ ID NO:64, fragments of SEQ ID NO:1, SEQ ID NO:2, SEQ ID NO:32, SEQ ID NO:33, SEQ ID NO:63 or SEQ ID NO:64, or variants thereof, to introns and exons of NtMNS1a, NtMNS1b and NtMNS2 comprising SEQ ID Nos:3 to 29, 34 to 60 and 65 to 91, and to combinations of introns and exons of NtMNS1a, NtMNS1b and NtMNS2, comprising SEQ ID Nos:3 to 29, 34 to 60 and 65 to 91. As will be understood by one skilled in the art, combinations of introns and exons in the context of the invention, refers to introns and exons directly linked to each other on the respective genomic polynucleotide, such as for example NtMNS1a exon 3 (SEQ ID NO:7) and intron 3 (SEQ ID NO:8) or NtMNS1a intron 2 (SEQ ID NO:6) and exon 3 (SEQ ID NO:7).

Meganucleases Binding to NtMNS Polynucleotides.

Aspects of the present invention further provide methods for modifying the expression of NtMNS polynucleotides and polypeptides, using a genome engineering or genome editing technology. Thus, in certain embodiments, meganucleases, such as non-natural or recombinant meganucleases, are used to specifically cause a double-stranded break at a single site or at relatively few sites in the genomic DNA coding for a NtMNS polypeptide to allow for the disruption of a NtMNS polynucleotide such as NtMNS1a, NtMNS1b or NtMNS2. The meganuclease may be an engineered meganuclease with altered DNA-recognition properties as described in WO07/047,859 which describes methods for the structure-based engineering of meganucleases derived from the naturally-occurring meganuclease I-CreI. Engineered meganucleases can be made to recognize and cut pre-determined 22 base pair DNA sequences. Meganuclease proteins can be delivered into cells by a variety of different mechanisms known in the art.

Various embodiments are directed to meganucleases that specifically bind to NtMNS1a, NtMNS1b and NtMNS2 polynucleotides, comprising SEQ ID NO:1, SEQ ID NO:2, SEQ ID NO:32, SEQ ID NO:33, SEQ ID NO:63 and SEQ ID NO:64, fragments of SEQ ID NO:1, SEQ ID NO:2, SEQ ID NO:32, SEQ ID NO:33, SEQ ID NO:63 or SEQ ID NO:64, or variants thereof; to introns and exons of NtMNS1a, NtMNS1b and NtMNS2 comprising SEQ ID Nos:3 to 29, 34 to 60 and 65 to 91, and to combinations of introns and exons of NtMNS1a, NtMNS1b and NtMNS2, comprising SEQ ID Nos:3 to 29, 34 to 60 and 65 to 91. As will be understood by one skilled in the art, combinations of introns and exons in the context of the invention, refers to introns and exons directly linked to each other on the respective genomic polynucleotide, such as for example NtMNS1a exon 3 (SEQ ID NO:7) and intron 3 (SEQ ID NO:8) or NtMNS1a intron 2 (SEQ ID NO:6) and exon 3 (SEQ ID NO:7).

Antibodies Binding to NtMNS Polypeptides.

In another embodiment, antibodies that are immunoreactive with NtMNS polypeptides, comprising NtMNS1a, NtMNS1b,NtMNS2 or NtMan1.4 and comprising SEQ ID NO: 31, SEQ ID NO: 95, SEQ ID NO: 62, SEQ ID NO: 97, SEQ ID NO: 93, and SEQ ID NO: 99, are provided herein. The NtMNS polypeptides, fragments, variants, fusion polypeptides, and the like, as set forth herein, can be employed as “immunogens” in producing antibodies immunoreactive therewith. Such antibodies specifically bind to the polypeptides via the antigen-binding sites of the antibody. Specifically binding antibodies are those that will specifically recognize and bind with NtMNS family polypeptides, homologues, and variants, but not with other molecules. In one embodiment, the antibodies are specific for polypeptides having an NtMNS1a, NtMNS1b or NtMNS2 amino acid sequence as set forth herein in SEQ ID NO: 31, SEQ ID NO: 95, SEQ ID NO: 62, SEQ ID NO: 97, SEQ ID NO: 93, and SEQ ID NO: 99, and do not cross-react with other polypeptides. The antibodies can also be used in assays to detect the presence of the NtMNS polypeptides or fragments, either in vitro or in vivo. The antibodies also can be employed in purifying polypeptides or fragments by immunoaffinity chromatography, or for modifying the expression of NtMNS polypeptides.

Transformation.

Transgenic and modified plant cells and plants comprising such cells, are described herein with modified alpha-mannosidase I activity as well as transgenic plant cells and plants with modified alpha-mannosidase I activity comprising one or more recombinant nucleic acids, such as heterologous polynucleotides. The heterologous polynucleotide can be the polynucleotide, a chimeric gene, a nucleic acid construct, a dsRNA, or an expression vector of the present invention. The heterologous polynucleotide can also be a construct coding for a heterologous protein for expression in a modified plant cell or plant according to the invention, for the manufacture of a pharmaceutical composition according to the invention.

A plant or plant cell can be transformed by having the recombinant nucleic acid integrated into its genome to become stably transformed. Stably transformed cells typically retain the introduced nucleic acid with each cell division. A plant or plant cell may also be transiently transformed such that the recombinant nucleic acid is not integrated into its genome. Transiently transformed cells typically lose all or some portion of the introduced recombinant nucleic acid with each cell division such that the introduced recombinant nucleic acid cannot be detected in daughter cells after a sufficient number of cell divisions.

Techniques for introducing nucleic acids into monocotyledonous and dicotyledonous plants and plant cells, are known in the art, and include, for example, Agrobacterium-mediated transformation and infiltration, viral vector-mediated transformation, electroporation and particle gun transformation. For example, U.S. Pat. No. 4,459,355 discloses a method for transforming susceptible plants, including dicots, with an Agrobacterium strain containing a Ti plasmid; U.S. Pat. No. 4,795,855 discloses transformation of woody plants with an Agrobacterium vector; U.S. Pat. No. 4,940,838 discloses a binary Agrobacterium vector; U.S. Pat. No. 4,945,050; and U.S. Pat. No. 5,015,580. If a cell or cultured tissue is used as the recipient tissue for transformation, the transformed cultured cells can be cultivated or transformed plant cells can be regenerated from transformed cultures or tissue, if desired, by techniques known to those skilled in the art. For the manufacture of pharmaceutical compositions comprising a heterologous protein or glycoprotein in plant cells, the heterologous polynucleotide or gene sequence coding for the protein, is placed under control of regulatory elements that are functional in the plant cell in a gene construct or transformation vector.

Regulatory Elements.

The choice of regulatory elements to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner. Some suitable regulatory regions initiate transcription only, or predominantly, in certain cell types.

Promoters.

Suitable promoters include tissue-specific promoters recognized by tissue-specific factors present in different tissues or cell types such as for example root-specific promoters, shoot-specific promoters, xylem-specific promoters, leaf specific promoters, or present during different developmental stages, or present in response to different environmental conditions. Suitable promoters include constitutive promoters that can be activated in most cell types without requiring specific inducers. Examples of suitable promoters for controlling NtNMS1a, NtMNS1b, NtMNS2, and NtMan1.4RNAi polynucleotide production, include the cauliflower mosaic virus 35S promoter, the Rubisco small subunit promoter, octopine synthase promoter, nopaline synthase promoter, or ubiquitin- or phaseolin-promoters. Persons skilled in the art are capable of generating multiple variations of recombinant promoters.

RNAi Expression Vectors Comprising NtMNS Constructs.

RNA Interference (“RNAi”) or RNA silencing is an evolutionarily conserved process by which specific mRNAs can be targeted for enzymatic degradation. A double-stranded RNA (dsRNA) must be introduced or produced by a cell for example by a dsRNA virus, or NtMNS RNAi polynucleotides, to initiate the RNAi pathway. The dsRNA can be converted into multiple siRNA duplexes of 21-23 bp length (“siRNAs”) by Rnases III, which are dsRNA-specific endonucleases. The siRNAs can be subsequently recognized by RNA-induced silencing complexes that promote the unwinding of siRNA through an ATP-dependent process. The unwound antisense strand of the siRNA guides the activated RNA-induced silencing complex to the targeted mRNA which can be NtNMS1a, NtMNS1b, NtMNS2, or NtMan1.4 RNA variants comprising a sequence complementary to the siRNA anti-sense strand.

NtNMS1a, NtMNS1b, NtMNS2, and NtMan1.4RNAi expression vectors comprising NtNMS1a, NtMNS1b, NtMNS2, or NtMan1.4 RNAi constructs encoding NtNMS1a, NtMNS1b, NtMNS2, or NtMan1.4RNAi polynucleotides, exhibit RNA interference activity by reducing the expression level of NtNMS1a, NtMNS1b, NtMNS2, and NtMan1.4 mRNAs; NtNMS1a, NtMNS1b, NtMNS2, or NtMan1.4 pre-mRNAs; or related NtNMS1a, NtMNS1b, NtMNS2, or NtMan1.4RNA variants. The expression vectors may comprise a promoter positioned upstream and operably-linked to a NtMNS RNAi construct, as further described herein. NtMNS RNAi expression vectors may comprise a suitable minimal core promoter, a NtMNS RNAi construct of interest, an upstream (5′) regulatory region, a downstream (3′) regulatory region, including transcription termination and polyadenylation signals, and other sequences known to persons skilled in the art, such as various selection markers.

In one embodiment, target NtNMS1a, NtMNS1b, NtMNS2, or NtMan1.4mRNA sequences are selected that are between about 14 and about 30 nucleotides in length that meet one or more of the above criteria. In another embodiment, target sequences are selected that are between about 16 and about 30 nucleotides in length that meet one or more of the above criteria. In a further embodiment, target sequences are selected that are between about 19 and about 30 nucleotides in length that meet one or more of the above criteria. In another embodiment, target sequences are selected that are between about 19 and about 25 nucleotides in length that meet one or more of the above criteria.

In an exemplary embodiment, the siRNA molecules comprise a specific antisense sequence that is complementary to at least 12, 13, 14, 15, 16, 17, 18, 19, 20, 21, 22, 23, 24, 25, 26, 27, 28, 29, 30, or more contiguous nucleotides of any one of the sequences as set forth in SEQ ID NO:30, SEQ ID NO: 94, SEQ ID NO:61, SEQ ID NO: 96, SEQ ID NO: 92, or SEQ ID NO: 98.

The specific antisense sequence comprised by the siRNA molecule can be identical or substantially identical to the complement of the target sequence. In one embodiment of the present invention, the specific antisense sequence comprised by the siRNA molecule is at least about 50%, 55%, 60%, 70%, 71%, 72%, 73%, 74%, but particularly at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the complement of the target mRNA sequence. Methods of determining sequence identity are known in the art and can be determined, for example, by using the BLASTN program of the University of Wisconsin Computer Group (GCG) software or provided on the NCBI website.

Expression Vectors for Reducing NtMNS Gene Expression by Co-Suppression.

Various compositions and methods are provided for modulating, including reducing, the endogenous expression levels for NtNMS1a, NtMNS1b, NtMNS2, and NtMan1.4genes by promoting co-suppression of NtNMS1a, NtMNS1b, NtMNS2, or NtMan1.4gene expression. The phenomenon of co-suppression occurs as a result of introducing multiple copies of a transgene into a plant cell host. Integration of multiple copies of a transgene can result in reduced expression of the transgene and the targeted endogenous gene. The degree of co-suppression is dependent on the degree of sequence identity between the transgene and the targeted endogenous gene. The silencing of both the endogenous gene and the transgene can occur by extensive methylation of the silenced loci, the endogenous promoter and endogenous gene of interest, that can preclude transcription. Alternatively, in some cases, co-suppression of the endogenous gene and the transgene can occur by post transcriptional gene silencing (“PTGS”), in which transcripts can be produced but enhanced rates of degradation preclude accumulation of transcripts. The mechanism for co-suppression by PTGS is thought to resemble RNA interference, in that RNA seems to be both an important initiator and a target in these processes, and may be mediated at least in part by the same molecular machinery, possibly through RNA-guided degradation of mRNAs.

Co-suppression of NtNMS1a, NtMNS1b, NtMNS2, or NtMan1.4 can be achieved by integrating multiple copies of the NtNMS1a, NtMNS1b, NtMNS2, or NtMan1.4 cDNA of SEQ ID NO:30, SEQ ID NO: 94, SEQ ID NO:61, SEQ ID NO: 96, SEQ ID NO: 92, or SEQ ID NO: 98, or fragments thereof, as transgenes, into the genome of a plant of interest. The host plant can be transformed with an expression vector comprising a promoter operably-linked to the NtNMS1a, NtMNS1b, NtMNS2, or NtMan1.4 cDNA or fragments thereof. Various embodiments are directed to expression vectors for promoting co-suppression of endogenous NtMNS genes comprising: a promoter operably linked to NtNMS1a, NtMNS1b, NtMNS2, or NtMan1.4, for example cDNA identified as SEQ ID NO:30, SEQ ID NO: 94, SEQ ID NO:61, SEQ ID NO: 96, SEQ ID NO: 92, or SEQ ID NO: 98, or a fragment thereof, such as any of SEQ ID Nos: 5, 7, 9, 11, 13, 15, 17, 19, 21, 23, 25, 27, 29, 34, 36, 38, 40, 42, 44, 46, 48, 50, 52, 54, 56, 58, 60, 65, 67, 69, 71, 73, 75, 77, 79, 81, 83, 85, 87, 89 or 91, or a variant thereof having at least about 50%, 55%, 60%, 70%, 71%, 72%, 73%, but particularly at least 74%, 75%, 80%, 85%, 90%, 95%, 96%, 97%, 98%, or 99% sequence identity thereto.

Various embodiments are directed to methods for modulating, reducing or inhibiting, the expression level of NtNMS1a, NtMNS1b, NtMNS2, and NtMan1.4 by integrating multiple copies of NtMNS1a, NtMNS1b or NtMNS2 identified as SEQ ID NO:30, SEQ ID NO: 94, SEQ ID NO:61, SEQ ID NO: 96, SEQ ID NO: 92, or SEQ ID NO: 98, or a fragment thereof, or a variant thereof having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto into a plant genome, comprising: transforming a plant cell host with an expression vector that comprises a promoter operably-linked to SEQ ID NO:30, SEQ ID NO: 94, SEQ ID NO:61, SEQ ID NO: 96, SEQ ID NO: 92, or SEQ ID NO: 98, or a fragment thereof, or a variant thereof having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

Expression Vectors for Reducing NtMNS Expression by Inhibition of Translation by Anti-Sense Agents.

Various compositions and methods are provided for reducing the endogenous expression level of NtNMS1a, NtMNS1b, NtMNS2, and NtMan1.4 by inhibiting the translation of NtNMS1a, NtMNS1b, NtMNS2, or NtMan1.4 mRNA. A host plant cell can be transformed with an expression vector comprising: a promoter operably-linked to NtNMS1a, NtMNS1b, NtMNS2, or NtMan1.4, or a variant or fragment thereof, positioned in anti-sense orientation with respect to the promoter to enable the expression of RNA polynucleotides having a sequence complementary to a portion of NtMNS1a NtNMS1a, NtMNS1b, NtMNS2, or NtMan1.4 m RNA.

Various expression vectors for inhibiting the translation of NtNMS1a, NtMNS1b, NtMNS2, or NtMan1.4 mRNA may comprise: a promoter operably-linked to NtNMS1a, NtMNS1b, NtMNS2, or NtMan1.4, identified as SEQ SEQ ID NO:30, SEQ ID NO: 94, SEQ ID NO:61, SEQ ID NO: 96, SEQ ID NO: 92, or SEQ ID NO: 98, or a fragment thereof, or a variant thereof having at least 50%, 55%, 60%, 70%, 71%, 72%, 73%, but particularly at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto in which the sequence is positioned in anti-sense orientation with respect to the promoter. The lengths of anti-sense NtNMS1a, NtMNS1b, NtMNS2, and NtMan1.4 RNA polynucleotides can vary, and may be from about 15-20 nucleotides, about 20-30 nucleotides, about 30-50 nucleotides, about 50-75 nucleotides, about 75-100 nucleotides, about 100-150 nucleotides, about 150-200 nucleotides, and about 200-300 nucleotides.

Other Compositions and Methods for Reducing NtMNS Expression.

Methods for obtaining conservative variants and more divergent variants of NtNMS1a, NtMNS1b, NtMNS2, and NtMan1.4 polynucleotides and polypeptides are known to persons skilled in the art. Any plant of interest can be genetically modified by various methods known to induce mutagenesis, including site-directed mutagenesis, oligonucleotide-directed mutagenesis, chemically-induced mutagenesis such as ethylmethane sulphonate, irradiation-induced mutagenesis, and other equivalent methods. Alternatively, NtNMS1a, NtMNS1b, NtMNS2, and NtMan1.4 genes can be targeted for inactivation by a method referred to as Targeting Induced Local Lesions IN Genomics (“TILLING”), which combines high-density point mutations with rapid sensitive detection of mutations. Typically, plant seeds are exposed to mutagens, such as ethylmethane sulphonate (EMS) or EMS alkylates guanine, which typically leads to mispairing. Suitable agents and methods are known to persons skilled in the art as described in McCallum et al., (2000), “Targeting Induced Local Lesions IN Genomics (TILLING) for Plant Functional Genomics,” Plant Physiology 123:439-442; McCallum et al., (2000) “Targeted screening for induced mutations,” Nature Biotechnology 18:455-457; and Colbert et al., (2001) “High-Throughput Screening for Induced Point Mutations,” Plant Physiology 126:480-484. Mutagens that create primarily point mutations and short deletions, insertions, transversions, transitions, including chemical mutagens or radiation, or all may be used to create the mutations. Mutagens include, but are not limited to, ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloro-ethyl)aminopropylamino]acridine dihydrochloride (ICR-170), and formaldehyde.

Mutagenesis of NtMNS Polynucleotides.

A pair of zinc fingers binding to an NtMNS polynucleotide of the present invention, can be used to make zinc-finger nuclease for modifying a NtMNS polynucleotide. The general use of zinc finger nuclease-mediated mutagenesis is known in the art and is described in, for example, WO02/057293, WO02/057294, WO00/041566, WO00/042219, and WO05/084190.

It is contemplated that a method for mutating a gene sequence, such as a genomic DNA sequence that encodes NtNMS1a, NtMNS1b, NtMNS2, or NtMan1.4, by zinc finger nuclease-mediated mutagenesis comprises optionally one or more of the following steps: (i) providing at least two zinc finger proteins that selectively bind different target sites in the gene sequence; (ii) constructing two expression constructs each encoding a different zinc finger nuclease that comprises one of the two different non-natural zinc finger proteins of step (i) and a nuclease, operably linked to expression control sequences operable in a plant cell; (iii) introducing the two expression constructs into a plant cell wherein the two different zinc finger nucleases are produced, such that a double stranded break is introduced in the genomic DNA sequence in the genome of the plant cell, at or near to at least one of the target sites. The introduction of the two expression constructs into the plant cell can be accomplished simultaneously or sequentially, optionally including selection of cells that took up the first construct.

A double stranded break (DSB) as used herein, refers to a break in both strands of the DNA or RNA. The double stranded break can occur on the genomic DNA sequence at a site that is not more than between 5 base pairs and 1500 base pairs, particularly not more than between 5 base pairs and 200 base pairs, particularly not more than between 5 base pairs and 20 base pairs removed from one of the target sites. The double stranded break can facilitate non-homologous end joining leading to a mutation in the genomic DNA sequence at or near the target site. “Non homologous end joining (NHEJ)” as used herein refers to a repair mechanism that repairs a double stranded break by direct ligation without the need for a homologous template, and can thus be mutagenic relative to the sequence before the double stranded break occurs.

The method can optionally further comprise the step of (iv) introducing into the plant cell a polynucleotide comprising at least a first region of homology to a nucleotide sequence upstream of the double-stranded break and a second region of homology to a nucleotide sequence downstream of the double-stranded break. The polynucleotide can comprise a nucleotide sequence that corresponds to the NtNMS1a, NtMNS1b, NtMNS2, or NtMan1.4 sequence that contains a deletion or an insertion of heterologous nucleotide sequences. The polynucleotide can thus facilitate homologous recombination at or near the target site resulting in the insertion of heterologous sequence into the genome or deletion of genomic DNA sequence from the genome. The resulting genomic DNA sequence in the plant cell can comprise a mutation that disrupts the enzyme activity of an expressed mutant NtNMS1a, NtMNS1b, NtMNS2, or NtMan1.4, an early translation stop codon, or a sequence motif that interferes with the proper processing of pre-mRNA into an mRNA resulting in reduced expression or inactivation of the gene. Methods to disrupt protein synthesis by mutating a gene sequence coding for a protein are known to those skilled in the art.

A zinc finger nuclease may be constructed by making a fusion of a first polynucleotide coding for a zinc finger protein that binds to NtNMS1a, NtMNS1b, NtMNS2, or NtMan1.4, and a second polynucleotide coding for a non-specific endonuclease such as, but not limited to, those of a Type IIS endonuclease. A Type IIS endonuclease is a restriction enzyme having a separate recognition domain and an endonuclease cleavage domain wherein the enzyme cleaves DNA at sites that are removed from the recognition site. Non-limiting examples of Type IIS endonucleases can be, but not limited to, AarI, BaeI, CdiI, DrdII, EciI, FokI, FauI, GdiII, HgaI, Ksp632I, MboII, Pfl1108I, Rle108I, RleAI, SapI, TspDTI or UbaPI. Methods for the design and construction of fusion proteins, methods for the selection and separation of the endonuclease domain from the sequence recognition domain of a Type IIS endonuclease, methods for the design and construction of a zinc finger nuclease comprising a fusion protein of a zinc finger protein and an endonuclease, are known in the art. In a specific embodiment, the nuclease domain in a zinc finger nuclease is FokI. A fusion protein between a zinc finger protein and the nuclease of FokI may comprise a spacer consisting of two basepairs or alternatively, the spacer can consist of three, four, five, six or more basepairs. In one embodiment, there is described a fusion protein with a seven basepair spacer such that the endonuclease of a first zinc finger nuclease can dimerize upon contacting a second zinc finger nuclease, wherein the two zinc finger proteins making up said zinc finger nucleases can bind upstream and downstream of the target DNA sequence. Upon dimerization, a zinc finger nuclease can introduce a double stranded break in a target nucleotide sequence which may be followed by non-homologous end joining or homologous recombination with an exogenous nucleotide sequence having homology to the regions flanking both sides of the double stranded break.

In yet another embodiment, there is provided a fusion protein comprising a zinc finger protein and an enhancer protein resulting in a zinc finger activator. A zinc finger activator can be used to up-regulate or activate transcription of NtNMS1a, NtMNS1b, NtMNS2, or NtMan1.4, comprising the steps of (i) engineering a zinc finger protein that binds a region within a promoter or a sequence operatively linked to a coding sequence of NtNMS1a, NtMNS1b, NtMNS2, or NtMan1.4, (ii) making a fusion protein between said zinc finger protein and a transcription activator, (iii) making an expression construct comprising a polynucleotide sequence coding for said zinc finger activator under control of a promoter active in a cell, such as plant cell, (iv) introducing said gene construct into the cell, and (v) culturing the cell and allowing the expression of the zinc finger activator, and (vi) characterizing the cell having an increased expression of NtNMS1a, NtMNS1b, NtMNS2, or NtMan1.4.

In yet another embodiment, the invention provides a fusion protein comprising a zinc finger protein and a gene repressor resulting in a zinc finger repressor. A zinc finger repressor can be used to down-regulate or repress the transcription of NtNMS1a, NtMNS1b, NtMNS2, or NtMan1.4, comprising the steps of (i) engineering a zinc finger protein that binds to a region within a promoter or a sequence operatively linked to NtNMS1a, NtMNS1b, NtMNS2, or NtMan1.4, and (ii) making a fusion protein between said zinc finger protein and a transcription repressor, and (iii) developing a gene construct comprising a polynucleotide sequence coding for said zinc finger repressor under control of a promoter active in a cell, such as a plant cell, and (iv) introducing said gene construct into the cell, and (v) providing for the expression of the zinc finger repressor, and (vi) characterizing the cell having reduced transcription of NtNMS1a, NtMNS1b, NtMNS2, or NtMan1.4.

In yet another embodiment, the invention provides a fusion protein comprising a zinc finger protein and a methylase resulting in a zinc finger methylase. The zinc finger methylase may be used to down-regulate or inhibit the expression of NtNMS1a, NtMNS1b, NtMNS2, or NtMan1.4 in a cell, such as plant cell, by methylating a region within the promoter region of NtNMS1a, NtMNS1b, NtMNS2, or NtMan1.4, comprising the steps of (i) engineering a zinc finger protein that can binds to a region within a promoter of NtNMS1a, NtMNS1b, NtMNS2, or NtMan1.4 as present upstream of the coding sequences in SEQ ID NO:1, SEQ ID NO:32 or SEQ ID NO:63, and (ii) making a fusion protein between said zinc finger protein and a methylase, and (iii) developing a gene construct containing a polynucleotide coding for said zinc finger methylase under control of a promoter active in the cell, and (iv) introducing said gene construct into the cell, and (v) allowing the expression of the zinc finger methylase, and (vi) characterizing the cell having reduced or essentially no expression of NtNMS1a, NtMNS1b, NtMNS2, or NtMan1.4 in the cell.

In various embodiments of the invention, a zinc finger protein may be selected according to methods of the present invention to bind to a regulatory sequence of NtMNS1a, NtMNS1b or NtMNS2. More specifically, the regulatory sequence may comprise a transcription initiation site, a start codon, a region of an exon, a boundary of an exon-intron, a terminator, or a stop codon. The zinc finger protein can be fused to a nuclease, an activator, or a repressor protein.

In various embodiments of the invention, a zinc finger nuclease introduces a double stranded break in a regulatory region, a coding region, or a non-coding region of a genomic DNA sequence of NtNMS1a, NtMNS1b, NtMNS2, or NtMan1.4, and leads to a reduction, an inhibition or a substantial inhibition of the level of expression of NtNMS1a, NtMNS1b, NtMNS2, or NtMan1.4, or a reduction, an inhibition or a substantial inhibition of the alpha-mannosidase I or mannose hydrolyzing activity of the protein encoded thereby.

The invention also provides a method for modifying a cell, such as a plant cell, wherein the genome of the plant cell is modified by zinc finger nuclease-mediated mutagenesis, comprising (a) identifying and making at least two non-natural zinc finger proteins that selectively bind different target sites for modification in the genomic nucleotide sequence; (b) expressing at least two fusion proteins each comprising a nuclease and one of the at least two non-natural zinc finger proteins in the plant cell, such that a double stranded break is introduced in the genomic nucleotide sequence in the plant genome, particularly at or close to a target site in the genomic nucleotide sequence; and, optionally (c) introducing into the cell a polynucleotide comprising a nucleotide sequence that comprises a first region of homology to a sequence upstream of the double-stranded break and a second region of homology to a region downstream of the double-stranded break, such that the polynucleotide recombines with DNA in the genome. Also described, are cells comprising one or more expression constructs that comprise nucleotide sequences that encode one or more of the fusion proteins. The general use of meganuclease-mediated mutagenesis is known in the art and described in patent publications, such as WO96/14408, WO03/025183, WO03/078619, WO04/067736, WO07/047,859 and WO09/059,195. In certain embodiments, meganucleases, such as recombinant meganucleases, are used to specifically cause a double-stranded break at a single site or at relatively few sites in the genomic DNA of a plant to allow for the disruption of NtNMS1a, NtMNS1b, NtMNS2, or NtMan1.4. The meganuclease may be an engineered meganuclease with altered DNA-recognition properties as described in WO07/047,859 describing methods for the structure-based engineering of meganucleases derived from the naturally-occurring meganuclease I-CreI.

A zinc finger nuclease or meganuclease protein or a pair of zinc finger proteins, can be provided to a plant cell via any suitable methods known in the art. For example, a zinc finger nuclease can be exogenously added to the plant cell and the plant cell is maintained under conditions such that the zinc finger protein of the zinc finger nuclease binds to the target nucleotide sequence, and modifies the target gene through the activity of the nuclease. Alternatively, a nucleotide sequence encoding a zinc finger protein can be expressed in a plant cell and the plant cell is maintained under conditions such that the expressed zinc finger protein binds to the target nucleotide sequence and regulates the expression of the target gene in the plant cell. A zinc finger nuclease may be expressed in a plant using any suitable plant expression vector. Typical vectors useful for expression of genes in higher plants are well known in the art.

Compositions and Methods for Modulating NtMNS Alpha-Mannosidase I Activity.

Embodiments of the present invention are directed to compositions and methods for producing non-natural or transgenic plants that have been modified to reduce or increase alpha-mannosidase I activity by reducing or increasing the activity of the protein encoded thereby, or the transcription of the genes coding for such proteins. The steady-state level of NtNMS1a, NtMNS1b, NtMNS2, or NtMan1.4RNA transcripts can be decreased or increased as compared to a control plant. Consequently, the number of functionally active NtNMS1a, NtMNS1b, NtMNS2, or NtMan1.4alpha-mannosidase I enzymes available for hydrolyzing mannoses of N-glycans of glycoproteins can be decreased or increased such that the level of mannoses on an N-glycan of a glycoprotein in the plant cell is increased or decreased.

The reduction in expression of NtMNS1aNtNMS1a, NtMNS1b, NtMNS2, or NtMan1.4 may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or up to 100%, which includes a reduction in transcriptional activity or protein expression.

The reduction in the activity of NtNMS1a, NtMNS1b, NtMNS2, or NtMan1.4polypeptide may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or up to 100%.

The increase in expression of NtMNS1a, NtMNS1b or NtMNS2 may be from about 10% to about 1000%, or an increase of at least 10%, at least 20%, at least 25%, at least 50%, at least 100%, at least 200%, at least 500%, at least 750% or up to 1000%, which includes an increase in transcriptional activity or protein expression.

The increase in the activity of NtNMS1a, NtMNS1b, NtMNS2, or NtMan1.4polypeptide may be from about 10% to about 1000%, or an increase of at least 10%, at least 20%, at least 25%, at least 50%, at least 100%, at least 200%, at least 500%, at least 750% or up to 1000%.

Inhibition refers to a reduction of from about 98% to about 100%, or a reduction of at least 98%, at least 99%, but particularly of 100%.

Constructs and Vectors.

Recombinant constructs provided herein can be used to transform plants or plant cells in order to express polynucleotides of the present invention. A recombinant nucleic acid construct can comprise a nucleic acid encoding a heterologous protein as described herein, operably linked to a regulatory region suitable for expressing the heterolous polypeptide in the plant or cell. Vectors containing recombinant nucleic acid constructs such as those described herein also are provided. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available.

The vectors can also include, for example, origins of replication, scaffold attachment regions (SARs) or markers. A marker gene can confer a selectable phenotype on a plant cell. For example, a marker can confer biocide resistance, such as resistance to an antibiotic (for example, kanamycin, G418, bleomycin, or hygromycin), or an herbicide (for example, glyphosate, chlorsulfuron or phosphinothricin). In addition, an expression vector can include a tag sequence designed to facilitate manipulation or detection (for example, purification or localization) of the expressed polypeptide. Tag sequences, such as luciferase, .beta.-glucuronidase (GUS), green fluorescent protein (GFP), glutathione S-transferase (GST), polyhistidine, c-myc or hemagglutinin sequences typically are expressed as a fusion with the encoded polypeptide. Such tags can be inserted anywhere within the polypeptide, including at either the carboxyl or amino terminus.

Transgenic or Non-Natural Plant Cells and Plants with Modified Alpha-Mannosidase I Activity.

Various embodiments are directed to transgenic and non-naturally occurring plants that are modified with respect to alpha-mannosidase I activity by various methods that can utilized for reducing or silencing NtMNS gene expression, and thereby, producing plants in which the expression level of NtMNS alpha-mannosidase I enzymes can be reduced within plant tissues of interest. Other embodiments are directed to plant cells and plants that are modified by various methods that can be utilized for increasing NtMNS expression resulting in increased levels of alpha-mannosidase I activity.

Plants suitable for genetic modification include monocotyledonous and dicotyledonous plants and plant cell systems, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae. Suitable species may include members of the genera Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Suitable species may include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (triticum-wheat.times.rye), bamboo, Helianthus annuus (sunflower), Carthamus tinctorius (safflower), Jatropha curcas (jatropha), Ricinus communis (castor), Elaeis guineensis (palm), Linum usitatissimum (flax), Brassica juncea, Beta vulgaris (sugarbeet), Manihot esculenta (cassava), Lycopersicon esculentum (tomato), Lactuca sativa (lettuce), Musa paradisiaca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Rosa spp. (rose), Dianthus caryophyllus (carnation), Petunia spp. (petunia), Poinsettia pulcherrima (poinsettia), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy), Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

Various embodiments are directed to transgenic and non-naturally occurring tobacco plants with modified NtNMS1a, NtMNS1b, NtMNS2, or NtMan1.4 gene expression level by various methods, and thereby, producing plants, such as tobacco plants, in which the expression level of NtNMS1a, NtMNS1b, NtMNS2, or NtMan1.4 alpha-mannosidase I enzymes can be reduced within plant tissues of interest or increased. The disclosed compositions and methods can be applied to any plant species of interest, including plants of the genus Nicotiana, various species of Nicotiana, including N. rustica and N. tabacum (for example LA B21, LN KY171, TI 1406, Basma, Galpao, Perique, Beinhart 1000-1, Petico, Delfield, Ottawa, Coker 48, Labu, Delhi, TI 115, Yellow Mammoth, Havana 307, Burley 1, Xanthi, Delgold, TI 90, Green Briar, TI 161, Kentucky 16, Maryland 201, Havana 38, Duquesne, Burley 49, CT 681, 81V9 MS, TI 170, Judy's Pride, TI 164, CT 572, TI 158, Kentucky 10, Cannelle, Bell C, Coker 371 Gold, Samsun, Turkish Samsun, Samsun NN, TI 94, Bell B, CT 157, TI 75, White Mammoth, Vinica, Kelly, Grande Rouge, Gold Dollar, Belgique 3007, White Gold, Hicks Broadleaf, Little Crittenden, Bonanza, Havana 425). Other species include N. acaulis, N. acuminata, N. acuminata var. multiflora, N. africana, N. alata, N. amplexicaulis, N. arentsii, N. attenuata, N. benavidesii, N. benthamiana, N. bigelovii, N. bonariensis, N. cavicola, N. clevelandii, N. cordifolia, N. corymbosa, N. debneyi, N. excelsior, N. forgetiana, N. fragrans, N. glauca, N. glutinosa, N. goodspeedii, N. gossei, N. hybrid, N. ingulba, N. kawakamii, N. knightiana, N. langsdorffii, N. linearis, N. longiflora, N. maritima, N. megalosiphon, N. miersii, N. noctiflora, N. nudicaulis, N. obtusifolia, N. occidentalis, N. occidentalis subsp. Hesperis, N. otophora, N. paniculata, N. pauciflora, N. petunioides, N. plumbaginifolia, N. quadrivalvis, N. raimondii, N. repanda, N. rosulata, N. rosulata subsp. Ingulba, N. rotundifolia, N. setchellii, N. simulans, N. solanifolia, N. spegazzinii, N. stocktonii, N. suaveolens, N. sylvestris, N. thyrsiflora, N. tomentosa, N. tomentosiformis, N. trigonophylla, N. umbratica, N. undulata, N. velutina, N. wigandioides, and N. x sanderae. The use of cultivars and elite cultivars is also contemplated herein.

Non-limiting examples of Nicotiana tabacum varieties, breeding lines, and cultivars that can be modified by the methods of the invention include N. tabacum accession PM016, PM021, PM92, PM102, PM132, PM204, PM205, PM215, PM216 or PM217 as deposited with NCIMB, Aberdeen, Scotland, or DAC Mata Fina, PO2, BY-64, AS44, RG17, RG8, HB04P, Basma Xanthi BX 2A, Coker 319, Hicks, McNair 944 (MN 944), Burley 21, K149, Yaka JB 125/3, Kasturi Mawar, NC 297, Coker 371 Gold, PO2, Wisliça, Simmaba, Turkish Samsun, AA37-1, B13P, F4 from the cross BU21 x Hoja Parado line 97, Samsun NN, Izmir, Xanthi NN, Karabalgar, Denizli and PO1.

Mutation Stacking.

Various embodiments are directed to transgenic and non-naturally occurring plants with modified NtNMS1a, NtMNS1b, NtMNS2, or NtMan1.4gene expression levels, and also modified to modulate the expression of (i) NtMNS1a and NtMNS1b or of (ii) NtMNS1a and NtMNS2, or of (iii) NtMNS1a and NtMan1.4, or of (iv) NtMNS1b and NtMNS2, or of (v) NtMNS1b and NtMan1.4, or of (vi) NtMNS2 and NtMan1.4 or of (vii) NtMNS1a and NtMNS1b and NtMNS2, or of (viii) NtMNS1a and NtMNS2 and NtMan1.4, or of (ix) NtMNS1a and NtMNS1b and NtMan1.4, or of (x) NtMNS1b and NtMNS2 and NtMan1.4; or of (xi) NtMNS1a and NtMNS1b and NtMNS2 and NtMan1.4; or more further endogenous genes of interest. Without limitation, examples of other modifications include plants that produce proteins that have favourable immunogenic properties for use in humans. For example, plants capable of producing proteins which substantially lack alpha-1,3-linked fucose residues and beta-1,2-linked xylose residues, on its N-glycans may be of use.

### Plant Breeding.

According to the invention, a tobacco plant carrying a mutant allele of NtMNS1a, NTMNS1b, NtMNS2, or NtMNS1.4 (or any of the combinations thereof as described herein in the various embodiments) can be used in a plant breeding program to create useful lines, varieties and hybrids. In particular, the mutant allele is introgressed into the varieties described above. Thus, methods for breeding plants are provided, that comprise crossing a mutant plant, a non-naturally occurring plant or a transgenic plant as described herein with a plant comprising a different genetic identity. The method may further comprises crossing the progeny plant with another plant, and optionally repeating the crossing until a progeny with the desirable genetic traits or genetic background is obtained. One purpose served by such breeding methods is to introduce a desirable genetic trait into other varieties, breeding lines, hybrids or cultivars, particularly those that are of commercial interest, such as those already containing an expressible polynucleotide encoding a heterologous protein. Another purpose is to facilitate stacking of genetic modifications of different genes in a single plant variety, lines, hybrids or cultivars. Intraspecific as well as interspecific matings are contemplated. The progeny plants that arise from such crosses, also referred to as breeding lines, are examples of non-naturally occurring plants of the invention.

In one embodiment, a method is provided for producing a non-naturally occurring tobacco plant comprising: (a) crossing a mutant or transgenic tobacco plant with a second tobacco plant to yield progeny tobacco seed; (b) growing the progeny tobacco seed, under plant growth conditions, to yield the non-naturally occurring tobacco plant. The method may further comprises: (c) crossing the previous generation of non-naturally occurring tobacco plant with itself or another tobacco plant to yield progeny tobacco seed; (d) growing the progeny tobacco seed of step (c) under plant growth conditions, to yield additional non-naturally occurring tobacco plants; and (e) repeating the crossing and growing steps of (c) and (d) multiple times to generate further generations of non-naturally occurring tobacco plants. The method may optionally comprises prior to step (a), a step of providing a parent plant which comprises a genetic identity that is characterized and that is not identical to the mutant or transgenic plant. In some embodiments, depending on the breeding program, the crossing and growing steps are repeated from 0 to 2 times, from 0 to 3 times, from 0 to 4 times, 0 to 5 times, from 0 to 6 times, from 0 to 7 times, from 0 to 8 times, from 0 to 9 times or from 0 to 10 times, in order to generate generations of non-naturally occurring tobacco plants. Backcrossing is an example of such a method wherein a progeny is crossed with one of its parents or another plant genetically similar to its parent, in order to obtain a progeny plant in the next generation that has a genetic identity which is closer to that of one of the parents. Techniques for plant breeding, particularly tobacco plant breeding, are well known and can be used in the methods of the invention. The invention further provides non-naturally occurring tobacco plants produced by these methods.

In some embodiments of the methods described herein, lines resulting from breeding and screening for variant genes are evaluated in the field using standard field procedures. Control genotypes including the original unmutagenized parent are included and entries are arranged in the field in a randomized complete block design or other appropriate field design. Statistical analyses of the data are performed to confirm the similarity of the selected lines to the parental line. Cytogenetic analyses of the selected plants are optionally performed to confirm the chromosome complement and chromosome pairing relationships.

DNA fingerprinting, single nucleotide polymorphism, microsatellite markers, or similar technologies may be used in a marker-assisted selection (MAS) breeding program to transfer or breed mutant alleles of a gene into other tobaccos, as described herein. For example, a breeder can create segregating populations from hybridizations of a genotype containing a mutant allele with an agronomically desirable genotype. Plants in the F2 or backcross generations can be screened using a marker developed from a genomic sequence or a fragment thereof, using one of the techniques listed herein. Plants identified as possessing the mutant allele can be backcrossed or self-pollinated to create a second population to be screened. Depending on the expected inheritance pattern or the MAS technology used, it may be necessary to self-pollinate the selected plants before each cycle of backcrossing to aid identification of the desired individual plants. Backcrossing or other breeding procedure can be repeated until the desired phenotype of the recurrent parent is recovered.

According to the disclosure, in a breeding program, successful crosses yield F1 plants that are fertile. Selected F1 plants can be crossed with one of the parents, and the first backcross generation plants are self-pollinated to produce a population that is again screened for variant gene expression (for example, the null version of the gene). The process of backcrossing, self-pollination, and screening is repeated, for example, at least 4 times until the final screening produces a plant that is fertile and reasonably similar to the recurrent parent. This plant, if desired, is self-pollinated and the progeny are subsequently screened again to confirm that the plant exhibits variant gene expression. In some embodiments, a plant population in the F2 generation is screened for variant gene expression, for example, a plant is identified that fails to express a polypeptide due to the absence of the gene according to standard methods, for example, by using a PCR method with primers based upon the nucleotide sequence information for the polynucleotides including NtMNS1a, NTMNS1b, NtMNS2, or NtMNS1.4 polynucleotide (or any of the combinations thereof) as described herein. Hybrid tobacco varieties can be produced by preventing self-pollination of female parent plants (that is, seed parents) of a first variety, permitting pollen from male parent plants of a second variety to fertilize the female parent plants, and allowing F1 hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), or transgenic male sterility wherein a transgene inhibits microsporogenesis and/or pollen formation, or self-incompatibility. Female parent plants containing CMS are particularly useful. In embodiments in which the female parent plants are CMS, pollen is harvested from male fertile plants and applied manually to the stigmas of CMS female parent plants, and the resulting F1 seed is harvested.

Varieties and lines described herein can be used to form single-cross tobacco F1 hybrids. In such embodiments, the plants of the parent varieties can be grown as substantially homogeneous adjoining populations to facilitate natural cross-pollination from the male parent plants to the female parent plants. The F1 seed formed on the female parent plants is selectively harvested by conventional means. One also can grow the two parent plant varieties in bulk and harvest a blend of F1 hybrid seed formed on the female parent and seed formed upon the male parent as the result of self-pollination. Alternatively, three-way crosses can be carried out wherein a single-cross F1 hybrid is used as a female parent and is crossed with a different male parent. As another alternative, double-cross hybrids can be created wherein the F1 progeny of two different single-crosses are themselves crossed.

A population of mutant, non-naturally occurring or transgenic plants can be screened or selected for those members of the population that have a desired trait or phenotype. For example, a population of progeny of a single transformation event can be screened for those plants having a desired level of expression or activity of NtMNS1a, NTMNS1b, NtMNS2, or NtMNS1.4 or the polypeptide encoded thereby. Physical and biochemical methods can be used to identify expression or activity levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining and enzyme assays also can be used to detect the presence or expression or activity of polypeptides or polynucleotides.

Mutant, non-naturally occurring or transgenic plant cells and plants are described herein comprising one or more recombinant polynucleotides—such as one or more isolated NtMNS1a, NTMNS1b, NtMNS2, or NtMNS1.4 polynucleotides (or a combination of two or more or three or more thereof), one or more polynucleotide constructs, one or more double-stranded RNAs, one or more conjugates or one or more vectors/expression vectors.

Without limitation, the plants described herein may be modified for other purposes either before or after the expression or activity has been modulated according to the present invention. An example of such modification is the introduction of an expressible polynucleotide encoding a heterologous protein of interest into the plant. The term “expressible” in the context of this invention refers to an operative linkage of a gene to regulatory elements that direct the expression of the protein or polypeptide encoded by the gene in plant cells, preferably comprised within a leaf.

Production of Heterologous Glycoproteins with Modified Mannose Content.

Various embodiments are directed to produce in a plant with modified alpha-mannosidase I activity, a heterologous protein that is suitable for use as a human therapeutic. Examples of a heterologous protein include but are not limited to a growth factor, receptor, ligand, signaling molecule; kinase, enzyme, hormone, tumor suppressor, blood clotting protein, cell cycle protein, metabolic protein, neuronal protein, cardiac protein, protein deficient in specific disease states, antibodies, antigens, proteins that provide resistance to diseases, proteins for replacement therapy of human genetic diseases, antimicrobial proteins, interferons, and cytokines. The terms “antibody” and “antibodies” refer to monoclonal antibodies, multispecific antibodies, human antibodies, humanized antibodies, camelised antibodies, chimeric antibodies, single-chain Fvs (scFv), single chain antibodies, single domain antibodies, domain antibodies (VH, VHH, VLA), Fab fragments, F(ab′) fragments, disulfide-linked Fvs (sdFv), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (for example, IgG, IgE, IgM, IgD, IgA and IgY), class (for example, IgG1, IgG2, IgG3, IgG4, IgA1 and IgA2) or subclass. Examples of an antibody or a fragment thereof that can be produced include abciximab, adalimumab, alemtuzumab, basiliximab, belimumab, bevaxizumab, brentuximab, canakinumab, cetuximab, certolizumab, daclizumab, denosumab, eculizumab, efalizumab, gemtuzumab, golimumab, ibritumomab, ipilimumab, natalizumab, ofatumumab, omalizumab, palivizumab, panitumumab, ranibizumab, rituximab, tocilizumab, tositumomab, trastuzumab, and antibodies that bind to the same antigenic determinant as the above-listed monoclonal antibodies, The amount of plant-specific immunogenic alpha-1,3-fucose and beta-1,2-xylose on an N-glycan of a glycoprotein from a plant, including a heterologous glycoprotein, can be reduced or eliminated by various methods without affecting the genes coding for the addition of such alpha-1,3-fucose and beta-1,2-xylose. A method to reduce or eliminate the addition of such saccharides onto an N-glycan of a glycoprotein in a plant cell comprises reducing, inhibiting or substantially inhibiting the enzyme activity of one or more alpha-mannosidase I enzymes of the present invention, in a plant or plant cell thereby preventing further processing of the N-glycan from high-mannose type N-glycan towards hybrid-type N-glycan and ultimately complex type N-glycans. In plant cells, complex type N-glycans contain an alpha-1,3-fucose and a beta-1,2-xylose. Hence, without being bound by theory, plants which are substantially inhibited for NtMNS1a, NtMNS1b, NtMNS2, and NtMan1.4, can be used to produce glycoproteins with altered immunogenic properties as well as improved efficacy. Uses of such plants include:

(a) Plants that are substantially inhibited in NtMNS1a, NtMNS1b, NtMNS2, and NtMan1.4, can be used for the manufacture of a heterologous glycoprotein that substantially lacks alpha-1,3-linked fucose and beta-1,2-linked xylose on its N-glycan. Glycoproteins produced by such plants will preferably have high-mannose N-glycans. High-mannose type N-glycans on antigens lead to increased binding to antigen-presenting cells. Certain antibodies with high-mannose type N-glycans have increased antibody-dependent cellular cytotoxicity.
(b) Plants that have increased activity of NtMNS1a, NtMNS1b, NtMNS2, or NtMan1.4, or a combination thereof, will have reduced high-mannose N-glycans and hence increased hybrid-type and complex and mature N-glycans on glycoproteins produced therein. Certain high-mannose type N-glycosylated glycoproteins are cleared quicker from the blood stream through increased binding to the high-mannose receptor. Reducing the amount of high-mannoses can reduce the clearing time and hence increase half-life.

### EXAMPLES

The following examples are provided as an illustration and not as a limitation. Unless otherwise indicated, the present invention employs conventional techniques and methods of molecular biology, plant biology, bioinformatics, and plant breeding.

### Example 1

### Identification of the Genomic Sequence of NtMNS1a, NtMNS1b and NtMNS2

The genomic sequences of NtMNS1a, NtMNS1b and NtMNS2 are identified by screening of a BAC library and sequencing three BAC clones containing part of the genome which includes NtMNS1a, NtMNS1b or NtMNS2, respectively. The sequences are set forth in the section SEQUENCE INFORMATION.

The deduced amino acid sequences of NtMNS1a, NtMNS1b and NtMNS2 are compared with other proteins or deduced protein sequences from NCBI and show that two proteins from A. thaliana, AtMNS1 (At1g51590) and AtMNS2 (At3g21160) share highest sequence identities and similarities (Table 1).

TABLE 1
Percentages identity of NtMNS1a, 1b and 2 proteins and Arabidopsis thaliana
AtMNS1 and AtMNS2 using the program EMBOSS needle for alignment.

| Sequence | | | | | | |
|---|---|---|---|---|---|---|
| Designation | NtMNS1b | NtMNS1a | NtMNS2 | AtMNS1 | AtMNS2 | SEQ ID NO. |
| NtMNS1b | 100 | 97.9 | 86.1 | 74.8 | 71.6 | 62 |
| NtMNS1a | 98.8 | 100 | 92.1 | 75.2 | 71.7 | 31 |
| NtMNS2 | 92.3 | 85.9 | 100 | 73.6 | 73 | 93 |

To estimate the percent sequence identities of the nucleotide sequences of the invention relative to publically known sequences, NCBI blastn was used to identify sequences in public databases that show homologies to input sequences. Blastn allows the usage of predefined sets of parameters for searches using megablast, dc-megablast, blastn and blastn-short. The following databases were searched: NCBI patent nucleotides, Non-redundant EBI patent nucleotides level 1, Non-redundant EBI patent nucleotides level 2, TAIR9 cdna models and NCBI nucleotide entries. Blast search results were limited to hits with e-values smaller or equal to 1. For each of the input nucleotide sequences, SEQ ID NO's:1 to 30, SEQ ID NO's:32 to 61 and SEQ ID NO's:63 to 92, the blastn search was done with the four sets of predefined parameters. For each input nucleotide sequence, local pairwise alignments using the EMBOSS water program were subsequently made with the sequences identified using any of the blastn searches. The number of identical basepairs in the best local alignment obtained was estimated and this was used to calculate the percentage of identity of the whole input sequence, SEQ ID NO's:1 to 30, SEQ ID NO's:32 to 61 and SEQ ID NO's:63 to 92, with the database sequence having best fit. The number of identical basepairs is divided by the total length of the sequence identified. Blast results are summarized in Table 2.

TABLE 2
Identity (%) of SEQ (SEQ ID NO:) and database entries (best match) using local
pairwise alignments using the program EMBOSS water, the sequence (SEQ) length in
basepairs and the number of identical basepairs in the best local alignment.

| SEQ | Identity | SEQ length | Database entry | Sequence Designation. |
|---|---|---|---|---|
| 1 | 72.01 | 14501 | gb\|AC235805.1\| | NtMNS1a with 5′ and 3′ UTR |
| 2 | 72.65 | 12162 | gb\|AC235805.1\| | NtMNS1a without 5′ and 3′ UTR |
| 3 | 85.62 | 153 | gb\|AC235805.1\| | NtMNS1a Exon 1 |
| 4 | 83.45 | 145 | gb\|AC212805.1\| | NtMNS1a Intron 1 |
| 5 | 87.5 | 48 | gb\|AC235805.1\| | NtMNS1a Exon 2 |
| 6 | 79.06 | 1251 | gb\|AC235805.1\| | NtMNS1a Intron 2 |
| 7 | 86.67 | 195 | gb\|AC235805.1\| | NtMNS1a Exon 3 |
| 8 | 72.27 | 3938 | emb\|AJ416571.1\| | NtMNS1a Intron 3 |
| 9 | 94.69 | 113 | gb\|AC235805.1\| | NtMNS1a Exon 4 |
| 10 | 76.26 | 396 | gb\|AC235805.1\| | NtMNS1a Intron 4 |
| 11 | 100 | 66 | gb\|AC235805.1\| | NtMNS1a Exon 5 |
| 12 | 83.33 | 114 | gb\|AC235805.1\| | NtMNS1a Intron 5 |
| 13 | 95.93 | 172 | gb\|AC235805.1\| | NtMNS1a Exon 6 |
| 14 | 78.74 | 508 | gb\|AC235805.1\| | NtMNS1a Intron 6 |
| 15 | 97.78 | 90 | gb\|AC235805.1\| | NtMNS1a Exon 7 |
| 16 | 79.86 | 139 | ref\|NG_027682.1\| | NtMNS1a Intron 7 |
| 17 | 95.2 | 125 | gb\|AC235805.1\| | NtMNS1a Exon 8 |
| 18 | 84.32 | 185 | gb\|AC235805.1\| | NtMNS1a Intron 8 |
| 19 | 100 | 66 | gb\|AC235805.1\| | NtMNS1a Exon 9 |
| 20 | 74.03 | 1656 | gb\|AC238342.1\| | NtMNS1a Intron 9 |
| 21 | 90.83 | 109 | gb\|AC235805.1\| | NtMNS1a Exon 10 |
| 22 | 91.01 | 89 | gb\|AC235805.1\| | NtMNS1a Intron 10 |
| 23 | 97.98 | 99 | gb\|AC235805.1\| | NtMNS1a Exon 11 |
| 24 | 74.49 | 886 | AT4G03300.1 | NtMNS1a Intron 11 |
| 25 | 95.06 | 81 | gb\|AC235805.1\| | NtMNS1a Exon 12 |
| 26 | 87.76 | 98 | gb\|AC235805.1\| | NtMNS1a Intron 12 |
| 27 | 97.66 | 171 | gb\|AC235805.1\| | NtMNS1a Exon 13 |
| 28 | 75.91 | 1017 | NRNL1: NRN_GP280038 | NtMNS1a Intron 13 |
| 29 | 89.29 | 252 | gb\|AC235805.1\| | NtMNS1a Exon 14 |
| 30 | 87.87 | 1740 | gb\|AC235805.1\| | NtMNS1a cDNA sequence |
| 32 | 74.46 | 12401 | gb\|AC235805.1\| | NtMNS1b with 5′ and 3′ UTR |
| 33 | 75.46 | 10393 | gb\|AC235805.1\| | NtMNS1b without 5′ and 3′ UTR |
| 34 | 86.27 | 153 | gb\|AC235805.1\| | NtMNS1b Exon 1 |
| 35 | 83.01 | 153 | gb\|AC026722.4\|AC026722 | NtMNS1b Intron 1 |
| 36 | 89.58 | 48 | gb\|AC235805.1\| | NtMNS1b Exon 2 |
| 37 | 78.21 | 1308 | gb\|AC235805.1\| | NtMNS1b Intron 2 |
| 38 | 85.64 | 195 | gb\|AC235805.1\| | NtMNS1b Exon 3 |
| 39 | 73.25 | 2071 | gb\|AC215449.3\| | NtMNS1b Intron 3 |
| 40 | 94.69 | 113 | gb\|AC235805.1\| | NtMNS1b Exon 4 |
| 41 | 78.43 | 394 | emb\|FN357487.1\| | NtMNS1b Intron 4 |
| 42 | 96.97 | 66 | gb\|AC235805.1\| | NtMNS1b Exon 5 |
| 43 | 84.21 | 114 | gb\|AC235805.1\| | NtMNS1b Intron 5 |
| 44 | 97.09 | 172 | gb\|AC235805.1\| | NtMNS1b Exon 6 |
| 45 | 80.08 | 487 | gb\|AC235805.1\| | NtMNS1b Intron 6 |
| 46 | 97.78 | 90 | gb\|AC235805.1\| | NtMNS1b Exon 7 |
| 47 | 82.19 | 146 | emb\|AL807388.8\| | NtMNS1b Intron 7 |
| 48 | 93.1 | 116 | gb\|AC235805.1\| | NtMNS1b Exon 8 |
| 49 | 83.73 | 252 | gb\|EA166365.1\| | NtMNS1b Intron 8 |
| 50 | 100 | 66 | gb\|AC235805.1\| | NtMNS1b Exon 9 |
| 51 | 75.84 | 1668 | gb\|AC238342.1\| | NtMNS1b Intron 9 |
| 52 | 90.83 | 109 | gb\|AC235805.1\| | NtMNS1b Exon 10 |
| 53 | 88.76 | 89 | gb\|AC235805.1\| | NtMNS1b Intron 10 |
| 54 | 97.98 | 99 | gb\|AC235805.1\| | NtMNS1b Exon 11 |
| 55 | 73.3 | 895 | gb\|AC235805.1\| | NtMNS1b Intron 11 |
| 56 | 97.53 | 81 | gb\|AC235805.1\| | NtMNS1b Exon 12 |
| 57 | 88.89 | 99 | gb\|AC235805.1\| | NtMNS1b Intron 12 |
| 58 | 96.49 | 171 | gb\|AC235805.1\| | NtMNS1b Exon 13 |
| 59 | 72.82 | 986 | gb\|AC125483.4\| | NtMNS1b Intron 13 |
| 60 | 90.08 | 252 | gb\|AC235805.1\| | NtMNS1b Exon 14 |
| 61 | 87.59 | 1740 | gb\|AC235805.1\| | NtMNS1b cDNA sequence |
| 63 | 71.53 | 11501 | gb\|AC235805.1\| | NtMNS2 with 5′ and 3′ UTR |
| 64 | 73.15 | 9385 | gb\|AC025294.14\|AC025294 | NtMNS2 without 5′ and 3′ UTR |
| 65 | 81.05 | 153 | dbj\|FU037911.1\| | NtMNS2 Exon 1 |
| 66 | 69.72 | 1255 | gb\|U35619.1\|NTU35619 | NtMNS2 Intron 1 |
| 67 | 89.58 | 48 | gb\|AC235805.1\| | NtMNS2 Exon 2 |
| 68 | 77.19 | 583 | dbj\|FU037651.1\| | NtMNS2 Intron 2 |
| 69 | 82.05 | 195 | emb\|AM423594.2\| | NtMNS2 Exon 3 |
| 70 | 74.69 | 1766 | gb\|AC235805.1\| | NtMNS2 Intron 3 |
| 71 | 92.92 | 113 | gb\|AC235805.1\| | NtMNS2 Exon 4 |
| 72 | 73.87 | 727 | emb\|AL606751.5\| | NtMNS2 Intron 4 |
| 73 | 93.94 | 66 | gb\|AC235805.1\| | NtMNS2 Exon 5 |
| 74 | 82.54 | 126 | emb\|CT033786.13\| | NtMNS2 Intron 5 |
| 75 | 90.7 | 172 | gb\|AC235805.1\| | NtMNS2 Exon 6 |
| 76 | 73.61 | 720 | AT3G30763.1 | NtMNS2 Intron 6 |
| 77 | 86.67 | 90 | gb\|AC235805.1\| | NtMNS2 Exon 7 |
| 78 | 76.58 | 158 | emb\|AL133319.24\| | NtMNS2 Intron 7 |
| 79 | 88 | 125 | gb\|AC235805.1\| | NtMNS2 Exon 8 |
| 80 | 76.71 | 146 | emb\|CU184877.6\| | NtMNS2 Intron 8 |
| 81 | 89.39 | 66 | gb\|AC235805.1\| | NtMNS2 Exon 9 |
| 82 | 75.16 | 1123 | NRNL1: NRN_EA741335 | NtMNS2 Intron 9 |
| 83 | 89.91 | 109 | gb\|AC235805.1\| | NtMNS2 Exon 10 |
| 84 | 83.16 | 95 | gb\|AC103335.7\| | NtMNS2 Intron 10 |
| 85 | 89.9 | 99 | gb\|AC235805.1\| | NtMNS2 Exon 11 |
| 86 | 74.51 | 412 | dbj\|BS000014.1\| | NtMNS2 Intron 11 |
| 87 | 90.48 | 84 | gb\|AC235805.1\| | NtMNS2 Exon 12 |
| 88 | 86.9 | 84 | gb\|AC236462.1\| | NtMNS2 Intron 12 |
| 89 | 93.57 | 171 | gb\|AC235805.1\| | NtMNS2 Exon 13 |
| 90 | 71.56 | 450 | AT3G46710.1 | NtMNS2 Intron 13 |
| 91 | 83.53 | 249 | gb\|AC235805.1\| | NtMNS2 Exon 14 |
| 92 | 78.05 | 1740 | emb\|GN102675.1\| | MNS2 cDNA sequence |
| 94 | | | | MNS1a cDNA sequence |
| 96 | | | | MNS1b cDNA sequence |
| 98 | | | | Man1.4 cDNA sequence |

### Example 2

### Search Protocol for the Selection of Zinc Finger Nuclease Target Sites

This example illustrates how to search the NtMNS genes (NtMNS1a, NtMNS1b, NtMNS2 genes) to screen for the occurrence of unique target sites within the given gene sequence compared to a given genome database to develop tools for modifying the expression of the gene. The target sites identified by methods of the invention, including those disclosed below, the sequence motifs, and use of any of the sites or motifs in modifying the corresponding gene sequence in a plant, such as tobacco, are encompassed in the invention.

### 2.1 Search Algorithm.

A computer program is developed that allows the screening of an input query (target) nucleotide sequence for the occurrence of two fixed-length substring DNA motifs separated by a given spacer size using a suffix array within a DNA database, such as for example the tobacco genome sequence assembly of Example 1. The suffix array construction and the search use the open source libdivsufsort library-2.0.0 (http://code.google.com/p/libdivsufsort/) which converts any input string directly into a Burrows-Wheeler transformed string. The program scans the full input (target) nucleotide sequence and returns all the substring combinations occurring less than a selected number of times in the selected DNA database.

### 2.2 Selection of Target Site for Zinc Finger Nuclease-Mediated Mutagenesis of a Query Sequence.

A zinc finger DNA binding domain recognizes a three basepair nucleotide sequence. A zinc finger nuclease comprises a zinc finger protein comprising one, two, three, four, five, six or more zinc finger DNA binding domains, and the non-specific nuclease of a Type IIS restriction enzyme. Zinc finger nucleases can be used to introduce a double-stranded break into a target sequence. To introduce a double-stranded break, a pair of zinc finger nucleases, one of which binds to the plus (upper) strand of the target sequence and the other to the minus (lower) strand of the same target sequence separated by 0, 1, 2, 3, 4, 5, 6 or more nucleotides is required. By using plurals of 3 for each of the two fixed-length substring DNA motifs, the program can be used to identify two zinc finger protein target sites separated by a given spacer length.

### 2.3 Program Inputs:

1. The target query DNA sequence
2. The DNA database to be searched
3. The fixed size of the first substring DNA motif
4. The fixed size of the spacer
5. The fixed size of the second substring DNA motif
6. The threshold number of occurrences of the combination of program inputs 3 and 5 separated by program input 4 in the chosen DNA database of program input 2

### 2.4 Program Output:

A list of nucleotide sequences with, for each sequence, the number of times the sequence occurs in the DNA database with a maximum of the program input 6 threshold.

### Example 3

### Targeting Ethyl Methanesulfonate-Induced Local Mutations in Tobacco

### 3.1 Mutagenesis.

M0 seeds of Nicotiana tabacum are mutagenized with ethyl methanesulfonate (EMS; C3H8O3S) to generate a population of plants with random point mutations. Various concentrations and incubation periods are tested. To estimate the effects of each treatment, the kill-curve is estimated in the M1 generation for each treatment and lethality is measured as complete seedling lost. Furthermore, fertility is measured as the capability of each plant to generate capsules and seeds and the number of chimeric plants is estimated. A plant is designated as chimeric if its phenotype shows an alteration of the leaf color, such as albino or yellow sectors, or deformity of the plant. M1 plants are self-fertilised and M2 seeds are harvested and sown. The M2 germplasm allows recessive and lethal alleles to be recovered as heterozygotes.

### 3.2 Mutation Detection.

DNA is extracted from individual M2 plants and their seeds are stored for future sampling. Target NtMNS1a, NtMNS1b or NtMNS2 gene fragments are amplified using specific primers and mutations in the target genes can be detected by sequencing. DNA from individual plants can also be selectively pooled before amplification. Alternatively, such DNA can be amplified with fluorescently labeled primers such that mismatched heteroduplexes are generated between wild type and mutant DNA. Heteroduplexes are then incubated with the endonuclease CEL1 that cleaves heteroduplex mismatched sites and the resultant cleavage products are run on a capillary ABI3730 sequencer and the fluorescently labelled traces analysed. The CEL1 assay is described by Olekowski et al. (1998, Nucleic Acids Res. 26: 4597-4602). The latter technology is also known as TILLING (Targeting Induced Local Lesions IN Genomes) and is a reverse genetics process. A modified TILLING process is described by Colbert et al. (2001, Plant Physiol. 126: 480-484. High-throughput screening for induced point mutations) and relies on the ability of a special enzyme to detect mismatches in normal and mutant DNA strands when they are annealed. Subsequent analysis of the individual plant DNA from the pooled DNA identifies the plant bearing the desired mutation.

### Example 4

### Transient Expression of Rituximab Monoclonal Antibody in Tobacco

This example shows how an antibody with modified mannose content on its N-glycan can be made in a tobacco plant with modified alpha-mannosidase I activity.

### 4.1 Construction of Rituximab Monoclonal Antibody Expression Vectors.

An expression cassette comprising the full coding sequences of the rituximab monoclonal antibody light and heavy chain as in CAS registry number 174722-31-7 or WO02/060955 was made by chemical synthesis with codons optimized for expression in a tobacco plant cell. The heavy chain sequence was synthesized with a patatin signal peptide and placed under control of the HT-CPMV promoter and HT-CPMV untranslated 5′ and 3′ UTR sequences as in patent WO09/087,391 and cauliflower mosaic virus 35S terminator sequence. The light chain with patatin signal peptide was placed under control of a plastocyanin promoter and terminator sequence as in patent WO01/25455. Both expression cassettes were cloned in the T-DNA of pCambia-2300 (GenBank: AF234315.1; Hajdukiewicz et al., 1994. Plant. Mol. Biol. 25: 989-994) to generate pCambia-Rituximab.

4.2 Infiltration of Nicotiana benthamiana Plants.

pCambia-Rituximab is introduced in Agrobacterium tumefaciens Agl1. Bacteria are grown in YEB-medium comprising 2 g/L Beef extract, 0.4 g/L Yeast extract, 2 g/L Bacto-Peptone, 2 g/L Sucrose, 0.1 g/L MgSO4 and proper antibiotics for selection of the respective Agrobacterium strain and binary vector, in an erlenmeyer at 28° C. and 250 rpm on a rotary shaker up to an OD600>1.6. The culture is then diluted 1:100 in fresh LB Broth Miller medium containing 10 mM 2-(N-morpholino)-ethanesulfonic acid (MES) and proper antibiotics and further grown at 28° C. and 250 rpm on a rotary shaker up to an OD600>2. After growth, bacteria are collected by centrifugation at 8′000 g and 4° C. for 15 min. Pelleted bacteria are resuspended in infiltration solution containing 10 mM MgCl2 and 5 mM MES, final pH 5.6, and OD600=2. Four weeks old Nicotiana benthamiana plants with modified alpha-mannosidase I activity are co-infiltrated with an Agrobacterium tumefaciens strain Agl1 containing the tomato bushy stunt virus (TBSV) p19 suppressor of gene silencing (Swiss-Prot P50625) and pCambia-Rituximab at 1:1 ratio and final OD600 nm=0.3. The coding sequence for the TBSV p19 suppressor of gene silencing is under control of a double cauliflower mosiac virus 35S promoter and terminator sequence in pBin19 (Bevan MW (1984) Binary Agrobacterium vectors for plant transformation. Nucleic Acids Res. 12: 8711-8721). Vacuum infiltration is performed with the bacteria inside a glass bell jar (Schott-Duran Mobilex 300 mm) using a V-710 Büchi pump connected to a V-855 regulator. Artificial lighting (80-100 μmol photon/cm²) is kept on during the whole infiltration process to ensure consistent light conditions. Following infiltration, plants are placed along with non-infiltrated control plants in the greenhouse until harvesting. Growth conditions such as fertilization, photoperiod and temperature are the same as used before infiltration. Water and fertilizer are administered to plants using a drip irrigation system.

### 4.3 Harvesting, Material Sampling and Analysis of Expression.

Six days after infiltration, leaf material are collected in a heat-sealable pouch, sealed and placed between layers of dry-ice for at least 10 minutes. After harvesting, all leaf samples are stored at −80° C. until further processing. Harvested leaves are homogenized to a fine powder using a coffee-grinder on dry-ice and extracted in 3 vol/wt extraction buffer containing 50 mM Tris (pH 7.4), 150 mM NaCl, 0.1% Triton X-100, 4M Urea and 2 mM DTT. The expression of rituximab monoclonal antibody is quantified in the soluble extracts by ELISA. Plates (Immulon 2HB, Thermofisher) are coated overnight at 4° C. with a capture antibody (Goat anti-mouse IgG1 heavy chain specific Sigma, #M8770) at a concentration of 2.5 ug/mL. A standard curve (4-80 ng/mL) is prepared using Mouse IgG1 control protein (Bethyl, #M110-102) in mock extract (prepared from leaf material infiltrated only with the p19 suppressor of gene silencing bacterial suspension). Soluble extracts are diluted 1:1000 in dilution buffer (50 mM Tris pH 7.4, 150 mM NaCl, 0.1% Triton X-100) and standards and samples were loaded in triplicate and incubated for 1 hour at 37° C. The antibody for detection is a peroxidase-conjugated goat anti-mouse IgG Fc-specific from Jackson ImmunoResearch (#115-035-205) which is used at a dilution of 1:40′000 and incubated for 1 hour at 37° C. Total soluble protein in the extracts is determined using the Coomassie-Plus Assay reagent from Pierce (#24236).

### 4.4 Analysis of N-Glycan Composition.

The N-glycan composition of the rituximab antibody in the plant cell extract is determined according to standard methods (Bakker et al. (2001) Proc. Natl. Acad. Sci. USA 98: 2899-2904).

### Example 5

### Cloning of Alpha-Mannosidase cDNA

5.1 Isolation of Ribonucleic Acid and cDNA Synthesis.

Leaves of Nicotiana tabacum plants grown in the greenhouse are ground in liquid nitrogen to a fine powder. RNA is extracted from 200 mg of ground leaf powder using the RNeasy RNA extraction kit from Qiagen (Qiagen AG, Hombrechtikon, Germany) according to the manufacturers recommendation. One microgram (1 μg) of total RNA is treated with DNaseI (New England Biolabs, Ipswich, USA) according to the manufacturer. cDNA is synthesized from 500 ng of DNaseI-treated-RNA using AMV Reverse Transcriptase (Invitrogen AG, Basel, Switzerland) according to the manufacturer.

### 5.2 Cloning by PCR.

First strand cDNA is diluted ten times and amplified by PCR using a Mastercycler gradient machine (Eppendorf). Reactions are performed in 50 μl containing 25 μl of 2× Phusion mastermix (Finnzyme), 20 μl of water, 1 μl of diluted cDNA and 2 μL of each primer (10 μM). Primers for amplifying NtMNS1a cDNA are:

| | Forward primer | | Reverse primer | |
|---|---|---|---|---|
| Final target | Code | Sequence (5′ to 3′) | Code | Sequence (5′ to 3′) |
| NtMNS2 | PC307F | ATGGGGAGGAGTAGATCGTCC | PC308R | CTACTTATTACCAAATCGGCCTTC |
| NtMNS1a | PC309F | ATGGCGAGGAGTAGATCGTCTT | PC310R | TTAGGTGCGACTAGCAATTTGC |

Thermocycler conditions are as recommended by the supplier using an annealing temperature of 58° C. Following PCR, the resulting product is adenylated at the 3′-end. 50 μl of 2× Taq Mastermix (New England Biolabs) is added to the PCR reaction mixes and incubated at 72° C. for 10 minutes. Resulting PCR products are purified using the QIAquick PCR Purification Kit (Qiagen). Purified products are cloned into pCR2.1-TOPO according to the manufacturer (Invitrogen) and transformed into TOP10 Escherichia coli cells according to standard protocols. DNA is isolated from individual clones and resulting plasmid DNA is sequenced according to standard protocols.

### 5.3 Sequence Analysis.

Polynucleotide sequences are compiled using Contig Express and AlignX (Vector NTI, Invitrogen). An MNS1a cDNA sequence is set forth below as SEQ ID NO: 30. The MNS1a cDNA sequence represents a sequence observed upon sequencing of the respective cDNA PCR fragment.

### Sequence Information

In the description and examples, reference is made to the following sequences that are also represented in the sequence listing:

(NtMNS1a with 5′ and 3′ UTR)
SEQ ID NO: 1
aaggaatattcagaggaatgttctatgtatttgtacttttaataggtaaggggtatgccc
catataagtaggaatagagagagaaagaaggggcatgtaatattttatcttgataagctc
tttctagaaaagtttactctcaagtaactacaaatactatctttacataagattcgattt
gttgttttgtccaagctttcccacatcaatccaataaagtatttgatattcccacgtttg
gttatcttacatcattatcagagagagaatcatccacctcgttatatatttgagtgaatt
attctctctatttacatttattgtcatttatcatatttattgcttatccttgttctccca
ttctttcataagaatatcattaaatatccatttggcatttaataactttaagtgcggttt
ccagactattactatccatcaatcttgggtctaggatttattatgtttaactataattta
ctcattatcatttatttaattgtttaacaaaaaggcttaagactttttggtcaaacaata
tggagtctgtaagtggggaggggcaaaagtgaaacactttattaacggcaagggcatttt
tgtacccaaatacaaacggagggcataattgctctattttcaatacttcagaggcctttt
ccataaattctttcttaaacttactcccactttaatgctctccttttcctaggtagagtc
agacctttatataatagtatctctatataacaacactttactataaaagcgaagcttttc
cggaaccaattttcatgttatgttataatatatgttctctataacaacacttcgctataa
catccaaaaatattaggaacaaacgaggctatcatagagatgtttgacattatatccgta
taaatatttgtcataaaaaaatatttttctaaaaaaatgtaccattgtgagattttttta
ggaaaggaaaaaatatttaccgaggattgaccaaatatattcgaagaaaaagatagtaat
ggatgggagaagacatagcttggtagcttagtcctaggtaaggtgggatgcttaatctta
aatggaagacaagtcaatgttacaccgaccgcgcatgattgataagagcagtattattac
cgtgtcttcactctttaccaaggctgaacgggtcttttacctaattaacgtcctgtagat
ttaggcgaggtttccttttgggaagtccagtagtcttggtcttcttggtcgttcctctcc
cccgatctattcaatctgcatcgggagatcgatctgcactttgattggtatattcataaa
aagtgggtggaATGGCGAGGAGTAGATCGTCTTCCACTACTTTCAGGTACATTAATCCGG
CTTACTATCTGAAACGGCCAAAGCGTCTGGCTTTGCTCTTCATCGTTTTTGTCTTCGCCA
CCTTCTTCTTTTGGGATCGACAAACTTTAGTCCGTGATCATCAGgttcttctcttcattt
tccatttgtttcaccgtcctttttctctgattctctttgtggaattcatgtttaattttg
gtttaaaagtttgtaaagtagcgttctttaattacaaaacaactatattctttatgtttt
tttttgcagGAAGAGATCTCTAAGTTGAATCATGAAGTGACGCAATTCGCAAATCTGgtt
agtggttatctgaattatctatagctgtggaattttttattttaataatcagcctactgc
ctttaattcttttgtggctgccgtccctcttcttgctttgtcggggaactgtatgctaga
gcgtcttttaatatgtgcctgtacaaagttgtaattactcgagctacctcctgttcttcc
ttcttcaaattaaatgtggttgagaatctgtttaactacttgtaatggggaaaaaacgat
aaacttactaattcaagttagatttaacatcaatgtctagagggatttatatggccagct
tggttatgaagcctgaatttgggtcgcttagcgaagagctaccatgtactgccatttcac
ctacttaatacctcaatctgcttaagtaaggctagtaccgcccaacactgaatttggttt
gcctagtgaagagtttctctgtctttcactgagcttaatacctcaatctgcttcagttag
ctcagggctagtactgcagtgttgagccctataaacgggcttggagtttaaaaaatattt
gtgccattaaagcttaggaccatgttacctagtttagatattataggaaatgaaaaagca
aaaaaagacgagctacgacccggcaaacagaaaaggaactcaactaaattagtcttaaga
aggtgatgcatctggctgagctcaaaccaggatgtaaagattagcggatggactgaccaa
acaagagatggtggatggagtaagagtcgagatgtcgcaatatacctatagtgcactata
gttcagcaccttttgtgttattccttagcattaaagggcgaggtaacagttggtggcaaa
aagtcctcactgcgcattggaatgcttcctcgttggggttaaggagaactggaagagtgt
tcaagtagacttgagagaccaagacccaatggcttaaaatgaggggatagaatactatat
atatacacatatatatatctatgtgtaatgaaacttcatgaaaatatctatgtgctatgt
acttcttttcttgtccgtcttgtttgtctaaaaatttggtggtttggtttgtattttctg
gaaaaagaagtacaaagaatggatatagcttgttatgatttatgccagtattattttcat
gtgtgcttgcttcacagtttacccatgttctgttgtttgcagtatagcatttaagctttt
gattttaaatattcaacttgtttgcatttattttggatactgttttagCTGGAAGATTTG
AAGAATGGTCGAGTCATGCCAGATAAAAAGATGAAATCTAGTGGCAAAGGTGGTCATGCA
GCAAAAAATATGGATTCACCAGATAATATCCTTGATGCTCAGCGAAGGGAGAAAGTGAAA
GATGCTATGCTTCATGCTTGGAGTTCTTATGAAAAATATGCATGGGGTCATGATGAATTA
CAGgtttggatgttacttcgaataagttattttttgtgttgttaatgttattattattat
tattttttgtgttgttaatgttgcctttgttttattgtatcttgtgatttcgcaattaga
tcattggtggaggaattctctactttttgatatacttcctgggggagttctctccctttt
gattaatacaatttaccttatctaaaaaaaatcattggtggaggcatatgtaaagaaatt
cccggaaaatgaatccgggacattccaatattctttttcctttttgtgtgttaaggggaa
atggggtataatagatgattagttaattacttaattaaatgagttagttgtaaatttaaa
aactatttaaaaattaaatgagttagttgtcgattgatgttctccattaccttttctttc
tttgttattttattttcctaagtgctataccttttgttgactagataagcatgtgacact
ctagtttttcaattacaatattctgtaggttagtttgcagcagcaacgacaaaaactatg
cctcaaaaatataaatcatcatgatctaggttgctctatttgggcccatttcatgtcaac
cttcaatagtttgggcttttctaacagtagagattctctacaattcctagtaacatacac
tttttttttaaaaagtaacacaaattcaaactttttgtttattatgtttttactcattcc
atcccatttcatgttccggtgtttgactgggtataaaatttaagaaataaggaagacttt
ttacatgtaatacaaatatatacaacataccaaaatgacctttactattaacatctaatg
aaaggaggtaacctaccgtaccttcgtgataaaaaagggttaccttatcctcccaaagaa
aaggttgtaagagttccgcatatcacttactatttctatctcctaataaaaaaatagttt
ttatatcaagtgggttcctaagaggttatgtcagtaagcataaaacgttattgctaggag
taaattgtttgcaattacaaaaatgtctcactcttttctggatagactaaaaaggaagga
atgccacatacaatgggacaggaggagtatatgttcttttcttcttatatcctgaccaag
tatattgatttagcatgttttgatgctctggatattgcaaatgactatgaaatagcgatt
acataagtggctaagacttggccttttaatttattcttttctagggtatgttttgatatg
attctctagatatttctgaattattgttagtgtcctggtagtgaggatagcaatttcatc
ttgcaaagttaatgcgcttgggttttaaaatacagacacctttatgctacctaaacggaa
gaacttcaatgttctgattttgcttaacatttggttgatttaaaattaaaacaaaagtac
atttgcgacaagtttcccgagaagctttgatgtcatattaaaattagaggaagtttgggg
tttagtctgtggagttgtatttctcaaaactggtctgctttatgctgaacagtctgttat
cgataaaagttgtctagctcagaagttcatgaaaatatggacttggactggataaacatt
tttttctgcccacctttgctgctacttgtgttaagaacaatatgtatatggaaagacact
tttcttacttttccttgaagattaagatgcaactgtctttgtaatttacataatcagcgc
tttctttggtgatatgatacaacaacaacaacatctccagtaatatcccacactatggag
gctatttccaatagaccctcggctcaagaaagcataagcaccacattaatggaaatataa
acaagaagggacagtaccaaaaagcgatataaaagcaaaataaaaacaacaagacagtaa
ggtgatcaacaatgaaagaaaacaacggttagtcataaaaacctactaccaacagaaagc
gagattgcgtgccaatactactgttatgagcactctagactacctactctactaccctaa
tcctcgacctccatatttttctatcaagggtcatgtcctcggtcagctgaagctgcgcga
tgtcttgcctattcacctctcccacttctttggcctacctctacctctccgtaggccttc
cgatgtcaacctctcacacctcctcaccggtgcgtctgtgctcctcctcctcacatgacc
aaaccacctaagccgcacttcccgcatcttgtcctcaacaggggccgcacccaccttgtc
ctgaataacctcatttttgatcctatctaacctgatgtgcccgcacatctatcttaatat
cctcatctctgctaccttcatcatctggacatgagcgatcttgactggtcaacactcagc
cccatacaacatcgttggtctgaccaccactctgtagaacttacctaagtttcggtggca
ccttcttgtcacataaaacaccggaagcgagtatccatttcatccatcccgccccaatac
gatgtgcgacatcttcatcaatctccccatccactgaataatagacccaaggtacttaaa
actccctctcctagggatgatctgcgagtccagcctcacctccccttcccctccttgagt
cttgccactgaacttacactccaagtattttgccttagtcctgctcaacttgaaaccttt
agattccagggtctgcctccatacctctaattgcgcgttcacaccgtcttgcgtctcatc
aatcaatacaatatcatctgcaaatagcatgcaccacggcacctccccttggatgtggcg
cgtcagtacgtccatcccagagcaaacaaaaaggggtttagtgctgacccctgatgcaac
cccatcacaaccggaaaatgattcgactccccacccaccgtcctcactcgggtctttact
ccattatacatgtccttaatcaacctaacgtaggcaacaggtacatccctagcctccaaa
catcctcacccttagctctaccactctctcccaaactttcatagtatggttaagcaactt
gataccccgatagttattgcaattttggatatcacgcttgttcttgtagacaggaaccat
tgtgctccacatccactcgtcgggcatcttcttcgttctaaaaatgacattaaataacct
agtgagccactccaagcctgccttgcccgcactcttccaaaacttcaccgggatttcatc
cagcccggtcgctttgcccctgctcatcttacgcatagccccctcaacttcatcaactct
aatccgcctacaataaccaaagtcacaacgactcccggagagttccaaatcacccattac
aatgctcctgtccccttccttgttcaagaaactatggaagtaggtctgccatctccgatg
gataagcccctcatccaacaaaactttaccttcttcgtcctatagaagaaggattttttt
acctatagaaggatatgttcttttgacaggtagcaagatatagtataccagtatcccttt
ttctgtcttaacacatacttctagaaaatattgacacaaaagttcataccttgcagcttc
agtaatgttcctatcatacccttgagtctgacttgaatgattgtatttatggaaaataaa
aggtatatataggatagggtaactaattcttgttgatttgtggacattggcttttgatca
tgtactatagtttcttgacaatcagaaaggaaatgacttcatgaaatctgttggacatat
cgtttttatttcgtttaaaattgaatatttttagaagttgatatacttgccttgattctg
cagttggtttctgctttgtgctcgtcgtatgatttacattacttctttagtgcacttatg
caaaattatttaacaattatgctgaaaatgtccaatctcagCCGCAGTCAAAGAATGGTG
TTGACAGTTTTGGTGGTCTTGGAGCAACCTTAATAGATTCTCTTGACACACTATATATCA
TGGGCCTGGATGAGCAGTTTCAGAGAGCTAGAGAgtgagtttattctcttcctcttctag
aatcatatgtattacttatggtacttgttttgtccgcagacaagagaaaaatgttaaact
aaatatagtgaaaattatcaaaagcaagacacactgtgtgttttcactaatttaaagtta
aaatgcaactgcaagattgctgtttcattcatttatggatttggtgccttgcatctgact
attgccagatgttgaagtgttaattttatcacttccagtttccttctcgttattaagcat
atttcctctaatctattgaatagtttttgcgaatgatgcagtatgttaggtttttaaact
ttccacatgtaattgttttcaatgaattattccacgtggctaatagtagctaacacttta
ctgatggcagATGGGTTGCAAACTCCTTGGATTTCAACAAGAACTATGATGCAAGTGTTT
TTGAGACAACCATAAGgttgctttataaggtttaatatgagttttttatgagttttcatt
atcctttctcagcttcaatgatatagcaccatgattcttgtatggttaactatgtttttc
aacatctcagGGTTGTAGGTGGGCTTCTTAGTACGTACGATCTATCTGGTGATAAGCTTT
TCCTTGATAAGGCTCAAGACATTGCTGACAGATTGTTGCCCGCATGGAATACAGAATCTG
GAATCCCTTACAACATTATCAACTTGGCAAATGGGAATCCACATAACCCTGGGTGGACAG
Gggtaagtttgaactctaataaattgcagttaatacccccccccccccccggttgatact
actccaatatcttctggcaaagaggatggagggatcagttatcacagaaaagggagggtg
gatgtgattaatactgtatgtgacaagttattagatttggttcctgattcttatgttccc
tgaagattgtggagggaacctgacacaggagaagagcatatatctattgggaggtttctg
aagaagaatcctctcttgaagtttccttataatatgttcaaagaacatttagtttgcttc
tctttgttcttttgctctcttccctgcattcgcctcccccctttcttttcaaagaacttg
tattcttacccgttttgtgaacatattgaccggatctaatagtgatctttctcctggaac
ttgtcaatattgcttatagtttctatagattgtatttttccagaggtggtttgtgcattt
ttttgaaattattgtgctctttgctctcagGGTGATAGTATCCTGGCAGATTCTGGTACT
GAGCAGCTTGAGTTTATTGCTCTTTCGCAGAGGACAGGAGACCCAAAATATCAACAAAAG
gtatgcctgagaaaatttcttaaaatataaactacattcatattcacataaaactacaac
ttgaaactatgatatgaaaattggtattgtgtagaattgattaagctacagactgttggg
tcaatctgtcctatttcagGTGGAGAATGTTATCTTAGAACTTAACAAAACTTTTCCAGA
TGATGGTTTGCTTCCAATATACATTAATCCACATAAAGGCACAACATCATACTCAACTAT
AACATTTGGGGCAATGGGCGACAGgtaatgaccttcgtttgtccattctagaatgatgcc
tgtgaaaacctgattgagtaggagtatttatccccaaaagaaaaaaagagggggagagcc
tttatcctatgcatttgtgtgaattggcatttagagcttccatgttttcttttcatatga
aaagttagtaaaagatttttttgtttcagCTTTTATGAATATTTACTCAAGGTCTGGATA
CAAGGAAACAGAACTGCTGCTGTGAGTCATTATAGgtaagcagcttaagttcacttatgt
ctgtttcgcttcagatattgttgtccttttaaagcttcaattcagtccatccggtgtttc
acttgatggttcctgtaggtataagtgcatatattaatacacttcctcagcctgaaatca
aatctgatcatgtcttgcgggaatgcatagaaatattcattgatagtgtttacagatttg
gagcatttagaatttcaagtaagaaatcttagaacaaggggaaaaaattttgcactaagg
ataaaaagctgacgtaaatgagatatggtgtcactgtgaatacataatatcagagctata
tgcttacaacagcagcaaatacttctcaatcgaagctagttgagaaattttgatgatatt
tcacagtcaggcctgaataaacttaattatgttttaactcgctcctcacgtgcgggcttg
atttcctttaatgagccaaacacgtggaaattctttttggtccttttagtggtgagccaa
acagttaggaggtgtgagaggttggccatggtgggtatgatgagaggtagagacatgcca
aaaaagtattggagagaggtgattaggtaggacacggcacaacttaagcttaccgaggac
gtgacccttgataggagggtgtggaggttgagaattagggtagaaggttagtaggtagtc
gagcattttcctttttctttcccataccggtagtattagtgttagtatggtattttttta
ttcttagattgctattaccacctattgtttgattgctatctttcaccttggttttcttaa
tatcttgttgttgctactgcttattgtcaccgcttcttttcatcgtttctttagtcaagg
gtctctcgaaaagagcctctcagccctctcagggtagaagtaaggtctttatacacatta
ccctccccagaccccacttgtgggaattcattgggtttgttgttgttgcatttattttat
cactttacgaggttctgtggaagcacattggataatgctcagaaaattctatgttgtggc
tttacattttctttaaggatggtgttgtccaggccagcttgcatggttgctgctttacat
tttattttttgataaatctttctatggcatatttatactattctcacatattttttactg
gttctaatcttcaaaaacattttattaattttctcgccagacacattaggagtagtcaaa
gtggggtagctggagtattaaactcatttatgctcctaagactctttctctaattggaag
ctttaactaaattttacagtggtatttgacgagagtttgaacttgaaatttcagatctaa
aaactgtgagtactagtggaatttgttacaagtggttgatctttcccttgaatccttttc
cttctggtgctagaatgcaggaagatgaaattggttatagtggaaaggttgtgctataag
tgctcagctagaacaaaaatggatctgtgatgtggaaaagaaaaaattatgtttgatgca
taaagcctttctgagacttgaaaagatttgaaaaatgtagtgattttgtttaaccttttt
atgtttcttttacaaaattttgcattcctctgtgtttctcaatataattcttctgctaat
tttgcaagcagGAAAATGTGGGAGACATCAATGAAAGGTCTTTTAAGCTTGGTCCGGAGA
ACAACTCCTTCGTCTTTTGCATATATTTGCGAGAAGATGGGAAGTTCTTTAAATGACAAG
gtgatgtataggcttttacacatatttggggagtctgagatgtgttaattcttgactttg
ttttatttacccttttggattttgtgcagATGGATGAACTTGCATGCTTTGCTCCTGGGA
TGTTAGCTTTAGGATCATCTGGTTATAGCCCTAATGAGGCTCAGAAGTTCTTATCACTGG
CTGAGGAGgtatttttaacttacggagcatcattacggaatgtgattttaggttcctatt
tgcgaaatgatctccatatgccctaattcgtatgtgtgccactatgttgattgaaagtga
taataagaaagagttatatctacagtcatatggaggaaaattgcgtcaaaagacctatac
ttctcggagttaatgtggatgtagctaaaaacaatacacaagaaaggatccatataagca
ataccaactaattgggattaaagatccatagagttctcgtgtttgctgttactccttttt
attttggttgaagttttgtgtaattgtttaactataagtgtgagatttagagaacatcta
gttttagtgaacccctgatagtattaatgaacccttatttattattggaatgaaatgggt
ttaagtagagtataatggatatagagaattcatataatcaactcttttactagtttaggt
ttgaggtttagttaattgatttgagaagtggtctctgtcgaaaaaggttttaggttttag
ttcaacttttgagcattagcgatggtgggctgtgggcaatgctctcctaccaccagatgt
tccctttcgttggctgttatagttagctgggggtgctgaaaggtgaagtgtgggataaga
accaagtgttagtgactcttaaatgtgttagggggctgggtgttggtcttagattgtgct
tgcctctatgatttgacttgcctttcatctataggtttccctttcacatgatgggaaggc
ccagaggatcagtggttcattctataggagcttttagtgactgcagtgctgtttcttgtt
gccagaaagttctagtattgcttttttgctgaatatcttaaccttctcttgcagCTTGCT
TGGACTTGCTATAATTTTTATCAGTCAACACCTACAAAACTGGCAGGAGAGAACTATTTT
TTTAATGCCGGCCAAgtcagttttttcattttagttcatggtgatgtttgtttttgttgt
ttgcttatggtaatagcttatttaaattcttcatcctgtttaatgctcttcagGATATGA
GTGTGGGCACATCATGGAATATATTAAGGCCAGAGACAGTTGAGTCGCTGTTTTACCTCT
GGCGTTTAACAGGAAACAAGACATACCAAGAGTGGGGTTGGAACATATTTCAAGCATTTG
AAAAGAACTCAAGGATAGAATCTGGATATGTTGGACTTAAAGATgtaaggacaaactcaa
ttctttcaactttggatagtacctacacctccattatcttctttctttaaatgccttcaa
atgctgcatctataattctgtttctggaggtaaaaaatctgctgttatttcctgtgttat
ttgttaaaaatttgcgcctcctcatgaagtacactctttttttgggtttagatatcgata
attgggatgtacatacatgaatgttatttttgtgctattgtttgatggaaaacttggtgc
tcctacttggtgttgtctctctcctcaccttaaacaccagctcgcttctaaacttcagtg
ttcttttttgggttttgcagtactcttattacaggcaggtttctcaaatttgatttattg
agcaacctttaatatttagtgaagtatgaaagtatgtaacgtttgaaacggtgtacctct
gtcagcccatccattacataattgtgcgcaaagagcaatattgagctagtgagcccctct
tttttttaattgctgagcctgatctttattttctcctactagaagctcaacttcagagct
acccttttttgttctatggatgctctcagtatttttattgcatcttctcctatttgaagc
taaatttgtcctgggatagcaaaaacttgactccattccttgtagcccaatgtttctttc
cagttataaagcaagttgtgaagataaaaatgaagtggagggattttgaaatacaaggtg
tctagtttcagataatgtataattaaattgttgcgactaactttagcatgcattattgct
aacttttatcacgtcgactggtcttcatgggcagctgtcaaaagtttgtctggaacctct
ataattcagggttttgtgcttgtaatttgtcggtatgactgctttttcgtgttattcaat
ggaggcatatatcataaatttggttgtgaagggaaggttttaatttcatatacagtatcg
ttgttgacttctgttttaacactttttttcggttttcccagGTCAACACTGGTGTCAAAG
ACAATATGATGCAAAGCTTCTTTCTTGCGGAGACTTTTAAATATCTCTATCTTCTTTTTT
CACCCTCATCAGTAATCTCTCTAGATGAGTGGGTTTTTAACACAGAAGCCCACCCCATAA
AAATTGTTACCCGGAATGATCGTGCTATGAATTCTGGAGGGTCAGGTGGACGGCAAGAAT
CAGATAGGCAATCACGAACCAGGAAAGAAGGTCGATTTCGTATTAATCATTAAtcaagct
gttgataaattataatgggattgaatgaccaagtggagtgcctcatgaaacttgcatctg
aggtaaaagaaggatctgcactctgcaactccagattggctggatgtattgctatattct
gtagcttattaaatgccaccacatggagcagtagttttatgtagcttagcttagctactt
tagattcgcttcttaaactggcgtgtattataggagattgcaatttttgccggcagctcc
atttttgggcttgatgagcaaattgctagtcgcacctaatttttcccttagaaagcaaaa
actcatttcaatgggcacaaaatatgacatttgtgttacccgagtttttttctttgacgt
tggggctgggtttgagttgtactacccctgagaattgacgtgtgtaaaggtatatgtatc
tgaatttgtgaatttacgatctctgtgacgctatatgtgtttcagatatatctgatacag
agtttaagaaaggactttaaaaacttgtaagagtaaaatgagaagtttacaattattgtc
ttgaaatatataaatgtactattcttttggtatggactaaaacggaaagggtgccgtaga
aaatggaatagagggagtacgtcttttagttacatacaagtactggagatttcactggtt
aggttcagcaagtcgtttggaaaaaaattatatacatactttatttggttaatttgttta
agtttaatgattagaccttttcgaacaatttcatttctcttggtttgactttggtatcgg
tttattattggtattaacaagaaaacatacgattttcaatgatcttagtatgtttaaagc
attaaaatcagtaaggtattgcgtcaaatatcatttttattttatatttctgcttttata
tagtatcgtttaatttactattaagtgaatgatatgaacataagattggtggcacaagtg
gcaagaaagtctctgttattatatgtttcacgagtacaggc
(NtMNS1a cDNA sequence)
SEQ ID NO: 30
ATGGCGAGGAGTAGATCGTCTTCCACTACTTTCAGGTACATTAATCCGGCTTACTATCTG
AAACGGCCAAAGCGTCTGGCTTTGCTCTTCATCGTTTTTGTCTTCGCCACCTTCTTCTTT
TGGGATCGACAAACTTTAGTCCGTGATCATCAGGAAGAGATCTCTAAGTTGAATCATGAA
GTGACGCAATTGCGAAATCTGCTGGAAGATTTGAAGAATGGTCGAGTCATGCCAGATAAA
AAGATGAAATCTAGTGGCAAAGGTGGTCATGCAGCAAAAAATATGGATTCACCAGATAAT
ATCCTTGATGCTCAGCGAAGGGAGAAAGTGAAAGATGCTATGCTTCATGCTTGGAGTTCT
TATGAAAAATATGCATGGGGTCATGATGAATTACAGCCGCAGTCAAAGAATGGTGTTGAC
AGTTTTGGTGGTCTTGGAGCAACCTTAATAGATTCTCTTGACACACTATATATCATGGGC
CTGGATGAGCAGTTTCAGAGAGCTAGAGAATGGGTTGCAAACTCCTTGGATTTCAACAAG
AACTATGATGCAAGTGTTTTTGAGACAACCATAAGGGTTGTAGGTGGGCTTCTTAGTACG
TACGATCTATCTGGTGATAAGCTTTTCCTTGATAAGGCTCAAGACATTGCTGACAGATTG
TTGCCCGCATGGAATACAGAATCTGGAATCCCTTACAACATTATCAACTTGGCAAATGGG
AATCCACATAACCCTGGGTGGACAGGGGGTGATAGTATCCTGGCAGATTCTGGTACTGAG
CAGCTTGAGTTTATTGCTCTTTCGCAGAGGACAGGAGACCCAAAATATCAACAAAAGGTG
GAGAATGTTATCTTAGAACTTAACAAAACTTTTCCAGATGATGGTTTGCTTCCAATATAC
ATTAATCCACATAAAGGCACAACATCATACTCAACTATAACATTTGGGGCAATGGGCGAC
AGCTTTTATGAATATTTACTCAAGGTCTGGATACAAGGAAACAGAACTGCTGCTGTGAGT
CATTATAGGAAAATGTGGGAGACATCAATGAAAGGTCTTTTAAGCTTGGTCCGGAGAACA
ACTCCTTCGTCTTTTGCATATATTTGCGAGAAGATGGGAAGTTCTTTAAATGACAAGATG
GATGAACTTGCATGCTTTGCTCCTGGGATGTTAGCTTTAGGATCATCTGGTTATAGCCCT
AATGAGGCTCAGAAGTTCTTATCACTGGCTGAGGAGCTTGCTTGGACTTGCTATAATTTT
TATCAGTCAACACCTACAAAACTGGCAGGAGAGAACTATTTTTTTAATGCCGGCCAAGAT
ATGAGTGTGGGCACATCATGGAATATATTAAGGCCAGAGACAGTTGAGTCGCTGTTTTAC
CTCTGGCGTTTAACAGGAAACAAGACATACCAAGAGTGGGGTTGGAACATATTTCAAGCA
TTTGAAAAGAACTCAAGGATAGAATCTGGATATGTTGGACTTAAAGATGTCAACACTGGT
GTCAAAGACAATATGATGCAAAGCTTCTTTCTTGCGGAGACTTTTAAATATCTCTATCTT
CTTTTTTCACCCTCATCAGTAATCTCTCTAGATGAGTGGGTTTTTAACACAGAAGCCCAC
CCCATAAAAATTGTTACCCGGAATGATCGTGCTATGAATTCTGGAGGGTCAGGTGGACGG
CAAGAATCAGATAGGCAATCACGAACCAGGAAAGAAGGTCGATTTCGTATTAATCATTAA
(NtMNS1a protein sequence)
SEQ ID NO: 31
MARSRSSSTTFRYINPAYYLKRPKRLALLFIVFVFATFFFWDRQTLVRDHQEEISKLNHE
VTQLRNLLEDLKNGRVMPDKKMKSSGKGGHAAKNMDSPDNILDAQRREKVKDAMLHAWSS
YEKYAWGHDELQPQSKNGVDSFGGLGATLIDSLDTLYIMGLDEQFQRAREWVANSLDFNK
NYDASVFETTIRVVGGLLSTYDLSGDKLFLDKAQDIADRLLPAWNTESGIPYNIINLANG
NPHNPGWTGGDSILADSGTEQLEFIALSQRTGDPKYQQKVENVILELNKTFPDDGLLPIY
INPHKGTTSYSTITFGAMGDSFYEYLLKVWIQGNRTAAVSHYRKMWETSMKGLLSLVRRT
TPSSFAYICEKMGSSLNDKMDELACFAPGMLALGSSGYSPNEAQKFLSLAEELAWTCYNF
YQSTPTKLAGENYFFNAGQDMSVGTSWNILRPETVESLFYLWRLTGNKTYQEWGWNIFQA
FEKNSRIESGYVGLKDVNTGVKDNMMQSFFLAETFKYLYLLFSPSSVISLDEWVFNTEAH
PIKIVTRNDRAMNSGGSGGRQESDRQSRTRKEGRFRINH*
(NtMNS1b with 5′ and 3′ UTR)
SEQ ID NO: 32
tgcgtcatttcgaagtctcaaattatggataaacaatacaatttttgtattttggacatt
atgaagtatgacataacatacatctgagtatqaatcaccttactattgaaaagaagtgcg
ttaacttgaggattaaataataatacatagaacgtcgactggttcaatgagtatctttgt
gcatgacgtaacaaacacatactatatcaatatcaaatgccttactttttaaatattatt
ccatcgataaaaataatttgaggattaagtaatacacatagacgagctactggtctttgg
gcggtaatttcccgatcaatttactgatttatttatccttcagcttcttccaacgtctta
ttaaatgaaatttaaggtgcatttgcaaagctacattaatactaggctttaattacatga
attggtctgtttttctagttaattgattaattggtcaatattgaattgattgcaattgaa
ggatatcattattttctccaactctttaggggtacaaaaattgcaggtaattatgtataa
tagttaaattcaaaatacgacttttaaatttatgtttatatttgttctctaatataaatt
ccttcttaaacttactcccattttaatgctctcctatttctatgtatccatataaatatt
tgtcataagaaaatattttctaaaaaaatgtatgattaaaagaatttttttagtaaagga
aaagatatttaccgtggattgaccaaatatattcgaagaaaaagatagaaatggatggga
gaagacaaagcttggtatgttagtcctaggtaaggtgggatgcctaatcttaaatggaag
acaagtcaatgttacaccgaccgcgcatgattgataagagtactattattaccgcgtttt
cactctttaccaaggctgaacgggtctttacctaattaacgtcctgtagatttaggcgag
gtttccttttgggaagtccagtagtcttggtcttcttggtcgttcctcttccccgatcta
ttcaatctqcatcqqaaqatcgatctgcacttcqatttactctqtttqqtatattcataa
attgggtggaATGGCGAGGAGTAGATCGTCTTCCACTACTTTCAGGTACATTAATCCGGC
TTACTATCTGAAACGGCCAAAGCGTCTGGCTTTGCTCTTCATCGTTTTTGTTTTCGCCAC
CTTCTTCTTTTGGGATCGACAAACTTTAGTCCGTGATCATCAGgttcttcttctctttca
ttttccaatttttttcaccgtcctttttctctgattattttctttgtggaattcatgttt
aattttggattaaagtttttaagttgcgttctttaattacaaaacaactatattctttat
gtttttttttttgcagGAAGAGATCTCTAAGTTGAATGATGAAGTGATGAAATTGCGAAA
TCTGgttagtggttatctgaattatctatagctgtggaattttttattttaataatcagc
ctactgtctttaattcttttgtggctgccattcctcttcttgctttgtcgggggactgta
tgctagagcgtcttttaatgtgtgccagactgccagtacaaagttgtaattactcgagct
acctcctgttcttccttcttcaaattagatgaggttgagaatctgattaactacttgtag
tgggggaaaaagataaacttactaattcatgttagatttaacatctgtgtgttaatatgg
gaaaaatattaatgtctagagggatttatatggccagcttggttatgaagcctgaatttg
gttcgcttagcgaagagctaccatgtaccacctttacacctacttaatacctcaatctgc
ttaagtaaggctagtactgcccaacactgaattcggtttgcctagtgaagagttctctgt
ctttcactgagcttaatacctcaatctgcttcagttagctcagggctagtactgcagtgt
tgggccctataaatgggcttggagtttaaaaaatatttgtggcattaaagcttaggacca
tcttaccatgtttagatattataggaaatgaaaaagcagaaaaagtcgagctacgacccg
gcaaacagaaaaggaacccaactaaattagtcttaagaaggtgatacatctggctcagct
caaaccaggatgtaaagattagccgatggactgaccaaacaagagatggtggatggagta
agagtcgagatgtcgcaatttacctatagtgcaccataggtcagcaccttttgtgttatt
cccttagcattaaagggagaggtaacagtaggtagcaaaaagtcctcgctgaggcatgta
gaatgcttcctcattggggttaaggagaactggaggagtgttcaagtagacttgagagta
ccaacacccaatggcttaaaatgatgggacagaatactctatacacacacacacacacac
acacacacatatatatatatatctatgtgtaatgaaacttcatgaaaatatctatgtgct
atgtacttctttctttgtccgtcttgtttgtctaaaagtttggtggtttggtttatattt
tctggaaaaagaagcacaaagaatggatatagctagttatgacttatgccagtattattt
tcatgtgtgcttgcttcgcagtttacccatcttctgttgtttgcagtatagcattcaagc
tttttattttaaatactcaacttgtttgcatttattttggatactgttttagCTGGAAGA
TTTGAAGAATGGTCGAGTCATGCCAGGTGAAAAGATGAAATCTAGTGGCAAAGGTGGTCA
TGCAGCAAAAAATATGGATTCACCAGATAATATCCTTGATGCTCAGCGAAGGGAGAAAGT
GAAAGATGCTATGCTTCATGCTTGGAGTTCTTATGAAAAATATGCATGGGGTCATGATGA
ATTACAGgtttggatgttactttgaataagttcttttttgtgttgttaatgttgcctttt
ttgttgtatcttgtgatttcgcatgttttgttgcctttttcctttttgtgtgttaagggg
aaatggggtataatagatgattagttaattacttaattaaatgagttagttgtaaattta
aaaaactatttaaaaattaaatgagttagttgtcaattgacgttctccattaccttttct
ttctttgttatttaattttcctaagtgctataccttttgttgactagataagcatgtgac
actctagtttttcagttacaatattctgtaggttagtttgcagcagcaatgacaaaaact
acgcctcaaaaatataaatcatcttgatatagtttgctctatttgggcccatttcatgtc
aaccttcaatagtttggggttttctaacagtagagattctctacaattcctagtaacata
cacttcttcttttgagaaaagtaacacaaattcaaactttttgtttattatgtttttact
cattccatcccatttcatgttccagtggttgactgggtattaaagttaagaaataaggaa
gactttttacacgtaatacaaatatatacaacataccaaaatgacctttactattaacat
ctaaatgaaaggaggtaacttaccttaccttcctgataaaaaaaggttaccttatcctcc
caaagaaaaggttgtaagagttccatatatcacttactatttctatctcctaataaaaaa
agtttttatattaagtgggttcctaagaggttatgtcagtaagcgtaaaacgttattgcg
aggagtaaattgtttgcaattacaaaaatgtctcactcttctctggatagactaaaaagg
aagtaatgccacataaaatgggacaggaggagtatatgttcttttcttcatatatcctga
ccaagtatattgatttagcatgttttgatgctctggatattgcaaatgactatgaaatag
caattaaatggctaagaattggccttttaatttgttcttttctagggtatgttttgacat
gattccctagatatttctgaattattgtgagtgtcctggtagtgaggatgacaatttcat
cttgcaaagttaatgcgcttgggctttaaaataccgacacctttatgctacctaaacgga
agaacttcaatgttctgattttgcttaacatttggttgatttaaaattaaaacaaaagta
catctgcgacaagtttccagagaagctttgatgtcaacttaaaattagaggaagtttggg
gtttaggctgtggagttgtatttctcaaaactggtctgctttatgctgaacagtgttatc
gataaaagtcgtctagctcagaagttcatgaaaatatggacttggacatggataaacatt
tttttgtgcccacctttgctgctacttgtgttaagaacaatatgtatatggaaagacact
tttcttacttttccttgaagattaagatgcaactgtctttgtaatttacataatcagcgc
tttctttggtgatatgatgtaacaattttttttacctatagaaggatatgttttttgata
ggtagcaggatatagtatcccttcatatgcaatcttattctactctctttcttctttttc
tgtctaaacacacaattctagaaaatattgacacaaaagttcataccttgcagcttcagt
aatgttcctatcatacccttgaggccgacttgaatgattgtatttatggaaaataaaagg
tatatgtaggatagggtaactaattcttgttgatttgtagacattggcttttgatcatgt
actatagtttcttgacaatcagaaaggaaatgacttcatgaaatctgttggacatatcct
ttttatttcgtttaaaattgaatatttttagaagttgatatacttgccttgattctgcag
ttggtttctgctttgtgcttgtcgtacgatttacattacttctttactgcacttgtgcaa
aattatttaataattatgctgaaaatgtccaatctcagCCGCAGTCAAAGAATGGTGTTG
ACAGTTTTGGTGGTCTTGGAGCAACCTTAATAGATTCTCTTGACACACTATATATCATGG
GCCTGGATGAGCAGTTTCAGAGAGCTAGAGAgtgagtttattctcttcctcttctagaat
catatgtattacttatggtacttgttttgtccgcagacaagagaaaaatgttaaactaaa
tatagtgaaaattatcaaatgcaagacactgtgtgttttcactaatttaaagttaaaatg
caactgcaagattgctgtttcatteatttatggatttgatgccttgcatctgaccgttgc
cagacgttgaagtgttaattttatcacttccagcttccttctcgttattaagcatatttt
ctctaatctattggatagtttttgcaaatgatgcagtatgttaggtattcaaactttcca
catgtaattgttttcaatgaattattccacgtggctaatagtggctaacactttactgat
ggcagATGGGTTGTGAACTCCTTGGATTTCAACAAGAACTATGATGCAAGTGTTTTTGAG
ACAACCATAAGgttgctttataaggtttaatatgagttttttatgagttttcgttatcct
ttctcagcttcaatgatatagcaccatgattcttgtatgattaattatgtttttcaacaa
ctcagGGTTGTAGGTGGGCTTCTTAGTACGTATGATCTATCTGGTGATAAGCTTTTCCTT
GATAAGGCTCAAGACATTGCTGACAGATTGTTGCCCGCATGGAATACAGAATCTGGAATC
CCTTACAACACTATCAACTTGGCTCATGGGAATCCACATAACCCTGGGTGGACAGGGgta
agtttgaactctaataaattgcagttaatccccccctgttgatactactccaatatcttc
tggcaaagaggatggagggatcagttatcccagaagggtggatgtgattaatactgtatg
tgacaagttattagatttggttcctgattcgttccctgaagattgtggagggagcccgac
ataggagaaagtatatatctattgggaggtttctgaagaagaatcctctctttaagtttc
cttataatatattcaaagaacatttagtttgcttctctttgttcttttgctcttttccct
gcattcacctcccccctttcttttcaaagaacttgtattcttacccatttaacaaacata
ttgactgatctaatagtgatctttctcctggaacttgtcaataatgcttatagtttctat
agattgtatttttccagaggtggtttgtgcatttttttgaaattgttgtgctctttgctc
tcagGGTGATAGTATCCTGGCAGATTCTGGTACTGAGCAGCTTGAGTTTATTGCTCTTTC
GCAGAGGACAGGAGACCCAAAATATCAACAAAAGgtatgcctgagaaaatttcttaaaat
acaaactacgttcatattctcataaaactacaacttgaaactatgatatgaaaattggta
ttgtgtaaaattgattaagctacagacttgggtcaatctgtcttatttcagGTGGAGAAT
GTTATCTTGGAACTTAACAAAACTTTTCCAGAGGATGGTTTGCTTCCAATATACATTAAT
CCACATAAAGGCACAACATCATACTCAACTATAACATTTGGGGCAATGGGCGACAGgtaa
atgaccatcgtttgtccattcttgcttccccggaccccgcgcatatcgggagcttagtgc
accgggctgccctttttttttgtccattctagaatgatgcctgtgaaaacctgattgagt
aggagtatttatccccaaaagaaaaaaagagggggagagcctttatcctatgcatttgtg
aattggcatttagagcttccatgttttcttttcatatgaaaagttagtaaaagatttttt
tgtttcagCTTTTATGAATATTTACTCAAGGTCTGGATACAAGGAAACAGAACTGCTGCT
GTGAGTCATTATAGgtaagcagcttaagttcacttctgtctgtttcgcttcagatattgt
tgtccttttaaagcttcaattcagtccatccggtgtttcacttgatggttcatgtaggtc
taagtgcatattttaatgcttaaacacttcctcagcctgaaatcaaatctgatcatgtgt
tgcgggaatgcatagaaatattcgttgacaatgtttacatatttggagcattttagaatt
tcaagtaagaaatcctagaacaaggaaaaaaattttgcactgaggataaaaaactgatgg
aaatgagatatggtgtcactgtgaatacataaaatcagagctatatacttacaacaacag
caaatacgcctcaatcgaaactagttgagaatttttgatgatatttcagtcaggcctgaa
taaacttaattatgttttaactcgctcctcacgtgcgggcttgattctttttggtctttt
tagtggtgagccaaacagttaggagatgtgagaggttggccttggtgggtacgaggagag
gtagaggcagacaaaagaagtattggggagaggccttggtgggtataaggagaggtagag
gcaggccaaagaagtattggggagaggtgattaggcaggacatgacgcaacttaagctta
ccgaggacatgacccttgataggagggtgtggcggtcgagaattagggtagaaggttagt
aggtagtcgagcattttcctttttctttcccatgccgatattattagtgttagtatgata
tctttttattcttagattgctattgctacctattgtttgattgctatctttcacttcaat
tttcttaatatcttgatgttgttactgtttattgccactgcttcttttcatcgtttcttt
agccaagggtttatcgaaaagagtccctctgccctctcagggtagaggtaaggtctgcat
acacactaccctacccaaaccccacttgtgtaaattcactgggtttgttattgttgcatt
tattccatcactttacgaggttctgtggaagcacattggataatgcacattggatataca
ttttctttaaggatggtgttgtccaggccagcttgcatggttgctgctttacatttaatt
ttttgataaatctttctatggcatatttatactattctcacatatattttttacttgttc
taagcttcaaaaactttttattaattgtctcgccagacacattaggagtagtcaaagtgg
ggtagctggagtattaaactcatttatgctcctaagactctttctctaattagaagcttt
aactaaattttacagtggtatttgacgagagtttgaacttgaaatttcagatctaagaac
tgtgagtactagtggaatttgttataagtggttggtctttcccttgaatacttttccttt
tctggtgctagaatgcaggaagatgaaattggttatagtggaaaggttgtggtataagtg
cttagctagaacaaaaatggatctgtgatgtggaaaagaaaaaaatatgtttgatgcata
aagcctttctgagacttgaaaaaatatgaagtgattttgtttaacctttttatgtttctt
ttacaaaattttgcattcctctgtgttcctcaatataattcttccactaattttgcaagc
agGAAAATGTGGGAGACATCAATGAAAGGTCTTTTAAGCTTGGTTCGGAGAACGACTCCT
TCGTCTTTTGCATATATTTGCGAGAAGATGGGAAGTTCTTTAAATGACAAGgtgatgtat
aggcatttacacatatttggggagtctgagatgtgttaattcttgactttgttttattta
cccttttggattttctgcagATGGATGAACTTGCATGCTTTGCTCCTGGGATGTTAGCTT
TAGGATCATCTGGTTATAGCCCTAATGAGGCTCAGAAGTTCTTATCACTGGCTGAGGAGg
tatttttaacttgcagagcatcattgcggaatgtgattttaggttcctatttgcgaaatg
atctccatatgccctaattcgtatgtgtgccactatgttgattgaaagtgataataagaa
agaggtatatctacagtcatatggaggaaaattgcgtcaaaagacctatacttctcggag
ttaaatgtggatgtagctaaaaacaatacacaagaaaggatccatataagcaataccaac
taattgggattaaagatccatagagttctcatgtttgetgttactcctttttattttggt
tgaagttttgtgtaattgtttaactataagtgtgagatttagagaacatctagttttagt
gaacccctgatagtattactgaacccttatttattattggaatgaaatggttttaagtag
agcataatggatacagagaattcatataatcaactctttactagtttaggtttgatgttt
agttaattgattaattgatttgagaagtggtctctgtcgaaaaaagttttaggttttatt
tcaacttttgagcattagcgatggtgggctgtgggcaatgctctcctaccaccagatgtt
cgctttcgttggctgttatagttagctgggggtgctgaaaggtgaagtgtgggataagaa
acaagtgttagtgactcatgaatgtgttagggggctgagtgttggtctttagattgtgct
tgcctctatgatttgacttgcctttcatctataggtttccctttcacatgatgggaaggc
ccagaggatcagtggttcattccataggagcttttagtgactgcagtgctgtttcttgtt
gccagaaagttctagtattgcttttttgctgaatatcttaaccttctcttgcagCTTGCT
TGGACTTGCTATAACTTTTACCAGTCAACACCTACAAAACTGGCAGGAGAGAACTATTTT
TTTAATGCCGGCCAAgtcagtttttttcattttagttcatggtgatgtttgtttttgttg
tttgcttatggtgataacttatttgaattgttcatcctatttaatgctcttcagGACATG
AGTGTGGGCACATCATGGAATATATTAAGGCCAGAGACAGTTGAGTCGCTGTTTTACCTC
TGGCGTTTAACAGGAAACAAGACATACCAAGAGTGGGGTTGGAACATATTTCAAGCATTT
GAAAAGAATTCAAGGATAGAATCTGGATATGTTGGACTTAAAGATgtaaggacaaactca
attctttcaactttggatagtacctacacctccattatcttctttctttaaatgccttca
aatgctgcatctttaataatatttcccgtgttctttgttaaaaaactcatgaaatacact
cttttttggattttgatattgataattgggatatacatacatgaatgttatttttatgct
attgtttgatggaaaacctggtgctcctacttggtgttttctctctccttcaccttgtaa
acaccagctcgcttctaaacttcagttttcttttttgggttttacagcactctaattaca
ggtaggtttctccaatttgatttattgagcaaccttctataattagtgaagtatgaaagt
atgtaacgtttgaaaaggtgtacctctgtcagcccatccgtccattacataattgtacac
aaagagcaacattggctagtgagccccccctttttttaattgctgcccctgatctttatc
ttctcctactagaagctcaacttcagagctacccttttttgttctatggatgctctcaat
atttctattgcatcttctcctatttgaagctaaatttgtcctgggacagcaaaaacttga
ctccgttcctcgtagcccaatgtttctttccagttataaagcaaattgtgaagataaaaa
tgaagtggagggattttgaaatacaaggtgtcgagtttcagagaatgtataattaagttg
ttgtgactaactttagcatacataattgccaacttttatcacgtcgactggtcttcatgg
gcagctgtcaaaagtttgtcgggaacctctagaactcagggttttgtgcttgtaatttgt
cggtatgactgctttttcgtgttcaatagggacatatatcactaaatttggttgtgaagg
gaaggttttaatttcatattcagtatcattgttgacctctcttttaacgctttttttttg
ggttttCCcagGTCAACACTGGTGTCAAAGACAATATGATGCAAAGCTTCTTTCTTGCGG
AGACTCTTAAATATCTCTATCTTCTTTTTTCACCCTCATCAGTAATATCCCTAGATGAGT
GGGTTTTTAACACAGAAGCCCACCCCATAAAAATTGTTACCCGGAATGATCATGCTATGA
GTTCTGGAGGTTCAGGTGGACGGCAAGAATCAGATAGGCAATCACGAACCAGGAAAGAAG
GTCGATTTCGCATTAATCATTAAtcaagctgttgataaactataatgggattcaatgacc
aagtggagtgcctcatgaaacttgcatctgaggtaaaagaaggatctgcactctgttaac
tccagattggctgggtgtattgctatattctgtagcttattaaatgcaccacatggagca
gtagttttatgtagcttagcttagctactttaagattcgcttcttaaactggcgtgtatt
ataggagattgcaatttttgccggcagctccacatttttgggcttgatgagcaaattgct
agtcgcacctaatttttcccttagaaagcaaaaactcatttcaatgggcacaaaatatga
catttgtgttcctgagtttttttctttgacgttggggctgggtttgtgttgtactacccc
tgagaattgacgtgtgtaaagttatatgtatctgaatttgtgaatttgcgatctctgtga
cactatgtgtttcagttatatctgatactcatttttatatacctgtatttgattggacac
ggagtttgcggctttaaacatgttaaaagcatgtcattaaaagtaaaataagaagtttca
gttaattgttgagtttttggcaaaaatcatcgttcaactatggctcaaaactagggtata
tccttgtgtaataatagtgaacaaaaaatatccctgaactattcaaaaaatggcaagaat
tccttctgttaatttcttacaaccaaaacatgtgccaagcacatgacctccccccccccc
cccaaatcccccttcactcctgattctatccctcccgaagctatcccgctcttccatatt
cagtgaaactaaggcttcaaaagctatacattctacgtttaacttcataaaataactaga
gcaacaagataagttattttcttgaacaagaattgaagcta
(NtMNS1b cDNA sequence)
SEQ ID NO:61
ATGGCGAGGAGTAGATCGTCTTCCACTACTTTCAGGTACATTAATCCGGCTTACTATCTG
AAACGGCCAAAGCGTCTGGCTTTGCTCTTCATCGTTTTTGTTTTCGCCACCTTCTTCTTT
TGGGATCGACAAACTTTAGTCCGTGATCATCAGGAAGAGATCTCTAAGTTGAATGATGAA
GTGATGAAATTGCGAAATCTGCTGGAAGATTTGAAGAATGGTCGAGTCATGCCAGGTGAA
AAGATGAAATCTAGTGGCAAAGGTGGTCATGCAGCAAAAAATATGGATTCACCAGATAAT
ATCCTTGATGCTCAGCGAAGGGAGAAAGTGAAAGATGCTATGCTTCATGCTTGGAGTTCT
TATGAAAAATATGCATGGGGTCATGATGAATTACAGCCGCAGTCAAAGAATGGTGTTGAC
AGTTTTGGTGGTCTTGGAGCAACCTTAATAGATTCTCTTGACACACTATATATCATGGGC
CTGGATGAGCAGTTTCAGAGAGCTAGAGAATGGGTTGTGAACTCCTTGGATTTCAACAAG
AACTATGATGCAAGTGTTTTTGAGACAACCATAAGGGTTGTAGGTGGGCTTCTTAGTACG
TATGATCTATCTGGTGATAAGCTTTTCCTTGATAAGGCTCAAGACATTGCTGACAGATTG
TTGCCCGCATGGAATACAGAATCTGGAATCCCTTACAACACTATCAACTTGGCTCATGGG
AATCCACATAACCCTGGGTGGACAGGGGGTGATAGTATCCTGGCAGATTCTGGTACTGAG
CAGCTTGAGTTTATTGCTCTTTCGCAGAGGACAGGAGACCCAAAATATCAACAAAAGGTG
GAGAATGTTATCTTGGAACTTAACAAAACTTTTCCAGAGGATGGTTTGCTTCCAATATAC
ATTAATCCACATAAAGGCACAACATCATACTCAACTATAACATTTGGGGCAATGGGCGAC
AGCTTTTATGAATATTTACTCAAGGTCTGGATACAAGGAAACAGAACTGCTGCTGTGAGT
CATTATAGGAAAATGTGGGAGACATCAATGAAAGGTCTTTTAAGCTTGGTTCGGAGAACG
ACTCCTTCGTCTTTTGCATATATTTGCGAGAAGATGGGAAGTTCTTTAAATGACAAGATG
GATGAACTTGCATGCTTTGCTCCTGGGATGTTAGCTTTAGGATCATCTGGTTATAGCCCT
AATGAGGCTCAGAAGTTCTTATCACTGGCTGAGGAGCTTGCTTGGACTTGCTATAACTTT
TACCAGTCAACACCTACAAAACTGGCAGGAGAGAACTATTTTTTTAATGCCGGCCAAGAC
ATGAGTGTGGGCACATCATGGAATATATTAAGGCCAGAGACAGTTGAGTCGCTGTTTTAC
CTCTGGCGTTTAACAGGAAACAAGACATACCAAGAGTGGGGTTGGAACATATTTCAAGCA
TTTGAAAAGAATTCAAGGATAGAATCTGGATATGTTGGACTTAAAGATGTCAACACTGGT
GTCAAAGACAATATGATGCAAAGCTTCTTTCTTGCGGAGACTCTTAAATATCTCTATCTT
CTTTTTTCACCCTCATCAGTAATATCCCTAGATGAGTGGGTTTTTAACACAGAAGCCCAC
CCCATAAAAATTGTTACCCGGAATGATCATGCTATGAGTTCTGGAGGTTCAGGTGGACGG
CAAGAATCAGATAGGCAATCACGAACCAGGAAAGAAGGTCGATTTCGCATTAATCATTAA
(NtMNS1b protein sequence)
SEQ ID NO: 62
MARSRSSSTTFRYINPAYYLKRPKRLALLFIVFVFATFFFWDRQTLVRDHQEEISKLNDE
VMKLRNLLEDLKNGRVMPGEKMKSSGKGGHAAKNMDSPDNILDAQRREKVKDAMLHAWSS
YEKYAWGHDELQPQSKNGVDSFGGLGATLIDSLDTLYIMGLDEQFQRAREWVVNSLDFNK
NYDASVFETTIRVVGGLLSTYDLSGDKLFLDKAQDIADRLLPAWNTESGIPYNTINLAHG
NPHNPGWTGGDSILADSGTEQLEFIALSQRTGDPKYQQKVENVILELNKTFPEDGLLPIY
INPHKGTTSYSTITFGAMGDSFYEYLLKVWIQGNRTAAVSHYRKMWETSMKGLLSLVRRT
TPSSFAYICEKMGSSLNDKMDELACFAPGMLALGSSGYSPNEAQKFLSLAEELAWTCYNF
YQSTPTKLAGENYFFNAGQDMSVGTSWNILRPETVESLFYLWRLTGNKTYQEWGWNIFQA
FEKNSRIESGYVGLKDVNTGVKDNMMQSFFLAETLKYLYLLFSPSSVISLDEWVFNTEAH
PIKIVTRNDHAMSSGGSGGRQESDRQSRTRKEGRFRINH*
(NtMNS2 with 5′ and 3′ UTR)
SEQ ID NO: 63
ccacagacggcgccaaactgtttgaccaaaaagcgctaagcttttcgttaaactaattaa
taaagaaaatggaagataaatcttaaccaaaaataattaactttagatctaagcatattg
aatgcaagaatcgaatgaggccgagcttatataacatttcttaggatgattaaaagacat
caaacgtaaataataagcttacatctttgatacattgttccgtacttgtaagagcaaaga
gggaaagtaagaaatgtcgttgataactgtgagatctatctttattgattcaacaatgac
gattacaaagttttaggcttttactttgttgttggaggtctcctcccgttcttctgttcc
tttttctctcttttttaggaacccccttttcttgcctttttctctcatatatatatatat
attaccaatctttccttttatccaacggtctttaaccagcataccttctcttggctatat
ttttccttactcgcctaagtattacgacatactttctaccgtataagccttctgatggct
cgatctcgatagtggccgagatactcatcattattatacctcgtaggtacaactatagct
tggtggatcctttactattccttttaacgataaccgacatgtggtcagattttgacctat
acaggctttggcattcttaagttggcaaacggcgtgtctggctctacgtcaatttagcac
caactaaagaagctaaaggaaaaattaagtcccaactattttagcagggtgttctcgttc
ccaacgaagtacactgtagctccaacctacgccctaacggctattggtctgtactgtttc
ttgttttaaatataagtaaaatatacttatttcctaatggactaatggagtctttcccct
ttgtttaacgaaccagtcctgatcttgatcgatcttteacttgatctcgctgataaacaa
aaacgatatagaggttaacaaaggttcctcttcgcccctctcctttctttgtatagtatt
gaaataaagagaagtaaaATGGGGAGGAGTAGATCGTCCGGCAATAGGTGGAGGTACATC
AATCCATCTTACTATTTGAAACGGCCTATGCGTCTCGCATTGCTTTTCATTGTTTTTGTA
TTTGGTACTTTCTTCTTTTGGGATCGACAAACTTTAGTCCGAGATCACCAGgtacttttg
tttttccctacttcattgtcaattcccttttattggaactaatcactcttaactcccgta
atttgggtaattggttctgccatcgatcgttttctttttaattatgagcaagtttgttgc
tttgttacaacaataacactgtctatgttttcctggagaatctatgtgttccaattgtag
aattgagagccccattggacgtagcaggcttgtattttgtatctgtattagtagaaaaaa
gggcagtccggcgcactaagctcccgctatgcgcggggtcggggaagggccggaccagta
gggtctatcgtacgcagcctgcatttatgcaagaggctgtttccatggctcgaacccgtg
acctcctggtcacatgacaacaactttaccggttagtagagtcctcaataaatttgatag
actatactttggaaaattcaaggtaatcagctttttactagatttatctcttgtgttttt
gtcgtaggtcattcatacaatgaatccaagtagaacttacaaaatgtattagcaagtetc
ttctcctatcaaagagttaactatcaacagcaacactgagtatggggattgaagagttct
cagcgatatttgatttttgattacatagactgagagatatataggcattcatttcagaga
tcttctagttgctgcaggacaatatctttgaggttcttattgataattgaaacttaagtt
ggtttacggtggtaatatcacccttgataaagataaattgtttagcaaggagcaacaaaa
acaactacaccttagtcccaaactaattcagaacttctgaagaagtgtggctgctgggat
tcgtgcccaggtctttacggccagaacttggaattctactatactagacccacgttgaaa
atagagatgcaacaagaagacttcctaggggtcacaaaacctagtacggcccttgtccaa
tcctaataccctagtgctttctatttatggttgcaagcaccctgggacatttggttttct
agctagtagtaccaaagttctagtgatttttgatgcttattgcctttcagtttatataga
attttcttcttctgtttcatggaatcttcttctgatgtagaagtttatttatatgtattc
ttcataagcaagctagtggttcttagatgcatttgttatttggatatttttgaagtttta
aattcagtttgtcttgcaatttgtcaatgaacttgtgattttgcagGAAGAGATCTCTAA
GTTGCATGAAGAAGTGATACGGTTGCAAAATCTGgtaagcagtttctgcttttcttttca
aaatctgaactgttatgtttaattttcacctcttctgtaaattttggcttgtggggaaaa
tctttatactagagcttcttataattttgctggtaagtagccctttcctccttccaactg
aatgaaaaagattgtttcactgtgtataattgaaaacctgatgaagttataattcttgca
attcggttcaagcatcatttatgttgtagtaaaaatactttatgcctatgggggagaggt
atttgaggacagcaatggtgaagatagtggtggtgcgggcaataggtttggcaacaatgg
cggtggaaggaatagatgggcccatctgtgctctggcaggtttatgctgcaactgatctt
attgttagggcttgctaggtcttttttgtaaaagaacatataacgaacatcacttgcttg
ggcaaagtccatctagttttctattgtttattgtagtcgctttcaaaattcttggtgttt
taaatatttcgttctgttttcttcatcatgatttaattgctggcttttgtttccatttat
ggtcttgtttactgtagCTGGAAGAGTTGAAGGATGGTCGAGGTATATCAGGTGAAAAGA
TGAATTTTAGTCGCAGTGGTGGTGATGTGGTGAAGAAAAAGGATTTCGCTGATGACCCCA
TTGATGCTCAACGAAGAGAAAAAGTGAAAGATGCTATGCTTCATGCCTGGAGTTCATATG
AAAAATATGCATGGGGCCATGATGAACTTCAGgtctggttgttgctactaataagtcttc
tttgtagaaatattgcctttgtgccattatgtttagtcacttagcagtcaaatctttggt
ggaggcatttcagttggccgttaaatgctttaccctgttgattaatttcttatattttct
ttctctacttggagtgattgtgatcactttgtatgccttacccttaagctgatcatttaa
atgcgagtcttcatattttcatcatccctaatatttgttgggaaaatgttggatcaagag
cttcatcccagtcgtagaataatttacattctgaaatgtaatttcatccttggtggagtc
tgttttaggtttatttggctacaagttgaagaataagttatacctacataggtatcgatc
ttatgtagttagttctttcctttgtacaaaataatatcttgtactcaagattactgatta
aaaaaaaaatcttgtactcaagggtttctcagataaaaaggagttacctcaaaatttaaa
tatgtgaaagggtgaagtctcaattaattaatgctcccactttttatatttgtttcaaat
actctcacttgacactattggtgaaattatggccattccaaagtgactaacactctagct
agaaacctttgctttttcttttaccttttaatttaattttgtccttttgctattgatcta
atggaaaaatcatagctttttactttgtagcatctcatttacccttatgtccactcttta
agtaaacataaagaagttacatattattatttctcatcccaagaatcctttcatgtcgaa
gtacggtttagaacactaggagttgtcgagatgtgggaagattattcatacaattggatt
ctcaaaaagtttatcaagaattttgagtatcctggtaatgaagataacgctatcatcttt
taagctctttctatgttaaagctttgagagaggagcattagtgcaatcaaaagtgaaaac
ttcagtcttctgcatttgcaataacttctatggggaaattttttaattgagcatggtaac
aggtattttattaacaattaaagtagtccttggcacaaacaaagttacaggacctcaaaa
gaaaaagaaacaaaaagatagtcttgtgctagttacaaaaatcgcaagatgtcaactaca
gaatctacattttctacaagattaaacaatcagttacggagaaagtaaactgtaataagt
attttgttgcacatgatatttcttgttcttcttaaaaagtctgtctgcgaggtaaaaact
tgtggaagtttgtttatgtttatggtatttgggctctgcttccgagtataatagcttcat
ggtgaacaaaaatcttattcttgatggaattgctagcttcatatatgatctattcgactc
tctacttccctattcctttttctttctttgaccgaacatgtgatgtaagatcatattcac
ccagaagcttatacgtgttagcaaaatattcctagacagaatctatatggaattggtatt
agttctcaatgacttttttttgtggtgactataatttaatgacagtcagaaaggaaatgt
aaaattgtaagagagatccctttttgttcgttgttcagtactgaatctaagaggataaat
tttccttgatacttttcgaactgtttctgctatgtgcttgtggaactttatactatatcc
tttattggtcatgtgcctgtattgatttgattgtcatgataaacctttgcaatgccagCC
ACAAACAAAGAAGGGTGTTGACAGTTTTGGTGGTCTTGGGGCAACATTAATAGATTCTCT
TGACACACTATATATCATGGGCCTGGATGAGCAGTTTCAGAGAGCTAGAGAgtgagttca
ttattctcttgcccctgaaagccccgaattatctttcttattctaattcaggaattagtt
gtattataacttaaaattttgtgattgctcttgattgtaccttttccctttctttctaga
ttgagagcttttttatgtgaaaaccagctttgtatatgtggatacattatcttctacttt
attttatttgacggtgatctcttccctgcacacagtaaccatggttgtctttgacaatat
tacttatggtcctagttttgttgtaaagaagaaaatgaattgtttactttttttttttta
atatgaccgggaatcaccagaatcaagtaattggtgcatgcgataatgttaaaatgcatc
tggggttagtaaaacattttatacttattgtcatctctctgattaatgtctgcagttctc
ctaactgccgcctcctcaacagccagagtccccaaagtcctcacccagtgagagactgct
tagagtcctgtgtttccttggattgtggatttgatgtctggcattttgactttccaaaat
aattgaagtgtcaatttcattatatcccttttacttctgggttttagggttatgtattag
gtgtactttctactctctctgaaacaatgttgccaggtgataggcatttgtaactttata
tatttttgtgcttcagttaagcgttcattgcttgtggctaacaagttgttgatggcagGT
GGGTTGCAAGCTCCTTGGATTTCAACAAGAATTATGATGCCAGTGTTTTTGAGACAACCA
TAAGgtttctttataaggtttaatatgcttttgtaatgagtttacttggattcctgatac
cttttatcagctttgacgatttgtttctatgttttttgtttcaatgtttctttatgtaat
tcaacaacagAGTTGTAGGTGGACTTCTTAGTGCATATGATCTCTCTGGTGATAAGCTTT
TCCTTGATAAGGCTAAAGATATTGCTGACAGACTGTTGCCTGCATGGAATACACCATCTG
GCATCCCTTACAACATTATCAACTTGTCACATGGGAATCCACATAATCTTGGGTGGACAG
Gggtaattttgaactataccaaattcaagttgatttccgctgtagtataactcatgtatc
tcatgctgaaaaggatatagggaattatcctaaattttatttgacgagtcatttgatgct
ttaccctgcatcaataggagaagagtatctaaaaggggaactgtgtgaatgaagaatcat
acgttattaaatgctctaattttctcataatatacttaaatgatcttatgatccaatcct
tgttttctctctttcttgcatctcctccaggcgttctcccaactgacttcagcttgctgg
gagaaacatgtctgttgcaacttagcaattgcagttctctaggaaactgtcccacatact
ctcaacttgtttgtgcacccagccatcttgtgatgatgtccttttgctgaaattttcacc
agtgggaatccaactctcttctttttaattgctttttatttcttttctttggggcatatt
aggaagctgcagggcttgtgcagtcactgcgatatatggttttttacttgttcttttcct
cttaaacgcttggacagagtctttttttgcacaccaatgacttatcttttgaaatctgaa
tatttcagtctcatggcatgtgatatatgatgcttaaatttctatgcacaaacacatata
tgtaattacatcgctgtagtctagtgtacatttggtgaaattattgtgctcccttctctc
agGGTAATAGTATCCTGGCAGATTCTGCCTCTGAGCAGCTTGAATTTATTGCTCTTTCGC
AACGGACAGGAGACTCAAAGTATCAACAGAAGgtatgtgccaatagaatttatctaaaag
tataacttcttgataactactagtaaataaaactacaattccaaaattggcatggtagac
aattgattaagctacacatacttgaaacgatgttctgctagtgactgaatggcatatgtt
cctatttcagGTGGAGAATGTTATCTTAGAACTTAATAGAACTTTTCCAGATGATGGTTT
GCTTCCAATACACATTAATCCCGAGAGAGGGACAACGTCATACTCCACTATAACGTTTGG
GGCCATGGGGGACAGgtagctttcatttatctttctccatatgacagatctgataatgtg
aacctaaagaggactggtatcaccatatccgtctgttcactggcatttggttttcctttg
tttcttttgtacatttagatagtaaaactatgtcgtttcagCTTTTATGAATATTTACTC
AAGGCCTGGATACAAGGAAACAAAACAGCTGCTGTGGGACACTACAGgtaagaagcttaa
gtttaaagtttctttatttttttactttacagttttcctattcaaaacttcaagtggttt
cctgttttgacatgatgagttgcagttctgatggatccgtaactgtaaagtgtgtaaact
aatgctagaatactttgtcgggcctgaattcaagtctttgtcatgcatcacggcctaaca
catagaaatactgttaaatgtttacatgtgtagagcactaccaagaaacccaatcagagg
aaacacgtgaattttgaccgaacatgaaaggaaaaaggaccattaaggagaaaaaaatga
caacttgctgaggagttgatttaatctaaatacataaaagtaggcctggattattagagc
tgttgctattatagtatcgttcgatatacatataaatatcgaagtaagagagattaaatt
tactgctacttttttaaaaaaaagaaatttcctgctatctttatatcattctgataaata
atacataatatcaaacctgagctgcatcgggagccttaatgatgacattgttatatactc
catcactttttcctagaagggcaaaacttaaaatcttgattaacatgtaactagagtact
ctttctgtgtcgcgttcttgcactcttgttacatcttccaagcatcactttagcatgttt
ccaaaaattcagatacgccaatcctaagtttcaaatactttgttttctaactttcttgct
agttaaactagattagtcaaaacgatcaaaatttagtgcaggatgtccttatggattatc
ttgattagcagctgtaagctcagttctgcagaaactaatttgaagaccaaagaactgggg
gtttatgggcagcgtctttcctttgagaagtgcaaagcgagctccttatcctttactgct
ctgaagtgcaggaagacgaaattggttattgtctgaaaactctgtgttataattgcttag
ttagaaccaaaaggatcagaaatgtggaccaagtcaaagtatgtcaatgcatatttcttt
cctgagacttctaaatgagtatgacgttcttttgcaaattgcaatctcaagtgtattaca
tagagttcttccatttaattttccaaacagAAAAATGTGGGAGACATCAATGAAAGGTCT
TTTAAGCTTGGTGCGGAGGACTACCCCATCATCTTTTGCTTATATTGGTGAGAAGATCGG
AAGTTCTTTAAATGACAAGgtgatgtatagggttcaaattggtagctgggagttgtgatg
atgtgtgttattcttatatcatgtttaatctacccttttctgaattctatatagATGGAT
GAACTTGCATGCTTCGCTCCAGGAATGTTAGCTTTAGGGTCGTCTGGTTATGGTCCTGAC
GAGTCTCAGAAGTTCTTATCACTGGCAGAAGAGgtaaatttgaacttgtacagcattaaa
ctatgttttgacttaagttcttatttgaccatcgatctctgatggagaagttttgcatca
actttgagtatgaggttgtttaggttacattggacattgttcggcctactccagatgatt
acttggtttactttaatttatttggtggggttatacagggtgaagcatgaaacaacctat
gaaataacatgtaggtcttgaatgtgggctacagtgcagattttatcattcaaccttcta
actttctctttcagataaaagggaaagaaggcacataggatcagtgggcttaatctattg
catattgactacttccattattgctcgttagaacaggaaacttgagtattgctattttac
tggatatgttgaccccttcttgcagCTTGCTTGGACTTGCTATAACTTCTACCAGTCAAC
ACCTACAAAATTGGCAGGAGAAAACTATTTCTTTAATGATGACGGGCAGgttgattttac
caattattttattggtacatatttgttattgttgtttgcttatgctgataaagtatttgt
gattgtttttcagGATATGACTGTGGGCACATCGTGGAACATACTAAGGCCAGAAACGGT
TGAGTCTCTATTTTACCTCTGGCGTTTAACTGGAAACAAGACATACCAAGAGTGGGGTTG
GAACATATTTCAAGCATTTGAAAAGAACTCGAGAATAGAGTCTGGATATGTTGGACTTAA
AGATgtaagtacaaactcagactcctaactctagttggtgattttgttaaagattaattc
atgtgaaagaatctgagcatccaacccaaaacttaaaaggcaatgggtggagtgatccag
gacattacccttaggggctgtttggttcaaaatatcccataatcttgggattagaacagg
gactataacctggataacttatcccaccttctatatgggataagggataagttattccaa
gattttggtataacaagaatatcaggtttagctaataactccaaccaaaacgggataagt
ttaatcccaaaatttataccaagataacccacctaatcccttqaaccaaacaacccctta
cataaccgatgaaagacaagtgtattctcggagtataacccgattctcgagatgttttgg
acatctatttttaacttgttggtgtttgtcccagGTTAATACCGGTGTGCAAGACGATAT
GATGCAAAGCTTTTTCCTTGCGGAGACTCTTAAATATCTCTACCTTCTTTTCTCACCCTC
TTCACTCATTCCACTAGATGAGTGGGTCTTCAACACAGAGGCCCACCCCATAAAAATTGT
TAGCCGGAATGATCGAGCAGTGAGTTCTGGAAGGTCAGTTGGACAAACCAAATCATATAG
GCGGCCACGGACCAGGAGAGAAGGCCGATTTGGTAATAAGTAGattcacaggtcatcatt
agtttagttgttgattgagaaggccaatttgagagttggaattcaagtgcagttttgctt
ggcacttcttcaaccagattgacgggattttcccccccaacattgataaaatgctcagta
taggagaagttatgagtatgtagcatagttatttagtttcctttttctatgttcccttaa
tactagcgactgtattctagtacaggtcataagggcatttggttgcgggtagctctacat
atttggggctggacgagtttttgtatatcatacctttttattttcgtttttcaaatacaa
caggtaaattctaatttcaaggactgttgacaacttttttgcacagttgcgctatggttg
atgatcaaatatatctcttgagtaacttttggttaaaaatagcacggtctacccagtttt
tagattggttattcaaaaatagccagcgtttgccaagtcattgaaaaataactactattt
tgctgctacagaaaccggtccaacataatatactggagtgtggtgcacctgtgtatgaac
ttccagcatattatgctggaccggtatattatactggaactccagtatattatgctggag
tatttttctggattttgaatagtgttttcgttcagatttatctttacataaaaagtggct
aaattttgattactcttgaaactgtgactattttttaatgaccacttgtaaatctgacta
tttttgaatttctccctaacttttgaggttagtgctgtgagcctgtctgggtaatattgg
gttggtttaatgtatctcagaatcgatgatagcaaaaatgatatcagttagctgctctaa
agggctgttatttaggagttagcaaatgtgtcctgaattttagttgtccagtttaatttt
tcgggacataaatattctgaattgtcctcaaattaagatttttagtttaagacaaaataa
gtattgactaatatttaaataaaaaccttaagaatggatgtttgtgtaattctctcctgg
agcttgttaagtcgcattcacatactattttacgttactcc
(NtMNS2 cDNA sequence)
SEQ ID NO: 92
ATGGGGAGGAGTAGATCGTCCGGCAATAGGTGGAGGTACATCAATCCATCTTACTATTTG
AAACGGCCTATGCGTCTCGCATTGCTTTTCATTGTTTTTGTATTTGGTACTTTCTTCTTT
TGGGATCGACAAACTTTAGTCCGAGATCACCAGGAAGAGATCTCTAAGTTGCATGAAGAA
GTGATACGGTTGCAAAATCTGCTGGAAGAGTTGAAGGATGGTCGAGGTATATCAGGTGAA
AAGATGAATTTTAGTCGCAGTGGTGGTGATGTGGTGAAGAAAAAGGATTTCGCTGATGAC
CCCATTGATGCTCAACGAAGAGAAAAAGTGAAAGATGCTATGCTTCATGCCTGGAGTTCA
TATGAAAAATATGCATGGGGCCATGATGAACTTCAGCCACAAACAAAGAAGGGTGTTGAC
AGTTTTGGTGGTCTTGGGGCAACATTAATAGATTCTCTTGACACACTATATATCATGGGC
CTGGATGAGCAGTTTCAGAGAGCTAGAGAGTGGGTTGCAAGCTCCTTGGATTTCAACAAG
AATTATGATGCCAGTGTTTTTGAGACAACCATAAGAGTTGTAGGTGGACTTCTTAGTGCA
TATGATCTCTCTGGTGATAAGCTTTTCCTTGATAAGGCTAAAGATATTGCTGACAGACTG
TTGCCTGCATGGAATACACCATCTGGCATCCCTTACAACATTATCAACTTGTCACATGGG
AATCCACATAATCTTGGGTGGACAGGGGGTAATAGTATCCTGGCAGATTCTGCCTCTGAG
CAGCTTGAATTTATTGCTCTTTCGCAACGGACAGGAGACTCAAAGTATCAACAGAAGGTG
GAGAATGTTATCTTAGAACTTAATAGAACTTTTCCAGATGATGGTTTGCTTCCAATACAC
ATTAATCCCGAGAGAGGGACAACGTCATACTCCACTATAACGTTTGGGGCCATGGGGGAC
AGCTTTTATGAATATTTACTCAAGGCCTGGATACAAGGAAACAAAACAGCTGCTGTGGGA
CACTACAGAAAAATGTGGGAGACATCAATGAAAGGTCTTTTAAGCTTGGTGCGGAGGACT
ACCCCATCATCTTTTGCTTATATTGGTGAGAAGATCGGAAGTTCTTTAAATGACAAGATG
GATGAACTTGCATGCTTCGCTCCAGGAATGTTAGCTTTAGGGTCGTCTGGTTATGGTCCT
GACGAGTCTCAGAAGTTCTTATCACTGGCAGAAGAGCTTGCTTGGACTTGCTATAACTTC
TACCAGTCAACACCTACAAAATTGGCAGGAGAAAACTATTTCTTTAATGATGACGGGCAG
GATATGACTGTGGGCACATCGTGGAACATACTAAGGCCAGAAACGGTTGAGTCTCTATTT
TACCTCTGGCGTTTAACTGGAAACAAGACATACCAAGAGTGGGGTTGGAACATATTTCAA
GCATTTGAAAAGAACTCGAGAATAGAGTCTGGATATGTTGGACTTAAAGATGTTAATACC
GGTGTGCAAGACGATATGATGCAAAGCTTTTTCCTTGCGGAGACTCTTAAATATCTCTAC
CTTCTTTTCTCACCCTCTTCACTCATTCCACTAGATGAGTGGGTCTTCAACACAGAGGCC
CACCCCATAAAAATTGTTAGCCGGAATGATCGAGCAGTGAGTTCTGGAAGGTCAGTTGGA
CAAACCAAATCATATAGGCGGCCACGGACCAGGAGAGAAGGCCGATTTGGTAATAAGTAG
(NtMNS2 protein sequence)
SEQ ID NO: 93
MGRSRSSGNRWRYINPSYYLKRPMRLALLFIVFVFGTFFFWDRQTLVRDHQEEISKLHEE
VIRLQNLLEELKDGRGISGEKMNFSRSGGDVVKKKDFADDPIDAQRREKVKDAMLHAWSS
YEKYAWGHDELQPQTKKGVDSFGGLGATLIDSLDTLYIMGLDEQFQRAREWVASSLDFNK
NYDASVFETTIRVVGGLLSAYDLSGDKLFLDKAKDIADRLLPAWNTPSGIPYNIINLSHG
NPHNLGWTGGNSILADSASEQLEFIALSQRTGDSKYQQKVENVILELNRTFPDDGLLPIH
INPERGTTSYSTITFGAMGDSFYEYLLKAWIQGNKTAAVGHYRKMWETSMKGLLSLVRRT
TPSSFAYIGEKIGSSLNDKMDELACFAPGMLALGSSGYGPDESQKFLSLAEELAWTCYNF
YQSTPTKLAGENYFFNDDGQDMTVGTSWNILRPETVESLFYLWRLTGNKTYQEWGWNIFQ
AFEKNSRIESGYVGLKDVNTGVQDDMMQSFFLAETLKYLYLLFSPSSLIPLDEWVFNTEA
HPIKIVSRNDRAVSSGRSVGQTKSYRRPRTRREGRFGNK*
(NtMNS1a cDNA sequence)
SEQ ID NO: 94
ATGGCGAGGAGTAGATCGTCTTCCACTACTTTCAGGTACATTAATCCGGCTTACTATCTGAAACGGCCAAAGCGTCT
GGCTTTGCTCTTCATCGTTTTTGTCTTCGCCACCTTCTTCTTTTGGGATCGACAAACTTTAGTCCGTGATCATCAGG
AAGAGATCTCTAAGTTGAATCATGAAGTGACGCAATTGCGAAATCTGCTGGAAGATTTGAAGAATGGTCGAGTCATG
CCAGATAAAAAGATGAAATCTAGTGGCAAAGGTGGTCATGCAGCAAAAAATATGGATTCACCAGATAATATCCTTGA
TGCTCAGCGAAGGGAGAAAGTGAAAGATGCTATGCTTCATGCTTGGAGTTCTTATGAAAAATATGCATGGGGTCATG
ATGAATTACAGCCGCAGTCAAAGAATGGTGTTGACAGTTTTGGTGGTCTTGGAGCAACCTTAATAGATTCTCTTGAC
ACACTATATATCATGGGCCTGGATGAGCAGTTTCAGAGAGCTAGAGAATGGGTTGCAAACTCCTTGGATTTCAACAA
GAACTATGATGCAAGTGTTTTTGAGACAACCATAAGGGTTGTAGGTGGGCTTCTTAGTACGTACGATCTATCTGGTG
ATAAGCTTTTCCTTGATAAGGCTCAAGACATTGCTGACAGATTGTTGCCCGCATGGAATACAGAATCTGGAATCCCT
TACAACATTATCAACTTGGCAAATGGGAATCCACATAACCCTGGGTGGACAGGGGGTGATAGTATCCTGGCAGATTC
TGGTACTGAGCAGCTTGAGTTTATTGCTCTTTCGCAGAGGACAGGAGACCCAAAATATCAACAAAAGGTGGAGAATG
TTATCTTAGAACTTAACAAAACTTTTCCAGATGATGGTTTGCTTCCAATATACATTAATCCACATAAAGGCACAACA
TCATACTCAACTATAACATTTGGGGCAATGGGCGACAGCTTTTATGAATATTTACTCAAGGTCTGGATACAAGGAAA
CAGAACTGCTGCTGTGAGTCATTATAGGAAAATGTGGGAGACATCAATGAAAGGTCTTTTAAGCTTGGTCCGGAGAA
CAACTCCTTCGTCTTTTGCATATATTTGCGAGAAGATGGGAAGTTCTTTAAATGACAAGATGGATGAACTTGCATGC
TTTGCTCCTGGGATGTTAGCTTTAGGATCATCTGGTTATAGCCCTAATGAGGCTCAGAAGTTCTTATCACTGGCTGA
GGAGCTTGCTTGGACTTGCTATAATTTTTATCAGTCAACACCTACAAAACTGGCAGGAGAGAACTATTTTTTTAATG
CCGGCCAAGATATGAGTGTGGGCACATCATGGAATATATTAAGGCCAGAGACAGTTGAGTCGCTGTTTTACCTCTGG
CGTTTAACAGGAAACAAGACATACCAAGAGTGGGGTTGGAACATATTTCAAGCATTTGAAAAGAACTCAAGGATAGA
ATCTGGATATGTTGGACTTAAAGATGTCAACACTGGTGTCAAAGACAATATGATGCAAAGCTTCTTTCTTGCGGAGA
CTTTTAAATATCTCTATCTTCTTTTTTCACCCTCATCAGTAATCTCTCTAGATGAGTGGGTTTTTAACACAGAAGCC
CACCCCATAAAAATTGTTACCCGGAATGATCGTGCTATGAATTCTGGAGGGTCAGGTGGACGGCAAGAATCAGATAG
GCAATCACGAACCAGGAAAGAAGATATATCTGATACAGAGTTTAAGAAAGGACTTTAA
(NtMNS1a protein sequence)
SEQ ID NO: 95
MARSRSSSTTFRYINPAYYLKRPKRLALLFIVFVFATFFFWDRQTLVRDHQEEISKLNHEVTQLRNLLEDLKNGRVM
PDKKMKSSGKGGHAAKNMDSPDNILDAQRREKVKDAMLHAWSSYEKYAWGHDELQPQSKNGVDSFGGLGATLIDSLD
TLYIMGLDEQFQRAREWVANSLDFNKNYDASVFETTIRVVGGLLSTYDLSGDKLFLDKAQDIADRLLPAWNTESGIP
YNIINLANGNPHNPGWTGGDSILADSGTEQLEFIALSQRTGDPKYQQKVENVILELNKTFPDDGLLPIYINPHKGTT
SYSTITFGAMGDSFYEYLLKVWIQGNRTAAVSHYRKMWETSMKGLLSLVRRTTPSSFAYICEKMGSSLNDKMDELAC
FAPGMLALGSSGYSPNEAQKFLSLAEELAWTCYNFYQSTPTKLAGENYFFNAGQDMSVGTSWNILRPETVESLFYLW
RLTGNKTYQEWGWNIFQAFEKNSRIESGYVGLKDVNTGVKDNMMQSFFLAETFKYLYLLFSPSSVISLDEWVFNTEA
HPIKIVTRNDRAMNSGGSGGRQESDRQSRTRKEDISDTEFKKGL*
(NtMNS1b cDNA sequence)
SEQ ID NO: 96
ATGGCGAGGAGTAGATCGTCTTCCACTACTTTCAGGTACATTAATCCGGCTTACTATCTGAAACGGCCAAAGCGTCT
GGCTTTGCTCTTCATCGTTTTTGTTTTCGCCACCTTCTTCTTTTGGGATCGACAAACTTTAGTCCGTGATCATCAGG
AAGAGATCTCTAAGTTGAATGATGAAGTGATGAAATTGCGAAATCTGCTGGAAGATTTGAAGAATGGTCGAGTCATG
CCAGGTGAAAAGATGAAATCTAGTGGCAAAGGTGGTCATGCAGCAAAAAATATGGATTCACCAGATAATATCCTTGA
TGCTCAGCGAAGGGAGAAAGTGAAAGATGCTATGCTTCATGCTTGGAGTTCTTATGAAAAATATGCATGGGGTCATG
ATGAATTACAGTCAAAGAATGGTGTTGACAGTTTTGGTGGTCTTGGAGCAACCTTAATAGATTCTCTTGACACACTA
TATATCATGGGCCTGGATGAGCAGTTTCAGAGAGCTAGAGAGGTTGTAGGTGGGCTTCTTAGTACGTATGATCTATC
TGGTGATAAGCTTTTCCTTGATAAGGCTCAAGACATTGCTGACAGATTGTTGCCCGCATGGAATACAGAATCTGGAA
TCCCTTACAACACTATCAACTTGGCTCATGGGAATCCACATAACCCTGGGTGGACAGGGGGTGATAGTATCCTGGCA
GATTCTGGTACTGAGCAGCTTGAGTTTATTGCTCTTTCGCAGAGGACAGGAGACCCAAAATATCAACAAAAGGTGGA
GAATGTTATCTTGGAACTTAACAAAACTTTTCCAGAGGATGGTTTGCTTCCAATATACATTAATCCACATAAAGGCA
CAACATCATACTCAACTATAACATTTGGGGCAATGGGCGACAGCTTTTATGAATATTTACTCAAGGTCTGGATACAA
GGAAACAGAACTGCTGCTGTGAGTCATTATAGGAAAATGTGGGAGACATCAATGAAAGGTCTTTTAAGCTTGGTTCG
GAGAACGACTCCTTCGTCTTTTGCATATATTTGCGAGAAGATGGGAAGTTCTTTAAATGACAAGATGGATGAACTTG
CATGCTTTGCTCCTGGGATGTTAGCTTTAGGATCATCTGGTTATAGCCCTAATGAGGCTCAGAAGTTCTTATCACTG
GCTGAGGAGCTTGCTTGGACTTGCTATAACTTTTACCAGTCAACACCTACAAAACTGGCAGGAGAGAACTATTTTTT
TAATGCCGGCCAGGACATGAGTGTGGGCACATCATGGAATATATTAAGGCCAGAGACAGTTGAGTCGCTGTTTTACC
TCTGGCGTTTAACAGGAAACAAGACATACCAAGAGTGGGGTTGGAACATATTTCAAGCATTTGAAAAGAATTCAAGG
ATAGAATCTGGATATGTTGGACTTAAAGATGTCAACACTGGTGTCAAAGACAATATGATGCAAAGCTTCTTTCTTGC
GGAGACTCTTAAATATCTCTATCTTCTTTTTTCACCCTCATCAGTAATATCCCTAGATGAGTGGGTTTTTAACACAG
AAGCCCACCCCATAAAAATTGTTACCCGGAATGATCATGCTATGAGTTCTGGAGGTTCAGGTGGACGGCAAGAATCA
GATAGGCAATCACGAACCAGGAAAGAAGGAGATTGCAATTTTTGCCGGCAGCTCCACATTTTTGGGCTTGATGAGCA
AATTGCTAGTCGCACCTAA
(NtMNS1b protein sequence)
SEQ ID NO: 97
MARSRSSSTTFRYINPAYYLKRPKRLALLFIVFVFATFFFWDRQTLVRDHQEEISKLNDEVMKLRNLLEDLKNGRVM
PGEKMKSSGKGGHAAKNMDSPDNILDAQRREKVKDAMLHAWSSYEKYAWGHDELQSKNGVDSFGGLGATLIDSLDTL
YIMGLDEQFQRAREVVGGLLSTYDLSGDKLFLDKAQDIADRLLPAWNTESGIPYNTINLAHGNPHNPGWTGGDSILA
DSGTEQLEFIALSQRTGDPKYQQKVENVILELNKTFPEDGLLPIYINPHKGTTSYSTITFGAMGDSFYEYLLKVWIQ
GNRTAAVSHYRKMWETSMKGLLSLVRRTTPSSFAYICEKMGSSLNDKMDELACFAPGMLALGSSGYSPNEAQKFLSL
AEELAWTCYNFYQSTPTKLAGENYFFNAGQDMSVGTSWNILRPETVESLFYLWRLTGNKTYQEWGWNIFQAFEKNSR
IESGYVGLKDVNTGVKDNMMQSFFLAETLKYLYLLFSPSSVISLDEWVFNTEAHPIKIVTRNDHAMSSGGSGGRQES
DRQSRTRKEGDCNFCRQLHIFGLDEQIASRT*
(NtMan1.4 cDNA sequence)
SEQ ID NO: 98
ATGGGGAGGAGTAGATCGTCCACCAATAGGTGGAGGTACATCAATCCATCTTACTATTTGAAACGCCCCAAGCGTCT
CGCATTGCTTTTCATTGTTTTCGTATTCGGTACATTCTTCTTTTGGGATCGACAAACGTTAGTCCGAGACCACCAGG
AAGAGATCTCTAAGTTGCATGAAGAAGTGATACGGTTGCAAAATCTGCTGGAAGAGTTGAAGAATGGTCGAGGTGTA
TCGGGTGAAAAGGTGAATTTTAGTCGCACTGGTGGTGATGTGCTGAAGAAAAAGGATTTCGCTGAAGACCCCATTGA
TGCTCAGCGAAGAGAAAAAGTGAAAGATGCTATGCTTCACGCCTGGAGTTCATATGAAAAATATGCCTGGGGCCACG
ATGAACTTCAGCCACAAACAAAGAAGGGTGTTGACAGTTTTGGTGGTCTTGGGGCCACATTAATAGATTCTCTTGAC
ACACTATATATCATGGGCCTGGATGAGCAGTTTCAGAGAGCTAGAGAGTGGGTTGCAAGCTCATTGGATTTCAACAA
GAATTATGATGCCAGTGTTTTTGAGACAACCATAAGAGTTGTTGGTGGACTTCTTAGTGCGTATGATCTCTCTGGTG
ATAAGCTTTTCCTTGATAAGGCTAAAGATATTGCTGACAGACTGTTGCCTGCATGGAATACACCATCTGGCATCCCT
TACAACATTATCAACTTGTCACATGGAAATCCGCATAATCCTGGGTGGACAGGGGGTAATAGTATCCTGGCAGATTC
TGCCTCTGAGCAGCTTGAATTTATTGCTCTTTCGCAAAGGACAGGAGACTCAAAGTATCAACAGAAGGTGGAGAATG
TTATCGTAGAACTTAATAGAACTTTTCCAGTTGATGGTTTGCTTCCAATACACATTAATCCCGAGAGAGGGACAACG
TCATACTCCACTATAACATTTGGGGCCATGGGGGACAGCTTTTATGAATATTTACTCAAGGTCTGGATACAAGGAAA
CAAAACAGCTGCTGTGGGACACTACAGAAAAATGTGGGAGACATCAATGAAAGGCCTTTTAAGCTTGGTGCGGAGGA
CTACCCCATCATCTTTTGCTTATATTGGTGAGAAGATCGGAAGTTCTTTAAATGACAAGATGGATGAACTTGCATGC
TTCGCTCCAGGAATGTTAGCTTTAGGGTCGTCTGGTTATGGTCCTGACGAGTCTCAGAAGTTCTTATCACTCGCAGA
AGAGCTTGCTTGGACTTGCTATAACTTCTACCAGTCAACACCTTCAAAATTGGCAGGAGAAAACTATTTCTTTAATG
ATGATGGGCAGGATATGACCGTGGGCACATCGTGGAACATACTAAGGCCAGAAACGGTTGAGTCTCTGTTTTACCTC
TGGCGTTTAACTGGAAACAAGACATACCAAGAGTGGGGTTGGAACATATTTCAAGCATTTGAAAAGAACTCGAGAAT
AGAGTCTGGATATGTTGGACTTAAAGATGTTAATACCGGTGTGCAAGACAATATGATGCAAAGCTTTTTCCTTGCGG
AGACTCTTAAATATCTCTACCTTCTTTTCTCACCCTCTTCAATCATTCCACTAGATGAGTGGGTCTTCAACACAGAG
GCCCACCCCATAAAAATTGTTAGCCGGAATGATCCAGCAGTCAGTTCTGGAAGGTCAGTTGGACAAACAAAATCATA
TAGGCGGCCACGGACCAGGAGAGAAGGCCGATTTGGTAATAAGTAG
(NtMan1.4 protein sequence)
SEQ ID NO: 99
MGRSRSSTNRWRYINPSYYLKRPKRLALLFIVFVFGTFFFWDRQTLVRDHQEEISKLHEEVIRLQNLLEELKNGRGV
SGEKVNFSRTGGDVLKKKDFAEDPIDAQRREKVKDAMLHAWSSYEKYAWGHDELQPQTKKGVDSFGGLGATLIDSLD
TLYIMGLDEQFQRAREWVASSLDFNKNYDASVFETTIRVVGGLLSAYDLSGDKLFLDKAKDIADRLLPAWNTPSGIP
YNIINLSHGNPHNPGWTGGNSILADSASEQLEFIALSQRTGDSKYQQKVENVIVELNRTFPVDGLLPIHINPERGTT
SYSTITFGAMGDSFYEYLLKVWIQGNKTAAVGHYRKMWETSMKGLLSLVRRTTPSSFAYIGEKIGSSLNDKMDELAC
FAPGMLALGSSGYGPDESQKFLSLAEELAWTCYNFYQSTPSKLAGENYFFNDDGQDMTVGTSWNILRPETVESLFYL
WRLTGNKTYQEWGWNIFQAFEKNSRIESGYVGLKDVNTGVQDNMMQSFFLAETLKYLYLLFSPSSIIPLDEWVFNTE
AHPIKIVSRNDPAVSSGRSVGQTKSYRRPRTRREGRFGNK*

### Deposit

The following seed samples were deposited with NCIMB, Ferguson Building, Craibstone Estate, Bucksburn, Aberdeen AB21 9YA, Scotland, UK on Jan. 6, 2011 under the provisions of the Budapest Treaty in the name of Philip Morris Products S.A:

| PM seed line | | |
|---|---|---|
| designation | Deposition date | Accession No |
| PM016 | 6 Jan. 2011 | NCIMB 41798 |
| PM021 | 6 Jan. 2011 | NCIMB 41799 |
| PM092 | 6 Jan. 2011 | NCIMB 41800 |
| PM102 | 6 Jan. 2011 | NCIMB 41801 |
| PM132 | 6 Jan. 2011 | NCIMB 41802 |
| PM204 | 6 Jan. 2011 | NCIMB 41803 |
| PM205 | 6 Jan. 2011 | NCIMB 41804 |
| PM215 | 6 Jan. 2011 | NCIMB 41805 |
| PM216 | 6 Jan. 2011 | NCIMB 41806 |
| PM217 | 6 Jan. 2011 | NCIMB 41807 |

1. A genetically modified Nicotiana tabacum plant cell, or a Nicotiana tabacum plant comprising the modified plant cells, wherein the modified plant cell comprises at least a modification of a first target nucleotide sequence in a genomic region comprising a coding sequence for an alpha-mannosidase I selected from the group consisting of NtMNS1a, NtMNS1b, NtMNS2, and NtMan1.4, and/or an allelic variant thereof, such that (i) the activity or the expression of alpha-mannosidase I in the modified plant cell is altered relative to an unmodified plant cell.

2. The modified Nicotiana tabacum plant cell or the Nicotiana tabacum plant of claim 1 comprising in addition to (a) the modification of a first target nucleotide sequence, (b) at least a modification of a second target nucleotide sequence in a genomic region comprising a coding sequence for an alpha-mannosidase I, or (c) at least a modification of a third target nucleotide sequence in a genomic region comprising a coding sequence for an alpha-mannosidase I, or (d) at least a modification of a fourth target nucleotide sequence in a genomic region comprising a coding sequence for an alpha-mannosidase I, or a combination of (a) and (b), (a) and (c), (a) and (d), (b) and (c), (b) and (d), or (c) and (d); or (a) and (b) and (c), (a) and (b) and (d), (a) and (c) and (d), or (b) and (c) and (d), or (a) and (b) and (c) and (d), wherein the alpha-mannosidase I is selected from the group consisting of NtMNS1a, NtMNS1b, NtMNS2, and NtMan1.4, and wherein the first, second, third and fourth alpha-mannosidases I are different from each other.

3. The modified Nicotiana tabacum plant cell or the Nicotiana tabacum plant of any one of the preceding claims, wherein the first, second, third and/or fourth target nucleotide sequence has
(i) at least 76% sequence identity to SEQ ID NO:1, SEQ ID NO: 2, SEQ ID NO: 32, SEQ ID NO: 33, SEQ ID NO: 63 or SEQ ID NO: 64; or a part thereof; and/or
(ii) at least 88% sequence identity to any of SEQ ID NO:30, SEQ ID NO: 94, SEQ ID NO:61, SEQ ID NO: 96, SEQ ID NO: 92, or SEQ ID NO: 98; or a part thereof.

4. The modified Nicotiana tabacum plant cell or the Nicotiana tabacum plant of claim 3, wherein the first, second, third and/or fourth target nucleotide sequence comprises, essentially comprises or consists of
(i) SEQ ID NO:1, SEQ ID NO: 2, SEQ ID NO: 32, SEQ ID NO: 33, SEQ ID NO: 63 or SEQ ID NO: 64; or a part thereof; and/or
(ii) SEQ ID NO:30, SEQ ID NO: 94, SEQ ID NO:61, SEQ ID NO: 96, SEQ ID NO: 92, or SEQ ID NO: 98; or a part thereof.

5. The modified Nicotiana tabacum plant cell or the Nicotiana tabacum plant of claim 1, wherein the activity or the expression of alpha-mannosidase I in the modified plant cell is (a) reduced or (b) increased relative to an unmodified plant cell.

6. Progeny of the modified Nicotiana tabacum plant according to any one of the preceding claims, wherein said progeny plant comprises a modification in at least one of the target sequences as defined in claim 1, wherein the activity or the expression of the alpha-mannosidase I is reduced relative to an unmodified plant cell.

7. A method for producing a heterologous protein, said method comprising:
(a) introducing into a modified Nicotiana tabacum plant cell or plant as defined in claim 1 an expression construct comprising a nucleotide sequence that encodes a heterologous glycoprotein, particularly an antigen for making a vaccine, a cytokine, a hormone, a coagulation protein, an apolipoprotein, an enzyme for replacement therapy in human, an immunoglobulin or a fragment thereof; and culturing the modified plant cell that comprises the expression construct such that the heterologous glycoprotein is produced, wherein said glycoprotein substantially lacks alpha-1,3-linked fucose and beta-1,2-linked xylose on its N-glycan as compared to a glycoprotein obtained from an unmodified plant cell, (b) optionally, regenerating a plant from the plant cell, and growing the plant and its progenies, and (c) optionally harvesting the glycoprotein.

8. A polynucleotide comprising a nucleotide sequence
(i) having at least 76% sequence identity to SEQ ID NO:1, SEQ ID NO: 2, SEQ ID NO: 32, SEQ ID NO: 33, SEQ ID NO: 63 or SEQ ID NO: 64; or a part thereof;
(ii) having at least 88% sequence identity to any of SEQ ID NO:30, SEQ ID NO: 94, SEQ ID NO:61, SEQ ID NO: 96, SEQ ID NO: 92, or SEQ ID NO: 98; or a part thereof;
(iii) encoding a polypeptide comprising a sequence having at least 83% sequence identity to SEQ ID NO: 31, SEQ ID NO: 95, SEQ ID NO: 62, SEQ ID NO: 97, SEQ ID NO: 93, or SEQ ID NO: 99, or a part thereof;
(iv) the complementary strand of which hybridizes to a nucleic acid probe consisting of the nucleotide sequence of any of (i)-(iii), or any of SEQ ID NO's: 3 to 29, SEQ ID NO's: 34, 35, 37 to 41, 43 to 49 and 51 to 60; or SEQ ID NO's: 65 to 91; and/or
(v) that deviates from the nucleotide sequence defined in any of (i)-(iv) by the degeneracy of the genetic code; or a part thereof,
wherein said nucleotide sequence, or a part thereof, encodes a polypeptide which exhibits mannose hydrolyzing activity.

9. A polypeptide having mannose hydrolyzing activity selected from the group consisting of:
(i) a polypeptide comprising an amino acid sequence having at least 83% sequence identity to any of the sequences set forth in SEQ ID NO: 31, SEQ ID NO: 95, SEQ ID NO: 62, SEQ ID NO: 97, SEQ ID NO: 93, or SEQ ID NO: 99, or a part thereof;
(ii) a polypeptide expressed by a nucleotide sequence according to (i)-(v) of claim 1; and
(iii) a polypeptide expressed by a nucleotide sequence set forth in SEQ ID NO: 2, SEQ ID NO: 30, SEQ ID NO: 33, SEQ ID NO: 94, SEQ ID NO: 61, SEQ ID NO: 64, SEQ ID NO: 96, SEQ ID NO: 92, SEQ ID NO: 98, or a part thereof.

10. Use of a polynucleotide as defined in claim 8, or a part thereof, for identifying a target site in
(a) a first target nucleotide sequence in a genomic region comprising a coding sequence for an alpha-mannosidase I; or
(b) the first target nucleotide sequence of a) and a second target nucleotide sequence in a genomic region comprising a coding sequence for an alpha-mannosidase I; or
(c) the first target nucleotide sequence of a), the second target nucleotide sequence of b) and a third target nucleotide sequence in a genomic region comprising a coding sequence for an alpha-mannosidase I;
(d) the first target nucleotide sequence of a), the second target nucleotide sequence of b) the third target nucleotide sequence of c) and a fourth target nucleotide sequence in a genomic region comprising a coding sequence for an alpha-mannosidase I; or
target nucleotide sequences a), b), c) and d);
for modification such that the activity or the expression of alpha-mannosidase I in the modified plant cell comprising the modification is altered relative to an unmodified plant cell, wherein the alpha-mannosidase I is selected from the group consisting of NtMNS1a, NtMNS1b, NtMNS2, and NtMan1.4, and wherein the first, second, third and fourth target alpha-mannosidases I are different from each other.

11. The use of claim 10 for making a non-natural meganuclease protein that selectively cleaves a genomic DNA molecule at a site within a nucleotide sequence as defined in claim 8.

12. The use of claim 10, for making a zinc finger nuclease that introduces a double-stranded break in at least one of the target nucleotide sequences as defined in claim 8.

13. A plant composition comprising a heterologous glycoprotein, obtainable from a plant comprising modified plant cells as defined in claim 1, wherein the glycoprotein substantially lacks alpha-1,3-linked fucose and beta-1,2-linked xylose on its N-glycan as compared to a glycoprotein obtained from an unmodified plant cell.

14. A method for producing a Nicotiana tabacum plant cell or a Nicotiana tabacum plant comprising the modified plant cells capable of producing humanized glycoproteins, the method comprising:
(i) modifying in the genome of a tobacco plant cell
(a) a first target nucleotide sequence in a genomic region comprising a coding sequence for an alpha-mannosidase I;
(b) the first target nucleotide sequence of a) and a second target nucleotide sequence in a genomic region comprising a coding sequence for an alpha-mannosidase I;
(c) the first target nucleotide sequence of a), the second target nucleotide sequence of b) and a third target nucleotide sequence in a genomic region comprising a coding sequence for an alpha-mannosidase I;
(d) the first target nucleotide sequence of a), the second target nucleotide sequence of b) and the third target nucleotide sequence of c) and a fourth target nucleotide sequence in a genomic region comprising a coding sequence for an alpha-mannosidase I; or
(e) all target nucleotide sequences a), b), c) and d);
(ii) identifying and, optionally, selecting a modified plant or plant cell comprising the modification in the target nucleotide sequence; and
(iii) optionally breeding the modified plant with another Nicotiana plant,
wherein the alpha-mannosidase I is selected from the group consisting of NtMNS1a, NtMNS1b, NtMNS2, and NtMan1.4, and wherein the first, second, third and fourth target alpha-mannosidases I are different from each other and wherein the activity or the expression of alpha-mannosidase I in the modified plant cell comprising the modification is altered relative to an unmodified plant cell such that the glycoproteins produced by said modified plant cell substantially lack alpha-1,3-linked fucose and beta-1,2-linked xylose on its N-glycan as compared to a glycoprotein obtained from an unmodified plant cell.

15. The method of claim 13, wherein the target nucleotide sequence comprises a nucleotide sequence as defined in claim 8.

16. The method of claim 14, wherein the modification of the genome of a tobacco plant or plant cell comprises
(a) identifying in the target nucleotide sequence of a Nicotiana tabacum plant or plant cell and, optionally, in at least one allelic variant thereof, a target site,
(b) designing, based on the nucleotide sequence as defined in claim 8, a mutagenic oligonucleotide capable of recognizing and binding at or adjacent to said target site, and
(c) binding the mutagenic oligonucleotide to the target nucleotide sequence in the genome of a tobacco plant or plant cell under conditions such that the genome is modified.

17. A plant composition comprising a heterologous glycoprotein, obtainable from a plant comprising modified plant cells as defined in claim 2, wherein the glycoprotein substantially lacks alpha-1,3-linked fucose and beta-1,2-linked xylose on its N-glycan as compared to a glycoprotein obtained from an unmodified plant cell.

18. A plant composition comprising a heterologous glycoprotein, obtainable from a plant comprising modified plant cells as defined in claim 3, wherein the glycoprotein substantially lacks alpha-1,3-linked fucose and beta-1,2-linked xylose on its N-glycan as compared to a glycoprotein obtained from an unmodified plant cell


SEQUENCE LISTING (103 sequences)

SEQ ID NO: 1 - DNA, 14501 bp, Nicotiana tabacum
  source 1..14501: /mol_type=“DNA” /organism=“Nicotiana tabacum”
aaggaatattcagaggaatgttctatgtatttgtacttttaataggtaaggggtatgccccatataagtaggaatagagagagaaagaaggggcatgtaatattttatcttgataagctctttctagaaaagtttactctcaagtaactacaaatactatctttacataagattcgatttgttgttttgtccaagctttcccacatcaatccaataaagtatttgatattcccacgtttggttatcttacatcattatcagagagagaatcatccacctcgttatatatttgagtgaattattctctctatttacatttattgtcatttatcatatttattgcttatccttgttctcccattctttcataagaatatcattaaatatccatttggcatttaataactttaagtgcggtttccagactattactatccatcaatcttgggtctaggatttattatgtttaactataatttactcattatcatttatttaattgtttaacaaaaaggcttaagactttttggtcaaacaatatggagtctgtaagtggggaggggcaaaagtgaaacactttattaacggcaagggcatttttgtacccaaatacaaacggagggcataattgctctattttcaatacttcagaggccttttccataaattctttcttaaacttactcccactttaatgctctccttttcctaggtagagtcagacctttatataatagtatctctatataacaacactttactataaaagcgaagcttttccggaaccaattttcatgttatgttataatatatgttctctataacaacacttcgctataacatccaaaaatattaggaacaaacgaggctatcatagagatgtttgacattatatccgtataaatatttgtcataaaaaaatatttttctaaaaaaatgtaccattgtgagatttttttaggaaaggaaaaaatatttaccgaggattgaccaaatatattcgaagaaaaagatagtaatggatgggagaagacatagcttggtagcttagtcctaggtaaggtgggatgcttaatcttaaatggaagacaagtcaatgttacaccgaccgcgcatgattgataagagcagtattattaccgtgtcttcactctttaccaaggctgaacgggtcttttacctaattaacgtcctgtagatttaggcgaggtttccttttgggaagtccagtagtcttggtcttcttggtcgttcctctcccccgatctattcaatctgcatcgggagatcgatctgcactttgattggtatattcataaaaagtgggtggaatggcgaggagtagatcgtcttccactactttcaggtacattaatccggcttactatctgaaacggccaaagcgtctggctttgctcttcatcgtttttgtcttcgccaccttcttcttttgggatcgacaaactttagtccgtgatcatcaggttcttctcttcattttccatttgtttcaccgtcctttttctctgattctctttgtggaattcatgtttaattttggtttaaaagtttgtaaagtagcgttctttaattacaaaacaactatattctttatgtttttttttgcaggaagagatctctaagttgaatcatgaagtgacgcaattgcgaaatctggttagtggttatctgaattatctatagctgtggaattttttattttaataatcagcctactgcctttaattcttttgtggctgccgtccctcttcttgctttgtcggggaactgtatgctagagcgtcttttaatatgtgcctgtacaaagttgtaattactcgagctacctcctgttcttccttcttcaaattaaatgtggttgagaatctgtttaactacttgtaatggggaaaaaacgataaacttactaattcaagttagatttaacatcaatgtctagagggatttatatggccagcttggttatgaagcctgaatttgggtcgcttagcgaagagctaccatgtactgccatttcacctacttaatacctcaatctgcttaagtaaggctagtaccgcccaacactgaatttggtttgcctagtgaagagtttctctgtctttcactgagcttaatacctcaatctgcttcagttagctcagggctagtactgcagtgttgagccctataaacgggcttggagtttaaaaaatatttgtgccattaaagcttaggaccatcttacctagtttagatattataggaaatgaaaaagcaaaaaaagacgagctacgacccggcaaacagaaaaggaactcaactaaattagtcttaagaaggtgatgcatctggctgagctcaaaccaggatgtaaagattagcggatggactgaccaaacaagagatggtggatggagtaagagtcgagatgtcgcaatatacctatagtgcactatagttcagcaccttttgtgttattccttagcattaaagggcgaggtaacagttggtggcaaaaagtcctcactgcgcattggaatgcttcctcgttggggttaaggagaactggaagagtgttcaagtagacttgagagaccaacacccaatggcttaaaatgaggggatagaatactatatatatacacatatatatatctatgtgtaatgaaacttcatgaaaatatctatgtgctatgtacttcttttcttgtccgtcttgtttgtctaaaaatttggtggtttggtttgtattttctggaaaaagaagtacaaagaatggatatagcttgttatgatttatgccagtattattttcatgtgtgcttgcttcacagtttacccatcttctgttgtttgcagtatagcatttaagcttttgattttaaatattcaacttgtttgcatttattttggatactgttttagctggaagatttgaagaatggtcgagtcatgccagataaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacaggtttggatgttacttcgaataagttattttttgtgttgttaatgttattattattattattttttgtgttgttaatgttgcctttgttttattgtatcttgtgatttcgcaattagatcattggtggaggaattctctactttttgatatacttcctgggggagttctctcccttttgattaatacaatttaccttatctaaaaaaaatcattggtggaggcatatgtaaagaaattcccggaaaatgaatccgggacattccaatattctttttcctttttgtgtgttaaggggaaatggggtataatagatgattagttaattacttaattaaatgagttagttgtaaatttaaaaactatttaaaaattaaatgagttagttgtcgattgatgttctccattaccttttctttctttgttattttattttcctaagtgctataccttttgttgactagataagcatgtgacactctagtttttcaattacaatattctgtaggttagtttgcagcagcaacgacaaaaactatgcctcaaaaatataaatcatcatgatctaggttgctctatttgggcccatttcatgtcaaccttcaatagtttgggcttttctaacagtagagattctctacaattcctagtaacatacactttttttttaaaaagtaacacaaattcaaactttttgtttattatgtttttactcattccatcccatttcatgttccggtgtttgactgggtataaaatttaagaaataaggaagactttttacatgtaatacaaatatatacaacataccaaaatgacctttactattaacatctaatgaaaggaggtaacctaccgtaccttcgtgataaaaaagggttaccttatcctcccaaagaaaaggttgtaagagttccgcatatcacttactatttctatctcctaataaaaaaatagtttttatatcaagtgggttcctaagaggttatgtcagtaagcataaaacgttattgctaggagtaaattgtttgcaattacaaaaatgtctcactcttttctggatagactaaaaaggaaggaatgccacatacaatgggacaggaggagtatatgttcttttcttcttatatcctgaccaagtatattgatttagcatgttttgatgctctggatattgcaaatgactatgaaatagcgattacataagtggctaagacttggccttttaatttattcttttctagggtatgttttgatatgattctctagatatttctgaattattgttagtgtcctggtagtgaggatagcaatttcatcttgcaaagttaatgcgcttgggttttaaaatacagacacctttatgctacctaaacggaagaacttcaatgttctgattttgcttaacatttggttgatttaaaattaaaacaaaagtacatttgcgacaagtttcccgagaagctttgatgtcatattaaaattagaggaagtttggggtttagtctgtggagttgtatttctcaaaactggtctgctttatgctgaacagtctgttatcgataaaagttgtctagctcagaagttcatgaaaatatggacttggactggataaacatttttttctgcccacctttgctgctacttgtgttaagaacaatatgtatatggaaagacacttttcttacttttccttgaagattaagatgcaactgtctttgtaatttacataatcagcgctttctttggtgatatgatacaacaacaacaacatctccagtaatatcccacactatggaggctatttccaatagaccctcggctcaagaaagcataagcaccacattaatggaaatataaacaagaagggacagtaccaaaaagcgatataaaagcaaaataaaaacaacaagacagtaaggtgatcaacaatgaaagaaaacaacggttagtcataaaaacctactaccaacagaaagcgagattgcgtgccaatactactgttatgagcactctagactacctactctactaccctaatcctcgacctccatatttttctatcaagggtcatgtcctcggtcagctgaagctgcgcgatgtcttgcctattcacctctcccacttctttggcctacctctacctctccgtaggccttccgatgtcaacctctcacacctcctcaccggtgcgtctgtgctcctcctcctcacatgaccaaaccacctaagccgcacttcccgcatcttgtcctcaacaggggccgcacccaccttgtcctgaataacctcatttttgatcctatctaacctgatgtgcccgcacatctatcttaatatcctcatctctgctaccttcatcatctggacatgagcgatcttgactggtcaacactcagccccatacaacatcgttggtctgaccaccactctgtagaacttacctaagtttcggtggcaccttcttgtcacataaaacaccggaagcgagtatccatttcatccatcccgccccaatacgatgtgcgacatcttcatcaatctccccatccactgaataatagacccaaggtacttaaaactccctctcctagggatgatctgcgagtccagcctcacctccccttcccctccttgagtcttgccactgaacttacactccaagtattttgccttagtcctgctcaacttgaaacctttagattccagggtctgcctccatacctctaattgcgcgttcacaccgtcttgcgtctcatcaatcaatacaatatcatctgcaaatagcatgcaccacggcacctccccttggatgtggcgcgtcagtacgtccatcccagagcaaacaaaaaggggtttagtgctgacccctgatgcaaccccatcacaaccggaaaatgattcgactccccacccaccgtcctcactcgggtctttactccattatacatgtccttaatcaacctaacgtaggcaacaggtacatccctagcctccaaacatcctcacccttagctctaccactctctcccaaactttcatagtatggttaagcaacttgataccccgatagttattgcaattttggatatcacgcttgttcttgtagacaggaaccattgtgctccacatccactcgtcgggcatcttcttcgttctaaaaatgacattaaataacctagtgagccactccaagcctgccttgcccgcactcttccaaaacttcaccgggatttcatccagcccggtcgctttgcccctgctcatcttacgcatagccccctcaacttcatcaactctaatccgcctacaataaccaaagtcacaacgactcccggagagttccaaatcacccattacaatgctcctgtccccttccttgttcaagaaactatggaagtaggtctgccatctccgatggataagcccctcatccaacaaaactttaccttcttcgtcctatagaagaaggatttttttacctatagaaggatatgttcttttgacaggtagcaagatatagtataccagtatccctttttctgtcttaacacatacttctagaaaatattgacacaaaagttcataccttgcagcttcagtaatgttcctatcatacccttgagtctgacttgaatgattgtatttatggaaaataaaaggtatatataggatagggtaactaattcttgttgatttgtggacattggcttttgatcatgtactatagtttcttgacaatcagaaaggaaatgacttcatgaaatctgttggacatatcgtttttatttcgtttaaaattgaatatttttagaagttgatatacttgccttgattctgcagttggtttctgctttgtgctcgtcgtatgatttacattacttctttagtgcacttatgcaaaattatttaacaattatgctgaaaatgtccaatctcagccgcagtcaaagaatggtgttgacagttttggtggtcttggagcaaccttaatagattctcttgacacactatatatcatgggcctggatgagcagtttcagagagctagagagtgagtttattctcttcctcttctagaatcatatgtattacttatggtacttgttttgtccgcagacaagagaaaaatgttaaactaaatatagtgaaaattatcaaaagcaagacacactgtgtgttttcactaatttaaagttaaaatgcaactgcaagattgctgtttcattcatttatggatttggtgccttgcatctgactattgccagatgttgaagtgttaattttatcacttccagtttccttctcgttattaagcatatttcctctaatctattgaatagtttttgcgaatgatgcagtatgttaggtttttaaactttccacatgtaattgttttcaatgaattattccacgtggctaatagtagctaacactttactgatggcagatgggttgcaaactccttggatttcaacaagaactatgatgcaagtgtttttgagacaaccataaggttgctttataaggtttaatatgagttttttatgagttttcattatcctttctcagcttcaatgatatagcaccatgattcttgtatggttaactatgtttttcaacatctcagggttgtaggtgggcttcttagtacgtacgatctatctggtgataagcttttccttgataaggctcaagacattgctgacagattgttgcccgcatggaatacagaatctggaatcccttacaacattatcaacttggcaaatgggaatccacataaccctgggtggacaggggtaagtttgaactctaataaattgcagttaatacccccccccccccccggttgatactactccaatatcttctggcaaagaggatggagggatcagttatcacagaaaagggagggtggatgtgattaatactgtatgtgacaagttattagatttggttcctgattcttatgttccctgaagattgtggagggaacctgacacaggagaagagcatatatctattgggaggtttctgaagaagaatcctctcttgaagtttccttataatatgttcaaagaacatttagtttgcttctctttgttcttttgctctcttccctgcattcgcctcccccctttcttttcaaagaacttgtattcttacccgttttgtgaacatattgaccggatctaatagtgatctttctcctggaacttgtcaatattgcttatagtttctatagattgtatttttccagaggtggtttgtgcatttttttgaaattattgtgctctttgctctcagggtgatagtatcctggcagattctggtactgagcagcttgagtttattgctctttcgcagaggacaggagacccaaaatatcaacaaaaggtatgcctgagaaaatttcttaaaatataaactacattcatattcacataaaactacaacttgaaactatgatatgaaaattggtattgtgtagaattgattaagctacagactgttgggtcaatctgtcctatttcaggtggagaatgttatcttagaacttaacaaaacttttccagatgatggtttgcttccaatatacattaatccacataaaggcacaacatcatactcaactataacatttggggcaatgggcgacaggtaatgaccttcgtttgtccattctagaatgatgcctgtgaaaacctgattgagtaggagtatttatccccaaaagaaaaaaagagggggagagcctttatcctatgcatttgtgtgaattggcatttagagcttccatgttttcttttcatatgaaaagttagtaaaagatttttttgtttcagcttttatgaatatttactcaaggtctggatacaaggaaacagaactgctgctgtgagtcattataggtaagcagcttaagttcacttatgtctgtttcgcttcagatattgttgtccttttaaagcttcaattcagtccatccggtgtttcacttgatggttcctgtaggtataagtgcatatattaatacacttcctcagcctgaaatcaaatctgatcatgtcttgcgggaatgcatagaaatattcattgatagtgtttacagatttggagcatttagaatttcaagtaagaaatcttagaacaaggggaaaaaattttgcactaaggataaaaagctgacgtaaatgagatatggtgtcactgtgaatacataatatcagagctatatgcttacaacagcagcaaatacttctcaatcgaagctagttgagaaattttgatgatatttcacagtcaggcctgaataaacttaattatgttttaactcgctcctcacgtgcgggcttgatttcctttaatgagccaaacacgtggaaattctttttggtccttttagtggtgagccaaacagttaggaggtgtgagaggttggccatggtgggtatgatgagaggtagagacatgccaaaaaagtattggagagaggtgattaggtaggacacggcacaacttaagcttaccgaggacgtgacccttgataggagggtgtggaggttgagaattagggtagaaggttagtaggtagtcgagcattttcctttttctttcccataccggtagtattagtgttagtatggtatttttttattcttagattgctattaccacctattgtttgattgctatctttcaccttggttttcttaatatcttgttgttgctactgcttattgtcaccgcttcttttcatcgtttctttagtcaagggtctctcgaaaagagcctctcagccctctcagggtagaagtaaggtctttatacacattaccctccccagaccccacttgtgggaattcattgggtttgttgttgttgcatttattttatcactttacgaggttctgtggaagcacattggataatgctcagaaaattctatgttgtggctttacattttctttaaggatggtgttgtccaggccagcttgcatggttgctgctttacattttattttttgataaatctttctatggcatatttatactattctcacatattttttactggttctaatcttcaaaaacattttattaattttctcgccagacacattaggagtagtcaaagtggggtagctggagtattaaactcatttatgctcctaagactctttctctaattggaagctttaactaaattttacagtggtatttgacgagagtttgaacttgaaatttcagatctaaaaactgtgagtactagtggaatttgttacaagtggttgatctttcccttgaatccttttccttctggtgctagaatgcaggaagatgaaattggttatagtggaaaggttgtgctataagtgctcagctagaacaaaaatggatctgtgatgtggaaaagaaaaaattatgtttgatgcataaagcctttctgagacttgaaaagatttgaaaaatgtagtgattttgtttaacctttttatgtttcttttacaaaattttgcattcctctgtgtttctcaatataattcttctgctaattttgcaagcaggaaaatgtgggagacatcaatgaaaggtcttttaagcttggtccggagaacaactccttcgtcttttgcatatatttgcgagaagatgggaagttctttaaatgacaaggtgatgtataggcttttacacatatttggggagtctgagatgtgttaattcttgactttgttttatttacccttttggattttgtgcagatggatgaacttgcatgctttgctcctgggatgttagctttaggatcatctggttatagccctaatgaggctcagaagttcttatcactggctgaggaggtatttttaacttacggagcatcattacggaatgtgattttaggttcctatttgcgaaatgatctccatatgccctaattcgtatgtgtgccactatgttgattgaaagtgataataagaaagagttatatctacagtcatatggaggaaaattgcgtcaaaagacctatacttctcggagttaatgtggatgtagctaaaaacaatacacaagaaaggatccatataagcaataccaactaattgggattaaagatccatagagttctcgtgtttgctgttactcctttttattttggttgaagttttgtgtaattgtttaactataagtgtgagatttagagaacatctagttttagtgaacccctgatagtattaatgaacccttatttattattggaatgaaatgggtttaagtagagtataatggatatagagaattcatataatcaactcttttactagtttaggtttgaggtttagttaattgatttgagaagtggtctctgtcgaaaaaggttttaggttttagttcaacttttgagcattagcgatggtgggctgtgggcaatgctctcctaccaccagatgttccctttcgttggctgttatagttagctgggggtgctgaaaggtgaagtgtgggataagaaccaagtgttagtgactcttaaatgtgttagggggctgggtgttggtcttagattgtgcttgcctctatgatttgacttgcctttcatctataggtttccctttcacatgatgggaaggcccagaggatcagtggttcattctataggagcttttagtgactgcagtgctgtttcttgttgccagaaagttctagtattgcttttttgctgaatatcttaaccttctcttgcagcttgcttggacttgctataatttttatcagtcaacacctacaaaactggcaggagagaactatttttttaatgccggccaagtcagttttttcattttagttcatggtgatgtttgtttttgttgtttgcttatggtaatagcttatttaaattcttcatcctgtttaatgctcttcaggatatgagtgtgggcacatcatggaatatattaaggccagagacagttgagtcgctgttttacctctggcgtttaacaggaaacaagacataccaagagtggggttggaacatatttcaagcatttgaaaagaactcaaggatagaatctggatatgttggacttaaagatgtaaggacaaactcaattctttcaactttggatagtacctacacctccattatcttctttctttaaatgccttcaaatgctgcatctataattctgtttctggaggtaaaaaatctgctgttatttcctgtgttatttgttaaaaatttgcgcctcctcatgaagtacactctttttttgggtttagatatcgataattgggatgtacatacatgaatgttatttttgtgctattgtttgatggaaaacttggtgctcctacttggtgttgtctctctcctcaccttaaacaccagctcgcttctaaacttcagtgttcttttttgggttttgcagtactcttattacaggcaggtttctcaaatttgatttattgagcaacctttaatatttagtgaagtatgaaagtatgtaacgtttgaaacggtgtacctctgtcagcccatccattacataattgtgcgcaaagagcaatattgagctagtgagcccctcttttttttaattgctgagcctgatctttattttctcctactagaagctcaacttcagagctacccttttttgttctatggatgctctcagtatttttattgcatcttctcctatttgaagctaaatttgtcctgggatagcaaaaacttgactccattccttgtagcccaatgtttctttccagttataaagcaagttgtgaagataaaaatgaagtggagggattttgaaatacaaggtgtctagtttcagataatgtataattaaattgttgcgactaactttagcatgcattattgctaacttttatcacgtcgactggtcttcatgggcagctgtcaaaagtttgtctggaacctctataattcagggttttgtgcttgtaatttgtcggtatgactgctttttcgtgttattcaatggaggcatatatcataaatttggttgtgaagggaaggttttaatttcatatacagtatcgttgttgacttctgttttaacactttttttcggttttcccaggtcaacactggtgtcaaagacaatatgatgcaaagcttctttcttgcggagacttttaaatatctctatcttcttttttcaccctcatcagtaatctctctagatgagtgggtttttaacacagaagcccaccccataaaaattgttacccggaatgatcgtgctatgaattctggagggtcaggtggacggcaagaatcagataggcaatcacgaaccaggaaagaaggtcgatttcgtattaatcattaatcaagctgttgataaattataatgggattgaatgaccaagtggagtgcctcatgaaacttgcatctgaggtaaaagaaggatctgcactctgcaactccagattggctggatgtattgctatattctgtagcttattaaatgccaccacatggagcagtagttttatgtagcttagcttagctactttagattcgcttcttaaactggcgtgtattataggagattgcaatttttgccggcagctccatttttgggcttgatgagcaaattgctagtcgcacctaatttttcccttagaaagcaaaaactcatttcaatgggcacaaaatatgacatttgtgttacccgagtttttttctttgacgttggggctgggtttgagttgtactacccctgagaattgacgtgtgtaaaggtatatgtatctgaatttgtgaatttacgatctctgtgacgctatatgtgtttcagatatatctgatacagagtttaagaaaggactttaaaaacttgtaagagtaaaatgagaagtttacaattattgtcttgaaatatataaatgtactattcttttggtatggactaaaacggaaagggtgccgtagaaaatggaatagagggagtacgtcttttagttacatacaagtactggagatttcactggttaggttcagcaagtcgtttggaaaaaaattatatacatactttatttggttaatttgtttaagtttaatgattagaccttttcgaacaatttcatttctcttggtttgactttggtatcggtttattattggtattaacaagaaaacatacgattttcaatgatcttagtatgtttaaagcattaaaatcagtaaggtattgcgtcaaatatcatttttattttatatttctgcttttatatagtatcgtttaatttactattaagtgaatgatatgaacataagattggtggcacaagtggcaagaaagtctctgttattatatgtttcacgagtacaggc

SEQ ID NO: 2 - DNA, 12162 bp, Nicotiana tabacum
  source 1..12162: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgtcttcgccaccttcttcttttgggatcgacaaactttagtccgtgatcatcaggttcttctcttcattttccatttgtttcaccgtcctttttctctgattctctttgtggaattcatgtttaattttggtttaaaagtttgtaaagtagcgttctttaattacaaaacaactatattctttatgtttttttttgcaggaagagatctctaagttgaatcatgaagtgacgcaattgcgaaatctggttagtggttatctgaattatctatagctgtggaattttttattttaataatcagcctactgcctttaattcttttgtggctgccgtccctcttcttgctttgtcggggaactgtatgctagagcgtcttttaatatgtgcctgtacaaagttgtaattactcgagctacctcctgttcttccttcttcaaattaaatgtggttgagaatctgtttaactacttgtaatggggaaaaaacgataaacttactaattcaagttagatttaacatcaatgtctagagggatttatatggccagcttggttatgaagcctgaatttgggtcgcttagcgaagagctaccatgtactgccatttcacctacttaatacctcaatctgcttaagtaaggctagtaccgcccaacactgaatttggtttgcctagtgaagagtttctctgtctttcactgagcttaatacctcaatctgcttcagttagctcagggctagtactgcagtgttgagccctataaacgggcttggagtttaaaaaatatttgtgccattaaagcttaggaccatcttacctagtttagatattataggaaatgaaaaagcaaaaaaagacgagctacgacccggcaaacagaaaaggaactcaactaaattagtcttaagaaggtgatgcatctggctgagctcaaaccaggatgtaaagattagcggatggactgaccaaacaagagatggtggatggagtaagagtcgagatgtcgcaatatacctatagtgcactatagttcagcaccttttgtgttattccttagcattaaagggcgaggtaacagttggtggcaaaaagtcctcactgcgcattggaatgcttcctcgttggggttaaggagaactggaagagtgttcaagtagacttgagagaccaacacccaatggcttaaaatgaggggatagaatactatatatatacacatatatatatctatgtgtaatgaaacttcatgaaaatatctatgtgctatgtacttcttttcttgtccgtcttgtttgtctaaaaatttggtggtttggtttgtattttctggaaaaagaagtacaaagaatggatatagcttgttatgatttatgccagtattattttcatgtgtgcttgcttcacagtttacccatcttctgttgtttgcagtatagcatttaagcttttgattttaaatattcaacttgtttgcatttattttggatactgttttagctggaagatttgaagaatggtcgagtcatgccagataaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacaggtttggatgttacttcgaataagttattttttgtgttgttaatgttattattattattattttttgtgttgttaatgttgcctttgttttattgtatcttgtgatttcgcaattagatcattggtggaggaattctctactttttgatatacttcctgggggagttctctcccttttgattaatacaatttaccttatctaaaaaaaatcattggtggaggcatatgtaaagaaattcccggaaaatgaatccgggacattccaatattctttttcctttttgtgtgttaaggggaaatggggtataatagatgattagttaattacttaattaaatgagttagttgtaaatttaaaaactatttaaaaattaaatgagttagttgtcgattgatgttctccattaccttttctttctttgttattttattttcctaagtgctataccttttgttgactagataagcatgtgacactctagtttttcaattacaatattctgtaggttagtttgcagcagcaacgacaaaaactatgcctcaaaaatataaatcatcatgatctaggttgctctatttgggcccatttcatgtcaaccttcaatagtttgggcttttctaacagtagagattctctacaattcctagtaacatacactttttttttaaaaagtaacacaaattcaaactttttgtttattatgtttttactcattccatcccatttcatgttccggtgtttgactgggtataaaatttaagaaataaggaagactttttacatgtaatacaaatatatacaacataccaaaatgacctttactattaacatctaatgaaaggaggtaacctaccgtaccttcgtgataaaaaagggttaccttatcctcccaaagaaaaggttgtaagagttccgcatatcacttactatttctatctcctaataaaaaaatagtttttatatcaagtgggttcctaagaggttatgtcagtaagcataaaacgttattgctaggagtaaattgtttgcaattacaaaaatgtctcactcttttctggatagactaaaaaggaaggaatgccacatacaatgggacaggaggagtatatgttcttttcttcttatatcctgaccaagtatattgatttagcatgttttgatgctctggatattgcaaatgactatgaaatagcgattacataagtggctaagacttggccttttaatttattcttttctagggtatgttttgatatgattctctagatatttctgaattattgttagtgtcctggtagtgaggatagcaatttcatcttgcaaagttaatgcgcttgggttttaaaatacagacacctttatgctacctaaacggaagaacttcaatgttctgattttgcttaacatttggttgatttaaaattaaaacaaaagtacatttgcgacaagtttcccgagaagctttgatgtcatattaaaattagaggaagtttggggtttagtctgtggagttgtatttctcaaaactggtctgctttatgctgaacagtctgttatcgataaaagttgtctagctcagaagttcatgaaaatatggacttggactggataaacatttttttctgcccacctttgctgctacttgtgttaagaacaatatgtatatggaaagacacttttcttacttttccttgaagattaagatgcaactgtctttgtaatttacataatcagcgctttctttggtgatatgatacaacaacaacaacatctccagtaatatcccacactatggaggctatttccaatagaccctcggctcaagaaagcataagcaccacattaatggaaatataaacaagaagggacagtaccaaaaagcgatataaaagcaaaataaaaacaacaagacagtaaggtgatcaacaatgaaagaaaacaacggttagtcataaaaacctactaccaacagaaagcgagattgcgtgccaatactactgttatgagcactctagactacctactctactaccctaatcctcgacctccatatttttctatcaagggtcatgtcctcggtcagctgaagctgcgcgatgtcttgcctattcacctctcccacttctttggcctacctctacctctccgtaggccttccgatgtcaacctctcacacctcctcaccggtgcgtctgtgctcctcctcctcacatgaccaaaccacctaagccgcacttcccgcatcttgtcctcaacaggggccgcacccaccttgtcctgaataacctcatttttgatcctatctaacctgatgtgcccgcacatctatcttaatatcctcatctctgctaccttcatcatctggacatgagcgatcttgactggtcaacactcagccccatacaacatcgttggtctgaccaccactctgtagaacttacctaagtttcggtggcaccttcttgtcacataaaacaccggaagcgagtatccatttcatccatcccgccccaatacgatgtgcgacatcttcatcaatctccccatccactgaataatagacccaaggtacttaaaactccctctcctagggatgatctgcgagtccagcctcacctccccttcccctccttgagtcttgccactgaacttacactccaagtattttgccttagtcctgctcaacttgaaacctttagattccagggtctgcctccatacctctaattgcgcgttcacaccgtcttgcgtctcatcaatcaatacaatatcatctgcaaatagcatgcaccacggcacctccccttggatgtggcgcgtcagtacgtccatcccagagcaaacaaaaaggggtttagtgctgacccctgatgcaaccccatcacaaccggaaaatgattcgactccccacccaccgtcctcactcgggtctttactccattatacatgtccttaatcaacctaacgtaggcaacaggtacatccctagcctccaaacatcctcacccttagctctaccactctctcccaaactttcatagtatggttaagcaacttgataccccgatagttattgcaattttggatatcacgcttgttcttgtagacaggaaccattgtgctccacatccactcgtcgggcatcttcttcgttctaaaaatgacattaaataacctagtgagccactccaagcctgccttgcccgcactcttccaaaacttcaccgggatttcatccagcccggtcgctttgcccctgctcatcttacgcatagccccctcaacttcatcaactctaatccgcctacaataaccaaagtcacaacgactcccggagagttccaaatcacccattacaatgctcctgtccccttccttgttcaagaaactatggaagtaggtctgccatctccgatggataagcccctcatccaacaaaactttaccttcttcgtcctatagaagaaggatttttttacctatagaaggatatgttcttttgacaggtagcaagatatagtataccagtatccctttttctgtcttaacacatacttctagaaaatattgacacaaaagttcataccttgcagcttcagtaatgttcctatcatacccttgagtctgacttgaatgattgtatttatggaaaataaaaggtatatataggatagggtaactaattcttgttgatttgtggacattggcttttgatcatgtactatagtttcttgacaatcagaaaggaaatgacttcatgaaatctgttggacatatcgtttttatttcgtttaaaattgaatatttttagaagttgatatacttgccttgattctgcagttggtttctgctttgtgctcgtcgtatgatttacattacttctttagtgcacttatgcaaaattatttaacaattatgctgaaaatgtccaatctcagccgcagtcaaagaatggtgttgacagttttggtggtcttggagcaaccttaatagattctcttgacacactatatatcatgggcctggatgagcagtttcagagagctagagagtgagtttattctcttcctcttctagaatcatatgtattacttatggtacttgttttgtccgcagacaagagaaaaatgttaaactaaatatagtgaaaattatcaaaagcaagacacactgtgtgttttcactaatttaaagttaaaatgcaactgcaagattgctgtttcattcatttatggatttggtgccttgcatctgactattgccagatgttgaagtgttaattttatcacttccagtttccttctcgttattaagcatatttcctctaatctattgaatagtttttgcgaatgatgcagtatgttaggtttttaaactttccacatgtaattgttttcaatgaattattccacgtggctaatagtagctaacactttactgatggcagatgggttgcaaactccttggatttcaacaagaactatgatgcaagtgtttttgagacaaccataaggttgctttataaggtttaatatgagttttttatgagttttcattatcctttctcagcttcaatgatatagcaccatgattcttgtatggttaactatgtttttcaacatctcagggttgtaggtgggcttcttagtacgtacgatctatctggtgataagcttttccttgataaggctcaagacattgctgacagattgttgcccgcatggaatacagaatctggaatcccttacaacattatcaacttggcaaatgggaatccacataaccctgggtggacaggggtaagtttgaactctaataaattgcagttaatacccccccccccccccggttgatactactccaatatcttctggcaaagaggatggagggatcagttatcacagaaaagggagggtggatgtgattaatactgtatgtgacaagttattagatttggttcctgattcttatgttccctgaagattgtggagggaacctgacacaggagaagagcatatatctattgggaggtttctgaagaagaatcctctcttgaagtttccttataatatgttcaaagaacatttagtttgcttctctttgttcttttgctctcttccctgcattcgcctcccccctttcttttcaaagaacttgtattcttacccgttttgtgaacatattgaccggatctaatagtgatctttctcctggaacttgtcaatattgcttatagtttctatagattgtatttttccagaggtggtttgtgcatttttttgaaattattgtgctctttgctctcagggtgatagtatcctggcagattctggtactgagcagcttgagtttattgctctttcgcagaggacaggagacccaaaatatcaacaaaaggtatgcctgagaaaatttcttaaaatataaactacattcatattcacataaaactacaacttgaaactatgatatgaaaattggtattgtgtagaattgattaagctacagactgttgggtcaatctgtcctatttcaggtggagaatgttatcttagaacttaacaaaacttttccagatgatggtttgcttccaatatacattaatccacataaaggcacaacatcatactcaactataacatttggggcaatgggcgacaggtaatgaccttcgtttgtccattctagaatgatgcctgtgaaaacctgattgagtaggagtatttatccccaaaagaaaaaaagagggggagagcctttatcctatgcatttgtgtgaattggcatttagagcttccatgttttcttttcatatgaaaagttagtaaaagatttttttgtttcagcttttatgaatatttactcaaggtctggatacaaggaaacagaactgctgctgtgagtcattataggtaagcagcttaagttcacttatgtctgtttcgcttcagatattgttgtccttttaaagcttcaattcagtccatccggtgtttcacttgatggttcctgtaggtataagtgcatatattaatacacttcctcagcctgaaatcaaatctgatcatgtcttgcgggaatgcatagaaatattcattgatagtgtttacagatttggagcatttagaatttcaagtaagaaatcttagaacaaggggaaaaaattttgcactaaggataaaaagctgacgtaaatgagatatggtgtcactgtgaatacataatatcagagctatatgcttacaacagcagcaaatacttctcaatcgaagctagttgagaaattttgatgatatttcacagtcaggcctgaataaacttaattatgttttaactcgctcctcacgtgcgggcttgatttcctttaatgagccaaacacgtggaaattctttttggtccttttagtggtgagccaaacagttaggaggtgtgagaggttggccatggtgggtatgatgagaggtagagacatgccaaaaaagtattggagagaggtgattaggtaggacacggcacaacttaagcttaccgaggacgtgacccttgataggagggtgtggaggttgagaattagggtagaaggttagtaggtagtcgagcattttcctttttctttcccataccggtagtattagtgttagtatggtatttttttattcttagattgctattaccacctattgtttgattgctatctttcaccttggttttcttaatatcttgttgttgctactgcttattgtcaccgcttcttttcatcgtttctttagtcaagggtctctcgaaaagagcctctcagccctctcagggtagaagtaaggtctttatacacattaccctccccagaccccacttgtgggaattcattgggtttgttgttgttgcatttattttatcactttacgaggttctgtggaagcacattggataatgctcagaaaattctatgttgtggctttacattttctttaaggatggtgttgtccaggccagcttgcatggttgctgctttacattttattttttgataaatctttctatggcatatttatactattctcacatattttttactggttctaatcttcaaaaacattttattaattttctcgccagacacattaggagtagtcaaagtggggtagctggagtattaaactcatttatgctcctaagactctttctctaattggaagctttaactaaattttacagtggtatttgacgagagtttgaacttgaaatttcagatctaaaaactgtgagtactagtggaatttgttacaagtggttgatctttcccttgaatccttttccttctggtgctagaatgcaggaagatgaaattggttatagtggaaaggttgtgctataagtgctcagctagaacaaaaatggatctgtgatgtggaaaagaaaaaattatgtttgatgcataaagcctttctgagacttgaaaagatttgaaaaatgtagtgattttgtttaacctttttatgtttcttttacaaaattttgcattcctctgtgtttctcaatataattcttctgctaattttgcaagcaggaaaatgtgggagacatcaatgaaaggtcttttaagcttggtccggagaacaactccttcgtcttttgcatatatttgcgagaagatgggaagttctttaaatgacaaggtgatgtataggcttttacacatatttggggagtctgagatgtgttaattcttgactttgttttatttacccttttggattttgtgcagatggatgaacttgcatgctttgctcctgggatgttagctttaggatcatctggttatagccctaatgaggctcagaagttcttatcactggctgaggaggtatttttaacttacggagcatcattacggaatgtgattttaggttcctatttgcgaaatgatctccatatgccctaattcgtatgtgtgccactatgttgattgaaagtgataataagaaagagttatatctacagtcatatggaggaaaattgcgtcaaaagacctatacttctcggagttaatgtggatgtagctaaaaacaatacacaagaaaggatccatataagcaataccaactaattgggattaaagatccatagagttctcgtgtttgctgttactcctttttattttggttgaagttttgtgtaattgtttaactataagtgtgagatttagagaacatctagttttagtgaacccctgatagtattaatgaacccttatttattattggaatgaaatgggtttaagtagagtataatggatatagagaattcatataatcaactcttttactagtttaggtttgaggtttagttaattgatttgagaagtggtctctgtcgaaaaaggttttaggttttagttcaacttttgagcattagcgatggtgggctgtgggcaatgctctcctaccaccagatgttccctttcgttggctgttatagttagctgggggtgctgaaaggtgaagtgtgggataagaaccaagtgttagtgactcttaaatgtgttagggggctgggtgttggtcttagattgtgcttgcctctatgatttgacttgcctttcatctataggtttccctttcacatgatgggaaggcccagaggatcagtggttcattctataggagcttttagtgactgcagtgctgtttcttgttgccagaaagttctagtattgcttttttgctgaatatcttaaccttctcttgcagcttgcttggacttgctataatttttatcagtcaacacctacaaaactggcaggagagaactatttttttaatgccggccaagtcagttttttcattttagttcatggtgatgtttgtttttgttgtttgcttatggtaatagcttatttaaattcttcatcctgtttaatgctcttcaggatatgagtgtgggcacatcatggaatatattaaggccagagacagttgagtcgctgttttacctctggcgtttaacaggaaacaagacataccaagagtggggttggaacatatttcaagcatttgaaaagaactcaaggatagaatctggatatgttggacttaaagatgtaaggacaaactcaattctttcaactttggatagtacctacacctccattatcttctttctttaaatgccttcaaatgctgcatctataattctgtttctggaggtaaaaaatctgctgttatttcctgtgttatttgttaaaaatttgcgcctcctcatgaagtacactctttttttgggtttagatatcgataattgggatgtacatacatgaatgttatttttgtgctattgtttgatggaaaacttggtgctcctacttggtgttgtctctctcctcaccttaaacaccagctcgcttctaaacttcagtgttcttttttgggttttgcagtactcttattacaggcaggtttctcaaatttgatttattgagcaacctttaatatttagtgaagtatgaaagtatgtaacgtttgaaacggtgtacctctgtcagcccatccattacataattgtgcgcaaagagcaatattgagctagtgagcccctcttttttttaattgctgagcctgatctttattttctcctactagaagctcaacttcagagctacccttttttgttctatggatgctctcagtatttttattgcatcttctcctatttgaagctaaatttgtcctgggatagcaaaaacttgactccattccttgtagcccaatgtttctttccagttataaagcaagttgtgaagataaaaatgaagtggagggattttgaaatacaaggtgtctagtttcagataatgtataattaaattgttgcgactaactttagcatgcattattgctaacttttatcacgtcgactggtcttcatgggcagctgtcaaaagtttgtctggaacctctataattcagggttttgtgcttgtaatttgtcggtatgactgctttttcgtgttattcaatggaggcatatatcataaatttggttgtgaagggaaggttttaatttcatatacagtatcgttgttgacttctgttttaacactttttttcggttttcccaggtcaacactggtgtcaaagacaatatgatgcaaagcttctttcttgcggagacttttaaatatctctatcttcttttttcaccctcatcagtaatctctctagatgagtgggtttttaacacagaagcccaccccataaaaattgttacccggaatgatcgtgctatgaattctggagggtcaggtggacggcaagaatcagataggcaatcacgaaccaggaaagaaggtcgatttcgtattaatcattaa

SEQ ID NO: 3 - DNA, 153 bp, Nicotiana tabacum
  source 1..153: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgtcttcgccaccttcttcttttgggatcgacaaactttagtccgtgatcatcag

SEQ ID NO: 4 - DNA, 145 bp, Nicotiana tabacum
  source 1..145: /mol_type=“DNA” /organism=“Nicotiana tabacum”
gttcttctcttcattttccatttgtttcaccgtcctttttctctgattctctttgtggaattcatgtttaattttggtttaaaagtttgtaaagtagcgttctttaattacaaaacaactatattctttatgtttttttttgcag

SEQ ID NO: 5 - DNA, 48 bp, Nicotiana tabacum
  source 1..48: /mol_type=“DNA” /organism=“Nicotiana tabacum”
gaagagatctctaagttgaatcatgaagtgacgcaattgcgaaatctg

SEQ ID NO: 6 - DNA, 1251 bp, Nicotiana tabacum
  source 1..1251: /mol_type=“DNA” /organism=“Nicotiana tabacum”
gttagtggttatctgaattatctatagctgtggaattttttattttaataatcagcctactgcctttaattcttttgtggctgccgtccctcttcttgctttgtcggggaactgtatgctagagcgtcttttaatatgtgcctgtacaaagttgtaattactcgagctacctcctgttcttccttcttcaaattaaatgtggttgagaatctgtttaactacttgtaatggggaaaaaacgataaacttactaattcaagttagatttaacatcaatgtctagagggatttatatggccagcttggttatgaagcctgaatttgggtcgcttagcgaagagctaccatgtactgccatttcacctacttaatacctcaatctgcttaagtaaggctagtaccgcccaacactgaatttggtttgcctagtgaagagtttctctgtctttcactgagcttaatacctcaatctgcttcagttagctcagggctagtactgcagtgttgagccctataaacgggcttggagtttaaaaaatatttgtgccattaaagcttaggaccatcttacctagtttagatattataggaaatgaaaaagcaaaaaaagacgagctacgacccggcaaacagaaaaggaactcaactaaattagtcttaagaaggtgatgcatctggctgagctcaaaccaggatgtaaagattagcggatggactgaccaaacaagagatggtggatggagtaagagtcgagatgtcgcaatatacctatagtgcactatagttcagcaccttttgtgttattccttagcattaaagggcgaggtaacagttggtggcaaaaagtcctcactgcgcattggaatgcttcctcgttggggttaaggagaactggaagagtgttcaagtagacttgagagaccaacacccaatggcttaaaatgaggggatagaatactatatatatacacatatatatatctatgtgtaatgaaacttcatgaaaatatctatgtgctatgtacttcttttcttgtccgtcttgtttgtctaaaaatttggtggtttggtttgtattttctggaaaaagaagtacaaagaatggatatagcttgttatgatttatgccagtattattttcatgtgtgcttgcttcacagtttacccatcttctgttgtttgcagtatagcatttaagcttttgattttaaatattcaacttgtttgcatttattttggatactgttttag

SEQ ID NO: 7 - DNA, 195 bp, Nicotiana tabacum
  source 1..195: /mol_type=“DNA” /organism=“Nicotiana tabacum”
ctggaagatttgaagaatggtcgagtcatgccagataaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacag

SEQ ID NO: 8 - DNA, 3938 bp, Nicotiana tabacum
  source 1..3938: /mol_type=“DNA” /organism=“Nicotiana tabacum”
gtttggatgttacttcgaataagttattttttgtgttgttaatgttattattattattattttttgtgttgttaatgttgcctttgttttattgtatcttgtgatttcgcaattagatcattggtggaggaattctctactttttgatatacttcctgggggagttctctcccttttgattaatacaatttaccttatctaaaaaaaatcattggtggaggcatatgtaaagaaattcccggaaaatgaatccgggacattccaatattctttttcctttttgtgtgttaaggggaaatggggtataatagatgattagttaattacttaattaaatgagttagttgtaaatttaaaaactatttaaaaattaaatgagttagttgtcgattgatgttctccattaccttttctttctttgttattttattttcctaagtgctataccttttgttgactagataagcatgtgacactctagtttttcaattacaatattctgtaggttagtttgcagcagcaacgacaaaaactatgcctcaaaaatataaatcatcatgatctaggttgctctatttgggcccatttcatgtcaaccttcaatagtttgggcttttctaacagtagagattctctacaattcctagtaacatacactttttttttaaaaagtaacacaaattcaaactttttgtttattatgtttttactcattccatcccatttcatgttccggtgtttgactgggtataaaatttaagaaataaggaagactttttacatgtaatacaaatatatacaacataccaaaatgacctttactattaacatctaatgaaaggaggtaacctaccgtaccttcgtgataaaaaagggttaccttatcctcccaaagaaaaggttgtaagagttccgcatatcacttactatttctatctcctaataaaaaaatagtttttatatcaagtgggttcctaagaggttatgtcagtaagcataaaacgttattgctaggagtaaattgtttgcaattacaaaaatgtctcactcttttctggatagactaaaaaggaaggaatgccacatacaatgggacaggaggagtatatgttcttttcttcttatatcctgaccaagtatattgatttagcatgttttgatgctctggatattgcaaatgactatgaaatagcgattacataagtggctaagacttggccttttaatttattcttttctagggtatgttttgatatgattctctagatatttctgaattattgttagtgtcctggtagtgaggatagcaatttcatcttgcaaagttaatgcgcttgggttttaaaatacagacacctttatgctacctaaacggaagaacttcaatgttctgattttgcttaacatttggttgatttaaaattaaaacaaaagtacatttgcgacaagtttcccgagaagctttgatgtcatattaaaattagaggaagtttggggtttagtctgtggagttgtatttctcaaaactggtctgctttatgctgaacagtctgttatcgataaaagttgtctagctcagaagttcatgaaaatatggacttggactggataaacatttttttctgcccacctttgctgctacttgtgttaagaacaatatgtatatggaaagacacttttcttacttttccttgaagattaagatgcaactgtctttgtaatttacataatcagcgctttctttggtgatatgatacaacaacaacaacatctccagtaatatcccacactatggaggctatttccaatagaccctcggctcaagaaagcataagcaccacattaatggaaatataaacaagaagggacagtaccaaaaagcgatataaaagcaaaataaaaacaacaagacagtaaggtgatcaacaatgaaagaaaacaacggttagtcataaaaacctactaccaacagaaagcgagattgcgtgccaatactactgttatgagcactctagactacctactctactaccctaatcctcgacctccatatttttctatcaagggtcatgtcctcggtcagctgaagctgcgcgatgtcttgcctattcacctctcccacttctttggcctacctctacctctccgtaggccttccgatgtcaacctctcacacctcctcaccggtgcgtctgtgctcctcctcctcacatgaccaaaccacctaagccgcacttcccgcatcttgtcctcaacaggggccgcacccaccttgtcctgaataacctcatttttgatcctatctaacctgatgtgcccgcacatctatcttaatatcctcatctctgctaccttcatcatctggacatgagcgatcttgactggtcaacactcagccccatacaacatcgttggtctgaccaccactctgtagaacttacctaagtttcggtggcaccttcttgtcacataaaacaccggaagcgagtatccatttcatccatcccgccccaatacgatgtgcgacatcttcatcaatctccccatccactgaataatagacccaaggtacttaaaactccctctcctagggatgatctgcgagtccagcctcacctccccttcccctccttgagtcttgccactgaacttacactccaagtattttgccttagtcctgctcaacttgaaacctttagattccagggtctgcctccatacctctaattgcgcgttcacaccgtcttgcgtctcatcaatcaatacaatatcatctgcaaatagcatgcaccacggcacctccccttggatgtggcgcgtcagtacgtccatcccagagcaaacaaaaaggggtttagtgctgacccctgatgcaaccccatcacaaccggaaaatgattcgactccccacccaccgtcctcactcgggtctttactccattatacatgtccttaatcaacctaacgtaggcaacaggtacatccctagcctccaaacatcctcacccttagctctaccactctctcccaaactttcatagtatggttaagcaacttgataccccgatagttattgcaattttggatatcacgcttgttcttgtagacaggaaccattgtgctccacatccactcgtcgggcatcttcttcgttctaaaaatgacattaaataacctagtgagccactccaagcctgccttgcccgcactcttccaaaacttcaccgggatttcatccagcccggtcgctttgcccctgctcatcttacgcatagccccctcaacttcatcaactctaatccgcctacaataaccaaagtcacaacgactcccggagagttccaaatcacccattacaatgctcctgtccccttccttgttcaagaaactatggaagtaggtctgccatctccgatggataagcccctcatccaacaaaactttaccttcttcgtcctatagaagaaggatttttttacctatagaaggatatgttcttttgacaggtagcaagatatagtataccagtatccctttttctgtcttaacacatacttctagaaaatattgacacaaaagttcataccttgcagcttcagtaatgttcctatcatacccttgagtctgacttgaatgattgtatttatggaaaataaaaggtatatataggatagggtaactaattcttgttgatttgtggacattggcttttgatcatgtactatagtttcttgacaatcagaaaggaaatgacttcatgaaatctgttggacatatcgtttttatttcgtttaaaattgaatatttttagaagttgatatacttgccttgattctgcagttggtttctgctttgtgctcgtcgtatgatttacattacttctttagtgcacttatgcaaaattatttaacaattatgctgaaaatgtccaatctcag

SEQ ID NO: 9 - DNA, 113 bp, Nicotiana tabacum
  source 1..113: /mol_type=“DNA” /organism=“Nicotiana tabacum”
ccgcagtcaaagaatggtgttgacagttttggtggtcttggagcaaccttaatagattctcttgacacactatatatcatgggcctggatgagcagtttcagagagctagaga

SEQ ID NO: 10 - DNA, 396 bp, Nicotiana tabacum
  source 1..396: /mol_type=“DNA” /organism=“Nicotiana tabacum”
gtgagtttattctcttcctcttctagaatcatatgtattacttatggtacttgttttgtccgcagacaagagaaaaatgttaaactaaatatagtgaaaattatcaaaagcaagacacactgtgtgttttcactaatttaaagttaaaatgcaactgcaagattgctgtttcattcatttatggatttggtgccttgcatctgactattgccagatgttgaagtgttaattttatcacttccagtttccttctcgttattaagcatatttcctctaatctattgaatagtttttgcgaatgatgcagtatgttaggtttttaaactttccacatgtaattgttttcaatgaattattccacgtggctaatagtagctaacactttactgatggcag

SEQ ID NO: 11 - DNA, 66 bp, Nicotiana tabacum
  source 1..66: /mol_type=“DNA” /organism=“Nicotiana tabacum”
atgggttgcaaactccttggatttcaacaagaactatgatgcaagtgtttttgagacaaccataag

SEQ ID NO: 12 - DNA, 114 bp, Nicotiana tabacum
  source 1..114: /mol_type=“DNA” /organism=“Nicotiana tabacum”
gttgctttataaggtttaatatgagttttttatgagttttcattatcctttctcagcttcaatgatatagcaccatgattcttgtatggttaactatgtttttcaacatctcag

SEQ ID NO: 13 - DNA, 172 bp, Nicotiana tabacum
  source 1..172: /mol_type=“DNA” /organism=“Nicotiana tabacum”
ggttgtaggtgggcttcttagtacgtacgatctatctggtgataagcttttccttgataaggctcaagacattgctgacagattgttgcccgcatggaatacagaatctggaatcccttacaacattatcaacttggcaaatgggaatccacataaccctgggtggacaggg

SEQ ID NO: 14 - DNA, 508 bp, Nicotiana tabacum
  source 1..508: /mol_type=“DNA” /organism=“Nicotiana tabacum”
gtaagtttgaactctaataaattgcagttaatacccccccccccccccggttgatactactccaatatcttctggcaaagaggatggagggatcagttatcacagaaaagggagggtggatgtgattaatactgtatgtgacaagttattagatttggttcctgattcttatgttccctgaagattgtggagggaacctgacacaggagaagagcatatatctattgggaggtttctgaagaagaatcctctcttgaagtttccttataatatgttcaaagaacatttagtttgcttctctttgttcttttgctctcttccctgcattcgcctcccccctttcttttcaaagaacttgtattcttacccgttttgtgaacatattgaccggatctaatagtgatctttctcctggaacttgtcaatattgcttatagtttctatagattgtatttttccagaggtggtttgtgcatttttttgaaattattgtgctctttgctctcag

SEQ ID NO: 15 - DNA, 90 bp, Nicotiana tabacum
  source 1..90: /mol_type=“DNA” /organism=“Nicotiana tabacum”
ggtgatagtatcctggcagattctggtactgagcagcttgagtttattgctctttcgcagaggacaggagacccaaaatatcaacaaaag

SEQ ID NO: 16 - DNA, 139 bp, Nicotiana tabacum
  source 1..139: /mol_type=“DNA” /organism=“Nicotiana tabacum”
gtatgcctgagaaaatttcttaaaatataaactacattcatattcacataaaactacaacttgaaactatgatatgaaaattggtattgtgtagaattgattaagctacagactgttgggtcaatctgtcctatttcag

SEQ ID NO: 17 - DNA, 125 bp, Nicotiana tabacum
  source 1..125: /mol_type=“DNA” /organism=“Nicotiana tabacum”
gtggagaatgttatcttagaacttaacaaaacttttccagatgatggtttgcttccaatatacattaatccacataaaggcacaacatcatactcaactataacatttggggcaatgggcgacag

SEQ ID NO: 18 - DNA, 185 bp, Nicotiana tabacum
  source 1..185: /mol_type=“DNA” /organism=“Nicotiana tabacum”
gtaatgaccttcgtttgtccattctagaatgatgcctgtgaaaacctgattgagtaggagtatttatccccaaaagaaaaaaagagggggagagcctttatcctatgcatttgtgtgaattggcatttagagcttccatgttttcttttcatatgaaaagttagtaaaagatttttttgtttcag

SEQ ID NO: 19 - DNA, 66 bp, Nicotiana tabacum
  source 1..66: /mol_type=“DNA” /organism=“Nicotiana tabacum”
cttttatgaatatttactcaaggtctggatacaaggaaacagaactgctgctgtgagtcattatag

SEQ ID NO: 20 - DNA, 1656 bp, Nicotiana tabacum
  source 1..1656: /mol_type=“DNA” /organism=“Nicotiana tabacum”
gtaagcagcttaagttcacttatgtctgtttcgcttcagatattgttgtccttttaaagcttcaattcagtccatccggtgtttcacttgatggttcctgtaggtataagtgcatatattaatacacttcctcagcctgaaatcaaatctgatcatgtcttgcgggaatgcatagaaatattcattgatagtgtttacagatttggagcatttagaatttcaagtaagaaatcttagaacaaggggaaaaaattttgcactaaggataaaaagctgacgtaaatgagatatggtgtcactgtgaatacataatatcagagctatatgcttacaacagcagcaaatacttctcaatcgaagctagttgagaaattttgatgatatttcacagtcaggcctgaataaacttaattatgttttaactcgctcctcacgtgcgggcttgatttcctttaatgagccaaacacgtggaaattctttttggtccttttagtggtgagccaaacagttaggaggtgtgagaggttggccatggtgggtatgatgagaggtagagacatgccaaaaaagtattggagagaggtgattaggtaggacacggcacaacttaagcttaccgaggacgtgacccttgataggagggtgtggaggttgagaattagggtagaaggttagtaggtagtcgagcattttcctttttctttcccataccggtagtattagtgttagtatggtatttttttattcttagattgctattaccacctattgtttgattgctatctttcaccttggttttcttaatatcttgttgttgctactgcttattgtcaccgcttcttttcatcgtttctttagtcaagggtctctcgaaaagagcctctcagccctctcagggtagaagtaaggtctttatacacattaccctccccagaccccacttgtgggaattcattgggtttgttgttgttgcatttattttatcactttacgaggttctgtggaagcacattggataatgctcagaaaattctatgttgtggctttacattttctttaaggatggtgttgtccaggccagcttgcatggttgctgctttacattttattttttgataaatctttctatggcatatttatactattctcacatattttttactggttctaatcttcaaaaacattttattaattttctcgccagacacattaggagtagtcaaagtggggtagctggagtattaaactcatttatgctcctaagactctttctctaattggaagctttaactaaattttacagtggtatttgacgagagtttgaacttgaaatttcagatctaaaaactgtgagtactagtggaatttgttacaagtggttgatctttcccttgaatccttttccttctggtgctagaatgcaggaagatgaaattggttatagtggaaaggttgtgctataagtgctcagctagaacaaaaatggatctgtgatgtggaaaagaaaaaattatgtttgatgcataaagcctttctgagacttgaaaagatttgaaaaatgtagtgattttgtttaacctttttatgtttcttttacaaaattttgcattcctctgtgtttctcaatataattcttctgctaattttgcaagcag

SEQ ID NO: 21 - DNA, 109 bp, Nicotiana tabacum
  source 1..109: /mol_type=“DNA” /organism=“Nicotiana tabacum”
gaaaatgtgggagacatcaatgaaaggtcttttaagcttggtccggagaacaactccttcgtcttttgcatatatttgcgagaagatgggaagttctttaaatgacaag

SEQ ID NO: 22 - DNA, 89 bp, Nicotiana tabacum
  source 1..89: /mol_type=“DNA” /organism=“Nicotiana tabacum”
gtgatgtataggcttttacacatatttggggagtctgagatgtgttaattcttgactttgttttatttacccttttggattttgtgcag

SEQ ID NO: 23 - DNA, 99 bp, Nicotiana tabacum
  source 1..99: /mol_type=“DNA” /organism=“Nicotiana tabacum”
atggatgaacttgcatgctttgctcctgggatgttagctttaggatcatctggttatagccctaatgaggctcagaagttcttatcactggctgaggag

SEQ ID NO: 24 - DNA, 886 bp, Nicotiana tabacum
  source 1..886: /mol_type=“DNA” /organism=“Nicotiana tabacum”
gtatttttaacttacggagcatcattacggaatgtgattttaggttcctatttgcgaaatgatctccatatgccctaattcgtatgtgtgccactatgttgattgaaagtgataataagaaagagttatatctacagtcatatggaggaaaattgcgtcaaaagacctatacttctcggagttaatgtggatgtagctaaaaacaatacacaagaaaggatccatataagcaataccaactaattgggattaaagatccatagagttctcgtgtttgctgttactcctttttattttggttgaagttttgtgtaattgtttaactataagtgtgagatttagagaacatctagttttagtgaacccctgatagtattaatgaacccttatttattattggaatgaaatgggtttaagtagagtataatggatatagagaattcatataatcaactcttttactagtttaggtttgaggtttagttaattgatttgagaagtggtctctgtcgaaaaaggttttaggttttagttcaacttttgagcattagcgatggtgggctgtgggcaatgctctcctaccaccagatgttccctttcgttggctgttatagttagctgggggtgctgaaaggtgaagtgtgggataagaaccaagtgttagtgactcttaaatgtgttagggggctgggtgttggtcttagattgtgcttgcctctatgatttgacttgcctttcatctataggtttccctttcacatgatgggaaggcccagaggatcagtggttcattctataggagcttttagtgactgcagtgctgtttcttgttgccagaaagttctagtattgcttttttgctgaatatcttaaccttctcttgcag

SEQ ID NO: 25 - DNA, 81 bp, Nicotiana tabacum
  source 1..81: /mol_type=“DNA” /organism=“Nicotiana tabacum”
cttgcttggacttgctataatttttatcagtcaacacctacaaaactggcaggagagaactatttttttaatgccggccaa

SEQ ID NO: 26 - DNA, 98 bp, Nicotiana tabacum
  source 1..98: /mol_type=“DNA” /organism=“Nicotiana tabacum”
gtcagttttttcattttagttcatggtgatgtttgtttttgttgtttgcttatggtaatagcttatttaaattcttcatcctgtttaatgctcttcag

SEQ ID NO: 27 - DNA, 171 bp, Nicotiana tabacum
  source 1..171: /mol_type=“DNA” /organism=“Nicotiana tabacum”
gatatgagtgtgggcacatcatggaatatattaaggccagagacagttgagtcgctgttttacctctggcgtttaacaggaaacaagacataccaagagtggggttggaacatatttcaagcatttgaaaagaactcaaggatagaatctggatatgttggacttaaagat

SEQ ID NO: 28 - DNA, 1017 bp, Nicotiana tabacum
  source 1..1017: /mol_type=“DNA” /organism=“Nicotiana tabacum”
gtaaggacaaactcaattctttcaactttggatagtacctacacctccattatcttctttctttaaatgccttcaaatgctgcatctataattctgtttctggaggtaaaaaatctgctgttatttcctgtgttatttgttaaaaatttgcgcctcctcatgaagtacactctttttttgggtttagatatcgataattgggatgtacatacatgaatgttatttttgtgctattgtttgatggaaaacttggtgctcctacttggtgttgtctctctcctcaccttaaacaccagctcgcttctaaacttcagtgttcttttttgggttttgcagtactcttattacaggcaggtttctcaaatttgatttattgagcaacctttaatatttagtgaagtatgaaagtatgtaacgtttgaaacggtgtacctctgtcagcccatccattacataattgtgcgcaaagagcaatattgagctagtgagcccctcttttttttaattgctgagcctgatctttattttctcctactagaagctcaacttcagagctacccttttttgttctatggatgctctcagtatttttattgcatcttctcctatttgaagctaaatttgtcctgggatagcaaaaacttgactccattccttgtagcccaatgtttctttccagttataaagcaagttgtgaagataaaaatgaagtggagggattttgaaatacaaggtgtctagtttcagataatgtataattaaattgttgcgactaactttagcatgcattattgctaacttttatcacgtcgactggtcttcatgggcagctgtcaaaagtttgtctggaacctctataattcagggttttgtgcttgtaatttgtcggtatgactgctttttcgtgttattcaatggaggcatatatcataaatttggttgtgaagggaaggttttaatttcatatacagtatcgttgttgacttctgttttaacactttttttcggttttcccag

SEQ ID NO: 29 - DNA, 252 bp, Nicotiana tabacum
  source 1..252: /mol_type=“DNA” /organism=“Nicotiana tabacum”
gtcaacactggtgtcaaagacaatatgatgcaaagcttctttcttgcggagacttttaaatatctctatcttcttttttcaccctcatcagtaatctctctagatgagtgggtttttaacacagaagcccaccccataaaaattgttacccggaatgatcgtgctatgaattctggagggtcaggtggacggcaagaatcagataggcaatcacgaaccaggaaagaaggtcgatttcgtattaatcattaa

SEQ ID NO: 30 - DNA, 1740 bp, Nicotiana tabacum
  source 1..1740: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgtcttcgccaccttcttcttttgggatcgacaaactttagtccgtgatcatcaggaagagatctctaagttgaatcatgaagtgacgcaattgcgaaatctgctggaagatttgaagaatggtcgagtcatgccagataaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacagccgcagtcaaagaatggtgttgacagttttggtggtcttggagcaaccttaatagattctcttgacacactatatatcatgggcctggatgagcagtttcagagagctagagaatgggttgcaaactccttggatttcaacaagaactatgatgcaagtgtttttgagacaaccataagggttgtaggtgggcttcttagtacgtacgatctatctggtgataagcttttccttgataaggctcaagacattgctgacagattgttgcccgcatggaatacagaatctggaatcccttacaacattatcaacttggcaaatgggaatccacataaccctgggtggacagggggtgatagtatcctggcagattctggtactgagcagcttgagtttattgctctttcgcagaggacaggagacccaaaatatcaacaaaaggtggagaatgttatcttagaacttaacaaaacttttccagatgatggtttgcttccaatatacattaatccacataaaggcacaacatcatactcaactataacatttggggcaatgggcgacagcttttatgaatatttactcaaggtctggatacaaggaaacagaactgctgctgtgagtcattataggaaaatgtgggagacatcaatgaaaggtcttttaagcttggtccggagaacaactccttcgtcttttgcatatatttgcgagaagatgggaagttctttaaatgacaagatggatgaacttgcatgctttgctcctgggatgttagctttaggatcatctggttatagccctaatgaggctcagaagttcttatcactggctgaggagcttgcttggacttgctataatttttatcagtcaacacctacaaaactggcaggagagaactatttttttaatgccggccaagatatgagtgtgggcacatcatggaatatattaaggccagagacagttgagtcgctgttttacctctggcgtttaacaggaaacaagacataccaagagtggggttggaacatatttcaagcatttgaaaagaactcaaggatagaatctggatatgttggacttaaagatgtcaacactggtgtcaaagacaatatgatgcaaagcttctttcttgcggagacttttaaatatctctatcttcttttttcaccctcatcagtaatctctctagatgagtgggtttttaacacagaagcccaccccataaaaattgttacccggaatgatcgtgctatgaattctggagggtcaggtggacggcaagaatcagataggcaatcacgaaccaggaaagaaggtcgatttcgtattaatcattaa

SEQ ID NO: 31 - protein, 579 aa, Nicotiana tabacum
  SOURCE 1..579: /mol_type=“protein” /organism=“Nicotiana tabacum”
MARSRSSSTTFRYINPAYYLKRPKRLALLFIVFVFATFFFWDRQTLVRDHQEEISKLNHEVTQLRNLLEDLKNGRVMPDKKMKSSGKGGHAAKNMDSPDNILDAQRREKVKDAMLHAWSSYEKYAWGHDELQPQSKNGVDSFGGLGATLIDSLDTLYIMGLDEQFQRAREWVANSLDFNKNYDASVFETTIRVVGGLLSTYDLSGDKLFLDKAQDIADRLLPAWNTESGIPYNIINLANGNPHNPGWTGGDSILADSGTEQLEFIALSQRTGDPKYQQKVENVILELNKTFPDDGLLPIYINPHKGTTSYSTITFGAMGDSFYEYLLKVWIQGNRTAAVSHYRKMWETSMKGLLSLVRRTTPSSFAYICEKMGSSLNDKMDELACFAPGMLALGSSGYSPNEAQKFLSLAEELAWTCYNFYQSTPTKLAGENYFFNAGQDMSVGTSWNILRPETVESLFYLWRLTGNKTYQEWGWNIFQAFEKNSRIESGYVGLKDVNTGVKDNMMQSFFLAETFKYLYLLFSPSSVISLDEWVFNTEAHPIKIVTRNDRAMNSGGSGGRQESDRQSRTRKEGRFRINH

SEQ ID NO: 32 - DNA, 12401 bp, Nicotiana tabacum
  source 1..12401: /mol_type=“DNA” /organism=“Nicotiana tabacum”
tgcgtcatttggaagtctcaaattatggataaacaatacaatttttgtattttggacattatgaagtatgacataacatacatctgagtatgaatcaccttactattgaaaagaagtgcgttaacttgaggattaaataataatacatagaacgtcgactggttcaatgagtatctttgtgcatgacgtaacaaacacatactatatcaatatcaaatgccttactttttaaatattattccatcgataaaaataatttgaggattaagtaatacacatagacgagctactggtctttgggcggtaatttcccgatcaatttactgatttatttatccttcagcttcttccaacgtcttattaaatgaaatttaaggtgcatttgcaaagctacattaatactaggctttaattacatgaattggtctgtttttctagttaattgattaattggtcaatattgaattgattgcaattgaaggatatcattattttctccaactctttaggggtacaaaaattgcaggtaattatgtataatagttaaattcaaaatacgacttttaaatttatgtttatatttgttctctaatataaattccttcttaaacttactcccattttaatgctctcctatttctatgtatccatataaatatttgtcataagaaaatattttctaaaaaaatgtatgattaaaagaatttttttagtaaaggaaaagatatttaccgtggattgaccaaatatattcgaagaaaaagatagaaatggatgggagaagacaaagcttggtatgttagtcctaggtaaggtgggatgcctaatcttaaatggaagacaagtcaatgttacaccgaccgcgcatgattgataagagtactattattaccgcgttttcactctttaccaaggctgaacgggtctttacctaattaacgtcctgtagatttaggcgaggtttccttttgggaagtccagtagtcttggtcttcttggtcgttcctcttccccgatctattcaatctgcatcggaagatcgatctgcacttcgatttactctgtttggtatattcataaattgggtggaatggcgaggagtagatcgtcttccactactttcaggtacattaatccggcttactatctgaaacggccaaagcgtctggctttgctcttcatcgtttttgttttcgccaccttcttcttttgggatcgacaaactttagtccgtgatcatcaggttcttcttctctttcattttccaatttttttcaccgtcctttttctctgattattttctttgtggaattcatgtttaattttggattaaagtttttaagttgcgttctttaattacaaaacaactatattctttatgtttttttttttgcaggaagagatctctaagttgaatgatgaagtgatgaaattgcgaaatctggttagtggttatctgaattatctatagctgtggaattttttattttaataatcagcctactgtctttaattcttttgtggctgccattcctcttcttgctttgtcgggggactgtatgctagagcgtcttttaatgtgtgccagactgccagtacaaagttgtaattactcgagctacctcctgttcttccttcttcaaattagatgaggttgagaatctgattaactacttgtagtgggggaaaaagataaacttactaattcatgttagatttaacatctgtgtgttaatatgggaaaaatattaatgtctagagggatttatatggccagcttggttatgaagcctgaatttggttcgcttagcgaagagctaccatgtaccacctttacacctacttaatacctcaatctgcttaagtaaggctagtactgcccaacactgaattcggtttgcctagtgaagagttctctgtctttcactgagcttaatacctcaatctgcttcagttagctcagggctagtactgcagtgttgggccctataaatgggcttggagtttaaaaaatatttgtggcattaaagcttaggaccatcttaccatgtttagatattataggaaatgaaaaagcagaaaaagtcgagctacgacccggcaaacagaaaaggaacccaactaaattagtcttaagaaggtgatacatctggctcagctcaaaccaggatgtaaagattagccgatggactgaccaaacaagagatggtggatggagtaagagtcgagatgtcgcaatttacctatagtgcaccataggtcagcaccttttgtgttattcccttagcattaaagggagaggtaacagtaggtagcaaaaagtcctcgctgaggcatgtagaatgcttcctcattggggttaaggagaactggaggagtgttcaagtagacttgagagtaccaacacccaatggcttaaaatgatgggacagaatactctatacacacacacacacacacacacacacatatatatatatatctatgtgtaatgaaacttcatgaaaatatctatgtgctatgtacttctttctttgtccgtcttgtttgtctaaaagtttggtggtttggtttatattttctggaaaaagaagcacaaagaatggatatagctagttatgacttatgccagtattattttcatgtgtgcttgcttcgcagtttacccatcttctgttgtttgcagtatagcattcaagctttttattttaaatactcaacttgtttgcatttattttggatactgttttagctggaagatttgaagaatggtcgagtcatgccaggtgaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacaggtttggatgttactttgaataagttcttttttgtgttgttaatgttgccttttttgttgtatcttgtgatttcgcatgttttgttgcctttttcctttttgtgtgttaaggggaaatggggtataatagatgattagttaattacttaattaaatgagttagttgtaaatttaaaaaactatttaaaaattaaatgagttagttgtcaattgacgttctccattaccttttctttctttgttatttaattttcctaagtgctataccttttgttgactagataagcatgtgacactctagtttttcagttacaatattctgtaggttagtttgcagcagcaatgacaaaaactacgcctcaaaaatataaatcatcttgatatagtttgctctatttgggcccatttcatgtcaaccttcaatagtttggggttttctaacagtagagattctctacaattcctagtaacatacacttcttcttttgagaaaagtaacacaaattcaaactttttgtttattatgtttttactcattccatcccatttcatgttccagtggttgactgggtattaaagttaagaaataaggaagactttttacacgtaatacaaatatatacaacataccaaaatgacctttactattaacatctaaatgaaaggaggtaacttaccttaccttcctgataaaaaaaggttaccttatcctcccaaagaaaaggttgtaagagttccatatatcacttactatttctatctcctaataaaaaaagtttttatattaagtgggttcctaagaggttatgtcagtaagcgtaaaacgttattgcgaggagtaaattgtttgcaattacaaaaatgtctcactcttctctggatagactaaaaaggaagtaatgccacataaaatgggacaggaggagtatatgttcttttcttcatatatcctgaccaagtatattgatttagcatgttttgatgctctggatattgcaaatgactatgaaatagcaattaaatggctaagaattggccttttaatttgttcttttctagggtatgttttgacatgattccctagatatttctgaattattgtgagtgtcctggtagtgaggatgacaatttcatcttgcaaagttaatgcgcttgggctttaaaataccgacacctttatgctacctaaacggaagaacttcaatgttctgattttgcttaacatttggttgatttaaaattaaaacaaaagtacatctgcgacaagtttccagagaagctttgatgtcaacttaaaattagaggaagtttggggtttaggctgtggagttgtatttctcaaaactggtctgctttatgctgaacagtgttatcgataaaagtcgtctagctcagaagttcatgaaaatatggacttggacatggataaacatttttttgtgcccacctttgctgctacttgtgttaagaacaatatgtatatggaaagacacttttcttacttttccttgaagattaagatgcaactgtctttgtaatttacataatcagcgctttctttggtgatatgatgtaacaattttttttacctatagaaggatatgttttttgataggtagcaggatatagtatcccttcatatgcaatcttattctactctctttcttctttttctgtctaaacacacaattctagaaaatattgacacaaaagttcataccttgcagcttcagtaatgttcctatcatacccttgaggccgacttgaatgattgtatttatggaaaataaaaggtatatgtaggatagggtaactaattcttgttgatttgtagacattggcttttgatcatgtactatagtttcttgacaatcagaaaggaaatgacttcatgaaatctgttggacatatcctttttatttcgtttaaaattgaatatttttagaagttgatatacttgccttgattctgcagttggtttctgctttgtgcttgtcgtacgatttacattacttctttactgcacttgtgcaaaattatttaataattatgctgaaaatgtccaatctcagccgcagtcaaagaatggtgttgacagttttggtggtcttggagcaaccttaatagattctcttgacacactatatatcatgggcctggatgagcagtttcagagagctagagagtgagtttattctcttcctcttctagaatcatatgtattacttatggtacttgttttgtccgcagacaagagaaaaatgttaaactaaatatagtgaaaattatcaaatgcaagacactgtgtgttttcactaatttaaagttaaaatgcaactgcaagattgctgtttcattcatttatggatttgatgccttgcatctgaccgttgccagacgttgaagtgttaattttatcacttccagcttccttctcgttattaagcatattttctctaatctattggatagtttttgcaaatgatgcagtatgttaggtattcaaactttccacatgtaattgttttcaatgaattattccacgtggctaatagtggctaacactttactgatggcagatgggttgtgaactccttggatttcaacaagaactatgatgcaagtgtttttgagacaaccataaggttgctttataaggtttaatatgagttttttatgagttttcgttatcctttctcagcttcaatgatatagcaccatgattcttgtatgattaattatgtttttcaacaactcagggttgtaggtgggcttcttagtacgtatgatctatctggtgataagcttttccttgataaggctcaagacattgctgacagattgttgcccgcatggaatacagaatctggaatcccttacaacactatcaacttggctcatgggaatccacataaccctgggtggacaggggtaagtttgaactctaataaattgcagttaatccccccctgttgatactactccaatatcttctggcaaagaggatggagggatcagttatcccagaagggtggatgtgattaatactgtatgtgacaagttattagatttggttcctgattcgttccctgaagattgtggagggagcccgacataggagaaagtatatatctattgggaggtttctgaagaagaatcctctctttaagtttccttataatatattcaaagaacatttagtttgcttctctttgttcttttgctcttttccctgcattcacctcccccctttcttttcaaagaacttgtattcttacccatttaacaaacatattgactgatctaatagtgatctttctcctggaacttgtcaataatgcttatagtttctatagattgtatttttccagaggtggtttgtgcatttttttgaaattgttgtgctctttgctctcagggtgatagtatcctggcagattctggtactgagcagcttgagtttattgctctttcgcagaggacaggagacccaaaatatcaacaaaaggtatgcctgagaaaatttcttaaaatacaaactacgttcatattctcataaaactacaacttgaaactatgatatgaaaattggtattgtgtaaaattgattaagctacagacttgggtcaatctgtcttatttcaggtggagaatgttatcttggaacttaacaaaacttttccagaggatggtttgcttccaatatacattaatccacataaaggcacaacatcatactcaactataacatttggggcaatgggcgacaggtaaatgaccatcgtttgtccattcttgcttccccggaccccgcgcatatcgggagcttagtgcaccgggctgccctttttttttgtccattctagaatgatgcctgtgaaaacctgattgagtaggagtatttatccccaaaagaaaaaaagagggggagagcctttatcctatgcatttgtgaattggcatttagagcttccatgttttcttttcatatgaaaagttagtaaaagatttttttgtttcagcttttatgaatatttactcaaggtctggatacaaggaaacagaactgctgctgtgagtcattataggtaagcagcttaagttcacttctgtctgtttcgcttcagatattgttgtccttttaaagcttcaattcagtccatccggtgtttcacttgatggttcatgtaggtctaagtgcatattttaatgcttaaacacttcctcagcctgaaatcaaatctgatcatgtgttgcgggaatgcatagaaatattcgttgacaatgtttacatatttggagcattttagaatttcaagtaagaaatcctagaacaaggaaaaaaattttgcactgaggataaaaaactgatggaaatgagatatggtgtcactgtgaatacataaaatcagagctatatacttacaacaacagcaaatacgcctcaatcgaaactagttgagaatttttgatgatatttcagtcaggcctgaataaacttaattatgttttaactcgctcctcacgtgcgggcttgattctttttggtctttttagtggtgagccaaacagttaggagatgtgagaggttggccttggtgggtacgaggagaggtagaggcagacaaaagaagtattggggagaggccttggtgggtataaggagaggtagaggcaggccaaagaagtattggggagaggtgattaggcaggacatgacgcaacttaagcttaccgaggacatgacccttgataggagggtgtggcggtcgagaattagggtagaaggttagtaggtagtcgagcattttcctttttctttcccatgccgatattattagtgttagtatgatatctttttattcttagattgctattgctacctattgtttgattgctatctttcacttcaattttcttaatatcttgatgttgttactgtttattgccactgcttcttttcatcgtttctttagccaagggtttatcgaaaagagtccctctgccctctcagggtagaggtaaggtctgcatacacactaccctacccaaaccccacttgtgtaaattcactgggtttgttattgttgcatttattccatcactttacgaggttctgtggaagcacattggataatgcacattggatatacattttctttaaggatggtgttgtccaggccagcttgcatggttgctgctttacatttaattttttgataaatctttctatggcatatttatactattctcacatatattttttacttgttctaagcttcaaaaactttttattaattgtctcgccagacacattaggagtagtcaaagtggggtagctggagtattaaactcatttatgctcctaagactctttctctaattagaagctttaactaaattttacagtggtatttgacgagagtttgaacttgaaatttcagatctaagaactgtgagtactagtggaatttgttataagtggttggtctttcccttgaatacttttccttttctggtgctagaatgcaggaagatgaaattggttatagtggaaaggttgtggtataagtgcttagctagaacaaaaatggatctgtgatgtggaaaagaaaaaaatatgtttgatgcataaagcctttctgagacttgaaaaaatatgaagtgattttgtttaacctttttatgtttcttttacaaaattttgcattcctctgtgttcctcaatataattcttccactaattttgcaagcaggaaaatgtgggagacatcaatgaaaggtcttttaagcttggttcggagaacgactccttcgtcttttgcatatatttgcgagaagatgggaagttctttaaatgacaaggtgatgtataggcatttacacatatttggggagtctgagatgtgttaattcttgactttgttttatttacccttttggattttctgcagatggatgaacttgcatgctttgctcctgggatgttagctttaggatcatctggttatagccctaatgaggctcagaagttcttatcactggctgaggaggtatttttaacttgcagagcatcattgcggaatgtgattttaggttcctatttgcgaaatgatctccatatgccctaattcgtatgtgtgccactatgttgattgaaagtgataataagaaagaggtatatctacagtcatatggaggaaaattgcgtcaaaagacctatacttctcggagttaaatgtggatgtagctaaaaacaatacacaagaaaggatccatataagcaataccaactaattgggattaaagatccatagagttctcatgtttgctgttactcctttttattttggttgaagttttgtgtaattgtttaactataagtgtgagatttagagaacatctagttttagtgaacccctgatagtattactgaacccttatttattattggaatgaaatggttttaagtagagcataatggatacagagaattcatataatcaactctttactagtttaggtttgatgtttagttaattgattaattgatttgagaagtggtctctgtcgaaaaaagttttaggttttatttcaacttttgagcattagcgatggtgggctgtgggcaatgctctcctaccaccagatgttcgctttcgttggctgttatagttagctgggggtgctgaaaggtgaagtgtgggataagaaacaagtgttagtgactcatgaatgtgttagggggctgagtgttggtctttagattgtgcttgcctctatgatttgacttgcctttcatctataggtttccctttcacatgatgggaaggcccagaggatcagtggttcattccataggagcttttagtgactgcagtgctgtttcttgttgccagaaagttctagtattgcttttttgctgaatatcttaaccttctcttgcagcttgcttggacttgctataacttttaccagtcaacacctacaaaactggcaggagagaactatttttttaatgccggccaagtcagtttttttcattttagttcatggtgatgtttgtttttgttgtttgcttatggtgataacttatttgaattgttcatcctatttaatgctcttcaggacatgagtgtgggcacatcatggaatatattaaggccagagacagttgagtcgctgttttacctctggcgtttaacaggaaacaagacataccaagagtggggttggaacatatttcaagcatttgaaaagaattcaaggatagaatctggatatgttggacttaaagatgtaaggacaaactcaattctttcaactttggatagtacctacacctccattatcttctttctttaaatgccttcaaatgctgcatctttaataatatttcccgtgttctttgttaaaaaactcatgaaatacactcttttttggattttgatattgataattgggatatacatacatgaatgttatttttatgctattgtttgatggaaaacctggtgctcctacttggtgttttctctctccttcaccttgtaaacaccagctcgcttctaaacttcagttttcttttttgggttttacagcactctaattacaggtaggtttctccaatttgatttattgagcaaccttctataattagtgaagtatgaaagtatgtaacgtttgaaaaggtgtacctctgtcagcccatccgtccattacataattgtacacaaagagcaacattggctagtgagccccccctttttttaattgctgcccctgatctttatcttctcctactagaagctcaacttcagagctacccttttttgttctatggatgctctcaatatttctattgcatcttctcctatttgaagctaaatttgtcctgggacagcaaaaacttgactccgttcctcgtagcccaatgtttctttccagttataaagcaaattgtgaagataaaaatgaagtggagggattttgaaatacaaggtgtcgagtttcagagaatgtataattaagttgttgtgactaactttagcatacataattgccaacttttatcacgtcgactggtcttcatgggcagctgtcaaaagtttgtcgggaacctctagaactcagggttttgtgcttgtaatttgtcggtatgactgctttttcgtgttcaatagggacatatatcactaaatttggttgtgaagggaaggttttaatttcatattcagtatcattgttgacctctcttttaacgctttttttttgggttttcccaggtcaacactggtgtcaaagacaatatgatgcaaagcttctttcttgcggagactcttaaatatctctatcttcttttttcaccctcatcagtaatatccctagatgagtgggtttttaacacagaagcccaccccataaaaattgttacccggaatgatcatgctatgagttctggaggttcaggtggacggcaagaatcagataggcaatcacgaaccaggaaagaaggtcgatttcgcattaatcattaatcaagctgttgataaactataatgggattcaatgaccaagtggagtgcctcatgaaacttgcatctgaggtaaaagaaggatctgcactctgttaactccagattggctgggtgtattgctatattctgtagcttattaaatgcaccacatggagcagtagttttatgtagcttagcttagctactttaagattcgcttcttaaactggcgtgtattataggagattgcaatttttgccggcagctccacatttttgggcttgatgagcaaattgctagtcgcacctaatttttcccttagaaagcaaaaactcatttcaatgggcacaaaatatgacatttgtgttcctgagtttttttctttgacgttggggctgggtttgtgttgtactacccctgagaattgacgtgtgtaaagttatatgtatctgaatttgtgaatttgcgatctctgtgacactatgtgtttcagttatatctgatactcatttttatatacctgtatttgattggacacggagtttgcggctttaaacatgttaaaagcatgtcattaaaagtaaaataagaagtttcagttaattgttgagtttttggcaaaaatcatcgttcaactatggctcaaaactagggtatatccttgtgtaataatagtgaacaaaaaatatccctgaactattcaaaaaatggcaagaattccttctgttaatttcttacaaccaaaacatgtgccaagcacatgacctccccccccccccccaaatcccccttcactcctgattctatccctcccgaagctatcccgctcttccatattcagtgaaactaaggcttcaaaagctatacattctacgtttaacttcataaaataactagagcaacaagataagttattttcttgaacaagaattgaagcta

SEQ ID NO: 33 - DNA, 10393 bp, Nicotiana tabacum
  source 1..10393: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgttttcgccaccttcttcttttgggatcgacaaactttagtccgtgatcatcaggttcttcttctctttcattttccaatttttttcaccgtcctttttctctgattattttctttgtggaattcatgtttaattttggattaaagtttttaagttgcgttctttaattacaaaacaactatattctttatgtttttttttttgcaggaagagatctctaagttgaatgatgaagtgatgaaattgcgaaatctggttagtggttatctgaattatctatagctgtggaattttttattttaataatcagcctactgtctttaattcttttgtggctgccattcctcttcttgctttgtcgggggactgtatgctagagcgtcttttaatgtgtgccagactgccagtacaaagttgtaattactcgagctacctcctgttcttccttcttcaaattagatgaggttgagaatctgattaactacttgtagtgggggaaaaagataaacttactaattcatgttagatttaacatctgtgtgttaatatgggaaaaatattaatgtctagagggatttatatggccagcttggttatgaagcctgaatttggttcgcttagcgaagagctaccatgtaccacctttacacctacttaatacctcaatctgcttaagtaaggctagtactgcccaacactgaattcggtttgcctagtgaagagttctctgtctttcactgagcttaatacctcaatctgcttcagttagctcagggctagtactgcagtgttgggccctataaatgggcttggagtttaaaaaatatttgtggcattaaagcttaggaccatcttaccatgtttagatattataggaaatgaaaaagcagaaaaagtcgagctacgacccggcaaacagaaaaggaacccaactaaattagtcttaagaaggtgatacatctggctcagctcaaaccaggatgtaaagattagccgatggactgaccaaacaagagatggtggatggagtaagagtcgagatgtcgcaatttacctatagtgcaccataggtcagcaccttttgtgttattcccttagcattaaagggagaggtaacagtaggtagcaaaaagtcctcgctgaggcatgtagaatgcttcctcattggggttaaggagaactggaggagtgttcaagtagacttgagagtaccaacacccaatggcttaaaatgatgggacagaatactctatacacacacacacacacacacacacacatatatatatatatctatgtgtaatgaaacttcatgaaaatatctatgtgctatgtacttctttctttgtccgtcttgtttgtctaaaagtttggtggtttggtttatattttctggaaaaagaagcacaaagaatggatatagctagttatgacttatgccagtattattttcatgtgtgcttgcttcgcagtttacccatcttctgttgtttgcagtatagcattcaagctttttattttaaatactcaacttgtttgcatttattttggatactgttttagctggaagatttgaagaatggtcgagtcatgccaggtgaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacaggtttggatgttactttgaataagttcttttttgtgttgttaatgttgccttttttgttgtatcttgtgatttcgcatgttttgttgcctttttcctttttgtgtgttaaggggaaatggggtataatagatgattagttaattacttaattaaatgagttagttgtaaatttaaaaaactatttaaaaattaaatgagttagttgtcaattgacgttctccattaccttttctttctttgttatttaattttcctaagtgctataccttttgttgactagataagcatgtgacactctagtttttcagttacaatattctgtaggttagtttgcagcagcaatgacaaaaactacgcctcaaaaatataaatcatcttgatatagtttgctctatttgggcccatttcatgtcaaccttcaatagtttggggttttctaacagtagagattctctacaattcctagtaacatacacttcttcttttgagaaaagtaacacaaattcaaactttttgtttattatgtttttactcattccatcccatttcatgttccagtggttgactgggtattaaagttaagaaataaggaagactttttacacgtaatacaaatatatacaacataccaaaatgacctttactattaacatctaaatgaaaggaggtaacttaccttaccttcctgataaaaaaaggttaccttatcctcccaaagaaaaggttgtaagagttccatatatcacttactatttctatctcctaataaaaaaagtttttatattaagtgggttcctaagaggttatgtcagtaagcgtaaaacgttattgcgaggagtaaattgtttgcaattacaaaaatgtctcactcttctctggatagactaaaaaggaagtaatgccacataaaatgggacaggaggagtatatgttcttttcttcatatatcctgaccaagtatattgatttagcatgttttgatgctctggatattgcaaatgactatgaaatagcaattaaatggctaagaattggccttttaatttgttcttttctagggtatgttttgacatgattccctagatatttctgaattattgtgagtgtcctggtagtgaggatgacaatttcatcttgcaaagttaatgcgcttgggctttaaaataccgacacctttatgctacctaaacggaagaacttcaatgttctgattttgcttaacatttggttgatttaaaattaaaacaaaagtacatctgcgacaagtttccagagaagctttgatgtcaacttaaaattagaggaagtttggggtttaggctgtggagttgtatttctcaaaactggtctgctttatgctgaacagtgttatcgataaaagtcgtctagctcagaagttcatgaaaatatggacttggacatggataaacatttttttgtgcccacctttgctgctacttgtgttaagaacaatatgtatatggaaagacacttttcttacttttccttgaagattaagatgcaactgtctttgtaatttacataatcagcgctttctttggtgatatgatgtaacaattttttttacctatagaaggatatgttttttgataggtagcaggatatagtatcccttcatatgcaatcttattctactctctttcttctttttctgtctaaacacacaattctagaaaatattgacacaaaagttcataccttgcagcttcagtaatgttcctatcatacccttgaggccgacttgaatgattgtatttatggaaaataaaaggtatatgtaggatagggtaactaattcttgttgatttgtagacattggcttttgatcatgtactatagtttcttgacaatcagaaaggaaatgacttcatgaaatctgttggacatatcctttttatttcgtttaaaattgaatatttttagaagttgatatacttgccttgattctgcagttggtttctgctttgtgcttgtcgtacgatttacattacttctttactgcacttgtgcaaaattatttaataattatgctgaaaatgtccaatctcagccgcagtcaaagaatggtgttgacagttttggtggtcttggagcaaccttaatagattctcttgacacactatatatcatgggcctggatgagcagtttcagagagctagagagtgagtttattctcttcctcttctagaatcatatgtattacttatggtacttgttttgtccgcagacaagagaaaaatgttaaactaaatatagtgaaaattatcaaatgcaagacactgtgtgttttcactaatttaaagttaaaatgcaactgcaagattgctgtttcattcatttatggatttgatgccttgcatctgaccgttgccagacgttgaagtgttaattttatcacttccagcttccttctcgttattaagcatattttctctaatctattggatagtttttgcaaatgatgcagtatgttaggtattcaaactttccacatgtaattgttttcaatgaattattccacgtggctaatagtggctaacactttactgatggcagatgggttgtgaactccttggatttcaacaagaactatgatgcaagtgtttttgagacaaccataaggttgctttataaggtttaatatgagttttttatgagttttcgttatcctttctcagcttcaatgatatagcaccatgattcttgtatgattaattatgtttttcaacaactcagggttgtaggtgggcttcttagtacgtatgatctatctggtgataagcttttccttgataaggctcaagacattgctgacagattgttgcccgcatggaatacagaatctggaatcccttacaacactatcaacttggctcatgggaatccacataaccctgggtggacaggggtaagtttgaactctaataaattgcagttaatccccccctgttgatactactccaatatcttctggcaaagaggatggagggatcagttatcccagaagggtggatgtgattaatactgtatgtgacaagttattagatttggttcctgattcgttccctgaagattgtggagggagcccgacataggagaaagtatatatctattgggaggtttctgaagaagaatcctctctttaagtttccttataatatattcaaagaacatttagtttgcttctctttgttcttttgctcttttccctgcattcacctcccccctttcttttcaaagaacttgtattcttacccatttaacaaacatattgactgatctaatagtgatctttctcctggaacttgtcaataatgcttatagtttctatagattgtatttttccagaggtggtttgtgcatttttttgaaattgttgtgctctttgctctcagggtgatagtatcctggcagattctggtactgagcagcttgagtttattgctctttcgcagaggacaggagacccaaaatatcaacaaaaggtatgcctgagaaaatttcttaaaatacaaactacgttcatattctcataaaactacaacttgaaactatgatatgaaaattggtattgtgtaaaattgattaagctacagacttgggtcaatctgtcttatttcaggtggagaatgttatcttggaacttaacaaaacttttccagaggatggtttgcttccaatatacattaatccacataaaggcacaacatcatactcaactataacatttggggcaatgggcgacaggtaaatgaccatcgtttgtccattcttgcttccccggaccccgcgcatatcgggagcttagtgcaccgggctgccctttttttttgtccattctagaatgatgcctgtgaaaacctgattgagtaggagtatttatccccaaaagaaaaaaagagggggagagcctttatcctatgcatttgtgaattggcatttagagcttccatgttttcttttcatatgaaaagttagtaaaagatttttttgtttcagcttttatgaatatttactcaaggtctggatacaaggaaacagaactgctgctgtgagtcattataggtaagcagcttaagttcacttctgtctgtttcgcttcagatattgttgtccttttaaagcttcaattcagtccatccggtgtttcacttgatggttcatgtaggtctaagtgcatattttaatgcttaaacacttcctcagcctgaaatcaaatctgatcatgtgttgcgggaatgcatagaaatattcgttgacaatgtttacatatttggagcattttagaatttcaagtaagaaatcctagaacaaggaaaaaaattttgcactgaggataaaaaactgatggaaatgagatatggtgtcactgtgaatacataaaatcagagctatatacttacaacaacagcaaatacgcctcaatcgaaactagttgagaatttttgatgatatttcagtcaggcctgaataaacttaattatgttttaactcgctcctcacgtgcgggcttgattctttttggtctttttagtggtgagccaaacagttaggagatgtgagaggttggccttggtgggtacgaggagaggtagaggcagacaaaagaagtattggggagaggccttggtgggtataaggagaggtagaggcaggccaaagaagtattggggagaggtgattaggcaggacatgacgcaacttaagcttaccgaggacatgacccttgataggagggtgtggcggtcgagaattagggtagaaggttagtaggtagtcgagcattttcctttttctttcccatgccgatattattagtgttagtatgatatctttttattcttagattgctattgctacctattgtttgattgctatctttcacttcaattttcttaatatcttgatgttgttactgtttattgccactgcttcttttcatcgtttctttagccaagggtttatcgaaaagagtccctctgccctctcagggtagaggtaaggtctgcatacacactaccctacccaaaccccacttgtgtaaattcactgggtttgttattgttgcatttattccatcactttacgaggttctgtggaagcacattggataatgcacattggatatacattttctttaaggatggtgttgtccaggccagcttgcatggttgctgctttacatttaattttttgataaatctttctatggcatatttatactattctcacatatattttttacttgttctaagcttcaaaaactttttattaattgtctcgccagacacattaggagtagtcaaagtggggtagctggagtattaaactcatttatgctcctaagactctttctctaattagaagctttaactaaattttacagtggtatttgacgagagtttgaacttgaaatttcagatctaagaactgtgagtactagtggaatttgttataagtggttggtctttcccttgaatacttttccttttctggtgctagaatgcaggaagatgaaattggttatagtggaaaggttgtggtataagtgcttagctagaacaaaaatggatctgtgatgtggaaaagaaaaaaatatgtttgatgcataaagcctttctgagacttgaaaaaatatgaagtgattttgtttaacctttttatgtttcttttacaaaattttgcattcctctgtgttcctcaatataattcttccactaattttgcaagcaggaaaatgtgggagacatcaatgaaaggtcttttaagcttggttcggagaacgactccttcgtcttttgcatatatttgcgagaagatgggaagttctttaaatgacaaggtgatgtataggcatttacacatatttggggagtctgagatgtgttaattcttgactttgttttatttacccttttggattttctgcagatggatgaacttgcatgctttgctcctgggatgttagctttaggatcatctggttatagccctaatgaggctcagaagttcttatcactggctgaggaggtatttttaacttgcagagcatcattgcggaatgtgattttaggttcctatttgcgaaatgatctccatatgccctaattcgtatgtgtgccactatgttgattgaaagtgataataagaaagaggtatatctacagtcatatggaggaaaattgcgtcaaaagacctatacttctcggagttaaatgtggatgtagctaaaaacaatacacaagaaaggatccatataagcaataccaactaattgggattaaagatccatagagttctcatgtttgctgttactcctttttattttggttgaagttttgtgtaattgtttaactataagtgtgagatttagagaacatctagttttagtgaacccctgatagtattactgaacccttatttattattggaatgaaatggttttaagtagagcataatggatacagagaattcatataatcaactctttactagtttaggtttgatgtttagttaattgattaattgatttgagaagtggtctctgtcgaaaaaagttttaggttttatttcaacttttgagcattagcgatggtgggctgtgggcaatgctctcctaccaccagatgttcgctttcgttggctgttatagttagctgggggtgctgaaaggtgaagtgtgggataagaaacaagtgttagtgactcatgaatgtgttagggggctgagtgttggtctttagattgtgcttgcctctatgatttgacttgcctttcatctataggtttccctttcacatgatgggaaggcccagaggatcagtggttcattccataggagcttttagtgactgcagtgctgtttcttgttgccagaaagttctagtattgcttttttgctgaatatcttaaccttctcttgcagcttgcttggacttgctataacttttaccagtcaacacctacaaaactggcaggagagaactatttttttaatgccggccaagtcagtttttttcattttagttcatggtgatgtttgtttttgttgtttgcttatggtgataacttatttgaattgttcatcctatttaatgctcttcaggacatgagtgtgggcacatcatggaatatattaaggccagagacagttgagtcgctgttttacctctggcgtttaacaggaaacaagacataccaagagtggggttggaacatatttcaagcatttgaaaagaattcaaggatagaatctggatatgttggacttaaagatgtaaggacaaactcaattctttcaactttggatagtacctacacctccattatcttctttctttaaatgccttcaaatgctgcatctttaataatatttcccgtgttctttgttaaaaaactcatgaaatacactcttttttggattttgatattgataattgggatatacatacatgaatgttatttttatgctattgtttgatggaaaacctggtgctcctacttggtgttttctctctccttcaccttgtaaacaccagctcgcttctaaacttcagttttcttttttgggttttacagcactctaattacaggtaggtttctccaatttgatttattgagcaaccttctataattagtgaagtatgaaagtatgtaacgtttgaaaaggtgtacctctgtcagcccatccgtccattacataattgtacacaaagagcaacattggctagtgagccccccctttttttaattgctgcccctgatctttatcttctcctactagaagctcaacttcagagctacccttttttgttctatggatgctctcaatatttctattgcatcttctcctatttgaagctaaatttgtcctgggacagcaaaaacttgactccgttcctcgtagcccaatgtttctttccagttataaagcaaattgtgaagataaaaatgaagtggagggattttgaaatacaaggtgtcgagtttcagagaatgtataattaagttgttgtgactaactttagcatacataattgccaacttttatcacgtcgactggtcttcatgggcagctgtcaaaagtttgtcgggaacctctagaactcagggttttgtgcttgtaatttgtcggtatgactgctttttcgtgttcaatagggacatatatcactaaatttggttgtgaagggaaggttttaatttcatattcagtatcattgttgacctctcttttaacgctttttttttgggttttcccaggtcaacactggtgtcaaagacaatatgatgcaaagcttctttcttgcggagactcttaaatatctctatcttcttttttcaccctcatcagtaatatccctagatgagtgggtttttaacacagaagcccaccccataaaaattgttacccggaatgatcatgctatgagttctggaggttcaggtggacggcaagaatcagataggcaatcacgaaccaggaaagaaggtcgatttcgcattaatcattaa

SEQ ID NO: 34 - DNA, 153 bp, Nicotiana tabacum
  source 1..153: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgttttcgccaccttcttcttttgggatcgacaaactttagtccgtgatcatcag

SEQ ID NO: 35 - DNA, 153 bp, Nicotiana tabacum
  source 1..153: /mol_type=“DNA” /organism=“Nicotiana tabacum”
gttcttcttctctttcattttccaatttttttcaccgtcctttttctctgattattttctttgtggaattcatgtttaattttggattaaagtttttaagttgcgttctttaattacaaaacaactatattctttatgtttttttttttgcag

SEQ ID NO: 36 - DNA, 48 bp, Nicotiana tabacum
  source 1..48: /mol_type=“DNA” /organism=“Nicotiana tabacum”
gaagagatctctaagttgaatgatgaagtgatgaaattgcgaaatctg

SEQ ID NO: 37 - DNA, 1308 bp, Nicotiana tabacum
  source 1..1308: /mol_type=“DNA” /organism=“Nicotiana tabacum”
gttagtggttatctgaattatctatagctgtggaattttttattttaataatcagcctactgtctttaattcttttgtggctgccattcctcttcttgctttgtcgggggactgtatgctagagcgtcttttaatgtgtgccagactgccagtacaaagttgtaattactcgagctacctcctgttcttccttcttcaaattagatgaggttgagaatctgattaactacttgtagtgggggaaaaagataaacttactaattcatgttagatttaacatctgtgtgttaatatgggaaaaatattaatgtctagagggatttatatggccagcttggttatgaagcctgaatttggttcgcttagcgaagagctaccatgtaccacctttacacctacttaatacctcaatctgcttaagtaaggctagtactgcccaacactgaattcggtttgcctagtgaagagttctctgtctttcactgagcttaatacctcaatctgcttcagttagctcagggctagtactgcagtgttgggccctataaatgggcttggagtttaaaaaatatttgtggcattaaagcttaggaccatcttaccatgtttagatattataggaaatgaaaaagcagaaaaagtcgagctacgacccggcaaacagaaaaggaacccaactaaattagtcttaagaaggtgatacatctggctcagctcaaaccaggatgtaaagattagccgatggactgaccaaacaagagatggtggatggagtaagagtcgagatgtcgcaatttacctatagtgcaccataggtcagcaccttttgtgttattcccttagcattaaagggagaggtaacagtaggtagcaaaaagtcctcgctgaggcatgtagaatgcttcctcattggggttaaggagaactggaggagtgttcaagtagacttgagagtaccaacacccaatggcttaaaatgatgggacagaatactctatacacacacacacacacacacacacacatatatatatatatctatgtgtaatgaaacttcatgaaaatatctatgtgctatgtacttctttctttgtccgtcttgtttgtctaaaagtttggtggtttggtttatattttctggaaaaagaagcacaaagaatggatatagctagttatgacttatgccagtattattttcatgtgtgcttgcttcgcagtttacccatcttctgttgtttgcagtatagcattcaagctttttattttaaatactcaacttgtttgcatttattttggatactgttttag

SEQ ID NO: 38 - DNA, 195 bp, Nicotiana tabacum
  source 1..195: /mol_type=“DNA” /organism=“Nicotiana tabacum”
ctggaagatttgaagaatggtcgagtcatgccaggtgaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacag

SEQ ID NO: 39 - DNA, 2071 bp, Nicotiana tabacum
  source 1..2071: /mol_type=“DNA” /organism=“Nicotiana tabacum”
gtttggatgttactttgaataagttcttttttgtgttgttaatgttgccttttttgttgtatcttgtgatttcgcatgttttgttgcctttttcctttttgtgtgttaaggggaaatggggtataatagatgattagttaattacttaattaaatgagttagttgtaaatttaaaaaactatttaaaaattaaatgagttagttgtcaattgacgttctccattaccttttctttctttgttatttaattttcctaagtgctataccttttgttgactagataagcatgtgacactctagtttttcagttacaatattctgtaggttagtttgcagcagcaatgacaaaaactacgcctcaaaaatataaatcatcttgatatagtttgctctatttgggcccatttcatgtcaaccttcaatagtttggggttttctaacagtagagattctctacaattcctagtaacatacacttcttcttttgagaaaagtaacacaaattcaaactttttgtttattatgtttttactcattccatcccatttcatgttccagtggttgactgggtattaaagttaagaaataaggaagactttttacacgtaatacaaatatatacaacataccaaaatgacctttactattaacatctaaatgaaaggaggtaacttaccttaccttcctgataaaaaaaggttaccttatcctcccaaagaaaaggttgtaagagttccatatatcacttactatttctatctcctaataaaaaaagtttttatattaagtgggttcctaagaggttatgtcagtaagcgtaaaacgttattgcgaggagtaaattgtttgcaattacaaaaatgtctcactcttctctggatagactaaaaaggaagtaatgccacataaaatgggacaggaggagtatatgttcttttcttcatatatcctgaccaagtatattgatttagcatgttttgatgctctggatattgcaaatgactatgaaatagcaattaaatggctaagaattggccttttaatttgttcttttctagggtatgttttgacatgattccctagatatttctgaattattgtgagtgtcctggtagtgaggatgacaatttcatcttgcaaagttaatgcgcttgggctttaaaataccgacacctttatgctacctaaacggaagaacttcaatgttctgattttgcttaacatttggttgatttaaaattaaaacaaaagtacatctgcgacaagtttccagagaagctttgatgtcaacttaaaattagaggaagtttggggtttaggctgtggagttgtatttctcaaaactggtctgctttatgctgaacagtgttatcgataaaagtcgtctagctcagaagttcatgaaaatatggacttggacatggataaacatttttttgtgcccacctttgctgctacttgtgttaagaacaatatgtatatggaaagacacttttcttacttttccttgaagattaagatgcaactgtctttgtaatttacataatcagcgctttctttggtgatatgatgtaacaattttttttacctatagaaggatatgttttttgataggtagcaggatatagtatcccttcatatgcaatcttattctactctctttcttctttttctgtctaaacacacaattctagaaaatattgacacaaaagttcataccttgcagcttcagtaatgttcctatcatacccttgaggccgacttgaatgattgtatttatggaaaataaaaggtatatgtaggatagggtaactaattcttgttgatttgtagacattggcttttgatcatgtactatagtttcttgacaatcagaaaggaaatgacttcatgaaatctgttggacatatcctttttatttcgtttaaaattgaatatttttagaagttgatatacttgccttgattctgcagttggtttctgctttgtgcttgtcgtacgatttacattacttctttactgcacttgtgcaaaattatttaataattatgctgaaaatgtccaatctcag

SEQ ID NO: 40 - DNA, 113 bp, Nicotiana tabacum; identical to SEQ ID NO: 9
  source 1..113: /mol_type=“DNA” /organism=“Nicotiana tabacum”

SEQ ID NO: 41 - DNA, 394 bp, Nicotiana tabacum
  source 1..394: /mol_type=“DNA” /organism=“Nicotiana tabacum”
gtgagtttattctcttcctcttctagaatcatatgtattacttatggtacttgttttgtccgcagacaagagaaaaatgttaaactaaatatagtgaaaattatcaaatgcaagacactgtgtgttttcactaatttaaagttaaaatgcaactgcaagattgctgtttcattcatttatggatttgatgccttgcatctgaccgttgccagacgttgaagtgttaattttatcacttccagcttccttctcgttattaagcatattttctctaatctattggatagtttttgcaaatgatgcagtatgttaggtattcaaactttccacatgtaattgttttcaatgaattattccacgtggctaatagtggctaacactttactgatggcag

SEQ ID NO: 42 - DNA, 66 bp, Nicotiana tabacum
  source 1..66: /mol_type=“DNA” /organism=“Nicotiana tabacum”
atgggttgtgaactccttggatttcaacaagaactatgatgcaagtgtttttgagacaaccataag

SEQ ID NO: 43 - DNA, 114 bp, Nicotiana tabacum
  source 1..114: /mol_type=“DNA” /organism=“Nicotiana tabacum”
gttgctttataaggtttaatatgagttttttatgagttttcgttatcctttctcagcttcaatgatatagcaccatgattcttgtatgattaattatgtttttcaacaactcag

SEQ ID NO: 44 - DNA, 172 bp, Nicotiana tabacum
  source 1..172: /mol_type=“DNA” /organism=“Nicotiana tabacum”
ggttgtaggtgggcttcttagtacgtatgatctatctggtgataagcttttccttgataaggctcaagacattgctgacagattgttgcccgcatggaatacagaatctggaatcccttacaacactatcaacttggctcatgggaatccacataaccctgggtggacaggg

SEQ ID NO: 45 - DNA, 487 bp, Nicotiana tabacum
  source 1..487: /mol_type=“DNA” /organism=“Nicotiana tabacum”
gtaagtttgaactctaataaattgcagttaatccccccctgttgatactactccaatatcttctggcaaagaggatggagggatcagttatcccagaagggtggatgtgattaatactgtatgtgacaagttattagatttggttcctgattcgttccctgaagattgtggagggagcccgacataggagaaagtatatatctattgggaggtttctgaagaagaatcctctctttaagtttccttataatatattcaaagaacatttagtttgcttctctttgttcttttgctcttttccctgcattcacctcccccctttcttttcaaagaacttgtattcttacccatttaacaaacatattgactgatctaatagtgatctttctcctggaacttgtcaataatgcttatagtttctatagattgtatttttccagaggtggtttgtgcatttttttgaaattgttgtgctctttgctctcag

SEQ ID NO: 46 - DNA, 90 bp, Nicotiana tabacum; identical to SEQ ID NO: 15
  source 1..90: /mol_type=“DNA” /organism=“Nicotiana tabacum”

SEQ ID NO: 47 - DNA, 146 bp, Nicotiana tabacum
  source 1..146: /mol_type=“DNA” /organism=“Nicotiana tabacum”
gtatgcctgagaaaatttcttaaaatacaaactacgttcatattctcataaaactacaacttgaaactatgatatgaaaattggtattgtgtaaaattgattaagctacagacttgggtcaatctgtcttatttcaggtggagaat

SEQ ID NO: 48 - DNA, 116 bp, Nicotiana tabacum
  source 1..116: /mol_type=“DNA” /organism=“Nicotiana tabacum”
gttatcttggaacttaacaaaacttttccagaggatggtttgcttccaatatacattaatccacataaaggcacaacatcatactcaactataacatttggggcaatgggcgacag

SEQ ID NO: 49 - DNA, 252 bp, Nicotiana tabacum
  source 1..252: /mol_type=“DNA” /organism=“Nicotiana tabacum”
gtaaatgaccatcgtttgtccattcttgcttccccggaccccgcgcatatcgggagcttagtgcaccgggctgccctttttttttgtccattctagaatgatgcctgtgaaaacctgattgagtaggagtatttatccccaaaagaaaaaaagagggggagagcctttatcctatgcatttgtgaattggcatttagagcttccatgttttcttttcatatgaaaagttagtaaaagatttttttgtttcag

SEQ ID NO: 50 - DNA, 66 bp, Nicotiana tabacum; identical to SEQ ID NO: 19
  source 1..66: /mol_type=“DNA” /organism=“Nicotiana tabacum”

SEQ ID NO: 51 - DNA, 1668 bp, Nicotiana tabacum
  source 1..1668: /mol_type=“DNA” /organism=“Nicotiana tabacum”
gtaagcagcttaagttcacttctgtctgtttcgcttcagatattgttgtccttttaaagcttcaattcagtccatccggtgtttcacttgatggttcatgtaggtctaagtgcatattttaatgcttaaacacttcctcagcctgaaatcaaatctgatcatgtgttgcgggaatgcatagaaatattcgttgacaatgtttacatatttggagcattttagaatttcaagtaagaaatcctagaacaaggaaaaaaattttgcactgaggataaaaaactgatggaaatgagatatggtgtcactgtgaatacataaaatcagagctatatacttacaacaacagcaaatacgcctcaatcgaaactagttgagaatttttgatgatatttcagtcaggcctgaataaacttaattatgttttaactcgctcctcacgtgcgggcttgattctttttggtctttttagtggtgagccaaacagttaggagatgtgagaggttggccttggtgggtacgaggagaggtagaggcagacaaaagaagtattggggagaggccttggtgggtataaggagaggtagaggcaggccaaagaagtattggggagaggtgattaggcaggacatgacgcaacttaagcttaccgaggacatgacccttgataggagggtgtggcggtcgagaattagggtagaaggttagtaggtagtcgagcattttcctttttctttcccatgccgatattattagtgttagtatgatatctttttattcttagattgctattgctacctattgtttgattgctatctttcacttcaattttcttaatatcttgatgttgttactgtttattgccactgcttcttttcatcgtttctttagccaagggtttatcgaaaagagtccctctgccctctcagggtagaggtaaggtctgcatacacactaccctacccaaaccccacttgtgtaaattcactgggtttgttattgttgcatttattccatcactttacgaggttctgtggaagcacattggataatgcacattggatatacattttctttaaggatggtgttgtccaggccagcttgcatggttgctgctttacatttaattttttgataaatctttctatggcatatttatactattctcacatatattttttacttgttctaagcttcaaaaactttttattaattgtctcgccagacacattaggagtagtcaaagtggggtagctggagtattaaactcatttatgctcctaagactctttctctaattagaagctttaactaaattttacagtggtatttgacgagagtttgaacttgaaatttcagatctaagaactgtgagtactagtggaatttgttataagtggttggtctttcccttgaatacttttccttttctggtgctagaatgcaggaagatgaaattggttatagtggaaaggttgtggtataagtgcttagctagaacaaaaatggatctgtgatgtggaaaagaaaaaaatatgtttgatgcataaagcctttctgagacttgaaaaaatatgaagtgattttgtttaacctttttatgtttcttttacaaaattttgcattcctctgtgttcctcaatataattcttccactaattttgcaagcag

SEQ ID NO: 52 - DNA, 109 bp, Nicotiana tabacum
  source 1..109: /mol_type=“DNA” /organism=“Nicotiana tabacum”
gaaaatgtgggagacatcaatgaaaggtcttttaagcttggttcggagaacgactccttcgtcttttgcatatatttgcgagaagatgggaagttctttaaatgacaag

SEQ ID NO: 53 - DNA, 89 bp, Nicotiana tabacum
  source 1..89: /mol_type=“DNA” /organism=“Nicotiana tabacum”
gtgatgtataggcatttacacatatttggggagtctgagatgtgttaattcttgactttgttttatttacccttttggattttctgcag

SEQ ID NO: 54 - DNA, 99 bp, Nicotiana tabacum; identical to SEQ ID NO: 23
  source 1..99: /mol_type=“DNA” /organism=“Nicotiana tabacum”

SEQ ID NO: 55 - DNA, 895 bp, Nicotiana tabacum
  source 1..895: /mol_type=“DNA” /organism=“Nicotiana tabacum”
gtatttttaacttgcagagcatcattgcggaatgtgattttaggttcctatttgcgaaatgatctccatatgccctaattcgtatgtgtgccactatgttgattgaaagtgataataagaaagaggtatatctacagtcatatggaggaaaattgcgtcaaaagacctatacttctcggagttaaatgtggatgtagctaaaaacaatacacaagaaaggatccatataagcaataccaactaattgggattaaagatccatagagttctcatgtttgctgttactcctttttattttggttgaagttttgtgtaattgtttaactataagtgtgagatttagagaacatctagttttagtgaacccctgatagtattactgaacccttatttattattggaatgaaatggttttaagtagagcataatggatacagagaattcatataatcaactctttactagtttaggtttgatgtttagttaattgattaattgatttgagaagtggtctctgtcgaaaaaagttttaggttttatttcaacttttgagcattagcgatggtgggctgtgggcaatgctctcctaccaccagatgttcgctttcgttggctgttatagttagctgggggtgctgaaaggtgaagtgtgggataagaaacaagtgttagtgactcatgaatgtgttagggggctgagtgttggtctttagattgtgcttgcctctatgatttgacttgcctttcatctataggtttccctttcacatgatgggaaggcccagaggatcagtggttcattccataggagcttttagtgactgcagtgctgtttcttgttgccagaaagttctagtattgcttttttgctgaatatcttaaccttctcttgcag

SEQ ID NO: 56 - DNA, 81 bp, Nicotiana tabacum
  source 1..81: /mol_type=“DNA” /organism=“Nicotiana tabacum”
cttgcttggacttgctataacttttaccagtcaacacctacaaaactggcaggagagaactatttttttaatgccggccaa

SEQ ID NO: 57 - DNA, 99 bp, Nicotiana tabacum
  source 1..99: /mol_type=“DNA” /organism=“Nicotiana tabacum”
gtcagtttttttcattttagttcatggtgatgtttgtttttgttgtttgcttatggtgataacttatttgaattgttcatcctatttaatgctcttcag

SEQ ID NO: 58 - DNA, 171 bp, Nicotiana tabacum
  source 1..171: /mol_type=“DNA” /organism=“Nicotiana tabacum”
gacatgagtgtgggcacatcatggaatatattaaggccagagacagttgagtcgctgttttacctctggcgtttaacaggaaacaagacataccaagagtggggttggaacatatttcaagcatttgaaaagaattcaaggatagaatctggatatgttggacttaaagat

SEQ ID NO: 59 - DNA, 986 bp, Nicotiana tabacum
  source 1..986: /mol_type=“DNA” /organism=“Nicotiana tabacum”
gtaaggacaaactcaattctttcaactttggatagtacctacacctccattatcttctttctttaaatgccttcaaatgctgcatctttaataatatttcccgtgttctttgttaaaaaactcatgaaatacactcttttttggattttgatattgataattgggatatacatacatgaatgttatttttatgctattgtttgatggaaaacctggtgctcctacttggtgttttctctctccttcaccttgtaaacaccagctcgcttctaaacttcagttttcttttttgggttttacagcactctaattacaggtaggtttctccaatttgatttattgagcaaccttctataattagtgaagtatgaaagtatgtaacgtttgaaaaggtgtacctctgtcagcccatccgtccattacataattgtacacaaagagcaacattggctagtgagccccccctttttttaattgctgcccctgatctttatcttctcctactagaagctcaacttcagagctacccttttttgttctatggatgctctcaatatttctattgcatcttctcctatttgaagctaaatttgtcctgggacagcaaaaacttgactccgttcctcgtagcccaatgtttctttccagttataaagcaaattgtgaagataaaaatgaagtggagggattttgaaatacaaggtgtcgagtttcagagaatgtataattaagttgttgtgactaactttagcatacataattgccaacttttatcacgtcgactggtcttcatgggcagctgtcaaaagtttgtcgggaacctctagaactcagggttttgtgcttgtaatttgtcggtatgactgctttttcgtgttcaatagggacatatatcactaaatttggttgtgaagggaaggttttaatttcatattcagtatcattgttgacctctcttttaacgctttttttttgggttttcccag

SEQ ID NO: 60 - DNA, 252 bp, Nicotiana tabacum
  source 1..252: /mol_type=“DNA” /organism=“Nicotiana tabacum”
gtcaacactggtgtcaaagacaatatgatgcaaagcttctttcttgcggagactcttaaatatctctatcttcttttttcaccctcatcagtaatatccctagatgagtgggtttttaacacagaagcccaccccataaaaattgttacccggaatgatcatgctatgagttctggaggttcaggtggacggcaagaatcagataggcaatcacgaaccaggaaagaaggtcgatttcgcattaatcattaa

SEQ ID NO: 61 - DNA, 1740 bp, Nicotiana tabacum
  source 1..1740: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgttttcgccaccttcttcttttgggatcgacaaactttagtccgtgatcatcaggaagagatctctaagttgaatgatgaagtgatgaaattgcgaaatctgctggaagatttgaagaatggtcgagtcatgccaggtgaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacagccgcagtcaaagaatggtgttgacagttttggtggtcttggagcaaccttaatagattctcttgacacactatatatcatgggcctggatgagcagtttcagagagctagagaatgggttgtgaactccttggatttcaacaagaactatgatgcaagtgtttttgagacaaccataagggttgtaggtgggcttcttagtacgtatgatctatctggtgataagcttttccttgataaggctcaagacattgctgacagattgttgcccgcatggaatacagaatctggaatcccttacaacactatcaacttggctcatgggaatccacataaccctgggtggacagggggtgatagtatcctggcagattctggtactgagcagcttgagtttattgctctttcgcagaggacaggagacccaaaatatcaacaaaaggtggagaatgttatcttggaacttaacaaaacttttccagaggatggtttgcttccaatatacattaatccacataaaggcacaacatcatactcaactataacatttggggcaatgggcgacagcttttatgaatatttactcaaggtctggatacaaggaaacagaactgctgctgtgagtcattataggaaaatgtgggagacatcaatgaaaggtcttttaagcttggttcggagaacgactccttcgtcttttgcatatatttgcgagaagatgggaagttctttaaatgacaagatggatgaacttgcatgctttgctcctgggatgttagctttaggatcatctggttatagccctaatgaggctcagaagttcttatcactggctgaggagcttgcttggacttgctataacttttaccagtcaacacctacaaaactggcaggagagaactatttttttaatgccggccaagacatgagtgtgggcacatcatggaatatattaaggccagagacagttgagtcgctgttttacctctggcgtttaacaggaaacaagacataccaagagtggggttggaacatatttcaagcatttgaaaagaattcaaggatagaatctggatatgttggacttaaagatgtcaacactggtgtcaaagacaatatgatgcaaagcttctttcttgcggagactcttaaatatctctatcttcttttttcaccctcatcagtaatatccctagatgagtgggtttttaacacagaagcccaccccataaaaattgttacccggaatgatcatgctatgagttctggaggttcaggtggacggcaagaatcagataggcaatcacgaaccaggaaagaaggtcgatttcgcattaatcattaa

SEQ ID NO: 62 - protein, 579 aa, Nicotiana tabacum
  SOURCE 1..579: /mol_type=“protein” /organism=“Nicotiana tabacum”
MARSRSSSTTFRYINPAYYLKRPKRLALLFIVFVFATFFFWDRQTLVRDHQEEISKLNDEVMKLRNLLEDLKNGRVMPGEKMKSSGKGGHAAKNMDSPDNILDAQRREKVKDAMLHAWSSYEKYAWGHDELQPQSKNGVDSFGGLGATLIDSLDTLYIMGLDEQFQRAREWVVNSLDFNKNYDASVFETTIRVVGGLLSTYDLSGDKLFLDKAQDIADRLLPAWNTESGIPYNTINLAHGNPHNPGWTGGDSILADSGTEQLEFIALSQRTGDPKYQQKVENVILELNKTFPEDGLLPIYINPHKGTTSYSTITFGAMGDSFYEYLLKVWIQGNRTAAVSHYRKMWETSMKGLLSLVRRTTPSSFAYICEKMGSSLNDKMDELACFAPGMLALGSSGYSPNEAQKFLSLAEELAWTCYNFYQSTPTKLAGENYFFNAGQDMSVGTSWNILRPETVESLFYLWRLTGNKTYQEWGWNIFQAFEKNSRIESGYVGLKDVNTGVKDNMMQSFFLAETLKYLYLLFSPSSVISLDEWVFNTEAHPIKIVTRNDHAMSSGGSGGRQESDRQSRTRKEGRFRINH

SEQ ID NO: 63 - DNA, 11501 bp, Nicotiana tabacum
  source 1..11501: /mol_type=“DNA” /organism=“Nicotiana tabacum”
ccacagacggcgccaaactgtttgaccaaaaagcgctaagcttttcgttaaactaattaataaagaaaatggaagataaatcttaaccaaaaataattaactttagatctaagcatattgaatgcaagaatcgaatgaggccgagcttatataacatttcttaggatgattaaaagacatcaaacgtaaataataagcttacatctttgatacattgttccgtacttgtaagagcaaagagggaaagtaagaaatgtcgttgataactgtgagatctatctttattgattcaacaatgacgattacaaagttttaggcttttactttgttgttggaggtctcctcccgttcttctgttcctttttctctcttttttaggaacccccttttcttgcctttttctctcatatatatatatatattaccaatctttccttttatccaacggtctttaaccagcataccttctcttggctatatttttccttactcgcctaagtattacgacatactttctaccgtataagccttctgatggctcgatctcgatagtggccgagatactcatcattattatacctcgtaggtacaactatagcttggtggatcctttactattccttttaacgataaccgacatgtggtcagattttgacctatacaggctttggcattcttaagttggcaaacggcgtgtctggctctacgtcaatttagcaccaactaaagaagctaaaggaaaaattaagtcccaactattttagcagggtgttctcgttcccaacgaagtacactgtagctccaacctacgccctaacggctattggtctgtactgtttcttgttttaaatataagtaaaatatacttatttcctaatggactaatggagtctttcccctttgtttaacgaaccagtcctgatcttgatcgatctttcacttgatctcgctgataaacaaaaacgatatagaggttaacaaaggttcctcttcgcccctctcctttctttgtatagtattgaaataaagagaagtaaaatggggaggagtagatcgtccggcaataggtggaggtacatcaatccatcttactatttgaaacggcctatgcgtctcgcattgcttttcattgtttttgtatttggtactttcttcttttgggatcgacaaactttagtccgagatcaccaggtacttttgtttttccctacttcattgtcaattcccttttattggaactaatcactcttaactcccgtaatttgggtaattggttctgccatcgatcgttttctttttaattatgagcaagtttgttgctttgttacaacaataacactgtctatgttttcctggagaatctatgtgttccaattgtagaattgagagccccattggacgtagcaggcttgtattttgtatctgtattagtagaaaaaagggcagtccggcgcactaagctcccgctatgcgcggggtcggggaagggccggaccagtagggtctatcgtacgcagcctgcatttatgcaagaggctgtttccatggctcgaacccgtgacctcctggtcacatgacaacaactttaccggttagtagagtcctcaataaatttgatagactatactttggaaaattcaaggtaatcagctttttactagatttatctcttgtgtttttgtcgtaggtcattcatacaatgaatccaagtagaacttacaaaatgtattagcaagtctcttctcctatcaaagagttaactatcaacagcaacactgagtatggggattgaagagttctcagcgatatttgatttttgattacatagactgagagatatataggcattcatttcagagatcttctagttgctgcaggacaatatctttgaggttcttattgataattgaaacttaagttggtttacggtggtaatatcacccttgataaagataaattgtttagcaaggagcaacaaaaacaactacaccttagtcccaaactaattcagaacttctgaagaagtgtggctgctgggattcgtgcccaggtctttacggccagaacttggaattctactatactagacccacgttgaaaatagagatgcaacaagaagacttcctaggggtcacaaaacctagtacggcccttgtccaatcctaataccctagtgctttctatttatggttgcaagcaccctgggacatttggttttctagctagtagtaccaaagttctagtgatttttgatgcttattgcctttcagtttatatagaattttcttcttctgtttcatggaatcttcttctgatgtagaagtttatttatatgtattcttcataagcaagctagtggttcttagatgcatttgttatttggatatttttgaagttttaaattcagtttgtcttgcaatttgtcaatgaacttgtgattttgcaggaagagatctctaagttgcatgaagaagtgatacggttgcaaaatctggtaagcagtttctgcttttcttttcaaaatctgaactgttatgtttaattttcacctcttctgtaaattttggcttgtggggaaaatctttatactagagcttcttataattttgctggtaagtagccctttcctccttccaactgaatgaaaaagattgtttcactgtgtataattgaaaacctgatgaagttataattcttgcaattcggttcaagcatcatttatgttgtagtaaaaatactttatgcctatgggggagaggtatttgaggacagcaatggtgaagatagtggtggtgcgggcaataggtttggcaacaatggcggtggaaggaatagatgggcccatctgtgctctggcaggtttatgctgcaactgatcttattgttagggcttgctaggtcttttttgtaaaagaacatataacgaacatcacttgcttgggcaaagtccatctagttttctattgtttattgtagtcgctttcaaaattcttggtgttttaaatatttcgttctgttttcttcatcatgatttaattgctggcttttgtttccatttatggtcttgtttactgtagctggaagagttgaaggatggtcgaggtatatcaggtgaaaagatgaattttagtcgcagtggtggtgatgtggtgaagaaaaaggatttcgctgatgaccccattgatgctcaacgaagagaaaaagtgaaagatgctatgcttcatgcctggagttcatatgaaaaatatgcatggggccatgatgaacttcaggtctggttgttgctactaataagtcttctttgtagaaatattgcctttgtgccattatgtttagtcacttagcagtcaaatctttggtggaggcatttcagttggccgttaaatgctttaccctgttgattaatttcttatattttctttctctacttggagtgattgtgatcactttgtatgccttacccttaagctgatcatttaaatgcgagtcttcatattttcatcatccctaatatttgttgggaaaatgttggatcaagagcttcatcccagtcgtagaataatttacattctgaaatgtaatttcatccttggtggagtctgttttaggtttatttggctacaagttgaagaataagttatacctacataggtatcgatcttatgtagttagttctttcctttgtacaaaataatatcttgtactcaagattactgattaaaaaaaaaatcttgtactcaagggtttctcagataaaaaggagttacctcaaaatttaaatatgtgaaagggtgaagtctcaattaattaatgctcccactttttatatttgtttcaaatactctcacttgacactattggtgaaattatggccattccaaagtgactaacactctagctagaaacctttgctttttcttttaccttttaatttaattttgtccttttgctattgatctaatggaaaaatcatagctttttactttgtagcatctcatttacccttatgtccactctttaagtaaacataaagaagttacatattattatttctcatcccaagaatcctttcatgtcgaagtacggtttagaacactaggagttgtcgagatgtgggaagattattcatacaattggattctcaaaaagtttatcaagaattttgagtatcctggtaatgaagataacgctatcatcttttaagctctttctatgttaaagctttgagagaggagcattagtgcaatcaaaagtgaaaacttcagtcttctgcatttgcaataacttctatggggaaattttttaattgagcatggtaacaggtattttattaacaattaaagtagtccttggcacaaacaaagttacaggacctcaaaagaaaaagaaacaaaaagatagtcttgtgctagttacaaaaatcgcaagatgtcaactacagaatctacattttctacaagattaaacaatcagttacggagaaagtaaactgtaataagtattttgttgcacatgatatttcttgttcttcttaaaaagtctgtctgcgaggtaaaaacttgtggaagtttgtttatgtttatggtatttgggctctgcttccgagtataatagcttcatggtgaacaaaaatcttattcttgatggaattgctagcttcatatatgatctattcgactctctacttccctattcctttttctttctttgaccgaacatgtgatgtaagatcatattcacccagaagcttatacgtgttagcaaaatattcctagacagaatctatatggaattggtattagttctcaatgacttttttttgtggtgactataatttaatgacagtcagaaaggaaatgtaaaattgtaagagagatccctttttgttcgttgttcagtactgaatctaagaggataaattttccttgatacttttcgaactgtttctgctatgtgcttgtggaactttatactatatcctttattggtcatgtgcctgtattgatttgattgtcatgataaacctttgcaatgccagccacaaacaaagaagggtgttgacagttttggtggtcttggggcaacattaatagattctcttgacacactatatatcatgggcctggatgagcagtttcagagagctagagagtgagttcattattctcttgcccctgaaagccccgaattatctttcttattctaattcaggaattagttgtattataacttaaaattttgtgattgctcttgattgtaccttttccctttctttctagattgagagcttttttatgtgaaaaccagctttgtatatgtggatacattatcttctactttattttatttgacggtgatctcttccctgcacacagtaaccatggttgtctttgacaatattacttatggtcctagttttgttgtaaagaagaaaatgaattgtttacttttttttttttaatatgaccgggaatcaccagaatcaagtaattggtgcatgcgataatgttaaaatgcatctggggttagtaaaacattttatacttattgtcatctctctgattaatgtctgcagttctcctaactgccgcctcctcaacagccagagtccccaaagtcctcacccagtgagagactgcttagagttctgtgtttccttggattgtggatttgatgtctggcattttgactttccaaaataattgaagtgtcaatttcattatatcccttttacttctgggttttagggttatgtattaggtgtactttctactctctctgaaacaatgttgccaggtgataggcatttgtaactttatatatttttgtgcttcagttaagcgttcattgcttgtggctaacaagttgttgatggcaggtgggttgcaagctccttggatttcaacaagaattatgatgccagtgtttttgagacaaccataaggtttctttataaggtttaatatgcttttgtaatgagtttacttggattcctgataccttttatcagctttgacgatttgtttctatgttttttgtttcaatgtttctttatgtaattcaacaacagagttgtaggtggacttcttagtgcatatgatctctctggtgataagcttttccttgataaggctaaagatattgctgacagactgttgcctgcatggaatacaccatctggcatcccttacaacattatcaacttgtcacatgggaatccacataatcttgggtggacaggggtaattttgaactataccaaattcaagttgatttccgctgtagtataactcatgtatctcatgctgaaaaggatatagggaattatcctaaattttatttgacgagtcatttgatgctttaccctgcatcaataggagaagagtatctaaaaggggaactgtgtgaatgaagaatcatacgttattaaatgctctaattttctcataatatacttaaatgatcttatgatccaatccttgttttctctctttcttgcatctcctccaggcgttctcccaactgacttcagcttgctgggagaaacatgtctgttgcaacttagcaattgcagttctctaggaaactgtcccacatactctcaacttgtttgtgcacccagccatcttgtgatgatgtccttttgctgaaattttcaccagtgggaatccaactctcttctttttaattgctttttatttcttttctttggggcatattaggaagctgcagggcttgtgcagtcactgcgatatatggttttttacttgttcttttcctcttaaacgcttggacagagtctttttttgcacaccaatgacttatcttttgaaatctgaatatttcagtctcatggcatgtgatatatgatgcttaaatttctatgcacaaacacatatatgtaattacatcgctgtagtctagtgtacatttggtgaaattattgtgctcccttctctcagggtaatagtatcctggcagattctgcctctgagcagcttgaatttattgctctttcgcaacggacaggagactcaaagtatcaacagaaggtatgtgccaatagaatttatctaaaagtataacttcttgataactactagtaaataaaactacaattccaaaattggcatggtagacaattgattaagctacacatacttgaaacgatgttctgctagtgactgaatggcatatgttcctatttcaggtggagaatgttatcttagaacttaatagaacttttccagatgatggtttgcttccaatacacattaatcccgagagagggacaacgtcatactccactataacgtttggggccatgggggacaggtagctttcatttatctttctccatatgacagatctgataatgtgaacctaaagaggactggtatcaccatatccgtctgttcactggcatttggttttcctttgtttcttttgtacatttagatagtaaaactatgtcgtttcagcttttatgaatatttactcaaggcctggatacaaggaaacaaaacagctgctgtgggacactacaggtaagaagcttaagtttaaagtttctttatttttttactttacagttttcctattcaaaacttcaagtggtttcctgttttgacatgatgagttgcagttctgatggatccgtaactgtaaagtgtgtaaactaatgctagaatactttgtcgggcctgaattcaagtctttgtcatgcatcacggcctaacacatagaaatactgttaaatgtttacatgtgtagagcactaccaagaaacccaatcagaggaaacacgtgaattttgaccgaacatgaaaggaaaaaggaccattaaggagaaaaaaatgacaacttgctgaggagttgatttaatctaaatacataaaagtaggcctggattattagagctgttgctattatagtatcgttcgatatacatataaatatcgaagtaagagagattaaatttactgctacttttttaaaaaaaagaaatttcctgctatctttatatcattctgataaataatacataatatcaaacctgagctgcatcgggagccttaatgatgacattgttatatactccatcactttttcctagaagggcaaaacttaaaatcttgattaacatgtaactagagtactctttctgtgtcgcgttcttgcactcttgttacatcttccaagcatcactttagcatgtttccaaaaattcagatacgccaatcctaagtttcaaatactttgttttctaactttcttgctagttaaactagattagtcaaaacgatcaaaatttagtgcaggatgtccttatggattatcttgattagcagctgtaagctcagttctgcagaaactaatttgaagaccaaagaactgggggtttatgggcagcgtctttcctttgagaagtgcaaagcgagctccttatcctttactgctctgaagtgcaggaagacgaaattggttattgtctgaaaactctgtgttataattgcttagttagaaccaaaaggatcagaaatgtggaccaagtcaaagtatgtcaatgcatatttctttcctgagacttctaaatgagtatgacgttcttttgcaaattgcaatctcaagtgtattacatagagttcttccatttaattttccaaacagaaaaatgtgggagacatcaatgaaaggtcttttaagcttggtgcggaggactaccccatcatcttttgcttatattggtgagaagatcggaagttctttaaatgacaaggtgatgtatagggttcaaattggtagctgggagttgtgatgatgtgtgttattcttatatcatgtttaatctacccttttctgaattctatatagatggatgaacttgcatgcttcgctccaggaatgttagctttagggtcgtctggttatggtcctgacgagtctcagaagttcttatcactggcagaagaggtaaatttgaacttgtacagcattaaactatgttttgacttaagttcttatttgaccatcgatctctgatggagaagttttgcatcaactttgagtatgaggttgtttaggttacattggacattgttcggcctactccagatgattacttggtttactttaatttatttggtggggttatacagggtgaagcatgaaacaacctatgaaataacatgtaggtcttgaatgtgggctacagtgcagattttatcattcaaccttctaactttctctttcagataaaagggaaagaaggcacataggatcagtgggcttaatctattgcatattgactacttccattattgctcgttagaacaggaaacttgagtattgctattttactggatatgttgaccccttcttgcagcttgcttggacttgctataacttctaccagtcaacacctacaaaattggcaggagaaaactatttctttaatgatgacgggcaggttgattttaccaattattttattggtacatatttgttattgttgtttgcttatgctgataaagtatttgtgattgtttttcaggatatgactgtgggcacatcgtggaacatactaaggccagaaacggttgagtctctattttacctctggcgtttaactggaaacaagacataccaagagtggggttggaacatatttcaagcatttgaaaagaactcgagaatagagtctggatatgttggacttaaagatgtaagtacaaactcagactcctaactctagttggtgattttgttaaagattaattcatgtgaaagaatctgagcatccaacccaaaacttaaaaggcaatgggtggagtgatccaggacattacccttaggggctgtttggttcaaaatatcccataatcttgggattagaacagggactataacctggataacttatcccaccttctatatgggataagggataagttattccaagattttggtataacaagaatatcaggtttagctaataactccaaccaaaacgggataagtttaatcccaaaatttataccgggataacccacctaatcccttgaaccaaacgaccccttacataaccgatgaaagacaagtgtattctcggagtataacccgattctcgagatgttttggacatctatttttaacttgttggtgtttgtcccaggttaataccggtgtgcaagacgatatgatgcaaagctttttccttgcggagactcttaaatatctctaccttcttttctcaccctcttcactcattccactagatgagtgggtcttcaacacagaggcccaccccataaaaattgttagccggaatgatcgagcagtgagttctggaaggtcagttggacaaaccaaatcatataggcggccacggaccaggagagaaggccgatttggtaataagtagattcacaggtcatcattagtttagttgttgattgagaaggccaatttgagagttggaattcaagtgcagttttgcttggcacttcttcaaccagattgacgggattttcccccccaacattgataaaatgctcagtataggagaagttatgagtatgtagcatagttatttagtttcctttttctatgttcccttaatactagcgactgtattctagtacaggtcataagggcatttggttgcgggtagctctacatatttggggctggacgagtttttgtatatcatacctttttattttcgtttttcaaatacaacaggtaaattctaatttcaaggactgttgacaacttttttgcacagttgcgctatggttgatgatcaaatatatctcttgagtaacttttggttaaaaatagcacggtctacccagtttttagattggttattcaaaaatagccagcgtttgccaagtcattgaaaaataactactattttgctgctacagaaaccggtccaacataatatactggagtgtggtgcacctgtgtatgaacttccagcatattatgctggaccggtatattatactggaactccagtatattatgctggagtatttttctggattttgaatagtgttttcgttcagatttatctttacataaaaagtggctaaattttgattacttttgaaactgtgactattttttaatgaccacttgtaaatctgactatttttgaatttctccctaacttttgaggttagtgctgtgagcctgtctgggtaatattgggttggtttaatgtatctcagaatcgatgatagcaaaaatgatatcagttagctgctctaaagggctgttatttaggagttagcaaatgtgtcctgaattttagttgtccagtttaatttttcgggacataaatattctgaattgtcctcaaattaagatttttagtttaagacaaaataagtattgactaatatttaaataaaaaccttaagaatggatgtttgtgtaattctctcctggagcttgttaagtcgcattcacatactattttacgttactcc

SEQ ID NO: 64 - DNA, 9385 bp, Nicotiana tabacum
  source 1..9385: /mol_type=“DNA” /organism=“Nicotiana tabacum”
atggggaggagtagatcgtccggcaataggtggaggtacatcaatccatcttactatttgaaacggcctatgcgtctcgcattgcttttcattgtttttgtatttggtactttcttcttttgggatcgacaaactttagtccgagatcaccaggtacttttgtttttccctacttcattgtcaattcccttttattggaactaatcactcttaactcccgtaatttgggtaattggttctgccatcgatcgttttctttttaattatgagcaagtttgttgctttgttacaacaataacactgtctatgttttcctggagaatctatgtgttccaattgtagaattgagagccccattggacgtagcaggcttgtattttgtatctgtattagtagaaaaaagggcagtccggcgcactaagctcccgctatgcgcggggtcggggaagggccggaccagtagggtctatcgtacgcagcctgcatttatgcaagaggctgtttccatggctcgaacccgtgacctcctggtcacatgacaacaactttaccggttagtagagtcctcaataaatttgatagactatactttggaaaattcaaggtaatcagctttttactagatttatctcttgtgtttttgtcgtaggtcattcatacaatgaatccaagtagaacttacaaaatgtattagcaagtctcttctcctatcaaagagttaactatcaacagcaacactgagtatggggattgaagagttctcagcgatatttgatttttgattacatagactgagagatatataggcattcatttcagagatcttctagttgctgcaggacaatatctttgaggttcttattgataattgaaacttaagttggtttacggtggtaatatcacccttgataaagataaattgtttagcaaggagcaacaaaaacaactacaccttagtcccaaactaattcagaacttctgaagaagtgtggctgctgggattcgtgcccaggtctttacggccagaacttggaattctactatactagacccacgttgaaaatagagatgcaacaagaagacttcctaggggtcacaaaacctagtacggcccttgtccaatcctaataccctagtgctttctatttatggttgcaagcaccctgggacatttggttttctagctagtagtaccaaagttctagtgatttttgatgcttattgcctttcagtttatatagaattttcttcttctgtttcatggaatcttcttctgatgtagaagtttatttatatgtattcttcataagcaagctagtggttcttagatgcatttgttatttggatatttttgaagttttaaattcagtttgtcttgcaatttgtcaatgaacttgtgattttgcaggaagagatctctaagttgcatgaagaagtgatacggttgcaaaatctggtaagcagtttctgcttttcttttcaaaatctgaactgttatgtttaattttcacctcttctgtaaattttggcttgtggggaaaatctttatactagagcttcttataattttgctggtaagtagccctttcctccttccaactgaatgaaaaagattgtttcactgtgtataattgaaaacctgatgaagttataattcttgcaattcggttcaagcatcatttatgttgtagtaaaaatactttatgcctatgggggagaggtatttgaggacagcaatggtgaagatagtggtggtgcgggcaataggtttggcaacaatggcggtggaaggaatagatgggcccatctgtgctctggcaggtttatgctgcaactgatcttattgttagggcttgctaggtcttttttgtaaaagaacatataacgaacatcacttgcttgggcaaagtccatctagttttctattgtttattgtagtcgctttcaaaattcttggtgttttaaatatttcgttctgttttcttcatcatgatttaattgctggcttttgtttccatttatggtcttgtttactgtagctggaagagttgaaggatggtcgaggtatatcaggtgaaaagatgaattttagtcgcagtggtggtgatgtggtgaagaaaaaggatttcgctgatgaccccattgatgctcaacgaagagaaaaagtgaaagatgctatgcttcatgcctggagttcatatgaaaaatatgcatggggccatgatgaacttcaggtctggttgttgctactaataagtcttctttgtagaaatattgcctttgtgccattatgtttagtcacttagcagtcaaatctttggtggaggcatttcagttggccgttaaatgctttaccctgttgattaatttcttatattttctttctctacttggagtgattgtgatcactttgtatgccttacccttaagctgatcatttaaatgcgagtcttcatattttcatcatccctaatatttgttgggaaaatgttggatcaagagcttcatcccagtcgtagaataatttacattctgaaatgtaatttcatccttggtggagtctgttttaggtttatttggctacaagttgaagaataagttatacctacataggtatcgatcttatgtagttagttctttcctttgtacaaaataatatcttgtactcaagattactgattaaaaaaaaaatcttgtactcaagggtttctcagataaaaaggagttacctcaaaatttaaatatgtgaaagggtgaagtctcaattaattaatgctcccactttttatatttgtttcaaatactctcacttgacactattggtgaaattatggccattccaaagtgactaacactctagctagaaacctttgctttttcttttaccttttaatttaattttgtccttttgctattgatctaatggaaaaatcatagctttttactttgtagcatctcatttacccttatgtccactctttaagtaaacataaagaagttacatattattatttctcatcccaagaatcctttcatgtcgaagtacggtttagaacactaggagttgtcgagatgtgggaagattattcatacaattggattctcaaaaagtttatcaagaattttgagtatcctggtaatgaagataacgctatcatcttttaagctctttctatgttaaagctttgagagaggagcattagtgcaatcaaaagtgaaaacttcagtcttctgcatttgcaataacttctatggggaaattttttaattgagcatggtaacaggtattttattaacaattaaagtagtccttggcacaaacaaagttacaggacctcaaaagaaaaagaaacaaaaagatagtcttgtgctagttacaaaaatcgcaagatgtcaactacagaatctacattttctacaagattaaacaatcagttacggagaaagtaaactgtaataagtattttgttgcacatgatatttcttgttcttcttaaaaagtctgtctgcgaggtaaaaacttgtggaagtttgtttatgtttatggtatttgggctctgcttccgagtataatagcttcatggtgaacaaaaatcttattcttgatggaattgctagcttcatatatgatctattcgactctctacttccctattcctttttctttctttgaccgaacatgtgatgtaagatcatattcacccagaagcttatacgtgttagcaaaatattcctagacagaatctatatggaattggtattagttctcaatgacttttttttgtggtgactataatttaatgacagtcagaaaggaaatgtaaaattgtaagagagatccctttttgttcgttgttcagtactgaatctaagaggataaattttccttgatacttttcgaactgtttctgctatgtgcttgtggaactttatactatatcctttattggtcatgtgcctgtattgatttgattgtcatgataaacctttgcaatgccagccacaaacaaagaagggtgttgacagttttggtggtcttggggcaacattaatagattctcttgacacactatatatcatgggcctggatgagcagtttcagagagctagagagtgagttcattattctcttgcccctgaaagccccgaattatctttcttattctaattcaggaattagttgtattataacttaaaattttgtgattgctcttgattgtaccttttccctttctttctagattgagagcttttttatgtgaaaaccagctttgtatatgtggatacattatcttctactttattttatttgacggtgatctcttccctgcacacagtaaccatggttgtctttgacaatattacttatggtcctagttttgttgtaaagaagaaaatgaattgtttacttttttttttttaatatgaccgggaatcaccagaatcaagtaattggtgcatgcgataatgttaaaatgcatctggggttagtaaaacattttatacttattgtcatctctctgattaatgtctgcagttctcctaactgccgcctcctcaacagccagagtccccaaagtcctcacccagtgagagactgcttagagttctgtgtttccttggattgtggatttgatgtctggcattttgactttccaaaataattgaagtgtcaatttcattatatcccttttacttctgggttttagggttatgtattaggtgtactttctactctctctgaaacaatgttgccaggtgataggcatttgtaactttatatatttttgtgcttcagttaagcgttcattgcttgtggctaacaagttgttgatggcaggtgggttgcaagctccttggatttcaacaagaattatgatgccagtgtttttgagacaaccataaggtttctttataaggtttaatatgcttttgtaatgagtttacttggattcctgataccttttatcagctttgacgatttgtttctatgttttttgtttcaatgtttctttatgtaattcaacaacagagttgtaggtggacttcttagtgcatatgatctctctggtgataagcttttccttgataaggctaaagatattgctgacagactgttgcctgcatggaatacaccatctggcatcccttacaacattatcaacttgtcacatgggaatccacataatcttgggtggacaggggtaattttgaactataccaaattcaagttgatttccgctgtagtataactcatgtatctcatgctgaaaaggatatagggaattatcctaaattttatttgacgagtcatttgatgctttaccctgcatcaataggagaagagtatctaaaaggggaactgtgtgaatgaagaatcatacgttattaaatgctctaattttctcataatatacttaaatgatcttatgatccaatccttgttttctctctttcttgcatctcctccaggcgttctcccaactgacttcagcttgctgggagaaacatgtctgttgcaacttagcaattgcagttctctaggaaactgtcccacatactctcaacttgtttgtgcacccagccatcttgtgatgatgtccttttgctgaaattttcaccagtgggaatccaactctcttctttttaattgctttttatttcttttctttggggcatattaggaagctgcagggcttgtgcagtcactgcgatatatggttttttacttgttcttttcctcttaaacgcttggacagagtctttttttgcacaccaatgacttatcttttgaaatctgaatatttcagtctcatggcatgtgatatatgatgcttaaatttctatgcacaaacacatatatgtaattacatcgctgtagtctagtgtacatttggtgaaattattgtgctcccttctctcagggtaatagtatcctggcagattctgcctctgagcagcttgaatttattgctctttcgcaacggacaggagactcaaagtatcaacagaaggtatgtgccaatagaatttatctaaaagtataacttcttgataactactagtaaataaaactacaattccaaaattggcatggtagacaattgattaagctacacatacttgaaacgatgttctgctagtgactgaatggcatatgttcctatttcaggtggagaatgttatcttagaacttaatagaacttttccagatgatggtttgcttccaatacacattaatcccgagagagggacaacgtcatactccactataacgtttggggccatgggggacaggtagctttcatttatctttctccatatgacagatctgataatgtgaacctaaagaggactggtatcaccatatccgtctgttcactggcatttggttttcctttgtttcttttgtacatttagatagtaaaactatgtcgtttcagcttttatgaatatttactcaaggcctggatacaaggaaacaaaacagctgctgtgggacactacaggtaagaagcttaagtttaaagtttctttatttttttactttacagttttcctattcaaaacttcaagtggtttcctgttttgacatgatgagttgcagttctgatggatccgtaactgtaaagtgtgtaaactaatgctagaatactttgtcgggcctgaattcaagtctttgtcatgcatcacggcctaacacatagaaatactgttaaatgtttacatgtgtagagcactaccaagaaacccaatcagaggaaacacgtgaattttgaccgaacatgaaaggaaaaaggaccattaaggagaaaaaaatgacaacttgctgaggagttgatttaatctaaatacataaaagtaggcctggattattagagctgttgctattatagtatcgttcgatatacatataaatatcgaagtaagagagattaaatttactgctacttttttaaaaaaaagaaatttcctgctatctttatatcattctgataaataatacataatatcaaacctgagctgcatcgggagccttaatgatgacattgttatatactccatcactttttcctagaagggcaaaacttaaaatcttgattaacatgtaactagagtactctttctgtgtcgcgttcttgcactcttgttacatcttccaagcatcactttagcatgtttccaaaaattcagatacgccaatcctaagtttcaaatactttgttttctaactttcttgctagttaaactagattagtcaaaacgatcaaaatttagtgcaggatgtccttatggattatcttgattagcagctgtaagctcagttctgcagaaactaatttgaagaccaaagaactgggggtttatgggcagcgtctttcctttgagaagtgcaaagcgagctccttatcctttactgctctgaagtgcaggaagacgaaattggttattgtctgaaaactctgtgttataattgcttagttagaaccaaaaggatcagaaatgtggaccaagtcaaagtatgtcaatgcatatttctttcctgagacttctaaatgagtatgacgttcttttgcaaattgcaatctcaagtgtattacatagagttcttccatttaattttccaaacagaaaaatgtgggagacatcaatgaaaggtcttttaagcttggtgcggaggactaccccatcatcttttgcttatattggtgagaagatcggaagttctttaaatgacaaggtgatgtatagggttcaaattggtagctgggagttgtgatgatgtgtgttattcttatatcatgtttaatctacccttttctgaattctatatagatggatgaacttgcatgcttcgctccaggaatgttagctttagggtcgtctggttatggtcctgacgagtctcagaagttcttatcactggcagaagaggtaaatttgaacttgtacagcattaaactatgttttgacttaagttcttatttgaccatcgatctctgatggagaagttttgcatcaactttgagtatgaggttgtttaggttacattggacattgttcggcctactccagatgattacttggtttactttaatttatttggtggggttatacagggtgaagcatgaaacaacctatgaaataacatgtaggtcttgaatgtgggctacagtgcagattttatcattcaaccttctaactttctctttcagataaaagggaaagaaggcacataggatcagtgggcttaatctattgcatattgactacttccattattgctcgttagaacaggaaacttgagtattgctattttactggatatgttgaccccttcttgcagcttgcttggacttgctataacttctaccagtcaacacctacaaaattggcaggagaaaactatttctttaatgatgacgggcaggttgattttaccaattattttattggtacatatttgttattgttgtttgcttatgctgataaagtatttgtgattgtttttcaggatatgactgtgggcacatcgtggaacatactaaggccagaaacggttgagtctctattttacctctggcgtttaactggaaacaagacataccaagagtggggttggaacatatttcaagcatttgaaaagaactcgagaatagagtctggatatgttggacttaaagatgtaagtacaaactcagactcctaactctagttggtgattttgttaaagattaattcatgtgaaagaatctgagcatccaacccaaaacttaaaaggcaatgggtggagtgatccaggacattacccttaggggctgtttggttcaaaatatcccataatcttgggattagaacagggactataacctggataacttatcccaccttctatatgggataagggataagttattccaagattttggtataacaagaatatcaggtttagctaataactccaaccaaaacgggataagtttaatcccaaaatttataccgggataacccacctaatcccttgaaccaaacgaccccttacataaccgatgaaagacaagtgtattctcggagtataacccgattctcgagatgttttggacatctatttttaacttgttggtgtttgtcccaggttaataccggtgtgcaagacgatatgatgcaaagctttttccttgcggagactcttaaatatctctaccttcttttctcaccctcttcactcattccactagatgagtgggtcttcaacacagaggcccaccccataaaaattgttagccggaatgatcgagcagtgagttctggaaggtcagttggacaaaccaaatcatataggcggccacggaccaggagagaaggccgatttggtaataagtag

SEQ ID NO: 65 - DNA, 153 bp, Nicotiana tabacum
  source 1..153: /mol_type=“DNA” /organism=“Nicotiana tabacum”
atggggaggagtagatcgtccggcaataggtggaggtacatcaatccatcttactatttgaaacggcctatgcgtctcgcattgcttttcattgtttttgtatttggtactttcttcttttgggatcgacaaactttagtccgagatcaccag

SEQ ID NO: 66 - DNA, 1255 bp, Nicotiana tabacum
  source 1..1255: /mol_type=“DNA” /organism=“Nicotiana tabacum”
gtacttttgtttttccctacttcattgtcaattcccttttattggaactaatcactcttaactcccgtaatttgggtaattggttctgccatcgatcgttttctttttaattatgagcaagtttgttgctttgttacaacaataacactgtctatgttttcctggagaatctatgtgttccaattgtagaattgagagccccattggacgtagcaggcttgtattttgtatctgtattagtagaaaaaagggcagtccggcgcactaagctcccgctatgcgcggggtcggggaagggccggaccagtagggtctatcgtacgcagcctgcatttatgcaagaggctgtttccatggctcgaacccgtgacctcctggtcacatgacaacaactttaccggttagtagagtcctcaataaatttgatagactatactttggaaaattcaaggtaatcagctttttactagatttatctcttgtgtttttgtcgtaggtcattcatacaatgaatccaagtagaacttacaaaatgtattagcaagtctcttctcctatcaaagagttaactatcaacagcaacactgagtatggggattgaagagttctcagcgatatttgatttttgattacatagactgagagatatataggcattcatttcagagatcttctagttgctgcaggacaatatctttgaggttcttattgataattgaaacttaagttggtttacggtggtaatatcacccttgataaagataaattgtttagcaaggagcaacaaaaacaactacaccttagtcccaaactaattcagaacttctgaagaagtgtggctgctgggattcgtgcccaggtctttacggccagaacttggaattctactatactagacccacgttgaaaatagagatgcaacaagaagacttcctaggggtcacaaaacctagtacggcccttgtccaatcctaataccctagtgctttctatttatggttgcaagcaccctgggacatttggttttctagctagtagtaccaaagttctagtgatttttgatgcttattgcctttcagtttatatagaattttcttcttctgtttcatggaatcttcttctgatgtagaagtttatttatatgtattcttcataagcaagctagtggttcttagatgcatttgttatttggatatttttgaagttttaaattcagtttgtcttgcaatttgtcaatgaacttgtgattttgcag

SEQ ID NO: 67 - DNA, 48 bp, Nicotiana tabacum
  source 1..48: /mol_type=“DNA” /organism=“Nicotiana tabacum”
gaagagatctctaagttgcatgaagaagtgatacggttgcaaaatctg

SEQ ID NO: 68 - DNA, 583 bp, Nicotiana tabacum
  source 1..583: /mol_type=“DNA” /organism=“Nicotiana tabacum”
gtaagcagtttctgcttttcttttcaaaatctgaactgttatgtttaattttcacctcttctgtaaattttggcttgtggggaaaatctttatactagagcttcttataattttgctggtaagtagccctttcctccttccaactgaatgaaaaagattgtttcactgtgtataattgaaaacctgatgaagttataattcttgcaattcggttcaagcatcatttatgttgtagtaaaaatactttatgcctatgggggagaggtatttgaggacagcaatggtgaagatagtggtggtgcgggcaataggtttggcaacaatggcggtggaaggaatagatgggcccatctgtgctctggcaggtttatgctgcaactgatcttattgttagggcttgctaggtcttttttgtaaaagaacatataacgaacatcacttgcttgggcaaagtccatctagttttctattgtttattgtagtcgctttcaaaattcttggtgttttaaatatttcgttctgttttcttcatcatgatttaattgctggcttttgtttccatttatggtcttgtttactgtag

SEQ ID NO: 69 - DNA, 195 bp, Nicotiana tabacum
  source 1..195: /mol_type=“DNA” /organism=“Nicotiana tabacum”
ctggaagagttgaaggatggtcgaggtatatcaggtgaaaagatgaattttagtcgcagtggtggtgatgtggtgaagaaaaaggatttcgctgatgaccccattgatgctcaacgaagagaaaaagtgaaagatgctatgcttcatgcctggagttcatatgaaaaatatgcatggggccatgatgaacttcag

SEQ ID NO: 70 - DNA, 1766 bp, Nicotiana tabacum
  source 1..1766: /mol_type=“DNA” /organism=“Nicotiana tabacum”
gtctggttgttgctactaataagtcttctttgtagaaatattgcctttgtgccattatgtttagtcacttagcagtcaaatctttggtggaggcatttcagttggccgttaaatgctttaccctgttgattaatttcttatattttctttctctacttggagtgattgtgatcactttgtatgccttacccttaagctgatcatttaaatgcgagtcttcatattttcatcatccctaatatttgttgggaaaatgttggatcaagagcttcatcccagtcgtagaataatttacattctgaaatgtaatttcatccttggtggagtctgttttaggtttatttggctacaagttgaagaataagttatacctacataggtatcgatcttatgtagttagttctttcctttgtacaaaataatatcttgtactcaagattactgattaaaaaaaaaatcttgtactcaagggtttctcagataaaaaggagttacctcaaaatttaaatatgtgaaagggtgaagtctcaattaattaatgctcccactttttatatttgtttcaaatactctcacttgacactattggtgaaattatggccattccaaagtgactaacactctagctagaaacctttgctttttcttttaccttttaatttaattttgtccttttgctattgatctaatggaaaaatcatagctttttactttgtagcatctcatttacccttatgtccactctttaagtaaacataaagaagttacatattattatttctcatcccaagaatcctttcatgtcgaagtacggtttagaacactaggagttgtcgagatgtgggaagattattcatacaattggattctcaaaaagtttatcaagaattttgagtatcctggtaatgaagataacgctatcatcttttaagctctttctatgttaaagctttgagagaggagcattagtgcaatcaaaagtgaaaacttcagtcttctgcatttgcaataacttctatggggaaattttttaattgagcatggtaacaggtattttattaacaattaaagtagtccttggcacaaacaaagttacaggacctcaaaagaaaaagaaacaaaaagatagtcttgtgctagttacaaaaatcgcaagatgtcaactacagaatctacattttctacaagattaaacaatcagttacggagaaagtaaactgtaataagtattttgttgcacatgatatttcttgttcttcttaaaaagtctgtctgcgaggtaaaaacttgtggaagtttgtttatgtttatggtatttgggctctgcttccgagtataatagcttcatggtgaacaaaaatcttattcttgatggaattgctagcttcatatatgatctattcgactctctacttccctattcctttttctttctttgaccgaacatgtgatgtaagatcatattcacccagaagcttatacgtgttagcaaaatattcctagacagaatctatatggaattggtattagttctcaatgacttttttttgtggtgactataatttaatgacagtcagaaaggaaatgtaaaattgtaagagagatccctttttgttcgttgttcagtactgaatctaagaggataaattttccttgatacttttcgaactgtttctgctatgtgcttgtggaactttatactatatcctttattggtcatgtgcctgtattgatttgattgtcatgataaacctttgcaatgccag

SEQ ID NO: 71 - DNA, 113 bp, Nicotiana tabacum
  source 1..113: /mol_type=“DNA” /organism=“Nicotiana tabacum”
ccacaaacaaagaagggtgttgacagttttggtggtcttggggcaacattaatagattctcttgacacactatatatcatgggcctggatgagcagtttcagagagctagaga

SEQ ID NO: 72 - DNA, 727 bp, Nicotiana tabacum
  source 1..727: /mol_type=“DNA” /organism=“Nicotiana tabacum”
gtgagttcattattctcttgcccctgaaagccccgaattatctttcttattctaattcaggaattagttgtattataacttaaaattttgtgattgctcttgattgtaccttttccctttctttctagattgagagcttttttatgtgaaaaccagctttgtatatgtggatacattatcttctactttattttatttgacggtgatctcttccctgcacacagtaaccatggttgtctttgacaatattacttatggtcctagttttgttgtaaagaagaaaatgaattgtttacttttttttttttaatatgaccgggaatcaccagaatcaagtaattggtgcatgcgataatgttaaaatgcatctggggttagtaaaacattttatacttattgtcatctctctgattaatgtctgcagttctcctaactgccgcctcctcaacagccagagtccccaaagtcctcacccagtgagagactgcttagagttctgtgtttccttggattgtggatttgatgtctggcattttgactttccaaaataattgaagtgtcaatttcattatatcccttttacttctgggttttagggttatgtattaggtgtactttctactctctctgaaacaatgttgccaggtgataggcatttgtaactttatatatttttgtgcttcagttaagcgttcattgcttgtggctaacaagttgttgatggcag

SEQ ID NO: 73 - DNA, 66 bp, Nicotiana tabacum
  source 1..66: /mol_type=“DNA” /organism=“Nicotiana tabacum”
gtgggttgcaagctccttggatttcaacaagaattatgatgccagtgtttttgagacaaccataag

SEQ ID NO: 74 - DNA, 126 bp, Nicotiana tabacum
  source 1..126: /mol_type=“DNA” /organism=“Nicotiana tabacum”
gtttctttataaggtttaatatgcttttgtaatgagtttacttggattcctgataccttttatcagctttgacgatttgtttctatgttttttgtttcaatgtttctttatgtaattcaacaacag

SEQ ID NO: 75 - DNA, 172 bp, Nicotiana tabacum
  source 1..172: /mol_type=“DNA” /organism=“Nicotiana tabacum”
agttgtaggtggacttcttagtgcatatgatctctctggtgataagcttttccttgataaggctaaagatattgctgacagactgttgcctgcatggaatacaccatctggcatcccttacaacattatcaacttgtcacatgggaatccacataatcttgggtggacaggg

SEQ ID NO: 76 - DNA, 720 bp, Nicotiana tabacum
  source 1..720: /mol_type=“DNA” /organism=“Nicotiana tabacum”
gtaattttgaactataccaaattcaagttgatttccgctgtagtataactcatgtatctcatgctgaaaaggatatagggaattatcctaaattttatttgacgagtcatttgatgctttaccctgcatcaataggagaagagtatctaaaaggggaactgtgtgaatgaagaatcatacgttattaaatgctctaattttctcataatatacttaaatgatcttatgatccaatccttgttttctctctttcttgcatctcctccaggcgttctcccaactgacttcagcttgctgggagaaacatgtctgttgcaacttagcaattgcagttctctaggaaactgtcccacatactctcaacttgtttgtgcacccagccatcttgtgatgatgtccttttgctgaaattttcaccagtgggaatccaactctcttctttttaattgctttttatttcttttctttggggcatattaggaagctgcagggcttgtgcagtcactgcgatatatggttttttacttgttcttttcctcttaaacgcttggacagagtctttttttgcacaccaatgacttatcttttgaaatctgaatatttcagtctcatggcatgtgatatatgatgcttaaatttctatgcacaaacacatatatgtaattacatcgctgtagtctagtgtacatttggtgaaattattgtgctcccttctctcag

SEQ ID NO: 77 - DNA, 90 bp, Nicotiana tabacum
  source 1..90: /mol_type=“DNA” /organism=“Nicotiana tabacum”
ggtaatagtatcctggcagattctgcctctgagcagcttgaatttattgctctttcgcaacggacaggagactcaaagtatcaacagaag

SEQ ID NO: 78 - DNA, 158 bp, Nicotiana tabacum
  source 1..158: /mol_type=“DNA” /organism=“Nicotiana tabacum”
gtatgtgccaatagaatttatctaaaagtataacttcttgataactactagtaaataaaactacaattccaaaattggcatggtagacaattgattaagctacacatacttgaaacgatgttctgctagtgactgaatggcatatgttcctatttcag

SEQ ID NO: 79 - DNA, 125 bp, Nicotiana tabacum
  source 1..125: /mol_type=“DNA” /organism=“Nicotiana tabacum”
gtggagaatgttatcttagaacttaatagaacttttccagatgatggtttgcttccaatacacattaatcccgagagagggacaacgtcatactccactataacgtttggggccatgggggacag

SEQ ID NO: 80 - DNA, 146 bp, Nicotiana tabacum
  source 1..146: /mol_type=“DNA” /organism=“Nicotiana tabacum”
gtagctttcatttatctttctccatatgacagatctgataatgtgaacctaaagaggactggtatcaccatatccgtctgttcactggcatttggttttcctttgtttcttttgtacatttagatagtaaaactatgtcgtttcag

SEQ ID NO: 81 - DNA, 66 bp, Nicotiana tabacum
  source 1..66: /mol_type=“DNA” /organism=“Nicotiana tabacum”
cttttatgaatatttactcaaggcctggatacaaggaaacaaaacagctgctgtgggacactacag

SEQ ID NO: 82 - DNA, 1123 bp, Nicotiana tabacum
  source 1..1123: /mol_type=“DNA” /organism=“Nicotiana tabacum”
gtaagaagcttaagtttaaagtttctttatttttttactttacagttttcctattcaaaacttcaagtggtttcctgttttgacatgatgagttgcagttctgatggatccgtaactgtaaagtgtgtaaactaatgctagaatactttgtcgggcctgaattcaagtctttgtcatgcatcacggcctaacacatagaaatactgttaaatgtttacatgtgtagagcactaccaagaaacccaatcagaggaaacacgtgaattttgaccgaacatgaaaggaaaaaggaccattaaggagaaaaaaatgacaacttgctgaggagttgatttaatctaaatacataaaagtaggcctggattattagagctgttgctattatagtatcgttcgatatacatataaatatcgaagtaagagagattaaatttactgctacttttttaaaaaaaagaaatttcctgctatctttatatcattctgataaataatacataatatcaaacctgagctgcatcgggagccttaatgatgacattgttatatactccatcactttttcctagaagggcaaaacttaaaatcttgattaacatgtaactagagtactctttctgtgtcgcgttcttgcactcttgttacatcttccaagcatcactttagcatgtttccaaaaattcagatacgccaatcctaagtttcaaatactttgttttctaactttcttgctagttaaactagattagtcaaaacgatcaaaatttagtgcaggatgtccttatggattatcttgattagcagctgtaagctcagttctgcagaaactaatttgaagaccaaagaactgggggtttatgggcagcgtctttcctttgagaagtgcaaagcgagctccttatcctttactgctctgaagtgcaggaagacgaaattggttattgtctgaaaactctgtgttataattgcttagttagaaccaaaaggatcagaaatgtggaccaagtcaaagtatgtcaatgcatatttctttcctgagacttctaaatgagtatgacgttcttttgcaaattgcaatctcaagtgtattacatagagttcttccatttaattttccaaacag

SEQ ID NO: 83 - DNA, 109 bp, Nicotiana tabacum
  source 1..109: /mol_type=“DNA” /organism=“Nicotiana tabacum”
aaaaatgtgggagacatcaatgaaaggtcttttaagcttggtgcggaggactaccccatcatcttttgcttatattggtgagaagatcggaagttctttaaatgacaag

SEQ ID NO: 84 - DNA, 95 bp, Nicotiana tabacum
  source 1..95: /mol_type=“DNA” /organism=“Nicotiana tabacum”
gtgatgtatagggttcaaattggtagctgggagttgtgatgatgtgtgttattcttatatcatgtttaatctacccttttctgaattctatatag

SEQ ID NO: 85 - DNA, 99 bp, Nicotiana tabacum
  source 1..99: /mol_type=“DNA” /organism=“Nicotiana tabacum”
atggatgaacttgcatgcttcgctccaggaatgttagctttagggtcgtctggttatggtcctgacgagtctcagaagttcttatcactggcagaagag

SEQ ID NO: 86 - DNA, 412 bp, Nicotiana tabacum
  source 1..412: /mol_type=“DNA” /organism=“Nicotiana tabacum”
gtaaatttgaacttgtacagcattaaactatgttttgacttaagttcttatttgaccatcgatctctgatggagaagttttgcatcaactttgagtatgaggttgtttaggttacattggacattgttcggcctactccagatgattacttggtttactttaatttatttggtggggttatacagggtgaagcatgaaacaacctatgaaataacatgtaggtcttgaatgtgggctacagtgcagattttatcattcaaccttctaactttctctttcagataaaagggaaagaaggcacataggatcagtgggcttaatctattgcatattgactacttccattattgctcgttagaacaggaaacttgagtattgctattttactggatatgttgaccccttcttgcag

SEQ ID NO: 87 - DNA, 84 bp, Nicotiana tabacum
  source 1..84: /mol_type=“DNA” /organism=“Nicotiana tabacum”
cttgcttggacttgctataacttctaccagtcaacacctacaaaattggcaggagaaaactatttctttaatgatgacgggcag

SEQ ID NO: 88 - DNA, 84 bp, Nicotiana tabacum
  source 1..84: /mol_type=“DNA” /organism=“Nicotiana tabacum”
gttgattttaccaattattttattggtacatatttgttattgttgtttgcttatgctgataaagtatttgtgattgtttttcag

SEQ ID NO: 89 - DNA, 171 bp, Nicotiana tabacum
  source 1..171: /mol_type=“DNA” /organism=“Nicotiana tabacum”
gatatgactgtgggcacatcgtggaacatactaaggccagaaacggttgagtctctattttacctctggcgtttaactggaaacaagacataccaagagtggggttggaacatatttcaagcatttgaaaagaactcgagaatagagtctggatatgttggacttaaagat

SEQ ID NO: 90 - DNA, 450 bp, Nicotiana tabacum
  source 1..450: /mol_type=“DNA” /organism=“Nicotiana tabacum”
gtaagtacaaactcagactcctaactctagttggtgattttgttaaagattaattcatgtgaaagaatctgagcatccaacccaaaacttaaaaggcaatgggtggagtgatccaggacattacccttaggggctgtttggttcaaaatatcccataatcttgggattagaacagggactataacctggataacttatcccaccttctatatgggataagggataagttattccaagattttggtataacaagaatatcaggtttagctaataactccaaccaaaacgggataagtttaatcccaaaatttataccgggataacccacctaatcccttgaaccaaacgaccccttacataaccgatgaaagacaagtgtattctcggagtataacccgattctcgagatgttttggacatctatttttaacttgttggtgtttgtcccag

SEQ ID NO: 91 - DNA, 249 bp, Nicotiana tabacum
  source 1..249: /mol_type=“DNA” /organism=“Nicotiana tabacum”
gttaataccggtgtgcaagacgatatgatgcaaagctttttccttgcggagactcttaaatatctctaccttcttttctcaccctcttcactcattccactagatgagtgggtcttcaacacagaggcccaccccataaaaattgttagccggaatgatcgagcagtgagttctggaaggtcagttggacaaaccaaatcatataggcggccacggaccaggagagaaggccgatttggtaataagtag

SEQ ID NO: 92 - DNA, 1740 bp, Nicotiana tabacum
  source 1..1740: /mol_type=“DNA” /organism=“Nicotiana tabacum”
atggggaggagtagatcgtccggcaataggtggaggtacatcaatccatcttactatttgaaacggcctatgcgtctcgcattgcttttcattgtttttgtatttggtactttcttcttttgggatcgacaaactttagtccgagatcaccaggaagagatctctaagttgcatgaagaagtgatacggttgcaaaatctgctggaagagttgaaggatggtcgaggtatatcaggtgaaaagatgaattttagtcgcagtggtggtgatgtggtgaagaaaaaggatttcgctgatgaccccattgatgctcaacgaagagaaaaagtgaaagatgctatgcttcatgcctggagttcatatgaaaaatatgcatggggccatgatgaacttcagccacaaacaaagaagggtgttgacagttttggtggtcttggggcaacattaatagattctcttgacacactatatatcatgggcctggatgagcagtttcagagagctagagagtgggttgcaagctccttggatttcaacaagaattatgatgccagtgtttttgagacaaccataagagttgtaggtggacttcttagtgcatatgatctctctggtgataagcttttccttgataaggctaaagatattgctgacagactgttgcctgcatggaatacaccatctggcatcccttacaacattatcaacttgtcacatgggaatccacataatcttgggtggacagggggtaatagtatcctggcagattctgcctctgagcagcttgaatttattgctctttcgcaacggacaggagactcaaagtatcaacagaaggtggagaatgttatcttagaacttaatagaacttttccagatgatggtttgcttccaatacacattaatcccgagagagggacaacgtcatactccactataacgtttggggccatgggggacagcttttatgaatatttactcaaggcctggatacaaggaaacaaaacagctgctgtgggacactacagaaaaatgtgggagacatcaatgaaaggtcttttaagcttggtgcggaggactaccccatcatcttttgcttatattggtgagaagatcggaagttctttaaatgacaagatggatgaacttgcatgcttcgctccaggaatgttagctttagggtcgtctggttatggtcctgacgagtctcagaagttcttatcactggcagaagagcttgcttggacttgctataacttctaccagtcaacacctacaaaattggcaggagaaaactatttctttaatgatgacgggcaggatatgactgtgggcacatcgtggaacatactaaggccagaaacggttgagtctctattttacctctggcgtttaactggaaacaagacataccaagagtggggttggaacatatttcaagcatttgaaaagaactcgagaatagagtctggatatgttggacttaaagatgttaataccggtgtgcaagacgatatgatgcaaagctttttccttgcggagactcttaaatatctctaccttcttttctcaccctcttcactcattccactagatgagtgggtcttcaacacagaggcccaccccataaaaattgttagccggaatgatcgagcagtgagttctggaaggtcagttggacaaaccaaatcatataggcggccacggaccaggagagaaggccgatttggtaataagtag

SEQ ID NO: 93 - protein, 579 aa, Nicotiana tabacum
  SOURCE 1..579: /mol_type=“protein” /organism=“Nicotiana tabacum”
MGRSRSSGNRWRYINPSYYLKRPMRLALLFIVFVFGTFFFWDRQTLVRDHQEEISKLHEEVIRLQNLLEELKDGRGISGEKMNFSRSGGDVVKKKDFADDPIDAQRREKVKDAMLHAWSSYEKYAWGHDELQPQTKKGVDSFGGLGATLIDSLDTLYIMGLDEQFQRAREWVASSLDFNKNYDASVFETTIRVVGGLLSAYDLSGDKLFLDKAKDIADRLLPAWNTPSGIPYNIINLSHGNPHNLGWTGGNSILADSASEQLEFIALSQRTGDSKYQQKVENVILELNRTFPDDGLLPIHINPERGTTSYSTITFGAMGDSFYEYLLKAWIQGNKTAAVGHYRKMWETSMKGLLSLVRRTTPSSFAYIGEKIGSSLNDKMDELACFAPGMLALGSSGYGPDESQKFLSLAEELAWTCYNFYQSTPTKLAGENYFFNDDGQDMTVGTSWNILRPETVESLFYLWRLTGNKTYQEWGWNIFQAFEKNSRIESGYVGLKDVNTGVQDDMMQSFFLAETLKYLYLLFSPSSLIPLDEWVFNTEAHPIKIVSRNDRAVSSGRSVGQTKSYRRPRTRREGRFGNK

SEQ ID NO: 94 - DNA, 1752 bp, Nicotiana tabacum
  source 1..1752: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgtcttcgccaccttcttcttttgggatcgacaaactttagtccgtgatcatcaggaagagatctctaagttgaatcatgaagtgacgcaattgcgaaatctgctggaagatttgaagaatggtcgagtcatgccagataaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacagccgcagtcaaagaatggtgttgacagttttggtggtcttggagcaaccttaatagattctcttgacacactatatatcatgggcctggatgagcagtttcagagagctagagaatgggttgcaaactccttggatttcaacaagaactatgatgcaagtgtttttgagacaaccataagggttgtaggtgggcttcttagtacgtacgatctatctggtgataagcttttccttgataaggctcaagacattgctgacagattgttgcccgcatggaatacagaatctggaatcccttacaacattatcaacttggcaaatgggaatccacataaccctgggtggacagggggtgatagtatcctggcagattctggtactgagcagcttgagtttattgctctttcgcagaggacaggagacccaaaatatcaacaaaaggtggagaatgttatcttagaacttaacaaaacttttccagatgatggtttgcttccaatatacattaatccacataaaggcacaacatcatactcaactataacatttggggcaatgggcgacagcttttatgaatatttactcaaggtctggatacaaggaaacagaactgctgctgtgagtcattataggaaaatgtgggagacatcaatgaaaggtcttttaagcttggtccggagaacaactccttcgtcttttgcatatatttgcgagaagatgggaagttctttaaatgacaagatggatgaacttgcatgctttgctcctgggatgttagctttaggatcatctggttatagccctaatgaggctcagaagttcttatcactggctgaggagcttgcttggacttgctataatttttatcagtcaacacctacaaaactggcaggagagaactatttttttaatgccggccaagatatgagtgtgggcacatcatggaatatattaaggccagagacagttgagtcgctgttttacctctggcgtttaacaggaaacaagacataccaagagtggggttggaacatatttcaagcatttgaaaagaactcaaggatagaatctggatatgttggacttaaagatgtcaacactggtgtcaaagacaatatgatgcaaagcttctttcttgcggagacttttaaatatctctatcttcttttttcaccctcatcagtaatctctctagatgagtgggtttttaacacagaagcccaccccataaaaattgttacccggaatgatcgtgctatgaattctggagggtcaggtggacggcaagaatcagataggcaatcacgaaccaggaaagaagatatatctgatacagagtttaagaaaggactttaa

SEQ ID NO: 95 - protein, 583 aa, Nicotiana tabacum
  SOURCE 1..583: /mol_type=“protein” /organism=“Nicotiana tabacum”
MARSRSSSTTFRYINPAYYLKRPKRLALLFIVFVFATFFFWDRQTLVRDHQEEISKLNHEVTQLRNLLEDLKNGRVMPDKKMKSSGKGGHAAKNMDSPDNILDAQRREKVKDAMLHAWSSYEKYAWGHDELQPQSKNGVDSFGGLGATLIDSLDTLYIMGLDEQFQRAREWVANSLDFNKNYDASVFETTIRVVGGLLSTYDLSGDKLFLDKAQDIADRLLPAWNTESGIPYNIINLANGNPHNPGWTGGDSILADSGTEQLEFIALSQRTGDPKYQQKVENVILELNKTFPDDGLLPIYINPHKGTTSYSTITFGAMGDSFYEYLLKVWIQGNRTAAVSHYRKMWETSMKGLLSLVRRTTPSSFAYICEKMGSSLNDKMDELACFAPGMLALGSSGYSPNEAQKFLSLAEELAWTCYNFYQSTPTKLAGENYFFNAGQDMSVGTSWNILRPETVESLFYLWRLTGNKTYQEWGWNIFQAFEKNSRIESGYVGLKDVNTGVKDNMMQSFFLAETFKYLYLLFSPSSVISLDEWVFNTEAHPIKIVTRNDRAMNSGGSGGRQESDRQSRTRKEDISDTEFKKGL

SEQ ID NO: 96 - DNA, 1713 bp, Nicotiana tabacum
  source 1..1713: /mol_type=“DNA” /organism=“Nicotiana tabacum”
atggcgaggagtagatcgtcttccactactttcaggtacattaatccggcttactatctgaaacggccaaagcgtctggctttgctcttcatcgtttttgttttcgccaccttcttcttttgggatcgacaaactttagtccgtgatcatcaggaagagatctctaagttgaatgatgaagtgatgaaattgcgaaatctgctggaagatttgaagaatggtcgagtcatgccaggtgaaaagatgaaatctagtggcaaaggtggtcatgcagcaaaaaatatggattcaccagataatatccttgatgctcagcgaagggagaaagtgaaagatgctatgcttcatgcttggagttcttatgaaaaatatgcatggggtcatgatgaattacagtcaaagaatggtgttgacagttttggtggtcttggagcaaccttaatagattctcttgacacactatatatcatgggcctggatgagcagtttcagagagctagagaggttgtaggtgggcttcttagtacgtatgatctatctggtgataagcttttccttgataaggctcaagacattgctgacagattgttgcccgcatggaatacagaatctggaatcccttacaacactatcaacttggctcatgggaatccacataaccctgggtggacagggggtgatagtatcctggcagattctggtactgagcagcttgagtttattgctctttcgcagaggacaggagacccaaaatatcaacaaaaggtggagaatgttatcttggaacttaacaaaacttttccagaggatggtttgcttccaatatacattaatccacataaaggcacaacatcatactcaactataacatttggggcaatgggcgacagcttttatgaatatttactcaaggtctggatacaaggaaacagaactgctgctgtgagtcattataggaaaatgtgggagacatcaatgaaaggtcttttaagcttggttcggagaacgactccttcgtcttttgcatatatttgcgagaagatgggaagttctttaaatgacaagatggatgaacttgcatgctttgctcctgggatgttagctttaggatcatctggttatagccctaatgaggctcagaagttcttatcactggctgaggagcttgcttggacttgctataacttttaccagtcaacacctacaaaactggcaggagagaactatttttttaatgccggccaggacatgagtgtgggcacatcatggaatatattaaggccagagacagttgagtcgctgttttacctctggcgtttaacaggaaacaagacataccaagagtggggttggaacatatttcaagcatttgaaaagaattcaaggatagaatctggatatgttggacttaaagatgtcaacactggtgtcaaagacaatatgatgcaaagcttctttcttgcggagactcttaaatatctctatcttcttttttcaccctcatcagtaatatccctagatgagtgggtttttaacacagaagcccaccccataaaaattgttacccggaatgatcatgctatgagttctggaggttcaggtggacggcaagaatcagataggcaatcacgaaccaggaaagaaggagattgcaatttttgccggcagctccacatttttgggcttgatgagcaaattgctagtcgcacctaa

SEQ ID NO: 97 - protein, 570 aa, Nicotiana tabacum
  SOURCE 1..570: /mol_type=“protein” /organism=“Nicotiana tabacum”
MARSRSSSTTFRYINPAYYLKRPKRLALLFIVFVFATFFFWDRQTLVRDHQEEISKLNDEVMKLRNLLEDLKNGRVMPGEKMKSSGKGGHAAKNMDSPDNILDAQRREKVKDAMLHAWSSYEKYAWGHDELQSKNGVDSFGGLGATLIDSLDTLYIMGLDEQFQRAREVVGGLLSTYDLSGDKLFLDKAQDIADRLLPAWNTESGIPYNTINLAHGNPHNPGWTGGDSILADSGTEQLEFIALSQRTGDPKYQQKVENVILELNKTFPEDGLLPIYINPHKGTTSYSTITFGAMGDSFYEYLLKVWIQGNRTAAVSHYRKMWETSMKGLLSLVRRTTPSSFAYICEKMGSSLNDKMDELACFAPGMLALGSSGYSPNEAQKFLSLAEELAWTCYNFYQSTPTKLAGENYFFNAGQDMSVGTSWNILRPETVESLFYLWRLTGNKTYQEWGWNIFQAFEKNSRIESGYVGLKDVNTGVKDNMMQSFFLAETLKYLYLLFSPSSVISLDEWVFNTEAHPIKIVTRNDHAMSSGGSGGRQESDRQSRTRKEGDCNFCRQLHIFGLDEQIASRT

SEQ ID NO: 98 - DNA, 1740 bp, Nicotiana tabacum
  source 1..1740: /mol_type=“DNA” /organism=“Nicotiana tabacum”
atggggaggagtagatcgtccaccaataggtggaggtacatcaatccatcttactatttgaaacgccccaagcgtctcgcattgcttttcattgttttcgtattcggtacattcttcttttgggatcgacaaacgttagtccgagaccaccaggaagagatctctaagttgcatgaagaagtgatacggttgcaaaatctgctggaagagttgaagaatggtcgaggtgtatcgggtgaaaaggtgaattttagtcgcactggtggtgatgtgctgaagaaaaaggatttcgctgaagaccccattgatgctcagcgaagagaaaaagtgaaagatgctatgcttcacgcctggagttcatatgaaaaatatgcctggggccacgatgaacttcagccacaaacaaagaagggtgttgacagttttggtggtcttggggccacattaatagattctcttgacacactatatatcatgggcctggatgagcagtttcagagagctagagagtgggttgcaagctcattggatttcaacaagaattatgatgccagtgtttttgagacaaccataagagttgttggtggacttcttagtgcgtatgatctctctggtgataagcttttccttgataaggctaaagatattgctgacagactgttgcctgcatggaatacaccatctggcatcccttacaacattatcaacttgtcacatggaaatccgcataatcctgggtggacagggggtaatagtatcctggcagattctgcctctgagcagcttgaatttattgctctttcgcaaaggacaggagactcaaagtatcaacagaaggtggagaatgttatcgtagaacttaatagaacttttccagttgatggtttgcttccaatacacattaatcccgagagagggacaacgtcatactccactataacatttggggccatgggggacagcttttatgaatatttactcaaggtctggatacaaggaaacaaaacagctgctgtgggacactacagaaaaatgtgggagacatcaatgaaaggccttttaagcttggtgcggaggactaccccatcatcttttgcttatattggtgagaagatcggaagttctttaaatgacaagatggatgaacttgcatgcttcgctccaggaatgttagctttagggtcgtctggttatggtcctgacgagtctcagaagttcttatcactcgcagaagagcttgcttggacttgctataacttctaccagtcaacaccttcaaaattggcaggagaaaactatttctttaatgatgatgggcaggatatgaccgtgggcacatcgtggaacatactaaggccagaaacggttgagtctctgttttacctctggcgtttaactggaaacaagacataccaagagtggggttggaacatatttcaagcatttgaaaagaactcgagaatagagtctggatatgttggacttaaagatgttaataccggtgtgcaagacaatatgatgcaaagctttttccttgcggagactcttaaatatctctaccttcttttctcaccctcttcaatcattccactagatgagtgggtcttcaacacagaggcccaccccataaaaattgttagccggaatgatccagcagtcagttctggaaggtcagttggacaaacaaaatcatataggcggccacggaccaggagagaaggccgatttggtaataagtag

SEQ ID NO: 99 - protein, 579 aa, Nicotiana tabacum
  SOURCE 1..579: /mol_type=“protein” /organism=“Nicotiana tabacum”
MGRSRSSTNRWRYINPSYYLKRPKRLALLFIVFVFGTFFFWDRQTLVRDHQEEISKLHEEVIRLQNLLEELKNGRGVSGEKVNFSRTGGDVLKKKDFAEDPIDAQRREKVKDAMLHAWSSYEKYAWGHDELQPQTKKGVDSFGGLGATLIDSLDTLYIMGLDEQFQRAREWVASSLDFNKNYDASVFETTIRVVGGLLSAYDLSGDKLFLDKAKDIADRLLPAWNTPSGIPYNIINLSHGNPHNPGWTGGNSILADSASEQLEFIALSQRTGDSKYQQKVENVIVELNRTFPVDGLLPIHINPERGTTSYSTITFGAMGDSFYEYLLKVWIQGNKTAAVGHYRKMWETSMKGLLSLVRRTTPSSFAYIGEKIGSSLNDKMDELACFAPGMLALGSSGYGPDESQKFLSLAEELAWTCYNFYQSTPSKLAGENYFFNDDGQDMTVGTSWNILRPETVESLFYLWRLTGNKTYQEWGWNIFQAFEKNSRIESGYVGLKDVNTGVQDNMMQSFFLAETLKYLYLLFSPSSIIPLDEWVFNTEAHPIKIVSRNDPAVSSGRSVGQTKSYRRPRTRREGRFGNK

SEQ ID NO: 100 - DNA, 21 bp, artificial sequences
  source 1..21: /mol_type=“DNA” /note=“forward primer” /organism=“artificial sequences”
atggggaggagtagatcgtcc

SEQ ID NO: 101 - DNA, 24 bp, artificial sequences
  source 1..24: /mol_type=“DNA” /note=“reverse primer” /organism=“artificial sequences”
ctacttattaccaaatcggccttc

SEQ ID NO: 102 - DNA, 22 bp, artificial sequences
  source 1..22: /mol_type=“DNA” /note=“forward primer” /organism=“artificial sequences”
atggcgaggagtagatcgtctt

SEQ ID NO: 103 - DNA, 22 bp, artificial sequences
  source 1..22: /mol_type=“DNA” /note=“reverse primer” /organism=“artificial sequences”
ttaggtgcgactagcaatttgc